(19) United States
(12) Patent Application Publication
(10) Pub. No.: US 2023/0002367 A1
(43) Pub. Date: Jan. 5, 2023

(54) BIFUNCTIONAL COMPOUNDS

(71) Applicants: Hoffmann-La Roche Inc., Little Falls, NJ (US); C4 THERAPEUTICS, INC., Watertown, MA (US)

(72) Inventors: Delphine GAUFRETEAU, Basel (CH); Roman HUTTER, Basel (CH); Eleonora JOVCHEVA, Basel (CH); Bernd KUHN, Basel (CH); Thomas LUEBBERS, Basel (CH); Rainer E. MARTIN, Basel (CH); Laetitia Janine MARTIN, Basel (CH); Barbara Johanna MUELLER, Basel (CH); Roger NORCROSS, Basel (CH); Fabienne RICKLIN, Basel (CH); Philipp SCHMID, Basel (CH); Jean-Yves WACH, Basel (CH); Juergen WICHMANN, Basel (CH); Martin DUPLESSIS, Watertown, MA (US); Kiel LAZARSKI, Watertown, MA (US); Yanke LIANG, Watertown, MA (US)

(21) Appl. No.: 17/771,127

(22) PCT Filed: Oct. 26, 2020
(86) PCT No.: PCT/US2020/057356

§ 371 Date: Apr. 22, 2022

(30) Foreign Application Priority Data
Oct. 28, 2019 (EP) 19205545.7

(51) Int. Cl. C07D 417/14; C07D 471/08; C07D 471/10; C07D 498/10; C07D 487/08

(52) U.S. Cl. CPC C07D 417/14; C07D 471/08; C07D 471/10; C07D 498/10; C07D 487/08

(57) ABSTRACT

The invention provides a bifunctional compound of formula (I), or a pharmaceutically acceptable salt thereof, wherein said Targeting Ligand, Linker and Degron are as described herein.

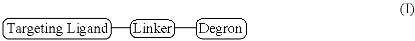

### FIELD OF THE INVENTION

The present invention relates novel bifunctional compounds, which function to recruit targeted proteins to E3 Ubiquitin Ligase for degradation, and methods of preparation and uses thereof. More specifically, the compounds of the present invention cause the degradation of SMARCA2 via the targeted ubiquitination of SMARCA2 protein and subsequent proteasomal degradation. The present compounds are thus useful for the treatment or prophylaxis of abnormal cellular proliferation, including tumors and cancer.

### BACKGROUND OF THE INVENTION

Most small molecule drugs bind enzymes or receptors in tight and well-defined pockets. On the other hand, protein-protein interactions are notoriously difficult to target using small molecules due to their large contact surfaces and the shallow grooves or flat interfaces involved. E3 ubiquitin ligases (of which hundreds are known in humans) confer substrate specificity for ubiquitination, and therefore, are more attractive therapeutic targets than general proteasome inhibitors due to their specificity for certain protein substrates. The development of ligands of E3 ligases has proven challenging, in part due to the fact that they must disrupt protein-protein interactions. However, recent developments have provided specific ligands which bind to these ligases. For example, since the discovery of nutlins, the first small molecule E3 ligase mouse double minute 2 homolog (MDM2) inhibitors, additional compounds have been reported that target MDM2 (i.e, human double minute 2 or HDM2) E3 ligases (J. Di, et al. Current Cancer Drug Targets (2011), 11(8), 987-994).

One E3 ligase with exciting therapeutic potential is the von Hippel-Lindau (VHL) tumor suppressor, the substrate recognition subunit of the E3 ligase complex VCB, which also consists of elongins B and C, Cul2 and Rbxl. The primary substrate of VHL is Hypoxia Inducible Factor 1a (HIF-1α), a transcription factor that upregulates genes such as the pro-angiogenic growth factor VEGF and the red blood cell inducing cytokine erythropoietin in response to low oxygen levels. The first small molecule ligands of Von Hippel Lindau (VHL) to the substrate recognition subunit of the E3 ligase were generated, and crystal structures were obtained confirming that the compound mimics the binding mode of the transcription factor HIF-Iα, the major substrate of VHL.

The field of targeted protein degradation promoted by small molecules has been intensively studied over the last years (e.g. Collins et al., Biochem J, 2017, 474(7), 1127-47). Bifunctional compounds, such as those that are described in U.S. Patent Application Publications 2016-0235730, function to recruit endogenous proteins to an E3 ubiquitin ligase for degradation.

The Switch/Sucrose Non Fermentable (SWI/SNF) is a multi-subunit complex that modulates chromatic structure through the activity of two mutually exlusive helicase/ATPase catalytic subunits SWI/SNF-Related, Matrix-Associated, Actin-Dependent Regulator of Chromatin, Subfamily A, Member 2 (SMARCA2, BRAHMA or BRM) and SWI/SNF-Related, Matrix-Associated, Actin-Dependent Regulator of Chromatin, Subfamily A, Member 4 (SMARCA4 or BRG1). The core and the regulatory subunits couple ATP hydrolysis to the perturbation of histone-DNA contacts, thereby providing access points to transcription factors and cognate DNA elements that facilitate gene activation and repression.

Mutations in the genes encoding the twenty canonical SWI/SNF subunits are observed in nearly 20% of all cancers with the highest frequency of mutations observed in rhabdoid tumors, female cancers (including ovarian, uterine, cerical and endometrial), lung adenocarcinoma, gastric adenocarcinoma, melanoma, esophageal, and renal clear cell carcinoma. Despite having a high degree of homology, and their presumed overlapping functions, SMARCA2 and SMARCA4 have been reported as having different roles in cancer. For example, SMARCA4 is frequently mutated in primary tumors, while SMARCA2 inactivation is infrequent in tumor development. In fact, numerous types of cancer have been shown to be SMARCA4-related (e.g., cancers having a SMARCA4-mutation or a SMARCA4-deficiency, such as lack of expression), including, e.g., lung cancer (such as non-small cell lung cancer).

SMARCA2 has been demonstrated as one of the top essential genes in SMARCA4-related or -mutant cancer cell lines. This is because SMARCA4-deficient patient populations or cells depend exclusively on SMARCA2 activity—i.e., there is a greater incorporation of SMARCA2 into the complex to compensate for the SMARCA4 deficiency. Thus, SMARCA2 may be targeted in SMARCA4-related/deficient cancers. The co-occurrence of the deficiency of the expression of two (or more) genes that leads to cell death is known as synthetic lethality. Accordingly, synthetic lethality can be leveraged in the treatment of certain SMARCA2/SMARCA4-related cancers.

There is an ongoing need for effective treatment for diseases that are treatable by inhibiting or degrading SMARCA2 (i.e., BRAHMA or BRM). However, non-specific effects, and the inability to target and modulate SMARCA2 remains an obstacle to the development of effective treatments. As such, small-molecule therapeutic agents that target SMARCA2 and that leverage or potentiate VHL's substrate specificity would be very useful.

### SUMMARY OF THE INVENTION

The present invention provides a bifunctional compound of formula (I)

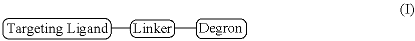

or a pharmaceutically acceptable salt thereof, wherein said Targeting Ligand, Linker and Degron are as described herein.

In a further aspect, the present invention provides compounds of formula (I) as defined herein, or pharmaceutically acceptable salts thereof, for use as therapeutically active substance.

In a further aspect, the present invention provides pharmaceutical compositions comprising a compound of formula (I) as defined herein, or a pharmaceutically acceptable salt thereof, and a therapeutically inert carrier.

In a further aspect, the present invention provides a compound of formula (I) as defined herein, or a pharmaceutically acceptable salt thereof, for use in the treatment of SMARCA2-mediated disorders, in particular cancer.

### DETAILED DESCRIPTION OF THE INVENTION

The present invention provides compounds of formula I and pharmaceutically acceptable salts thereof, the preparation of the above mentioned compounds, medicaments containing them and their manufacture as well as the use of the above mentioned compounds in the therapeutic and/or prophylactic treatment of cancer.

### Definitions

The following definitions of the general terms used in the present description apply irrespectively of whether the terms in question appear alone or in combination with other groups.

Unless otherwise stated, the following terms used in this Application, including the specification and claims, have the definitions given below. It must be noted that, as used in the specification and the appended claims, the singular forms “a”, “an,” and “the” include plural referents unless the context clearly dictates otherwise.

The term “Targeting Ligand” (or target protein moiety or target protein ligand or ligand) as used herein refers to a small molecule of formula (TL) as defined herein, which is capable of binding to or binds to a target protein of interest, such as to SMARCA2.

The term “Linker” as used herein refers to a chemical moiety selected from formulae L-1 to L-23 as define herein that serves to link a Targeting Ligand with a Degron.

The Degron is a compound that serves to link a targeted protein, through the Linker and Targeting Ligand, to a ubiquitin ligase for proteosomal degradation. In certain embodiments, the Degron is a compound that is capable of binding to or binds to a ubiquitin ligase. In further embodiments, the Degron is a compound that is capable of binding to or binds to a E3 Ubiquitin Ligase. In further embodiments, the Degron is a compound that is capable of binding to or binds to VHL (von Hippel-Lindau tumor suppressor).

The term “SMARCA2” refers to Switch/Sucrose Non Fermentable (SWI/SNF)-Related, Matrix-Associated, Actin-Dependent Regulator of Chromatin, Subfamily A, Member 2 (SMARCA2) (i.e, BRAHMA or BRM).

The term “alkyl”, alone or in combination with other groups, stands for a hydrocarbon radical which may be linear or branched, with single or multiple branching, wherein the alkyl group in general comprises 1 to 6 carbon atoms (C₁₋₆-alkyl), for example, methyl (Me), ethyl (Et), propyl, isopropyl (i-propyl), n-butyl, i-butyl (isobutyl), 2-butyl (sec-butyl), t-butyl (tert-butyl), isopentyl, 2-ethyl-propyl (2-methyl-propyl), 1,2-dimethyl-propyl and the like. A specific group is methyl.

The term “alkyldiyl” as used herein refers to a saturated linear or branched-chain divalent hydrocarbon radical of about one to six carbon atoms (C₁-C₆). Examples of alkyldiyl groups include, but are not limited to, methylene (—CH₂—), ethylene (—CH₂CH₂—), propylene (—CH₂CH₂CH₂—), and the like. An alkyldiyl group may also be referred to as an “alkylene” group.

The term “alkynyldiyl” as used herein refers to a saturated linear or branched-chain divalent hydrocarbon radical of about two to six carbon atoms (C₂-C₆) comprising at least one carbon-carbon triple bond. Examples of alkynyldiyl groups include, but are not limited to, ethynylene, propynylene, and the like. An alkyldiyl group may also be referred to as an “alkynylene” group.

The term “haloalkyl”, alone or in combination with other groups, refers to alkyl as defined herein, which is substituted by one or multiple halogen, particularly 1-5 halogen, more particularly 1-3 halogen. Particular halogen is fluoro. Examples include 2,2,2-trifluoroethyl, trifluoromethyl, difluoromethyl, fluoromethyl and the like.

The term “haloaloxy”, alone or in combination with other groups, refers to alkoxy as defined herein, which is substituted by one or multiple halogen, particularly 1-5 halogen, more particularly 1-3 halogen. Particular halogen is fluoro. Examples include 2,2,2-trifluoroethoxy, trifluoromethoxy, difluoromethoxy, fluoromethoxy and the like.

The term “cycloalkyl” denotes a monovalent saturated monocyclic or bicyclic hydrocarbon group of 3 to 10 ring carbon atoms, particularly a monovalent saturated monocyclic hydrocarbon group of 3 to 8 ring carbon atoms. Bicyclic means consisting of two carbocycles having one or more carbon atoms in common, while one carbocycle is saturated, the other one may be aromatic. Particular cycloalkyl groups are monocyclic. Examples for monocyclic cycloalkyl are “C₃₋₇cycloalkyl” such as cyclopropyl, cyclobutanyl, cyclopentyl, cyclohexyl or cycloheptyl. Examples for saturated bicyclic cycloalkyl are bicyclo[2.2.1]heptanyl, or bicyclo[2.2.2]octanyl. Examples for bicyclic cycloalkyl wherein one ring is aromatic are 1H-indenyl or 1,2,3,4-tetrahydronaphthalenyl.

The term “hydroxy”, alone or in combination with other groups, refers to OH.

The term “amino”, alone or in combination with other groups, refers to NH₂.

The term “cyano”, alone or in combination with other groups, refers to CN (i.e. nitrile).

The term “carbonyl”, alone or in combination with other groups, refers to C(═O).

The term “halogen”, alone or in combination with other groups, denotes chloro (Cl), iodo (I), fluoro (F) and bromo (Br). A specific group is F.

The term “heteroaryl” denotes a monovalent heterocyclic mono- or bicyclic ring system of 5 to 14 ring atoms, comprising 1, 2, 3 or 4 heteroatoms selected from N, O and S, the remaining ring atoms being carbon and in which at least one ring is aromatic. Examples of heteroaryl moieties include pyrrolyl, furanyl, thienyl, imidazolyl, oxazolyl, thiazolyl, triazolyl, oxadiazolyl, thiadiazolyl, tetrazolyl, pyridinyl, pyrazinyl, pyrazolyl, pyridazinyl, pyrimidinyl, triazinyl, azepinyl, diazepinyl, isoxazolyl, benzofuranyl, isothiazolyl, benzothienyl, indolinyl, indolyl, isoindolyl, isobenzofuranyl, benzimidazolyl, benzoxazolyl, benzoisoxazolyl, benzothiazolyl, benzoisothiazolyl, benzooxadiazolyl, benzothiadiazolyl, benzotriazolyl, purinyl, quinolinyl, isoquinolinyl, quinazolinyl, quinoxalinyl, or 2,3-dihydropyrrolo[2,3-b]pyridinyl. Specific examples include benzimidazolyl, pyridinyl, thiazolyl, indolinyl, 1,2,3,4-tetrahydroquinolinyl, 3,4-dihydroquinolinyl, benzofuranyl, furanyl, imidazolyl, isoindolyl, and quinolinyl.

The term “heterocyclyl” denotes a monovalent saturated or partly unsaturated mono- or bicyclic ring system of 3 to 14 ring atoms, comprising 1, 2, or 3 ring heteroatoms selected from N, O and S, the remaining ring atoms being carbon. Examples for monocyclic saturated heterocyclyl include azetidinyl, pyrrolidinyl, tetrahydrofuranyl, tetrahydrothienyl, pyrazolidinyl, imidazolidinyl, oxazolidinyl, isoxazolidinyl, thiazolidinyl, piperidinyl, tetrahydropyranyl, tetrahydrothiopyranyl, piperazinyl, morpholinyl, thiomorpholinyl, 1,1-dioxo-thiomorpholin-4-yl, azepanyl, diazepanyl, homopiperazinyl, or oxazepanyl. Examples for bicyclic saturated heterocyclyl include 8-aza-bicyclo[3.2.1]octyl, quinuclidinyl, 8-oxa-3-aza-bicyclo[3.2.1]octyl, 9-aza-bicyclo[3.3.1]nonyl, 3-oxa-9-aza-bicyclo[3.3.1]nonyl, or 3-thia-9-aza-bicyclo[3.3.1]nonyl. Examples for partly unsaturated heterocyclyl include dihydrofuryl, imidazolinyl, dihydro-oxazolyl, tetrahydro-pyridinyl, or dihydropyranyl. Specific examples include piperazinyl, piperidinyl, pyrrolidinyl and 3,8-diazabicyclo[3.2.1]octanyl.

It is to be understood that when multi point attachments are drawn in heterocycles or heteroaromatic compounds, the point of attachment may also be at a heteroatom, in particular at a nitrogen atom. Thus, for example the following structure

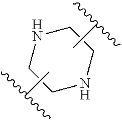

includes a piperazine ring that is bound to further molecular entities through its two nitrogen atoms:

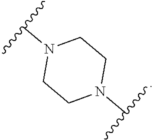 [SMILES: *N1CCN(*)CC1]

The term “alkoxy”, alone or in combination with other groups, stands for an —O—C₁₋₆-alkyl radical which may be linear or branched, with single or multiple branching, wherein the alkyl group in general comprises 1 to 6 carbon atoms (C₁₋₆-alkoxy), for example, methoxy (OMe, MeO), ethoxy (OEt), propoxy, isopropoxy (i-propoxy), n-butoxy, i-butoxy (iso-butoxy), 2-butoxy (sec-butoxy), t-butoxy (tert-butoxy), isopentyloxy (i-pentyloxy) and the like. Particular “C₁₋₆-alkoxy” are groups with 1 to 4 carbon atoms. A specific group is methoxy.

The term “aryl” denotes a monovalent aromatic carbocyclic mono- or bicyclic ring system comprising 6 to 10 carbon ring atoms. Examples of aryl moieties include phenyl (Ph), and naphthyl. Specific “aryl” is phenyl.

The term “pharmaceutically acceptable” denotes an attribute of a material which is useful in preparing a pharmaceutical composition that is generally safe, non-toxic, and neither biologically nor otherwise undesirable and is acceptable for veterinary as well as human pharmaceutical use.

The term “a pharmaceutically acceptable salt” refers to a salt that is suitable for use in contact with the tissues of humans and animals. Examples of suitable salts with inorganic and organic acids are, but are not limited to acetic acid, citric acid, formic acid, fumaric acid, hydrochloric acid, lactic acid, maleic acid, malic acid, methane-sulfonic acid, nitric acid, phosphoric acid, p-toluenesulphonic acid, succinic acid, sulfuric acid (sulphuric acid), tartaric acid, trifluoroacetic acid and the like. Particular acids are formic acid, trifluoroacetic acid and hydrochloric acid. Specific acids are hydrochloric acid, trifluoroacetic acid and fumaric acid.

The term “as defined herein” and “as described herein” when referring to a variable incorporates by reference the broad definition of the variable as well as particularly, more particularly and most particularly definitions, if any.

The terms “treating”, “contacting” and “reacting” when referring to a chemical reaction means adding or mixing two or more reagents under appropriate conditions to produce the indicated and/or the desired product. It should be appreciated that the reaction which produces the indicated and/or the desired product may not necessarily result directly from the combination of two reagents which were initially added, i.e., there may be one or more intermediates which are produced in the mixture which ultimately leads to the formation of the indicated and/or the desired product.

The term “aromatic” denotes the conventional idea of aromaticity as defined in the literature, in particular in IUPAC—Compendium of Chemical Terminology, 2^{nd} Edition, A. D. McNaught & A. Wilkinson (Eds). Blackwell Scientific Publications, Oxford (1997).

The term “therapeutically inert carrier” denotes any ingredient having no therapeutic activity and being non-toxic such as disintegrators, binders, fillers, solvents, buffers, tonicity agents, stabilizers, antioxidants, surfactants or lubricants used in formulating pharmaceutical products.

Whenever a chiral carbon is present in a chemical structure, it is intended that all stereoisomers associated with that chiral carbon are encompassed by the structure as pure stereoisomers as well as mixtures thereof.

All separate embodiments may be combined.

The term “treatment” as used herein includes: (1) inhibiting the state, disorder or condition (e.g. arresting, reducing or delaying the development of the disease, or a relapse thereof in case of maintenance treatment, of at least one clinical or subclinical symptom thereof); and/or (2) relieving the condition (i.e., causing regression of the state, disorder or condition or at least one of its clinical or subclinical symptoms). The benefit to a patient to be treated is either statistically significant or at least perceptible to the patient or to the physician. However, it will be appreciated that when a medicament is administered to a patient to treat a disease, the outcome may not always be effective treatment.

The term “cancer” refers to a disease characterized by the presence of a neoplasm or tumor resulting from abnormal uncontrolled growth of cells (such cells being “cancer cells”). As used herein, the term cancer explicitly includes, but is not limited to, hepatocellular cancer, malignancies and hyperproliferative disorders of the colon (colon cancer), lung cancer, breast cancer, prostate cancer, melanoma, and ovarian cancer.

### Compounds of the Invention

In a first aspect (A1), the present invention provides a compound of formula (I)

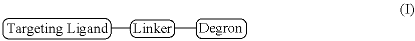

or a pharmaceutically acceptable salt thereof, wherein:
said targeting ligand is of formula (TL):

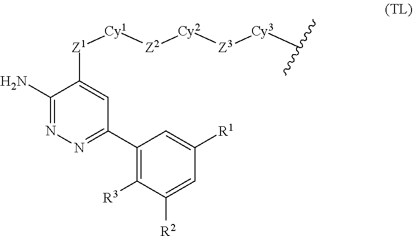

wherein:
R¹ and R² are each independently selected from hydrogen and halogen;
R³ is selected from hydroxy and amino;
Z¹ is:
   (i) absent;
   (ii) —O—;
   (iii) —O—C₁-C₆-alkyldiyl-;
   (iv) —O(CH₂)ₙCH(R⁴)(CH₂)ₚ—; or
   (v) C₂-C₆-alkynyldiyl;
Cy¹ is:
   (i) absent;
   (ii) C₆-C₁₀-aryl optionally substituted with 1-3 substitutents R⁵;
   (iii) C₃-C₁₀-cycloalkyl optionally substituted with 1-3 substitutents R⁶;
   (iv) 5-14 membered heteroaryl optionally substituted with 1-3 substitutents R⁷; or
   (v) 3-14 membered heterocyclyl optionally substituted with 1-3 substitutents R⁸;
Z² is:
   (i) absent;
   (ii) —C(O)NH—C₁-C₆-alkyldiyl-;
   (iii) —CH(R⁹)—;
   (iv) C₁-C₆-alkyldiyl;
   (v) —C₁-C₆-alkyldiyl-N(C₁-C₆-alkyl)-C₁-C₆-alkyldiyl-C(O)—;
   (vi) —O—; or
   (vii) —O—C₁-C₆-alkyldiyl-;
Cy² is:
   (i) absent;
   (ii) C₆-C₁₀-aryl substituted with optionally substituted with 1-3 substitutents R¹⁰; or
   (iii) 3-14 membered heterocyclyl optionally substituted with 1-3 substitutents R¹¹;
Z³ is:
   (i) absent;
   (ii) carbonyl;
   (iii) C₁-C₆-alkyldiyl;
   (iv) —O—; or
   (v) —O—C₁-C₆-alkyldiyl-;
Cy³ is:
   (i) absent; or
   (ii) 3-14 membered heterocyclyl optionally substituted with 1-3 substitutents R²;
n and p are each independently an integer selected from 0, 1, 2, 3, 4, 5 and 6;
R⁴ and R⁹ are independently selected from C₁-C₆-alkyl and C₆-C₁₀-aryl;
R⁵, R⁶, R⁷, R⁸, R¹⁰, R¹¹ and R¹² are independently selected from halogen, cyano, C₁-C₆-alkyl, C₁-C₆-alkoxy, halo-C₁-C₆-alkyl, halo-C₁-C₆-alkoxy and C₆-C₁₀-aryl; and
the wavy line indicates the point of attachment to the linker;
said linker is selected from formulae (L-1), (L-2), (L-3), (L-4), (L-5), (L-6), (L-7) and (L-8):

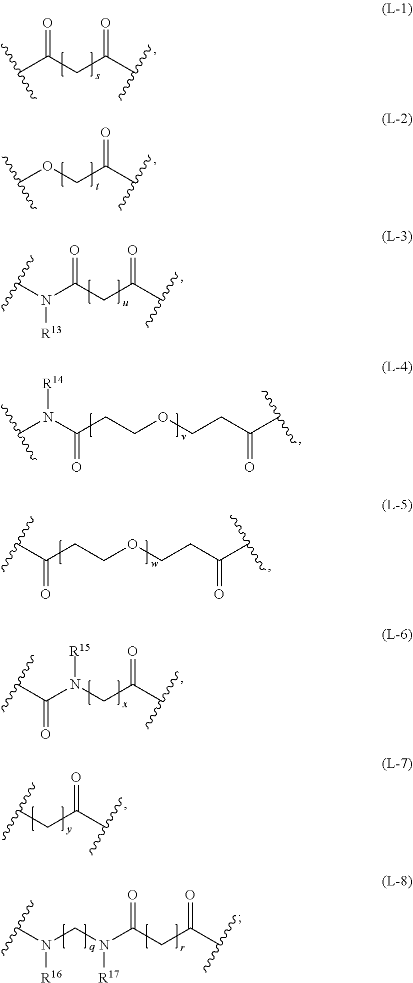

wherein:
R¹³, R¹⁴, R¹⁵ and R¹⁶ are independently selected from hydrogen and C₁-C₆-alkyl;
q, r, s, t, u, v, w, x and y are independently an integer selected from 1, 2, 3, 4, 5, 6, 7, 8, 9, 10, 11 and 12; and
a wavy line indicates the point of attachment to the targeting ligand or the degron; and
said degron is of formula (DG-1) or (DG-2):

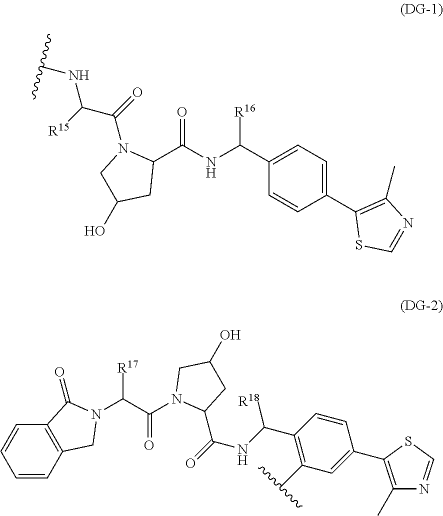

wherein:
R¹⁵, R¹⁶, R¹⁷ and R¹⁸ are independently selected from hydrogen and C₁-C₆-alkyl; and the wavy line indicates the point of attachment to the linker.

In one embodiment, the present invention provides a compound of formula (I) as described herein, or a pharmaceutically acceptable salt thereof, wherein said targeting ligand is of formula (TL), wherein:
R¹ is selected from hydrogen and halogen;
R² is hydrogen;
R³ is selected from hydroxy and amino;
Z¹ is:
   (i) absent;
   (ii) —O—;
   (iii) —O—C₁-C₆-alkyldiyl-;
   (iv) —O(CH₂)ₙCH(R⁴)(CH₂)ₚ—; or
   (v) C₂-C₆-alkynyldiyl;
Cy¹ is:
   (i) absent;
   (ii) C₆-C₁₀-aryl optionally substituted with R⁵;
   (iii) C₃-C₁₀-cycloalkyl;
   (iv) 5-14 membered heteroaryl; or
   (v) 3-14 membered heterocyclyl optionally substituted R⁸;
Z² is:
   (i) absent;
   (ii) —C(O)NH—C₁-C₆-alkyldiyl-;
   (iii) —CH(R⁹)—;
   (iv) C₁-C₆-alkyldiyl;
   (v) —C₁-C₆-alkyldiyl-N(C₁-C₆-alkyl)-C₁-C₆-alkyldiyl-C(O)—;
   (vi) —O—; or
   (vii) —O—C₁-C₆-alkyldiyl-;
Cy² is:
   (i) absent;
   (ii) C₆-C₁₀-aryl; or
   (iii) 3-14 membered heterocyclyl;
Z³ is:
   (i) absent;
   (ii) carbonyl;
   (iii) C₁-C₆-alkyldiyl; or
   (iv) —O—C₁-C₆-alkyldiyl-;
Cy³ is:
   (i) absent; or
   (ii) 3-14 membered heterocyclyl;
n and p are each independently an integer selected from 0 and 1;
R⁴ is selected from C₁-C₆-alkyl and C₆-C₁₀-aryl;
R⁵ is halogen;
R⁸ is selected from C₁-C₆-alkyl and C₆-C₁₀-aryl;
R⁹ is C₆-C₁₀-aryl; and
the wavy line indicates the point of attachment to the linker.

In a preferred embodiment, the present invention provides a compound of formula (I) as described herein, or a pharmaceutically acceptable salt thereof, wherein said targeting ligand is of formula (TL), wherein:
R¹ and R² are both hydrogen;
R³ is hydroxy;
Z¹ is:
   (i) absent;
   (ii) —O—; or
   (iii) —O—C₁-C₆-alkyldiyl-;
Cy¹ is:
   (i) C₆-C₁₀-aryl; or
   (ii) 3-14 membered heterocyclyl;
Z² is:
   (i) absent;
   (ii) —O—; or
   (iii) —O—C₁-C₆-alkyldiyl-;
Cy² is:
   (i) absent;
   (ii) C₆-C₁₀-aryl; or
   (iii) 3-14 membered heterocyclyl;
Z³ is:
   (i) absent;
   (ii) carbonyl; or
   (iii) —O—C₁-C₆-alkyldiyl-;
Cy³ is:
   (i) absent; or
   (ii) 3-14 membered heterocyclyl; and
the wavy line indicates the point of attachment to the linker.

In a particularly preferred embodiment, the present invention provides a compound of formula (I) as described herein, or a pharmaceutically acceptable salt thereof, wherein said targeting ligand is of formula (TL), wherein:
R¹ and R² are both hydrogen;
R³ is hydroxy;
Z¹ is:
   (i) absent;
   (ii) —O—; or
   (iii) —OCH₂CH₂—;
Cy¹ is selected from the group consisting of:

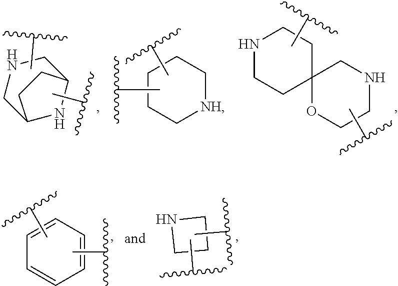

wherein a wavy line indicates the point of attachment to Z¹ or Z²;
Z² is:
   (i) absent;
   (ii) —O—; or
   (iii) —OCH₂CH₂—;
Cy² is absent or selected from the group consisting of:

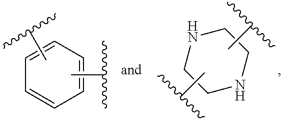

wherein a wavy line indicates the point of attachment to Z² or Z³;
Z³ is:
   (i) absent;
   (ii) carbonyl; or
   (iii) —OCH₂CH₂—; and
Cy³ is absent or

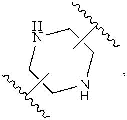

wherein a wavy line indicates the point of attachment to Z³ or the linker.

In one embodiment, the present invention provides a compound of formula (I) as described herein, or a pharmaceutically acceptable salt thereof, wherein said linker is selected from formulae (L-1), (L-2), (L-3), (L-4), (L-5), (L-6), (L-7) and (L-8), wherein:
R¹³ is selected from hydrogen and C₁-C₆-alkyl;
R¹⁴, R¹⁶ and R¹⁷ are independently C₁-C₆-alkyl;
R¹⁵ is hydrogen;
q is 3;
r is an integer selected from 5, 8 and 10;
s is an integer selected from 2, 5, 6, 7, 8, 9, 10 and 12;
t is an integer selected from 9 and 10;
u is an integer selected from 5, 8, 10 and 12;
v is an integer selected from 1 and 2;
w is an integer selected from 1, 2 and 3;
x is an integer selected from 6 and 9;
y is 7; and
a wavy line indicates the point of attachment to the targeting ligand or the degron.

In a preferred embodiment, the present invention provides a compound of formula (I) as described herein, or a pharmaceutically acceptable salt thereof, wherein said linker is of formula (L-1), wherein s is an integer selected from 5, 8, 9, 10 and 12; and a wavy line indicates the point of attachment to the targeting ligand or the degron.

In one embodiment, the present invention provides a compound of formula (I) as described herein, or a pharmaceutically acceptable salt thereof, wherein said degron is of formula (DG-1) or (DG-2), wherein:
R¹⁵ is C₁-C₆-alkyl;
R¹⁶ is selected from hydrogen and C₁-C₆-alkyl;
R¹⁷ is C₁-C₆-alkyl;
R¹⁸ is hydrogen; and
the wavy line indicates the point of attachment to the linker.

In a preferred embodiment, the present invention provides a compound of formula (I) as described herein, or a pharmaceutically acceptable salt thereof, wherein said degron is of formula (DG-1) wherein:
R¹⁵ is C₁-C₆-alkyl;
R¹⁶ is selected from hydrogen and C₁-C₆-alkyl; and
the wavy line indicates the point of attachment to the linker.

In a particularly preferred embodiment, the present invention provides a compound of formula (I) as described herein, or a pharmaceutically acceptable salt thereof, wherein said degron is of formula (DG-1) wherein:
R¹⁵ is tert-butyl or isopropyl;
R¹⁶ is selected from hydrogen and methyl; and
the wavy line indicates the point of attachment to the linker.

In one embodiment, the present invention provides a compound of formula (I) as described herein, or a pharmaceutically acceptable salt thereof, wherein said targeting ligand is of formula (TL), wherein:
R¹ is selected from hydrogen and halogen;
R² is hydrogen;
R³ is selected from hydroxy and amino;
Z¹ is:
   (i) absent;
   (ii) —O—;
   (iii) —O—C₁-C₆-alkyldiyl-;
   (iv) —O(CH₂)ₙCH(R⁴)(CH₂)ₚ—; or
   (v) C₂-C₆-alkynyldiyl;
Cy¹ is:
   (i) absent;
   (ii) C₆-C₁₀-aryl optionally substituted with R⁵;
   (iii) C₃-C₁₀-cycloalkyl;
   (iv) 5-14 membered heteroaryl; or
   (v) 3-14 membered heterocyclyl optionally substituted R⁸;
Z² is:
   (i) absent;
   (ii) —C(O)NH—C₁-C₆-alkyldiyl-;
   (iii) —CH(R⁹)—;
   (iv) C₁-C₆-alkyldiyl;
   (v) —C₁-C₆-alkyldiyl-N(C₁-C₆-alkyl)-C₁-C₆-alkyldiyl-C(O)—;
   (vi) —O—; or
   (vii) —O—C₁-C₆-alkyldiyl-;
Cy² is:
   (i) absent;
   (ii) C₆-C₁₀-aryl; or
   (iii) 3-14 membered heterocyclyl;
Z³ is:
   (i) absent;
   (ii) carbonyl;
   (iii) C₁-C₆-alkyldiyl; or
   (iv) —O—C₁-C₆-alkyldiyl-;
Cy³ is:
   (i) absent; or
   (ii) 3-14 membered heterocyclyl;
n and p are each independently an integer selected from 0 and 1;
R⁴ is selected from C₁-C₆-alkyl and C₆-C₁₀-aryl;
R⁵ is halogen;
R⁸ is selected from C₁-C₆-alkyl and C₆-C₁₀-aryl;
R⁹ is C₆-C₁₀-aryl; and
the wavy line indicates the point of attachment to the linker;
said linker is selected from formulae (L-1), (L-2), (L-3), (L-4), (L-5), (L-6), (L-7) and (L-8), wherein:
R¹³ is selected from hydrogen and C₁-C₆-alkyl;
R¹⁴, R¹⁶ and R¹⁷ are independently C₁-C₆-alkyl;
R¹⁵ is hydrogen;
q is 3;
r is an integer selected from 5, 8 and 10;
s is an integer selected from 2, 5, 6, 7, 8, 9, 10 and 12;
t is an integer selected from 9 and 10;
u is an integer selected from 5, 8, 10 and 12;
v is an integer selected from 1 and 2;
w is an integer selected from 1, 2 and 3;
x is an integer selected from 6 and 9;
y is 7; and
a wavy line indicates the point of attachment to the targeting ligand or the degron; and
said degron is of formula (DG-1) or (DG-2), wherein:
R¹⁵ is C₁-C₆-alkyl;
R¹⁶ is selected from hydrogen and C₁-C₆-alkyl;
R¹⁷ is C₁-C₆-alkyl;
R¹⁸ is hydrogen; and
the wavy line indicates the point of attachment to the linker.

In a preferred embodiment, the present invention provides a compound of formula (I) as described herein, or a pharmaceutically acceptable salt thereof, wherein said targeting ligand is of formula (TL), wherein:
R¹ and R² are both hydrogen;
R³ is hydroxy;
Z¹ is:
   (i) absent;
   (ii) —O—; or
   (iii) —O—C₁-C₆-alkyldiyl-;
Cy¹ is:
   (i) C₆-C₁₀-aryl; or
   (ii) 3-14 membered heterocyclyl;
Z² is:
   (i) absent;
   (ii) —O—; or
   (iii) —O—C₁-C₆-alkyldiyl-;
Cy² is:
   (i) absent;
   (ii) C₆-C₁₀-aryl; or
   (iii) 3-14 membered heterocyclyl;
Z³ is:
   (i) absent;
   (ii) carbonyl; or
   (iii) —O—C₁-C₆-alkyldiyl-;
Cy³ is:
   (i) absent; or
   (ii) 3-14 membered heterocyclyl; and
the wavy line indicates the point of attachment to the linker;
said linker is of formula (L-1), wherein s is an integer selected from 5, 8, 9, 10 and 12; and
a wavy line indicates the point of attachment to the targeting ligand or the degron; and
said degron is of formula (DG-1) wherein:
R¹⁵ is C₁-C₆-alkyl;
R¹⁶ is selected from hydrogen and C₁-C₆-alkyl; and
the wavy line indicates the point of attachment to the linker.

In a particularly preferred embodiment, the present invention provides a compound of formula (I) as described herein, or a pharmaceutically acceptable salt thereof, wherein said targeting ligand is of formula (TL), wherein:
R¹ and R² are both hydrogen;
R³ is hydroxy;
Z¹ is:
   (i) absent;
   (ii) —O—; or
   (iii) —OCH₂CH₂—;
Cy¹ is selected from the group consisting of:

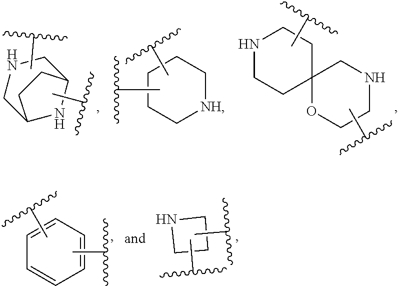

wherein a wavy line indicates the point of attachment to Z¹ or Z²;
Z² is:
   (i) absent;
   (ii) —O—; or
   (iii) —OCH₂CH₂—;
Cy² is absent or selected from the group consisting of:

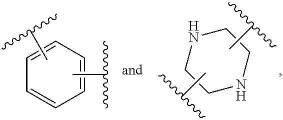

wherein a wavy line indicates the point of attachment to Z² or Z³;
Z³ is:
   (i) absent;
   (ii) carbonyl; or
   (iii) —OCH₂CH₂—; and
Cy³ is absent or

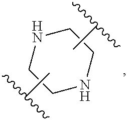

wherein a wavy line indicates the point of attachment to Z³ or the linker;
said linker is of formula (L-1), wherein s is an integer selected from 5, 8, 9, 10 and 12; and
a wavy line indicates the point of attachment to the targeting ligand or the degron; and
said degron is of formula (DG-1) wherein:
R¹⁵ is tert-butyl or isopropyl;
R¹⁶ is selected from hydrogen and methyl; and
the wavy line indicates the point of attachment to the linker.

In one embodiment, the present invention provides a compound of formula (I) as described herein, or a pharmaceutically acceptable salt thereof, wherein said compound of formula (I) is selected from Examples 1 to 111.

In one embodiment, the present invention provides a compound of formula (I) as described herein, or a pharmaceutically acceptable salt thereof, wherein said compound of formula (I) is selected from Examples 11, 32, 33, 37, 45, 48, 56, 58, 78, 79, 96 and 101.

In one embodiment, the present invention provides pharmaceutically acceptable salts or esters of the compounds of formula (I) as described herein. In a particular embodiment, the present invention provides pharmaceutically acceptable salts of the compounds according to formula (I) as described herein. In a further particular embodiment, the present invention provides pharmaceutically acceptable esters of the compounds according to formula (I) as described herein. In yet a further particular embodiment, the present invention provides compounds according to formula (I) as described herein.

Furthermore, the invention includes all optical isomers, i.e. diastereoisomers, diastereomeric mixtures, racemic mixtures, all their corresponding enantiomers and/or tautomers as well as their solvates of the compounds of formula I.

The compounds of formula I may contain one or more asymmetric centers and can therefore occur as racemates, racemic mixtures, single enantiomers, diastereomeric mixtures and individual diastereomers. Additional asymmetric centers may be present depending upon the nature of the various substituents on the molecule. Each such asymmetric center will independently produce two optical isomers and it is intended that all of the possible optical isomers and diastereomers in mixtures and as pure or partially purified compounds are included within this invention. The present invention is meant to encompass all such isomeric forms of these compounds. The independent syntheses of these diastereomers or their chromatographic separations may be achieved as known in the art by appropriate modification of the methodology disclosed herein. Their absolute stereochemistry may be determined by the x-ray crystallography of crystalline products or crystalline intermediates which are derivatized, if necessary, with a reagent containing an asymmetric center of known absolute configuration. If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography.

In the embodiments, where optically pure enantiomers are provided, optically pure enantiomer means that the compound contains >90% of the desired isomer by weight, particularly >95% of the desired isomer by weight, or more particularly >99% of the desired isomer by weight, said weight percent based upon the total weight of the isomer(s) of the compound. Chirally pure or chirally enriched compounds may be prepared by chirally selective synthesis or by separation of enantiomers. The separation of enantiomers may be carried out on the final product or alternatively on a suitable intermediate.

In some embodiments, the compounds of formula (I) are isotopically-labeled by having one or more atoms therein replaced by an atom having a different atomic mass or mass number. Such isotopically-labeled (i.e., radiolabeled) compounds of formula (I) are considered to be within the scope of this disclosure. Examples of isotopes that can be incorporated into the compounds of formula (I) include isotopes of hydrogen, carbon, nitrogen, oxygen, phosphorous, sulfur, fluorine, chlorine, and iodine, such as, but not limited to, ²H, ³H, ¹¹C, ¹³C, ¹⁴c, ¹³N ¹⁵N, ¹⁵O, ¹⁷O, ¹⁸O, ³¹P, ³²P ³⁵S, ¹⁸F, ³⁶Cl, ¹²³I, and ¹²⁵I, respectively. Certain isotopically-labeled compounds of formula (I), for example, those incorporating a radioactive isotope, are useful in drug and/or substrate tissue distribution studies. The radioactive isotopes tritium, i.e. ³H, and carbon-14, i.e., ¹⁴C, are particularly useful for this purpose in view of their ease of incorporation and ready means of detection. For example, a compound of formula (I) can be enriched with 1, 2, 5, 10, 25, 50, 75, 90, 95, or 99 percent of a given isotope.

Substitution with heavier isotopes such as deuterium, i.e. ²H, may afford certain therapeutic advantages resulting from greater metabolic stability, for example, increased in vivo half-life or reduced dosage requirements.

Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of formula (I) can generally be prepared by conventional techniques known to those skilled in the art or by processes analogous to those described in the Examples as set out below using an appropriate isotopically-labeled reagent in place of the non-labeled reagent previously employed.

### Processes of Manufacturing

The preparation of compounds of formula (I) of the present invention may be carried out in sequential or convergent synthetic routes. Syntheses of the invention are shown in the following general schemes. The skills required for carrying out the reaction and purification of the resulting products are known to those persons skilled in the art. The substituents and indices used in the following description of the processes have the significance given herein, unless indicated to the contrary.

If one of the starting materials, intermediates or compounds of formula (I) contain one or more functional groups which are not stable or are reactive under the reaction conditions of one or more reaction steps, appropriate protective groups (as described e.g., in “Protective Groups in Organic Chemistry” by T. W. Greene and P. G. M. Wutts, 5th Ed., 2014, John Wiley & Sons, N.Y.) can be introduced before the critical step applying methods well known in the art. Such protective groups can be removed at a later stage of the synthesis using standard methods described in the literature.

If starting materials or intermediates contain stereogenic centers, compounds of formula (I) can be obtained as mixtures of diastereomers or enantiomers, which can be separated by methods well known in the art e.g., chiral HPLC, chiral SFC or chiral crystallization. Racemic compounds can e.g., be separated into their antipodes via diastereomeric salts by crystallization with optically pure acids or by separation of the antipodes by specific chromatographic methods using either a chiral adsorbent or a chiral eluent. It is equally possible to separate starting materials and intermediates containing stereogenic centers to afford diastereomerically/enantiomerically enriched starting materials and intermediates. Using such diastereomerically/enantiomerically enriched starting materials and intermediates in the synthesis of compounds of formula (I) will typically lead to the respective diastereomerically/enantiomerically enriched compounds of formula (I).

A person skilled in the art will acknowledge that in the synthesis of compounds of formula (I)—insofar not desired otherwise—an “orthogonal protection group strategy” will be applied, allowing the cleavage of several protective groups one at a time each without affecting other protective groups in the molecule. The principle of orthogonal protection is well known in the art and has also been described in literature (e.g. Barany and R. B. Merrifield, J. Am. Chem. Soc. 1977, 99, 7363; H. Waldmann et al., Angew. Chem. Int. Ed. Engl. 1996, 35, 2056).

A person skilled in the art will acknowledge that the sequence of reactions may be varied depending on reactivity and nature of the intermediates.

In more detail, the compounds of formula (I) can be manufactured by the methods given below, by the methods given in the examples or by analogous methods. Appropriate reaction conditions for the individual reaction steps are known to a person skilled in the art. Also, for reaction conditions described in literature affecting the described reactions see for example: Comprehensive Organic Transformations: A Guide to Functional Group Preparations, 2nd Edition, Richard C. Larock. John Wiley & Sons, New York, N.Y. 1999). It was found convenient to carry out the reactions in the presence or absence of a solvent. There is no particular restriction on the nature of the solvent to be employed, provided that it has no adverse effect on the reaction or the reagents involved and that it can dissolve the reagents, at least to some extent. The described reactions can take place over a wide range of temperatures, and the precise reaction temperature is not critical to the invention. It is convenient to carry out the described reactions in a temperature range between −78° C. to reflux. The time required for the reaction may also vary widely, depending on many factors, notably the reaction temperature and the nature of the reagents. However, a period of from 0.5 hours to several days will usually suffice to yield the described intermediates and compounds. The reaction sequence is not limited to the one displayed in the schemes, however, depending on the starting materials and their respective reactivity, the sequence of reaction steps can be freely altered.

If starting materials or intermediates are not commercially available or their synthesis not described in literature, they can be prepared in analogy to existing procedures for close analogues or as outlined in the experimental section.

A bifunctional protein degrader molecule of formula (I), or their pharmaceutical acceptable salts, polymorphic forms, prodrugs, solvate forms and isotope containing derivatives thereof, may be prepared by the general approach described below (scheme 1), together with synthetic methods known in the art of organic chemistry, or modifications and derivatizations that are familiar to those of ordinary skill in the art. The compounds of formula (I) can be prepared in a modular fashion by coupling the targeting ligand (TL) with a linker and then subsequently with the degron (scheme 2).

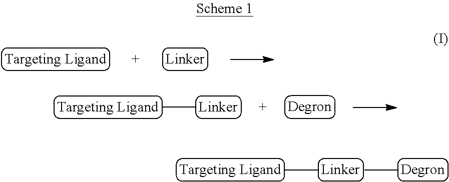

The targeting ligand, the linker and the degron contain moieties with suitable reacting groups that would be necessary to enable the synthetic chemistry to connect the targeting ligand, the linker and the degron together into a bifunctional degrader molecule of Formula (I) via covalent bond formation chemistries. These chemistries, depending on specific reacting groups, include but not limited to, amide formation, ester formation, carbamate formation, urea formation, ether formation, amine formation, sulfonamide formation and various C—C, C═C bond formation. The stage 1 and stage 2 transformations in Scheme 1 and scheme 2 may involve 1 or multiple synthetic steps. These are routine methods known in the art such as those methods disclosed in standard reference books such as the Compendium of Organic Synthetic Methods, Vol. I-VI (Wiley-Interscience); or the Comprehensive Organic Transformations, by R. C. Larock (Wiley-Interscience). Unless otherwise indicated, the substituents in the schemes are defined as above. Isolation and purification of the products is accomplished by standard procedures, which are known to a chemist of ordinary skills.

The sequence of the coupling for the preparation of bifunctional protein degrader molecule of formula (I) may be reversed as shown below (scheme 2).

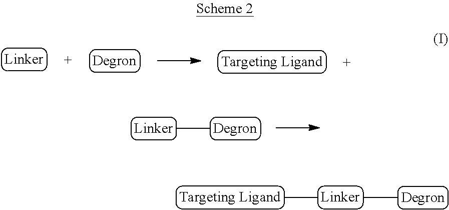

As an example of this general concept the synthesis of example 1 is described in the following scheme 3 in a general fashion employing linker (L-1) and degron (DG-1).

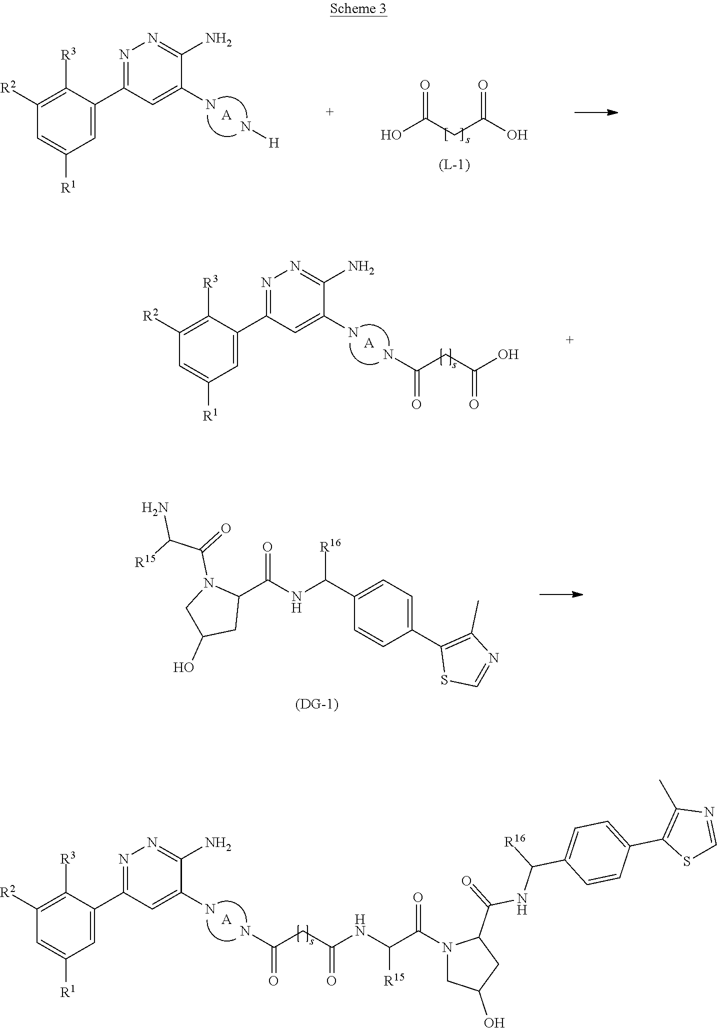

The target ligand is coupled with a diacid (linker (L-1)) in the presence of an activating reagent (e.g. HATU), eventually a base (e.g. hünig base) in an apolar solvent (e.g. dichloromethane or DMF) under cooling in an ice bath or under elevated temperature. This procedure is repeated with the degron to yield compound of the general formula (I).

The targeting ligand of formula (TL) can be prepared according to scheme 4 in which CA is the connecting atom of the building block BB to the pyridazine ring. CA maybe connected via single bond to the BB or maybe part of a ring system of the BB. The stage 1, 2 and stage 3 transformations in Scheme 4 may involve 1 or multiple synthetic steps.

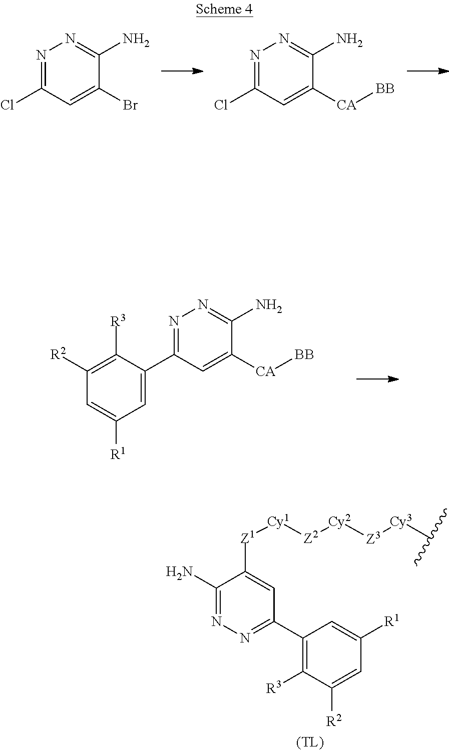

In certain examples, for the chemistry described in Scheme 4, CA is a nitrogen atom. In a typical procedure, 3-amino-4-bromo-6-chloro-pyridazine is reacted with a primary or secondary amine containing intermediate in a suitable solvent. Suitable solvents include, but are not limited to, water, ethers such as THF, glyme, and the like; chlorinated solvents such as DCM, 1,2-dichloroethane (DCE) or CHCl₃ and the like; toluene, benzene and the like; DMF, NMP, DMSO MeCN. If desired, mixtures of these solvents are used. To facilitate the reaction a base may be added. Suitable bases include, but are not limited to, Cs₂CO₃, K₂CO₃ and the like; TEA, DIPEA and the like. The above process may be carried out at temperatures between about 20° C. and about 200° C. Preferentially, the reaction is carried out between about 50° C. and about 130° C.

The amine maybe part of a heterocyclic ring as described in scheme 5, which may be additionally protected with a protecting group PG. After the nucelophilic aromatic substitution as described above a suzuki reaction with an appropriately substituted phenylboronic acid is performed under palladium catalysis in the presence of a base, eventually in the presence of a ligand like an phosphine or phosphite in an inert organic solvent under elevated temperature. Subsequent deprotection may provide the targeting ligand (TL). Non-limiting examples of these heterocyclic rings are depicted in Schemes 5a and 5b.

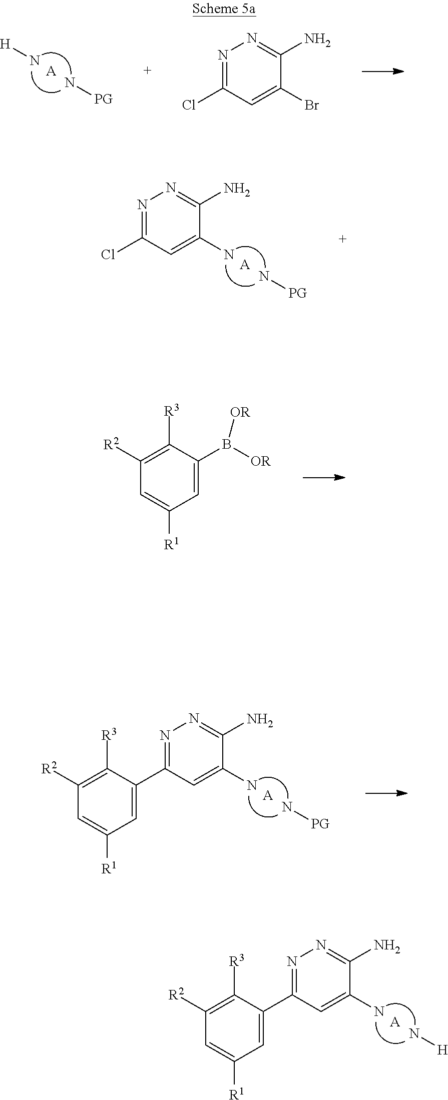

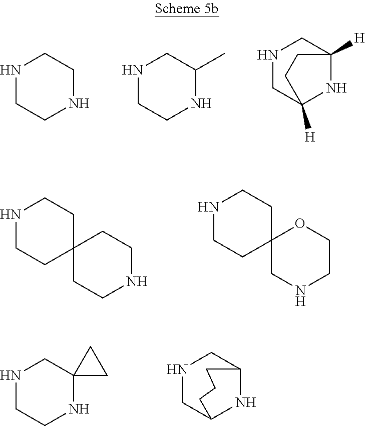

The obtained compounds (schemes 5a,b) can be further elaborated to different targeting ligands (TL) by amide formation, alkylation (schemes 6a,b), carbamate formation, urea formation, sulfonamide formation and various C—N, bond formation.

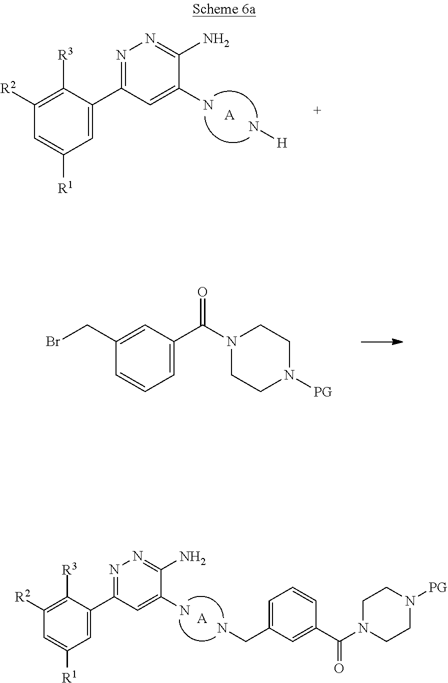

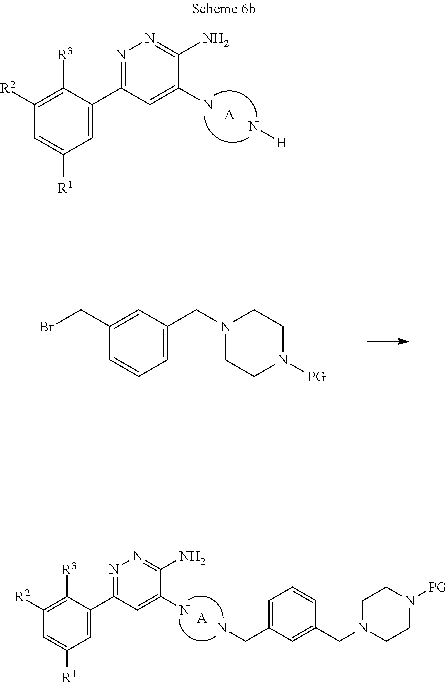

N-aryl-substituted heterocyclic moieties are introduced by palladium or copper-catalyzed coupling of a suitable aryl halogenide with a protected bis-amino-heterocyclic group and after deprotection subsequent nucleophilic substitution and Suzuki reaction (Scheme 7).

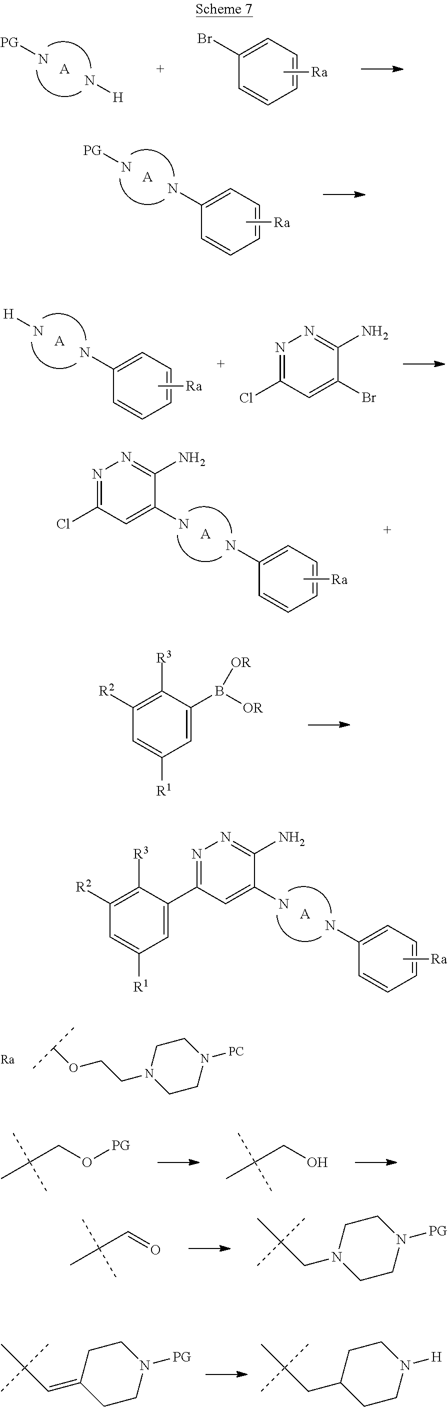

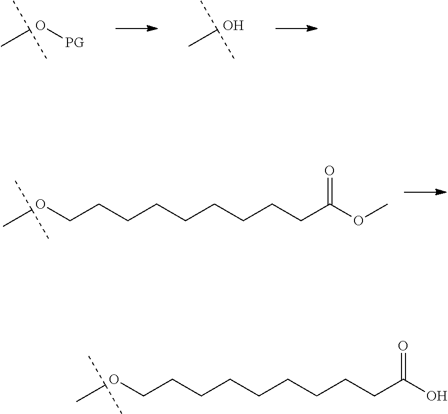

The starting arylbromides used in scheme 7 can be prepared using standard chemistry, e.g. as shown in scheme 8.

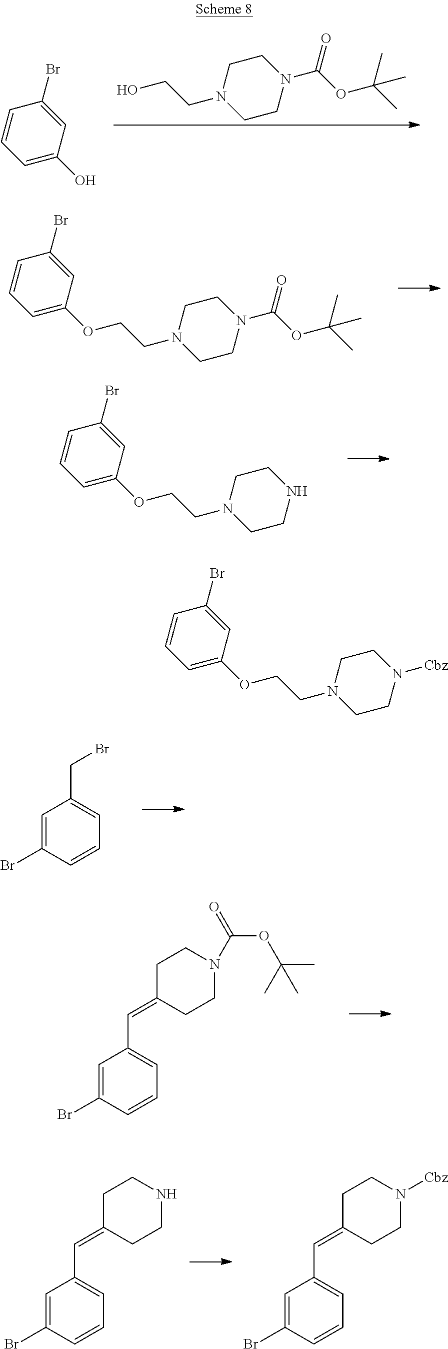

The exit vector Ra could not only be located on the aromatic ring as shown in Scheme 8, but could also be present on the heterocyclic compound as described in schemes 9 and 10.

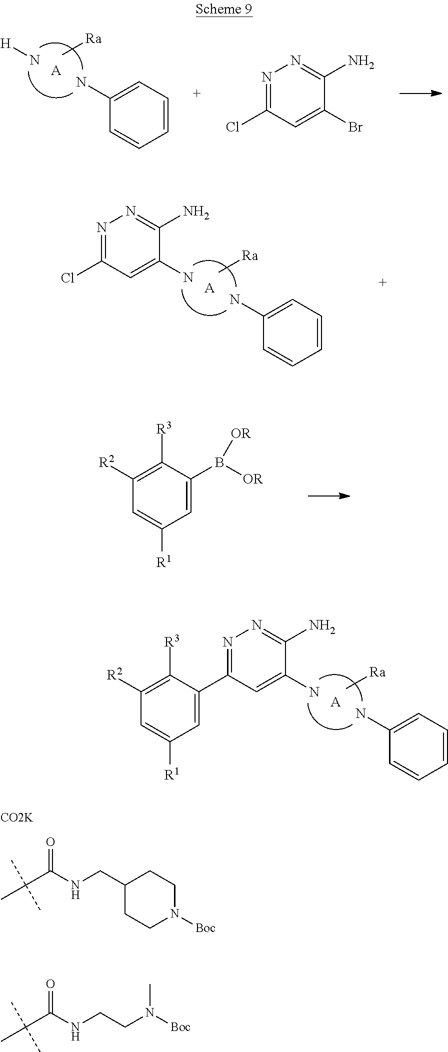

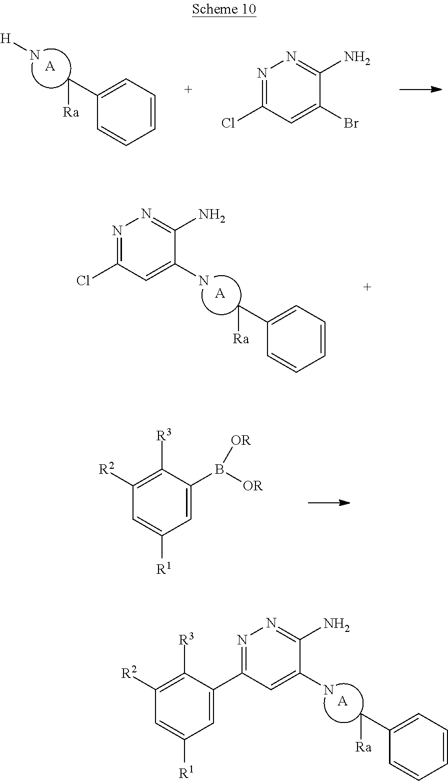

A non-limiting example used as the amine starting material in the described synthesis in scheme 10 is depicted in scheme 11.

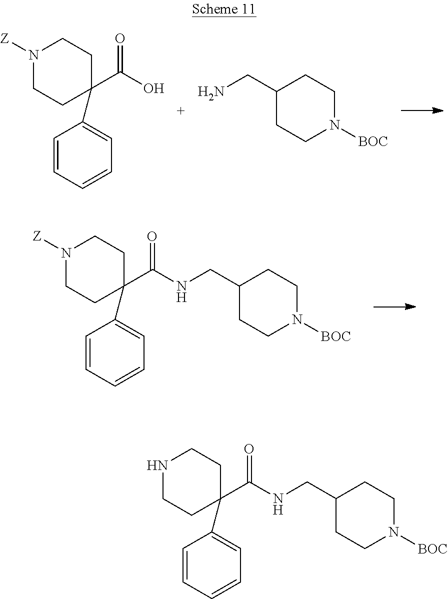

The aromatic ring can be attached via a linker X to the heterocyclic ring system (scheme 12).

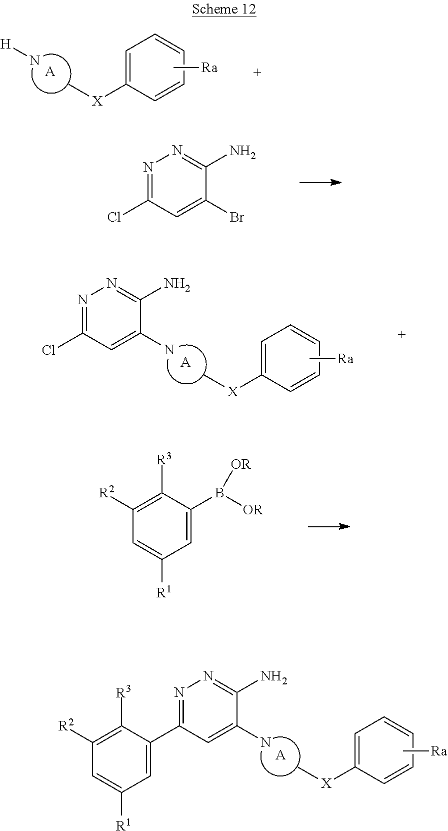

Non-limiting examples for the synthesis of these kind of heterocyclic ring systems are depicted in schemes 13a,b,c.

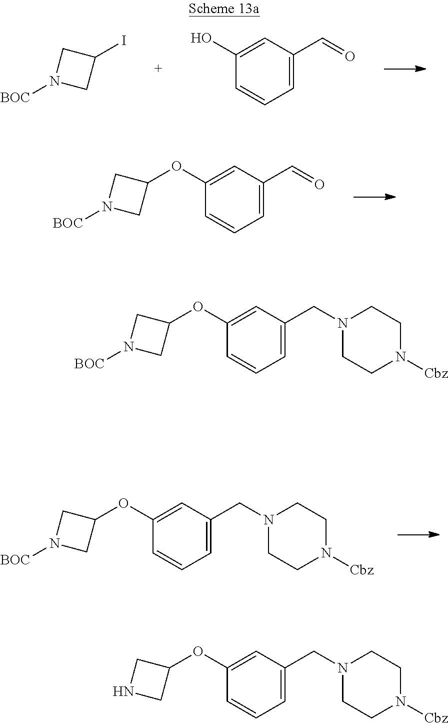

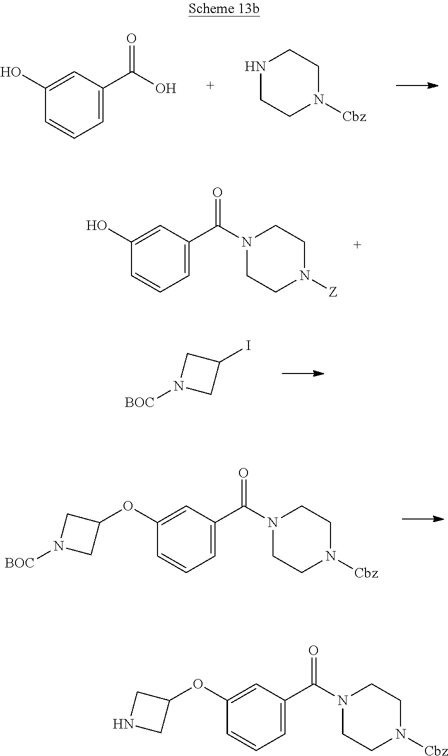

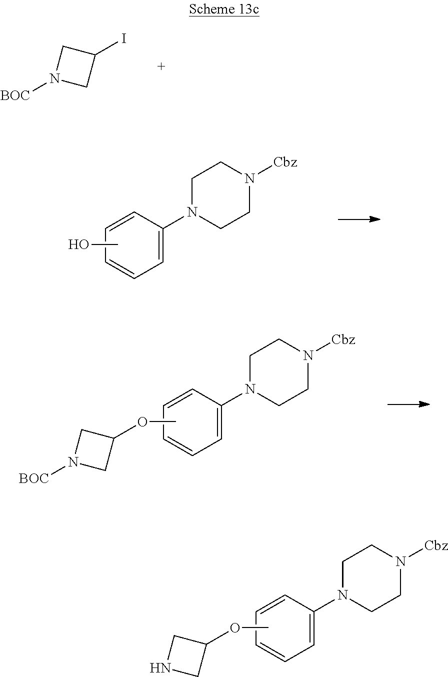

The heterocyclic amine may have as an exit vector an exocyclic protected amino group as depicted in scheme 14.

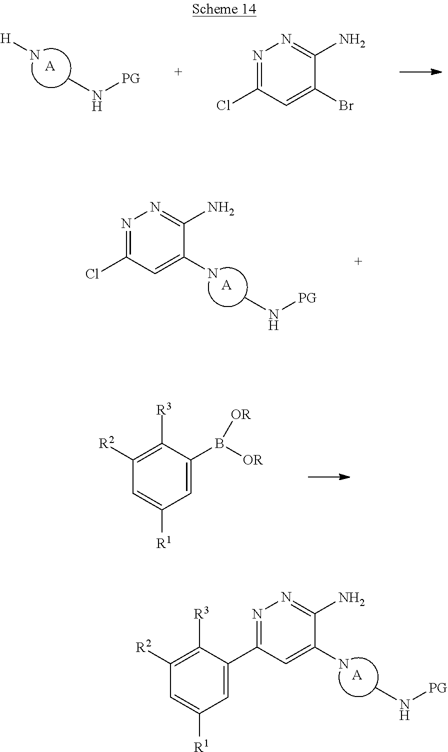

In certain examples, for the chemistry described in Scheme 15, CA is a carbon atom. In a typical procedure, 3-amino-4-bromo-6-chloro-pyridazine is reacted with a boron-containing moiety, preferably an aryl- (scheme 16), heteroaryl- (Scheme 17) or vinyl-boronic acid (scheme 15), boronic ester or boronic salt in a suitable solvent under metal catalysis preferably palladium catalysts (Suzuki coupling). Suitable solvents include, but are not limited to, water, ethers such as THF, glyme, and the like; chlorinated; chlorinated solvents such as DCM, 1,2-dichloroethane (DCE) or CHCl₃ and the like; toluene, benzene and the like; methanol, ethanol, isopropanol and the like; DMF, NMP, DMSO, MeCN. If desired, mixtures of these solvents are used. A base is added to the reaction. Suitable bases include, but are not limited to, sodium tert-butylate, cesium, potassium carbonate, sodium hydrogen carbonate etc. The above process may be carried out at temperatures between 20° C. and about 150° C. eventually in a microwave oven. Preferably, the reaction is carried out between 60° C. and 120° C.

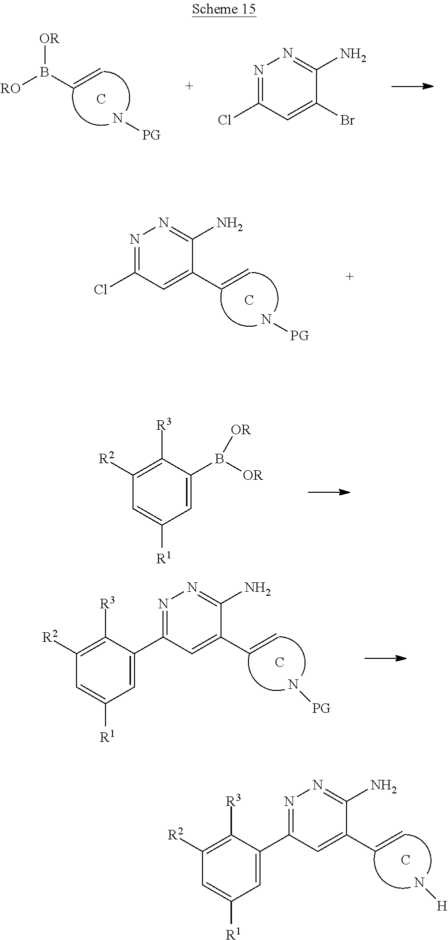

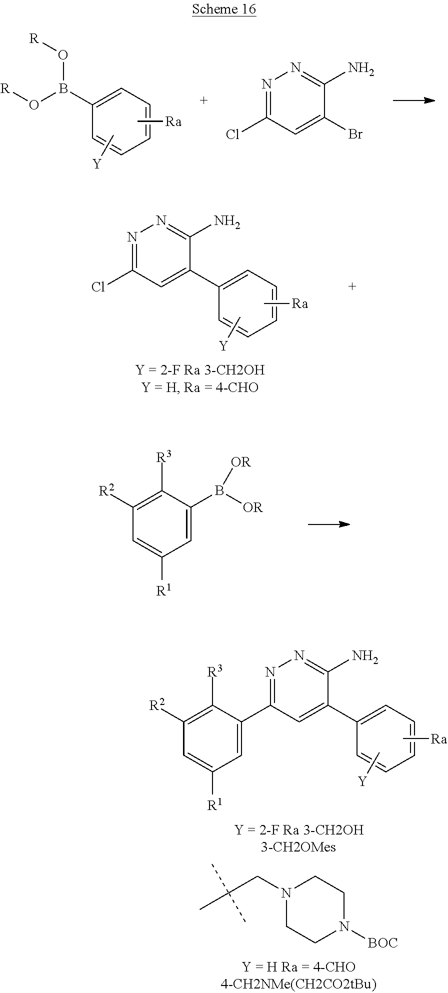

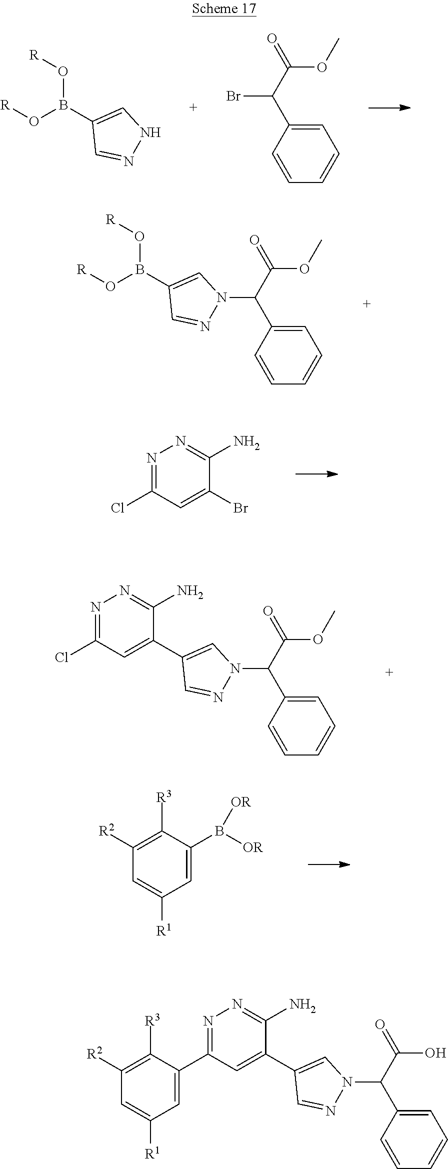

In certain examples, for the chemistry described in Scheme 18, CA is a carbon atom as part of a terminal alkyne group (Stille coupling). In a typical procedure, 3-amino-4-bromo-6-chloro-pyridazine is reacted with an alkyne containing aryl-moiety (scheme 18) in a suitable solvent under metal catalysis preferably palladium catalysts, with or without the presence of a copper salt (e.g. CuI) and a base preferably a tertiary amine. Suitable solvents include, but are not limited to, water, ethers such as THF, glyme, and the like; chlorinated; chlorinated solvents such as DCM, 1,2-dichloroethane (DCE) or CHCl₃ and the like; toluene, benzene and the like; methanol, ethanol, isopropanol and the like; DMF, NMP, DMSO, MeCN. If desired, mixtures of these solvents are used. The above process may be carried out at temperatures between 20° C. and about 150° C. eventually in a microwave oven. Preferably, the reaction is carried out between 60° C. and 120° C.

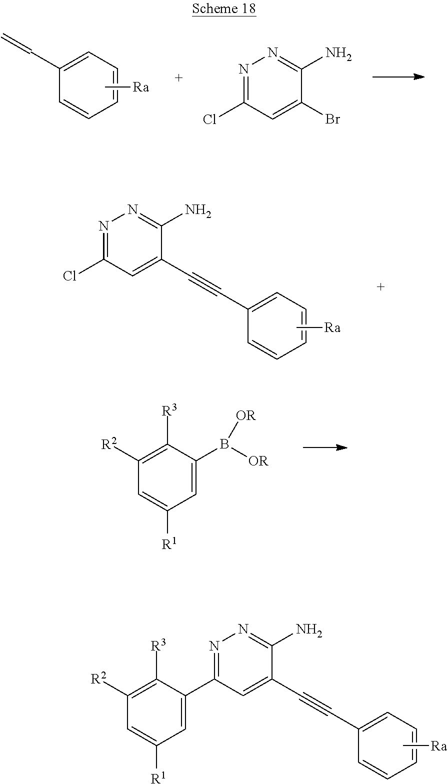

The synthesis of possible starting materials with suitable exit vectors Ra are depicted in schemes 19a,b.

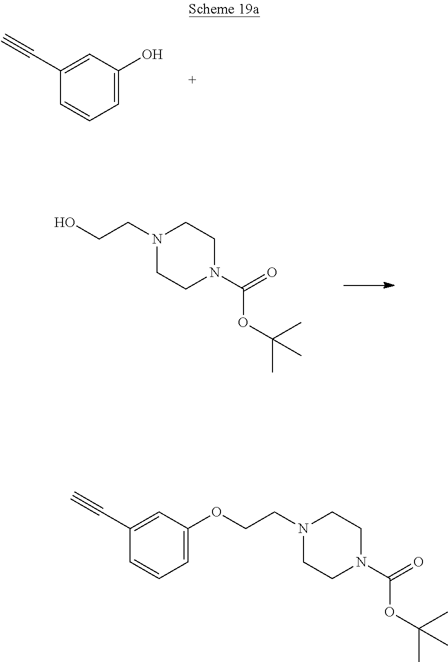

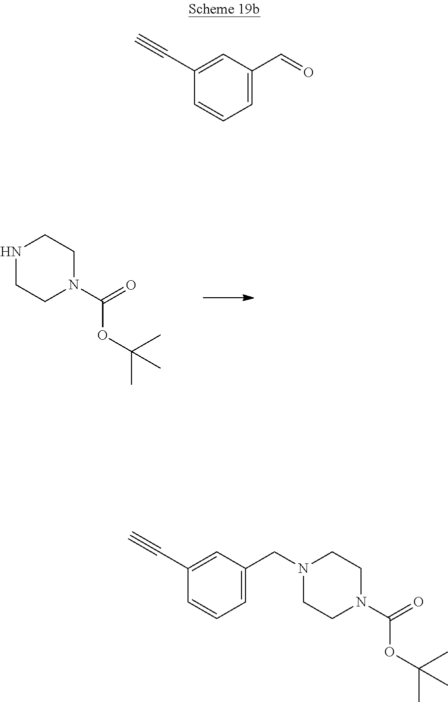

In certain examples, for the chemistry described in Scheme 20, CA is an oxygen atom. In a typical procedure, 3-amino-4-bromo-6-chloro-pyridazine is reacted with an alcohol-containing intermediate in a suitable solvent in the presence of a strong base, like sodium hydride, potassium hexamethyl disilazane or potassium tert-butylate at 0 C celsius, room temperature or elevated temperature. Suitable solvents include, but are not limited to ethers such as THF, glyme, and the like; DMF, NMP, DMSO, MeCN and the like.

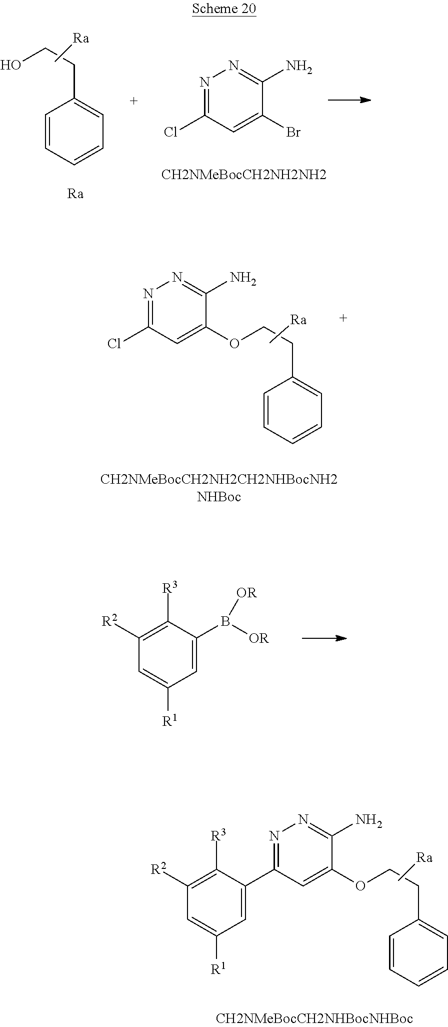

Examples for the synthesis of these primary alcohols containing suitable exit vectors Ra are described in schemes 21a,b,c.

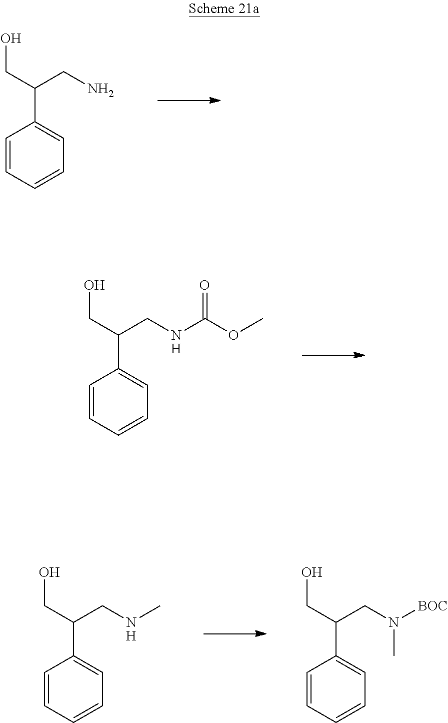

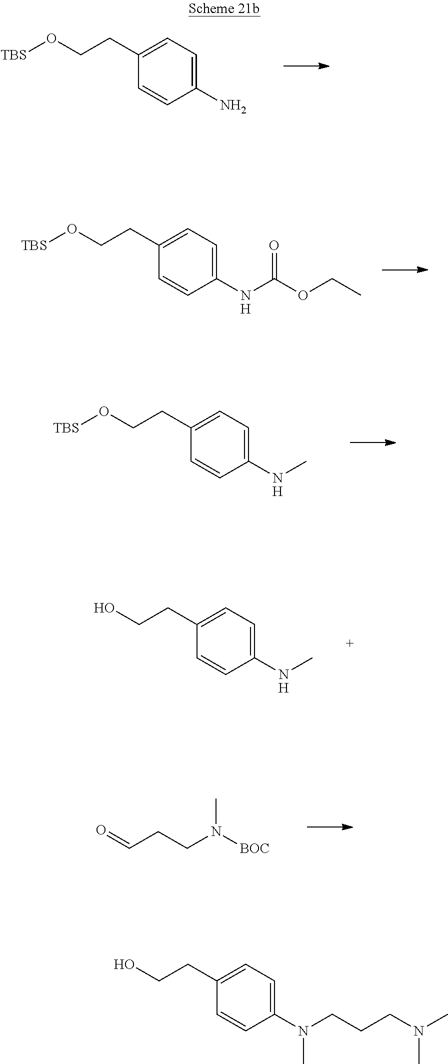

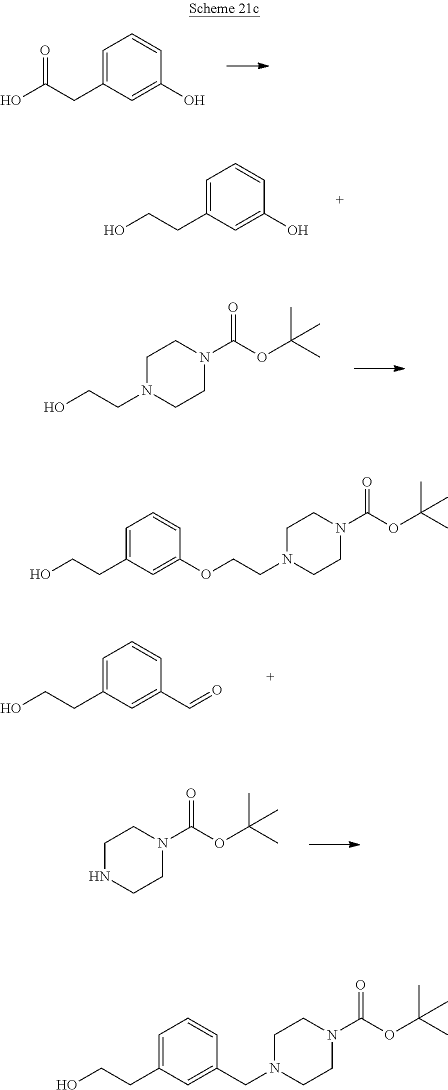

A suitable substituted secondary alcohol (scheme 22) can be used in this chemistry in a similar way as described above.

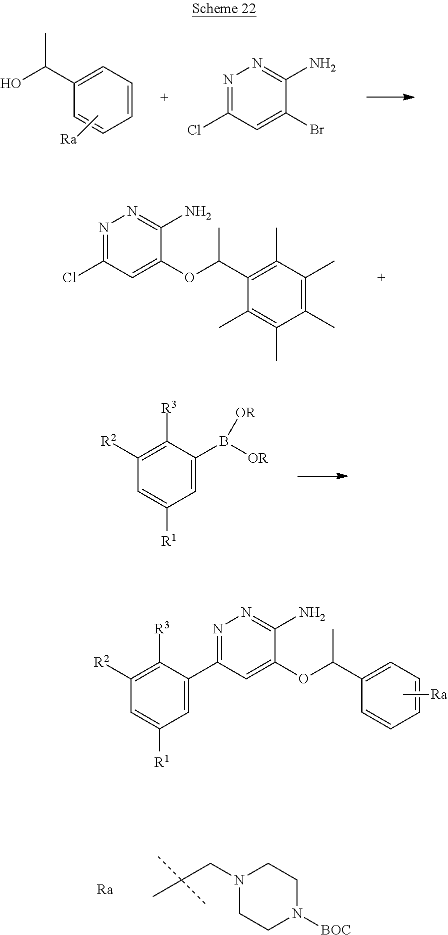

The synthesis of a suitable substrate with an appriate exit vector Ra is described in scheme 23.

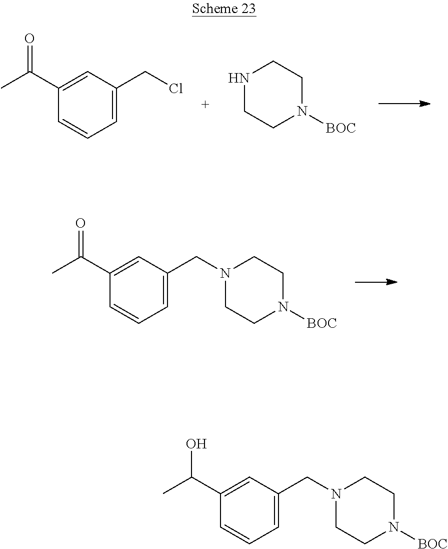 [SMILES: CC(=O)c1cccc(CCl)c1 ; CC(C)(C)OC(=O)N1CCNCC1 ; CC(=O)c1cccc(CN2CCN(C(=O)OC(C)(C)C)CC2)c1 ; CC(O)c1cccc(CN2CCN(C(=O)OC(C)(C)C)CC2)c1]

The secondary alcohol may be part of a heterycyclic ring as described in scheme 24.

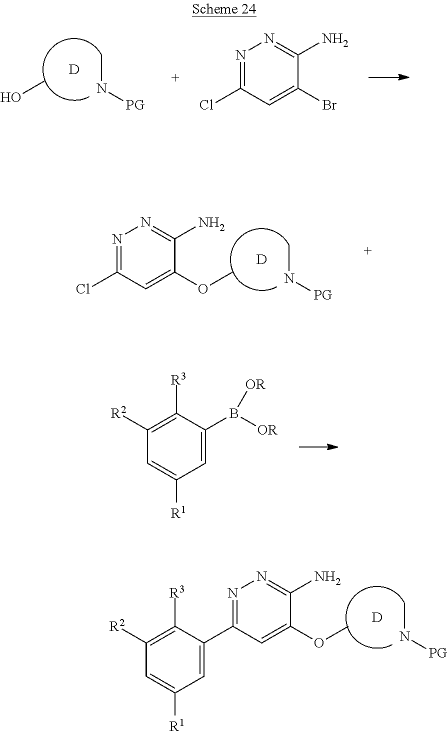

In addition, this heteryclyclic ring may be substituted further with an aromatic ring system connected through an atom Z to the heterylcyclic ring. The aromatic group is substituted with an appriate exit vector Ra (scheme 25).

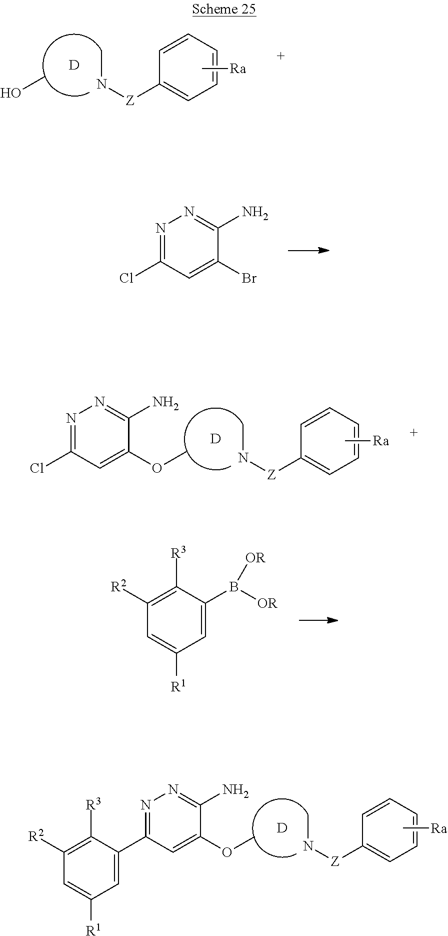

Syntheses of non-limiting suitable substrates with appriate exit vectors Ra are described in schemes 26a,b,c.

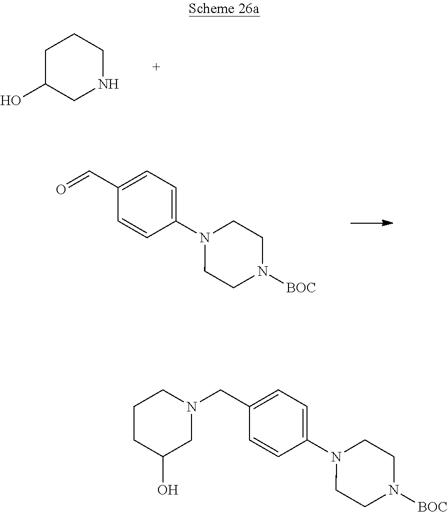

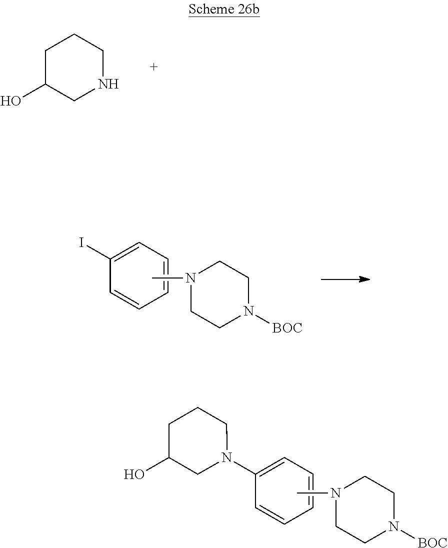

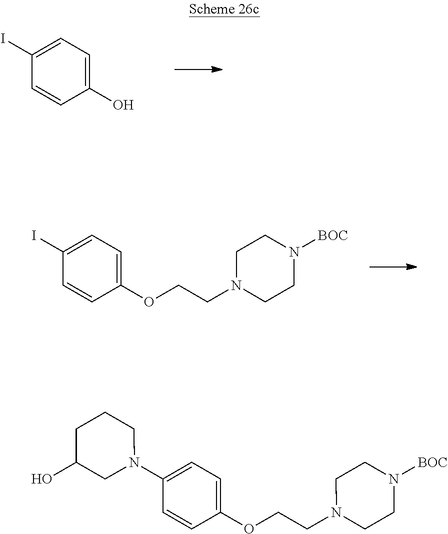

The secondary alcohol may be part of a heterycyclic ring with an exocyclic amino group, which functions as an attachment point for the linker and the ligase moiety as described in scheme 27.

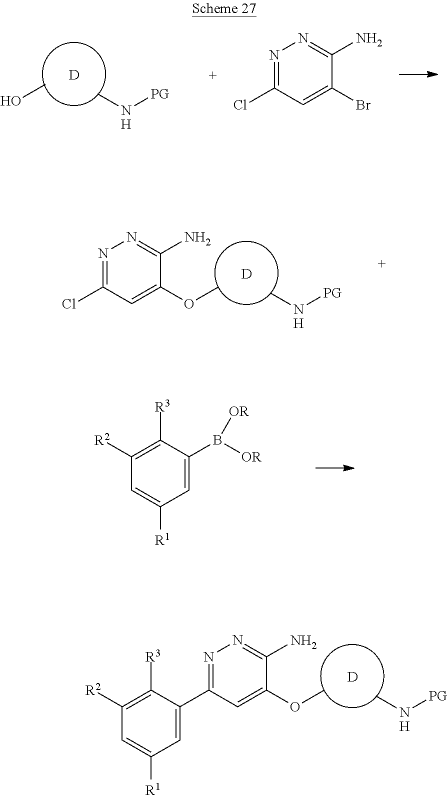

It will be appreciated that the compounds of general formula I in this invention may be derivatised at functional groups to provide derivatives which are capable of conversion back to the parent compound in vivo.

### Using the Compounds of the Invention

The compounds of Formula I can be used in an effective amount to treat a host, including a human, affected by SMARCA2-mediated disorders. More particularly, the compounds of Formula I can be used in an effective amount to treat a subject, in particular a human, affected by cancer.

In one aspect, the present invention provides a compound of formula (I) described herein, or a pharmaceutically acceptable salt thereof, for use as therapeutically active substance.

In a further aspect, the present invention provides a compound of formula (I) described herein, or a pharmaceutically acceptable salt thereof, for use in the treatment of SMARCA2-mediated disorders.

In a further aspect, the present invention provides a method of treating SMARCA2-mediated disorders in a subject, comprising administering a compound of formula (I) as described herein, or a pharmaceutically acceptable salt thereof, to the subject.

In a further aspect, the present invention provides the use of a compound of formula (I) described herein, or a pharmaceutically acceptable salt thereof, in a method of treating SMARCA2-mediated disorders in a subject.

In a further aspect, the present invention provides the use of a compound of formula (I) described herein, or a pharmaceutically acceptable salt thereof, for the manufacture of a medicament for treating SMARCA2-mediated disorders in a subject.

The term “SMARCA2-mediated disorder” is characterized by the participation of the SMARCA2 protein in the inception, manifestation of one or more symptoms or disease markers, severity, or progression of a disorder.

SMARCA2-mediated disorders include cancers, including, but not limited to acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, diffuse large B-cell lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, glioblastoma, gliosarcoma, heavy chain disease, hemangioblastoma, hepatoma, hepatocellular cancer, hormone insensitive prostate cancer, leiomyosarcoma, leukemia, liposarcoma, liver cancer, lung cancer, lymphagioendotheliosarcoma, lymphangiosarcoma, lymphoblastic leukemia, lymphoma (Hodgkin's and non-Hodgkin's; Burkitt's), malignancies and hyperproliferative disorders of the bladder, breast, colon, lung, ovaries, pancreas, prostate, skin and uterus, lymphoid malignancies of T-cell or B-cell origin, medullary carcinoma, medulloblastoma, melanoma, meningioma, mesothelioma, multiple myeloma, myelogenous leukemia, myeloma, myxosarcoma, neuroblastoma, NUT midline carcinoma (NMC), non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, malignant rhabdoid tumor (MRT), rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer and Wilms' tumor.

### Co-Administration of Compounds of Formula (I) and Other Agents

The compounds of formula (I) or salts thereof or a compound disclosed herein or a pharmaceutically acceptable salt thereof may be employed alone or in combination with other agents for treatment. For example, the second agent of the pharmaceutical combination formulation or dosing regimen may have complementary activities to the compound of formula (I) such that they do not adversely affect each other. The compounds may be administered together in a unitary pharmaceutical composition or separately. In one embodiment a compound or a pharmaceutically acceptable salt can be co-administered with a cytotoxic agent to treat proliferative diseases and cancer.

The term “co-administering” refers to either simultaneous administration, or any manner of separate sequential administration, of a compound of formula (I) or a salt thereof or a compound disclosed herein or a pharmaceutically acceptable salt thereof and a further active pharmaceutical ingredient or ingredients, including cytotoxic agents and radiation treatment. If the administration is not simultaneous, the compounds are administered in a close time proximity to each other. Furthermore, it does not matter if the compounds are administered in the same dosage form, e.g. one compound may be administered topically and another compound may be administered orally.

Typically, any agent that has activity against a SMARCA2-mediated disease or condition being treated may be co-administered. Examples of such agents can be found in Cancer Principles and Practice of Oncology by V. T. Devita and S. Heilman (editors), 6th edition (Feb. 15, 2001), Lippincott Williams & Wilkins Publishers. A person of ordinary skill in the art would be able to discern which combinations of agents would be useful based on the particular characteristics of the drugs and the disease involved.

In one aspect, the present invention provides a pharmaceutical composition described herein, further comprising an additional therapeutic agent.

In one embodiment, said additional therapeutic agent is a chemotherapeutic agent.

In one embodiment, said additional therapeutic agent is a cytotoxic agent.

The term “cytotoxic agent” as used herein refers to a substance that inhibits or prevents a cellular function and/or causes cell death or destruction. Cytotoxic agents include, but are not limited to, radioactive isotopes (At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents; growth inhibitory agents; enzymes and fragments thereof such as nucleolytic enzymes; and toxins such as small molecule toxins or enzymatically active toxins of bacterial, fungal, plant or animal origin, including fragments and/or variants thereof.

Exemplary cytotoxic agents can be selected from anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, inhibitors of LDH-A; inhibitors of fatty acid biosynthesis; cell cycle signaling inhibitors; HDAC inhibitors, proteasome inhibitors; and inhibitors of cancer metabolism.

“Chemotherapeutic agent” includes chemical compounds useful in the treatment of cancer. Examples of chemotherapeutic agents include erlotinib (TARCEVA®, Genentech/OSI Pharm.), bortezomib (VELCADE®, Millennium Pharm.), disulfiram, epigallocatechin gallate, salinosporamide A, carfilzomib, 17-AAG (geldanamycin), radicicol, lactate dehydrogenase A (LDH-A), fulvestrant (FASLODEX®, AstraZeneca), sunitib (SUTENT®, Pfizer/Sugen), letrozole (FEMARA®, Novartis), imatinib mesylate (GLEEVEC®, Novartis), finasunate (VATALANIB®, Novartis), oxaliplatin (ELOXATIN®, Sanofi), 5-FU (5-fluorouracil), leucovorin, Rapamycin (Sirolimus, RAPAMUNE®, Wyeth), Lapatinib (TYKERB®, GSK572016, Glaxo Smith Kline), Lonafamib (SCH 66336), sorafenib (NEXAVAR®, Bayer Labs), gefitinib (IRESSA®, AstraZeneca), AG1478, alkylating agents such as thiotepa and CYTOXAN® cyclosphosphamide; alkyl sulfonates such as busulfan, improsulfan and piposulfan; Eiziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin I and cryptophycin 8); adrenocorticosteroids (including prednisone and prednisolone); cyproterone acetate; 5a-reductases including finasteride and dutasteride); vorinostat, romidepsin, panobinostat, valproic acid, mocetinostat dolastatin; aldesleukin, talc duocarmycin (including the synthetic analogs, KW-2189 and CBI-TM I); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin γ\hat{ι}I and calicheamicin coll (Angew Chem. Inti. Ed. Engl. 1994 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, 111.), and TAXOTERE® (docetaxel, doxetaxel; Sanofi-Aventis); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-I I; topoisomerase inhibitor RFS 2000; difluoromethylomithine (DMFO); retinoids such as retinoic acid; and pharmaceutically acceptable salts, acids and derivatives of any of the above.

Chemotherapeutic agent also includes (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY1 17018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN®, rIL-2; a topoisomerase I inhibitor such as LURTOTECAN®; ABARELIX® rmRH; and (ix) pharmaceutically acceptable salts, acids and derivatives of any of the above.

Chemotherapeutic agent also includes antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idee), pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth). Additional humanized monoclonal antibodies with therapeutic potential as agents in combination with the compounds of the invention include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695, Wyeth Research and Abbott Laboratories) which is a recombinant exclusively human-sequence, full-length IgGi λ antibody genetically modified to recognize interleukin-12 p40 protein.

Chemotherapeutic agent also includes “EGFR inhibitors,” which refers to compounds that bind to or otherwise interact directly with EGFR and prevent or reduce its signaling activity, and is alternatively referred to as an “EGFR antagonist.” Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C₂₂₅ or Cetuximab; ERBUTIX®) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see WO98/50433, Abgenix/Amgen); EMD 55900 (Stragliotto et al. Eur. J. Cancer 32A:636-640 (1996)); EMD7200 (matuzumab) a humanized EGFR antibody directed against EGFR that competes with both EGF and TGF-alpha for EGFR binding (EMD/Merck); human EGFR antibody, HuMax-EGFR (GenMab); fully human antibodies known as ElI, E2.4, E2.5, E6.2, E6.4, E2.11, E6.3 and E7.6.3 and described in U.S. Pat. No. 6,235,883; MDX-447 (Medarex Inc); and mAb 806 or humanized mAb 806 (Johns et al, J. Biol. Chem. 279(29):30375-30384 (2004)). The anti-EGFR antibody may be conjugated with a cytotoxic agent, thus generating an immunoconjugate (see, e.g., EP659,439A2, Merck Patent GmbH). EGFR antagonists include small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037. Particular small molecule EGFRantagonists include OSI-774 (CP-358774, erlotinib, TARCEVA® Genentech/OSI Pharmaceuticals); PD 183805 (Cl 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(I-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); dual EGFR/HER2 tyrosine kinase inhibitors such as lapatinib (TYKERB®, GSK572016 or N-[3-chloro-4-[(3 fluorophenyl)methoxy]phenyl]-6[5[[[2methylsulfonyl)ethyl]amino]methyl]-2-furanyl]-4-quinazolinamine).

Chemotherapeutic agents also include “tyrosine kinase inhibitors” including the EGFR-targeted drugs noted in the preceding paragraph; small molecule FIER2 tyrosine kinase inhibitor such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GSK572016; available from Glaxo-SmithKline), an oral HER2 and EGFR tyrosine kinase inhibitor; PKI-166 (available from Novartis); pan-HER inhibitors such as canertinib (CI-1033; Pharmacia); Raf-I inhibitors such as antisense agent ISIS-5132 available from ISIS Pharmaceuticals which inhibit Raf-I signaling; non-HER targeted TK inhibitors such as imatinib mesylate (GLEEVEC®, available from Glaxo SmithKline); multi-targeted tyrosine kinase inhibitors such as sunitinib (SUTENT®, available from Pfizer); VEGF receptor tyrosine kinase inhibitors such as vatalanib (PTK787/ZK222584, available from Novartis/Schering AG); MAPK extracellular regulated kinase I inhibitor Cl-1040 (available from Pharmacia); quinazolines, such as PD 153035, 4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d]pyrimidines; curcumin (diferuloyl methane, 4,5-bis(4-fluoroanilino)phthalimide); tyrphostines containing nitrothiophene moieties; PD-0183805 (Wamer-Lamber); antisense molecules (e.g. those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); imatinib mesylate (GLEEVEC®); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-ICl I (Imclone), rapamycin (sirolimus, RAPAMUNE®); or as described in any of the following patent publications: U.S. Pat. No. 5,804,396; WO 1999/09016 (American Cyanamid); WO 1998/43960 (American Cyanamid); WO 1997/38983 (Warner Lambert); WO 1999/06378 (Warner Lambert); WO 1999/06396 (Warner Lambert); WO 1996/30347 (Pfizer, Inc); WO 1996/33978 (Zeneca); WO 1996/3397 (Zeneca) and WO 1996/33980 (Zeneca).

Chemotherapeutic agents also include dexamethasone, interferons, colchicine, metoprine, cyclosporine, amphotericin, metronidazole, alemtuzumab, alitretinoin, allopurinol, amifostine, arsenic trioxide, asparaginase, BCG live, bevacuzimab, bexarotene, cladribine, clofarabine, darbepoetin alfa, denileukin, dexrazoxane, epoetin alfa, elotinib, filgrastim, histrelin acetate, ibritumomab, interferon alfa-2a, interferon alfa-2b, lenalidomide, levamisole, mesna, methoxsalen, nandrolone, nelarabine, nofetumomab, oprelvekin, palifermin, pamidronate, pegademase, pegaspargase, pegfilgrastim, pemetrexed disodium, plicamycin, porfimer sodium, quinacrine, rasburicase, sargramostim, temozolomide, VM-26, 6-TG, toremifene, tretinoin, ATRA, valrubicin, zoledronate, and zoledronic acid, and pharmaceutically acceptable salts thereof.

Chemotherapeutic agents also include hydrocortisone, hydrocortisone acetate, cortisone acetate, tixocortol pivalate, triamcinolone acetonide, triamcinolone alcohol, mometasone, amcinonide, budesonide, desonide, fluocinonide, fluocinolone acetonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate and fluprednidene acetate; immune selective anti-inflammatory peptides (ImSAIDs) such as phenylalanine-glutamine-glycine (PEG) and its D-isomeric form (feG) (IMULAN BioTherapeutics, LLC); anti-rheumatic drugs such as azathioprine, ciclosporin (cyclosporine A), D-penicillamine, gold salts, hydroxychloroquine, leflunomideminocycline, sulfasalazine, tumor necrosis factor alpha (TNFa) blockers such as etanercept (Enbrel), infliximab (Remicade), adalimumab (Humira), certolizumab pegol (Cimzia), golimumab (Simponi), Interleukin I (IL-I) blockers such as anakinra (Kineret), T cell costimulation blockers such as abatacept (Orencia), Interleukin 6 (IL-6) blockers such as tocilizumab (ACTEMERA®); Interleukin 13 (IL-13) blockers such as lebrikizumab; Interferon alpha (IFN) blockers such as Rontalizumab; Beta 7 integrin blockers such as rhuMAb Beta7; IgE pathway blockers such as Anti-Ml prime; Secreted homotrimeric LTa3 and membrane bound heterotrimer LTa I/β2 blockers such as Anti-lymphotoxin alpha (LTa); radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); miscellaneous investigational agents such as thioplatin, PS-341, phenylbutyrate, ET-18-OCH3, or famesyl transferase inhibitors (L-739749, L-744832); polyphenols such as quercetin, resveratrol, piceatannol, epigallocatechine gallate, theaflavins, flavanols, procyanidins, betulinic acid and derivatives thereof; autophagy inhibitors such as chloroquine; delta-9-tetrahydrocannabinol (dronabinol, MARINOL®); beta-lapachone; lapachol; colchicines; betulinic acid; acetylcamptothecin, scopolectin, and 9-aminocamptothecin); podophyllotoxin; tegafur (UFTORAL®); bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTQNEL®); and epidermal growth factor receptor (EGF-R); vaccines such as THERATOPE® vaccine; perifosine, COX-2 inhibitor (e.g. celecoxib or etoricoxib), proteosome inhibitor (e.g. PS341); CCI-779; tipifamib (RI 1577); orafenib, ABT510; Bcl-2 inhibitor such as oblimersen sodium (GENASENSE®); pixantrone; famesyltransferase inhibitors such as lonafamib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

### Pharmaceutical Compositions and Administration

The compounds of formula I and the pharmaceutically acceptable salts can be used as therapeutically active substances, e.g. in the form of pharmaceutical preparations. The pharmaceutical preparations can be administered orally, e.g. in the form of tablets, coated tablets, dragées, hard and soft gelatin capsules, solutions, emulsions or suspensions. The administration can, however, also be effected rectally, e.g. in the form of suppositories, or parenterally, e.g. in the form of injection solutions.

The compounds of formula I and the pharmaceutically acceptable salts thereof can be processed with pharmaceutically inert, inorganic or organic carriers for the production of pharmaceutical preparations. Lactose, corn starch or derivatives thereof, talc, stearic acids or its salts and the like can be used, for example, as such carriers for tablets, coated tablets, dragées and hard gelatin capsules. Suitable carriers for soft gelatin capsules are, for example, vegetable oils, waxes, fats, semi-solid and liquid polyols and the like. Depending on the nature of the active substance no carriers are however usually required in the case of soft gelatin capsules. Suitable carriers for the production of solutions and syrups are, for example, water, polyols, glycerol, vegetable oil and the like. Suitable carriers for suppositories are, for example, natural or hardened oils, waxes, fats, semi-liquid or liquid polyols and the like.

The pharmaceutical preparations can, moreover, contain pharmaceutically acceptable auxiliary substances such as preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances.

Medicaments containing a compound of formula I or a pharmaceutically acceptable salt thereof and a therapeutically inert carrier are also provided by the present invention, as is a process for their production, which comprises bringing one or more compounds of formula I and/or pharmaceutically acceptable salts thereof and, if desired, one or more other therapeutically valuable substances into a galenical administration form together with one or more therapeutically inert carriers.

The dosage can vary within wide limits and will, of course, have to be adjusted to the individual requirements in each particular case. In the case of oral administration, the dosage for adults can vary from about 0.01 mg to about 1000 mg per day of a compound of general formula I or of the corresponding amount of a pharmaceutically acceptable salt thereof. The daily dosage may be administered as single dose or in divided doses and, in addition, the upper limit can also be exceeded when this is found to be indicated.

The following examples illustrate the present invention without limiting it, but serve merely as representative thereof. The pharmaceutical preparations conveniently contain about 1-500 mg, particularly 1-100 mg, of a compound of formula I. Examples of compositions according to the invention are:

### Example A

Tablets of the following composition are manufactured in the usual manner:

TABLE 2
possible tablet composition

mg/tablet

| ingredient | 5 | 25 | 100 | 500 |
|---|---|---|---|---|
| Compound of formula I | 5 | 25 | 100 | 500 |
| Lactose Anhydrous DTG | 125 | 105 | 30 | 150 |
| Sta-Rx 1500 | 6 | 6 | 6 | 60 |
| Microcrystalline Cellulose | 30 | 30 | 30 | 450 |
| Magnesium Stearate | 1 | 1 | 1 | 1 |
| Total | 167 | 167 | 167 | 831 |

### Manufacturing Procedure

1. Mix ingredients 1, 2, 3 and 4 and granulate with purified water.
2. Dry the granules at 50° C.
3. Pass the granules through suitable milling equipment.
4. Add ingredient 5 and mix for three minutes; compress on a suitable press.

### Example B-1

Capsules of the following composition are manufactured:

TABLE 3
possible capsule ingredient composition

mg/capsule

| ingredient | 5 | 25 | 100 | 500 |
|---|---|---|---|---|
| Compound of formula I | 5 | 25 | 100 | 500 |
| Hydrous Lactose | 159 | 123 | 148 | — |
| Corn Starch | 25 | 35 | 40 | 70 |
| Talk | 10 | 15 | 10 | 25 |
| Magnesium Stearate | 1 | 2 | 2 | 5 |
| Total | 200 | 200 | 300 | 600 |

### Manufacturing Procedure

1. Mix ingredients 1, 2 and 3 in a suitable mixer for 30 minutes.
2. Add ingredients 4 and 5 and mix for 3 minutes.
3. Fill into a suitable capsule.

The compound of formula I, lactose and corn starch are firstly mixed in a mixer and then in a comminuting machine. The mixture is returned to the mixer; the talc is added thereto and mixed thoroughly. The mixture is filled by machine into suitable capsules, e.g. hard gelatin capsules.

### Example B-2

Soft Gelatin Capsules of the following composition are manufactured:

TABLE 4
possible soft gelatin capsule ingredient composition

| ingredient | mg/capsule |
|---|---|
| Compound of formula I | 5 |
| Yellow wax | 8 |
| Hydrogenated Soy bean oil | 8 |
| Partially hydrogenated plant oils | 34 |
| Soy bean oil | 110 |
| Total | 165 |

TABLE 5
possible soft gelatin capsule composition

| ingredient | mg/capsule |
|---|---|
| Gelatin | 75 |
| Glycerol 85% | 32 |
| Karion 83 | 8 (dry matter) |
| Titan dioxide | 0.4 |
| Iron oxide yellow | 1.1 |
| Total | 116.5 |

### Manufacturing Procedure

The compound of formula I is dissolved in a warm melting of the other ingredients and the mixture is filled into soft gelatin capsules of appropriate size. The filled soft gelatin capsules are treated according to the usual procedures.

### Example C

Suppositories of the following composition are manufactured:

TABLE 6
possible suppository composition

| ingredient | mg/supp. |
|---|---|
| Compound of formula I | 15 |
| Suppository mass | 1285 |
| Total | 1300 |

### Manufacturing Procedure

The suppository mass is melted in a glass or steel vessel, mixed thoroughly and cooled to 45° C. Thereupon, the finely powdered compound of formula I is added thereto and stirred until it has dispersed completely. The mixture is poured into suppository moulds of suitable size, left to cool; the suppositories are then removed from the moulds and packed individually in wax paper or metal foil.

### Example D

Injection solutions of the following composition are manufactured:

TABLE 7
possible injection solution composition

| ingredient | mg/injection solution. |
|---|---|
| Compound of formula I | 3 |
| Polyethylene Glycol 400 | 150 |
| acetic acid | q.s. ad pH 5.0 |
| water for injection solutions | ad 1.0 ml |

### Manufacturing Procedure

The compound of formula I is dissolved in a mixture of Polyethylene Glycol 400 and water for injection (part). The pH is adjusted to 5.0 by acetic acid. The volume is adjusted to 1.0 ml by addition of the residual amount of water. The solution is filtered, filled into vials using an appropriate overage and sterilized.

### Example E

Sachets of the following composition are manufactured:

TABLE 8
possible sachet composition

| ingredient | mg/sachet |
|---|---|
| Compound of formula I | 50 |
| Lactose, fine powder | 1015 |
| Microcrystalline cellulose (AVICEL PH 102) | 1400 |
| Sodium carboxymethyl cellulose | 14 |
| Polyvinylpyrrolidon K 30 | 10 |
| Magnesium stearate | 10 |
| Flavoring additives | 1 |
| Total | 2500 |

### Manufacturing Procedure

The compound of formula I is mixed with lactose, microcrystalline cellulose and sodium carboxymethyl cellulose and granulated with a mixture of polyvinylpyrrolidone in water. The granulate is mixed with magnesium stearate and the flavoring additives and filled into sachets.

### EXAMPLES

The invention will be more fully understood by reference to the following examples. The claims should not, however, be construed as limited to the scope of the examples.

In case the preparative examples are obtained as a mixture of enantiomers, the pure enantiomers can be separated by methods described herein or by methods known to the man skilled in the art, such as e.g., chiral chromatography (e.g., chiral SFC or chiral HPLC) or crystallization.

All reaction examples and intermediates were prepared under a nitrogen atmosphere if not specified otherwise.

### SMARCA2 HiBiT and SMARCA4 Degradation Assay (Cellular)

### Generation of HT1080 Cell Lines Stably Expressing SMARCA2 HiBiT or SMARC4 HiBiT

For quantitative cellular degradation of the target protein degradation mediated by the bifunctional degraders described here, HiBiT was appendant to the gene sequence of the targeted proteins, SMARCA2 or SMARCA4, in HT1080 parental cell line using CRISPR-mediate HiBiT tagging technology, as described by Promega.

RNP Complex Assembly and Delivery. RNA Complexes were assembled and delivered by electroporation into cells, as previously described. Briefly, 16 g (100 pmol) Cas9 and 10.8 g of sgRNA were incubated for 10-15 minutes at room temperature. Cells were resuspended in 20 L of SF 4D-nucleofector solution (Amaxa SF cell line4D Nucleofector X kit (Lonza, V4XC-2032). RNP complex and 16.6 pmol of DNA oligo were the electroporated into cells using FF-113 program (Amaxa 4D Nucleofector). Following electroporation, cells were incubated at room temperature for 5 minutes and then transferred to a six-well plate for culturing. At 24-48 h postelectroporation, cells were analyzed for insertion with Nano-Glo® HiBiT Lytic Detection System.

Lytic HiBiT Detection. Nano-Glo® HiBiT Lytic Detection System was used to assess luminescence for each guide RNA tested (ACS Chem. Biol. 2018, 13, 467-474). Unedited cells were used as negative control for background. Following successful detection of the HiBiT luminescence signal in the pool, the pool of cells was subjected for single cell sorting (SH800S Cell Sorter, Sony Biotechnology). Only clones that gave a highest HiBiT luminescence signal were further expanded in cell culture and were used in the SMARCA2 HiBiT and SMARCA4 HiBiT degradation assay (cellular).

### 1.1 Materials

SMARCA2 HiBiT and SMARCA4 HiBiT HT1080 cell lines were generated in house as described herein. HT1080 parental cell line, as well as SMARCA2 HiBiT HT1080 and SMARCA4 HiBiT HT1080 cell lines were routinely cultured in the following medium: Earle's MEM (Gibco, #41090) containing 10% serum (VWR, #97068-085) and only up to passage 20. For the assay, SMARCA2 HiBiT HT1080 and SMARCA4 HiBiT HT1080 cells are plated for treatment in Earle's MEM (Gibco, #51200) containing 10% serum (VWR, #97068-085) and 1× Glutamax (Gibco, #35050-038). Assay plates used were Corning® 384-well Flat Clear Bottom White Polystyrene TC-treated Microplates (Corning #3765). Cells for lysed in Nano-Glo® HiBiT Lytic Reagent, Nano-Glo® HiBiT Lytic Detection System, Promega, (#N3050).

1.3 SMARCA2 HiBiT and SMARCA4 HiBiT Degradation Assay (Cellular)

Briefly, a day before compound treatment, cells were seeded onto 384-well plate at the density of 1500 cell/well in Earle's MEM (Gibco, #51200) containing 10% serum (VWR, #97068-085) and 1× Glutamax (Gibco, #35050-038). The following day, test compounds were added to the 384-well plate from a top concentration of 10 μM with 11 points, half log titration in duplicates. Additionally, the negative control cells were treated with vehicle alone. The plates were incubated at 37° C. with 5% CO₂ for duration of the assay (6 hours or 16 hours). After the desired incubation time, cells were lyased by addition of Nano-Glo® HiBiT Lytic Reagent (prepared according the manufacture recommendations and added to the cells in ratio 1:1, v/v). Microplates were agitated on plate shaker at 400 rpm for 2 minutes, and incubated for another 10 min in dark at room temperature. A white light-reflecting film was applied to the bottom of the 384 well plates before reading. Finally, luminescence signal was acquired on with PHERAstar® FSX plate reader (BMG Labtech, Germany).

Quantification of luminescence responses measured in the presence of compound were normalized to a high signal/no degradation control (untreated cells+lytic detection reagent) and a low signal/full degradation control (untreated cells, no lytic detection reagent). Data were analyzed with a 4-parameter logistic fit to generate sigmoidal dose-response curves. The DC₅₀ is the concentration of compound at which exactly 50% of the total cellular SMARCA2 or SMARCA4 has been degraded. The Emax, or maximum effect of each compound, represents the amount of residual protein remaining in the cell following compound treatment.

TABLE 1

| | HiBit A549 | HiBit A549 |
|---|---|---|
| | SMARCA2 | SMARCA2 |
| Ex. | IP (nM) | Emax (%) |
| 1 | 2778 | 52 |
| 2 | 478 | 38 |
| 3 | 479 | 51 |
| 4 | 2927 | 36 |
| 5 | 2515 | 31 |
| 6 | 2911 | 54 |
| 7 | 357 | 39 |
| 8 | 610 | 48 |
| 9 | 733 | 58 |
| 10 | 286 | 19 |
| 11 | 104 | 20 |
| 12 | 2460 | 43 |
| 13 | 954 | 28 |
| 14 | 995 | 38 |
| 15 | 487 | 24 |
| 16 | 872 | 33 |
| 17 | 2824 | 50 |
| 18 | 848 | 63 |
| 19 | 754 | 18 |
| 20 | 313 | 33 |
| 21 | 1005 | 19 |
| 22 | 455 | 27 |
| 23 | 428 | 38 |
| 24 | 2850 | 42 |
| 25 | 348 | 19 |
| 26 | 1435 | 60 |
| 27 | 330 | 33 |
| 28 | 515 | 46 |
| 29 | 1109 | 30 |
| 30 | 1157 | 65 |
| 31 | 1921 | 30 |
| 32 | 119 | 20 |
| 33 | 73 | 22 |
| 34 | 1105 | 47 |
| 35 | 952 | 39 |
| 36 | 838 | 49 |
| 37 | 73 | 29 |
| 38 | 589 | 70 |
| 39 | 426 | 48 |
| 40 | 349 | 46 |
| 41 | 204 | 63 |
| 42 | 561 | 71 |
| 43 | 749 | 23 |
| 44 | 205 | 21 |
| 45 | 120 | 33 |
| 46 | 2605 | 51 |
| 47 | 733 | 55 |
| 48 | 135 | 22 |
| 49 | 1661 | 60 |
| 50 | 2150 | 63 |
| 51 | 1646 | 48 |
| 52 | 533 | 56 |
| 53 | 240 | 44 |
| 54 | 503 | 35 |
| 55 | 2581 | 27 |
| 56 | 91 | 20 |
| 57 | 736 | 35 |
| 58 | 197 | 22 |
| 59 | 305 | 23 |
| 60 | 300 | 24 |
| 61 | 519 | 59 |
| 62 | 2606 | 54 |
| 63 | 798 | 48 |
| 64 | 1265 | 31 |
| 65 | 1034 | 24 |
| 66 | 2024 | 49 |
| 67 | 839 | 29 |
| 68 | 2316 | 50 |
| 69 | 2113 | 60 |
| 70 | 1177 | 50 |
| 71 | 522 | 38 |
| 72 | 421 | 35 |
| 73 | 1652 | 29 |
| 74 | 575 | 63 |
| 75 | 777 | 26 |
| 76 | 207 | 31 |
| 77 | 244 | 24 |
| 78 | 63 | 20 |
| 79 | 78 | 21 |
| 80 | 383 | 25 |
| 81 | 504 | 34 |
| 82 | 586 | 62 |
| 83 | 789 | 44 |
| 84 | 902 | 38 |
| 85 | 2187 | 31 |
| 86 | 223 | 25 |
| 87 | 1299 | 51 |
| 88 | 423 | 29 |
| 89 | 1011 | 56 |
| 90 | 1321 | 34 |
| 91 | 600 | 44 |
| 92 | 560 | 45 |
| 93 | 579 | 55 |
| 94 | 693 | 43 |
| 95 | 2444 | 50 |
| 96 | 200 | 28 |
| 97 | 1149 | 40 |
| 98 | 1654 | 28 |
| 99 | 827 | 59 |
| 100 | 998 | 62 |
| 101 | 101 | 38 |
| 102 | 992 | 59 |
| 103 | 1895 | 60 |
| 104 | 375 | 54 |
| 105 | 923 | 40 |
| 106 | 346 | 39 |
| 107 | 82^{a} | 18^{a} |
| 108 | 68^{a} | 7.9^{a} |
| 109 | 285^{a} | 23^{a} |
| 110 | 760 | 76 |

^{a}different HIBIT SMARCA2 clone

### Compound Syntheses

### Ligase 1a: tert-butyl (2S,4R)-2-[(4-bromophenyl)methylcarbamoyl]-4-hydroxy-pyrrolidine-1-carboxylate

To a solution of N-Boc-4-hydroxy-L-proline (60 g, 259.46 mmol, 1.0 eq), N,N-diisopropylethylamine (135.58 mL, 778.38 mmol, 3.0 eq) in DMF (500 mL) was added O-(7-azabenzotriazol-1-yl)-N,N,N′,N′-tetramethyluronium hexafluorophosphate (118.39 g, 311.35 mmol, 1.2 eq) at 25° C. After stirring for 0.5 h, 4-bromobenzylamine (53.1 g, 285.41 mmol, 1.1 eq) was added to the mixture at 0° C. Then the mixture was stirred at 25° C. for 0.5 h. The mixture was diluted with water and extracted with ethyl acetate. The combined organic phase was washed with saturated sodium bicarbonate, water and brine, then dried over anhydrous sodium sulfate and evaporated to dryness. The product was triturated in methyl tert-butyl ether (500 mL) to afford the title compound (80 g, 201 mmol, 77% yield) as a yellow solid. MS (ESI): 421.0 ([M+Na]+).

### Ligase 1b: tert-butyl (2S,4R)-4-hydroxy-2-[[4-(4-methylthiazol-5-yl)phenyl]methylcarbamoyl]pyrrolidine-1-carboxylate

A mixture of Ligase 1a (40 g, 100.18 mmol, 1.0 eq), 4-methylthiazole (39.7 g, 400.72 mmol, 4.0 eq), palladium (II) acetate (1.1 g, 5.01 mmol, 0.05 eq) and potassium acetate (39.3 g, 400.72 mmol, 4.0 eq) in NMP (200 mL) was stirred under N₂ atmosphere at 120° C. for 16 h. After cooling to ambient temperature, water was added and the product was extracted with ethyl acetate. The combined organic phase was washed with brine, dried over magnesium sulfate and evaporated to dryness. The residue was purified to afford the title compound (40 g, 96 mmol, 95% yield). MS (ESI): 418.1 ([M+H]⁺).

### Ligase 1c: (2S,4R)-4-hydroxy-N-[[4-(4-methylthiazol-5-yl)phenyl]methyl]pyrrolidine-2-carboxamide

To a solution of Ligase 1b (40.0 g, 95.8 mmol, 1.0 eq) in methanol (100 mL) and DCM (100 mL) was added 4N hydrochloric acid in 1,4-dioxane (200 mL). The mixture was stirred at room temperature for 2 h. The mixture was concentrated in vacuum to give a white solid, which was triturated in DCM to afford the title compound as the hydrochloric salt (23 g, 72 mmol, 75% yield) as a white solid. MS (ESI): 318.1 ([M+H]⁺).

### Ligase 1d: tert-butyl ((S)-1-((2S,4R)-4-hydroxy-2-((4-(4-methylthiazol-5-yl)benzyl)carbamoyl) pyrrolidin-1-yl)-3,3-dimethyl-1-oxobutan-2-yl)carbamate

To a solution of (S)-2-((tert-butoxycarbonyl)amino)-3,3-dimethylbutanoic acid (13 g, 56.21 mmol, 1.0 eq), DIPEA (29.37 mL, 168.62 mmol, 3.0 eq) in DMF (150 mL) was added HATU (25.65 g, 67.45 mmol, 1.2 eq) at 0° C. After stirring for 0.5 h, Ligase 1c (17.84 g, 56.21 mmol, 1.0 eq) was added to the mixture and it was stirred at 25° C. for 1.5 h. The mixture was poured into water, extracted with ethyl acetate and washed with brine. The organic phase was concentrated in vacuum to afford the title compound (20 g, 37 mmol, 65% yield) as a yellow oil. MS (ESI): 531.5 ([M+H]⁺).

### Ligase 1: (2S,4R)-1-((S)-2-amino-3,3-dimethylbutanoyl)-4-hydroxy-N-(4-(4-methylthiazol-5-yl)benzyl)pyrrolidine-2-carboxamide

To a solution of Ligase 1d (15 g, 28.27 mmol, 1.0 eq) in methanol (100 mL) was added HCl in 1,4-dioxane (50 mL, 4N). The mixture was stirred at ambient temperature for 2 h. The mixture was concentrated and purified by prep-HPLC to afford the title compound (13 g, 27.8 mmol, 78% yield) as a light yellow oil, hydrochloride salt. MS (ESI): 431.3 ([M+H]⁺).

### Ligase 2a: methyl 8-[[(1R)-1-[(2S,4R)-4-hydroxy-2-[[4-(4-methylthiazol-5-yl)phenyl]methylcarbamoyl]pyrrolidine-1-carbonyl]-2,2-dimethyl-propyl]amino]-8-oxo-octanoate

To a solution of Ligase 1 hydrochloride salt (400 mg, 0.860 mmol, 1.0 eq), triethylamine (0.48 mL, 3.43 mmol, 4.0 eq) and suberic acid monomethyl ester (193.45 mg, 1.03 mmol, 1.2 eq) in DMF (10 mL) was added 1-propanephosphonic anhydride (1.09 g, 1.71 mmol, 2.0 eq) and it was stirred at 25° C. for 2 h. The mixture was poured into water, then extracted with ethyl acetate. The organic phase was concentrated in vacuum to afford the title compound (100 mg, 0.17 mmol, 16% yield). MS (ESI): 601.4 ([M+H]⁺).

### Ligase 2: 8-[[(1R)-1-[(2S,4R)-4-hydroxy-2-[[4-(4-methylthiazol-5-yl)phenyl]methylcarbamoyl]pyrrolidine-1-carbonyl]-2,2-dimethyl-propyl]amino]-8-oxo-octanoic acid

A solution of Ligase 2a (100 mg, 0.170 mmol, 1.0 eq) and lithium hydroxide (10 mg, 0.420 mmol, 2.5 eq) in THF/H₂O (20 mL, 1:1) was stirred at 25° C. for 2 h. The mixture was poured into HCl solution (0.5N), extracted with ethyl acetate. The combined organic phase was concentrated in vacuum to afford the title compound (60 mg, 0.10 mmol, 58% yield). MS (ESI): 587.4 ([M+H]⁺).

### Ligase 3: 7-[[(1R)-1-[(2S,4R)-4-hydroxy-2-[[4-(4-methylthiazol-5-yl)phenyl]methylcarbamoyl]pyrrolidine-1-carbonyl]-2,2-dimethyl-propyl]amino]-7-oxo-heptanoic acid

A solution of Ligase 1 hydrochloride (500 mg, 1.07 mmol, 1.0 eq), N,N-diisopropylethylamine (0.28 mL, 1.61 mmol, 1.5 eq), 0-(7-azabenzotriazol-1-yl)-N,N,N′,N′-tetramethyluronium hexafluorophosphate (407 mg, 1.07 mmol, 1.0 eq) and pimelic acid (343 mg, 2.14 mmol, 2.0 eq) in DMF (10 mL) was stirred at 25° C. for 2 h. The mixture was poured into water, then extracted with ethyl acetate. The combined organic phase was concentrated in vacuum to afford the title compound (50 mg, 0.09 mmol, 2.4% yield). MS (ESI): 573.3 ([M+H]⁺).

### Ligase 4: 10-[[(1R)-1-[(2S,4R)-4-hydroxy-2-[[4-(4-methylthiazol-5-yl)phenyl]methylcarbamoyl]pyrrolidine-1-carbonyl]-2,2-dimethyl-propyl]amino]-10-oxo-decanoic acid

The title compound (50 mg, 0.080 mmol, 7% yield) was prepared in analogy to Ligase 3 using sebacic acid. MS (ESI): 615.4 ([M+H]⁺).

### Ligase 5: 11-[[(1R)-1-[(2S,4R)-4-hydroxy-2-[[4-(4-methylthiazol-5-yl)phenyl]methylcarbamoyl]pyrrolidine-1-carbonyl]-2,2-dimethyl-propyl]amino]-11-oxo-undecanoic acid

The title compound (50 mg, 0.080 mmol, 2.4% yield) was prepared in analogy to Ligase 3 using undecanedioic acid. MS (ESI): 615.4 ([M+H]⁺).

### Ligase 6a: 2-hydroxy-4-(4-methylthiazol-5-yl)benzonitrile

A mixture of palladium (II) acetate (1.3 g, 5.81 mmol, 0.05 eq), 2-bromo-4-hydroxy-benzonitrile (23 g, 116.15 mmol, 1.0 eq), 4-methylthiazole (23 g, 232.3 mmol, 2.0 eq), potassium acetate (14.52 mL, 232.3 mmol, 2.0 eq) in 1-methyl-2-pyrrolidinone (300 mL) was stirred at 110° C. for 3 h. The mixture was poured into water then extracted with ethyl acetate. The combined organic phase was concentrated in vacuum, then purified by silica gel column to afford the title compound (20 g, 92.48 mmol, 46% yield) as a yellow oil. MS (ESI): 217.2 ([M+H]⁺).

### Ligase 6b: 2-(aminomethyl)-5-(4-methylthiazol-5-yl)phenol

A mixture of lithium aluminum hydride (9.48 g, 249.7 mmol, 3.0 eq) and Ligase 6a (18 g, 83.23 mmol, 1.0 eq) in THF (400 mL) was stirred at 0° C. for 3 h. To the mixture was added water (10 mL), followed by 15% NaOH (10 mL) and water again (30 mL). The mixture was filtered and concentrated in vacuum to afford the title compound (15 g, 68.0 mmol, 73% yield) as a red oil. MS (ESI): 221.1 ([M+H]⁺).

### Ligase 6c: tert-butyl (2S,4R)-4-hydroxy-2-[[2-hydroxy-4-(4-methylthiazol-5-yl)phenyl]methyl carbamoyl]pyrrolidine-1-carboxylate

A mixture of O-(7-azabenzotriazol-1-yl)-N,N,N′,N′-tetramethyluronium hexafluorophosphate (7.78 g, 20.47 mmol, 1.1 eq), Ligase 6b (4.1 g, 18.61 mmol, 1.0 eq), Boc-Hyp-OH (4.3 g, 18.61 mmol, 1.0 eq), N,N-diisopropylethylamine (7.2 g, 55.84 mmol, 3.0 eq) in DMF (40 mL) was stirred at 25° C. for 1 h. The mixture was concentrated in vacuum, then purified by flash chromatography to afford the title compound (3 g, 6.9 mmol, 13% yield) as a yellow oil. MS (ESI): 434.3 ([M+H]⁺).

### Ligase 6d: (2S,4R)-4-hydroxy-N-[[2-hydroxy-4-(4-methylthiazol-5-yl)phenyl]methyl]pyrrolidine-2-carboxamide

A mixture of Ligase 6c (100 mg, 0.230 mmol, 1.0 eq) and 4 M hydrochloride in dioxane (5.0 mL, 20 mmol, 86.7 eq) in DCM (10 mL) was stirred at 25° C. for 1 h. Then the mixture was concentrated in vacuum to afford the title compound (20 mg, 0.060 mmol, 26% yield). MS (ESI): 334.3 ([M+H]⁺).

### Ligase 6e: (2S)-3-methyl-2-(1-oxoisoindolin-2-yl)butanoic acid

A mixture of L-valine (4.37 g, 37.28 mmol, 1.0 eq), O-phthalaldehyde (5.0 g, 37.28 mmol, 1.0 eq) in MeCN (300 mL) was stirred at 80° C. for 5 h. After 5 h, a white solid was precipitated from mother liquid, so the mixture was filtered to afford the title compound (2 g, 8.5 mmol, 23% yield) as a yellow solid.

### Ligase 6f: (2S,4R)-4-hydroxy-N-[[2-hydroxy-4-(4-methylthiazol-5-yl)phenyl]methyl]-1-[(2S)-3-methyl-2-(1-oxoisoindolin-2-yl)butanoyl]pyrrolidine-2-carboxamide

A mixture of Ligase 6e (49 mg, 0.210 mmol, 1.0 eq), Ligase 6d (70 mg, 0.210 mmol, 1.0 eq), 0-(7-azabenzotriazol-1-yl)-N,N,N′,N′-tetramethyluronium hexafluorophosphate (96 mg, 0.250 mmol, 1.2 eq), and N,N-diisopropylethylamine (81 mg, 0.630 mmol, 3.0 eq) in DMF (5 mL) was stirred at 25° C. for 2 h. Then the mixture was concentrated in vacuum, the residue was purified by flash chromatography to afford the title compound (60 mg, 0.110 mmol, 52% yield) as a yellow solid. MS (ESI): 549.4 ([M+H]⁺).

### Ligase 6g: benzyl 11-hydroxyundecanoate

A mixture of 11-hydroxyundecanoic acid (500 mg, 2.47 mmol, 1.0 eq) and potassium carbonate (512 mg, 3.71 mmol, 1.5 eq) in acetonitrile (10 mL) was stirred at 50° C. for 12 h. Then the mixture was filtered and the filtrate was concentrated in vacuum. The residue was purified by flash chromatography to afford the title compound (700 mg, 2.39 mmol, 96%) as a yellow oil.

### Ligase 6h: benzyl 11-methylsulfonyloxyundecanoate

A mixture of Ligase 6g (742 mg, 2.54 mmol, 1.0 eq), methanesulfonyl chloride (0.29 mL, 3.81 mmol, 1.5 eq) and triethylamine (0.53 mL, 3.81 mmol, 1.5 eq) in DCM (30 mL) was stirred at 0° C. for 1 h. Then the mixture was concentrated in vacuum to afford the title compound (400 mg, 1.08 mmol, 42.5% yield) as a yellow oil.

### Ligase 6i: benzyl 11-[2-[[[(2S,4R)-4-hydroxy-1-[(2S)-3-methyl-2-(1-oxoisoindolin-2-yl)butanoyl]pyrrolidine-2-carbonyl]amino]methyl]-5-(4-methylthiazol-5-yl)phenoxy]undecanoate

A mixture of Ligase 6f (50 mg, 0.090 mmol, 1.0 eq), Ligase 6h (25 mg, 0.070 mmol, 0.74 eq) and potassium carbonate (25 mg, 0.180 mmol, 2.0 eq) in DMF (10 mL) was stirred at 80° C. for 12 h. Then the mixture was purified by flash chromatography to afford the title compound (50 mg, 0.06 mmol, 9.3% yield) as a yellow oil. MS (ESI): 823.5 ([M+H]⁺).

### Ligase 6: 11-[2-[[[(2S,4R)-4-hydroxy-1-[(2S)-3-methyl-2-(1-oxoisoindolin-2-yl)butanoyl]pyrrolidine-2-carbonyl]amino]methyl]-5-(4-methylthiazol-5-yl)phenoxy]undecanoic acid

A mixture of Ligase 6i (40 mg, 0.050 mmol, 1.0 eq) and lithium hydroxide (10 mg, 0.420 mmol, 8.59 eq) in THF (10 mL) was stirred at 25° C. for 12 h. Then the mixture was purified by flash chromatography to afford the title compound (30 mg, 0.040 mmol, 46% yield) as a yellow oil. MS (ESI): 733.3 ([M+H]⁺).

### Ligase 7: 11-(((S)-1-((2S,4R)-4-hydroxy-2-(((S)-1-(4-(4-methylthiazol-5-yl)phenyl)ethyl)carbamoyl) pyrrolidin-1-yl)-3-methyl-1-oxobutan-2-yl)amino)-11-oxoundecanoic acid

A mixture of undecanedioic acid (150 mg, 0.700 mmol, 1.5 eq), DIPEA (300 mg, 2.32 mmol, 5.0 eq) and HATU (212 mg, 0.560 mmol, 1.2 eq) in DMF (3 mL) was stirred at 25° C. for 0.5 h, then Ligase 1 (200 mg, 0.460 mmol, 1.0 eq) was added and the reaction mixture was stirred at 25° C. for 1.5 h. The reaction mixture was purified by prep-HPLC to afford the title compound (10 mg, 0.016 mmol, 3.4% yield) as a white solid. MS (ESI): 629.8 ([M+H]⁺).

### Ligase 8a: tert-butyl (7-(((S)-1-((2S,4R)-4-hydroxy-2-(((S)-1-(4-(4-methylthiazol-5-yl)phenyl)ethyl) carbamoyl)pyrrolidin-1-yl)-3,3-dimethyl-1-oxobutan-2-yl)amino)-7-oxoheptyl)carbamate

To a solution of 7-((tert-butoxycarbonyl)amino)heptanoic acid (331 mg, 1.35 mmol, 3.0 eq), DIPEA (290 mg, 2.25 mmol, 5.0 eq), HATU (256 mg, 0.670 mmol, 1.5 eq) in DMF (5 mL) was added Ligase 1 (200 mg, 0.450 mmol, 1.0 eq) at 25° C., the mixture was stirred at 25° C. for 12 h. The reaction mixture was purified by prep-HPLC to afford the title compound (100 mg, 0.148 mmol, 38.8% yield) as a white solid. MS (ESI): 672.4 ([M+H]⁺).

### Ligase 8: (2S,4R)-1-((S)-2-(7-aminoheptanamido)-3,3-dimethylbutanoyl)-4-hydroxy-N-((S)-1-(4-(4-methylthiazol-5-yl)phenyl)ethyl)pyrrolidine-2-carboxamide

A mixture of Ligase 8a (100 mg, 0.150 mmol, 1.0 eq) in 4 M HCl in dioxane (2.0 mL, 8 mmol, 53 eq) and DCM (10 mL) was stirred at 25° C. for 1 h. The reaction mixture was concentrated to afford the title compound (60 mg, 0.105 mmol, 70.5% yield) as a white hydrochloride salt. MS (ESI): 572.4 ([M+H]⁺).

### Ligase 9a: tert-butyl (10-(((S)-1-((2S,4R)-4-hydroxy-2-(((S)-1-(4-(4-methylthiazol-5-yl)phenyl)ethyl) carbamoyl)pyrrolidin-1-yl)-3,3-dimethyl-1-oxobutan-2-yl)amino)-10-oxodecyl)carbamate

To a solution of 10-(tert-butoxycarbonylamino)decanoic acid (78 mg, 0.270 mmol, 1.2 eq), DIPEA (145 mg, 1.12 mmol, 5.0 eq), HATU (128.29 mg, 0.340 mmol, 1.5 eq) in DMF (5 mL) was added Ligase 1 (100 mg, 0.220 mmol, 1.0 eq) at 25° C., the mixture was stirred at 25° C. for 1 h. The reaction mixture was purified by prep-HPLC to afford the title compound (60 mg, 0.084 mmol, 37% yield) as a white solid. MS (ESI): 714.2 ([M+H]⁺).

### Ligase 9: (2S,4R)-1-((S)-2-(10-aminodecanamido)-3,3-dimethylbutanoyl)-4-hydroxy-N-((S)-1-(4-(4-methylthiazol-5-yl)phenyl)ethyl)pyrrolidine-2-carboxamide

The title compound (30 mg, 0.049 mmol, 53% yield) was prepared in analogy to Ligase 8 as a white solid. MS (ESI): 614.5 ([M+H]⁺).

### Ligase 10: 9-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-9-oxononanoic acid (CAS: 2172819-76-8)

### Ligase 11: 7-oxo-7-[[(1S)-2,2-dimethyl-1-[(2S,4R)-4-hydroxy-2-[[(1S)-1-[4-(4-methylthiazol-5-yl)phenyl]ethyl]carbamoyl]pyrrolidine-1-carbonyl]propyl]amino]heptanoic acid (CAS: 2229976-21-8)

### Ligase 12: 12-oxo-12-[[(1S)-2,2-dimethyl-1-[(2S,4R)-4-hydroxy-2-[[(1S)-1-[4-(4-methylthiazol-5-yl)phenyl]methyl]carbamoyl]pyrrolidine-1-carbonyl]propyl]amino]dodecanoic acid

The title compound was prepared in analogy to Ligase 3 using dodecanedioic acid.

### Ligase 13: 14-oxo-14-[[(1S)-2,2-dimethyl-1-[(2S,4R)-4-hydroxy-2-[[(1S)-1-[4-(4-methylthiazol-5-yl)phenyl]ethyl]carbamoyl]pyrrolidine-1-carbonyl]propyl]amino]tetradecanoic acid

The title compound was prepared in analogy to Ligase 3 using tetradecanedioic acid.

### Ligase 14: 3-[2-[3-oxo-3-[[(1S)-2,2-dimethyl-1-[(2S,4R)-4-hydroxy-2-[[(1S)-1-[4-(4-methylthiazol-5-yl)phenyl]ethyl]carbamoyl]pyrrolidine-1-carbonyl]propyl]amino]propoxy]ethoxy]propanoic acid

The title compound was prepared in analogy to Ligase 3 using -[2-(2-carboxyethoxy)ethoxy]propanoic acid.

### Ligase 15: 10-oxo-10-[[(1S)-2,2-dimethyl-1-[(2S,4R)-4-hydroxy-2-[[(1S)-1-[4-(4-methylthiazol-5-yl)phenyl]ethyl]carbamoyl]pyrrolidine-1-carbonyl]propyl]amino]decanoic acid

The title compound was prepared in analogy to Ligase 3 using decanedioic acid.

### Ligase 16: 3-[3-oxo-3-[[(1S)-2,2-dimethyl-1-[(2S,4R)-4-hydroxy-2-[[(1S)-1-[4-(4-methylthiazol-5-yl)phenyl]ethyl]carbamoyl]pyrrolidine-1-carbonyl]propyl]amino]propoxy]propanoic acid

The title compound was prepared in analogy to Ligase 3 using 3-(2-carboxyethoxy)propanoic acid.

### Ligase 17: 11-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-11-oxo-undecanoic acid

The title compound was prepared in analogy to Ligase 3 using undecanedioic acid.

### Ligase 18: 4-oxo-4-[[(1S)-2,2-dimethyl-1-[(2S,4R)-4-hydroxy-2-[[(1S)-1-[4-(4-methylthiazol-5-yl)phenyl]ethyl]carbamoyl]pyrrolidine-1-carbonyl]propyl]amino]butanoic acid

The title compound was prepared in analogy to Ligase 3 using succinic acid.

### Ligase 19: 3-[2-[2-[3-[[(1S)-1-[(2S,4R)-4-hydroxy-2-[[(1SR)-1-[4-(4-methylthiazol-5-yl)phenyl]ethyl]carbamoyl]pyrrolidine-1-carbonyl]-2,2-dimethyl-propyl]amino]-3-oxo-propoxy]ethoxy]ethoxy]propanoic acid

The title compound was prepared in analogy to Ligase 3 using 3-[2-[2-(2-carboxyethoxy)ethoxy]ethoxy]propanoic acid.

### Ligase 20: 12-[[(1S)-1-[(2S,4R)-4-hydroxy-2-[[(1SR)-1-[4-(4-methylthiazol-5-yl)phenyl]ethyl]carbamoyl]pyrrolidine-1-carbonyl]-2,2-dimethyl-propyl]amino]-12-oxo-dodecanoic acid

The title compound was prepared in analogy to Ligase 3 using dodecanedioic acid.

### Ligase 21: (2S,4R)-1-((S)-3,3-dimethyl-2-(8-(piperazin-1-yl)octanamido)butanoyl)-4-hydroxy-N-(4-(4-methylthiazol-5-yl)benzyl)pyrrolidine-2-carboxamide hydrochloride

### Intermediate 2a: tert-butyl 4-[2-(3-bromophenoxy)ethyl]piperazine-1-carboxylate

To a solution of 3-bromophenol (1.5 g, 8.67 mmol, 1.0 eq), tert-butyl 4-(2-hydroxyethyl)piperazine-1-carboxylate (2.2 g, 9.54 mmol, 1.1 eq) and triphenylphosphine (2.5 g, 9.54 mmol, 1.1 eq) in THF (25 mL) was added di-tert-butyl azodicarboxylate (2.2 g, 9.54 mmol, 1.1 eq). The reaction mixture was stirred for 2 h at room temperature. The reaction mixture was poured in EtOAc and washed with H₂O and brine. The organic layer was dried over Na₂SO₄ and concentrated. The crude material was purified by flash chromatography (Heptane/EtOAc 0-50) to afford the title compound (2.93 g, 7.6 mmol, 88% yield) as a colorless oil. MS (ESI): 387.1 ([M+H]⁺).

### Intermediate 2b: 1-[2-(3-bromophenoxy)ethyl]piperazine

To a solution of Intermediate 2a (16 g, 41.53 mmol, 1.0 eq) in HCl/dioxane (50.0 mL, 41.53 mmol, 1.0 eq) was stirred at 25° C. for 2 h. The reaction mixture was concentrated. The crude product was basefied to pH=7 with NaHCO₃ solution, extracted with ethyl acetate, and the combined organic layers were washed with brine. The combined organic layers were dried over sodium sulfate, and then concentrated to afford the title compound (11 g, 38.57 mmol, 93% yield) as a light yellow oil.

### Intermediate 2c: benzyl 4-[2-(3-bromophenoxy)ethyl]piperazine-1-carboxylate

To a solution of Intermediate 2b (11 g, 38.57 mmol, 1.0 eq), triethylamine (16.13 mL, 115.72 mmol, 3.0 eq) in DCM (200 mL) was added benzyl chloroformate (7.9 g, 46.29 mmol, 1.2 eq). The reaction was stirred at 25° C. for 15 h. The mixture was diluted with water and extracted with ethyl acetate. The combined organic phase was washed with brine, dried over sodium sulfate, filtered and concentrated under reduced pressure. The residue was purified by prep-HPLC (FA) to afford the title compound (13.3 g, 31.72 mmol, 82% yield) as a yellow oil.

### Intermediate 13a: tert-butyl 4-[(3-bromophenyl)methylene]piperidine-1-carboxylate

A mixture of 3-bromobenzyl bromide (6.2 g, 24.81 mmol, 1.0 eq) and triethyl phosphite (4.68 mL, 27.29 mmol, 1.1 eq) was heated at 90° C. for 12 h, cooled to 0° C. DME (40 mL), 1-Boc-4-piperidone (5.73 g, 28.78 mmol, 1.16 eq) and sodium hydride 60% in oil (1.19 g, 29.77 mmol, 1.2 eq) were added and stirred at 20° C. for 2 h. The mixture was concentrated to remove solvent, poured into water and extracted with EtOAc, concentrated and purified by silica (PE/EtOAc=20:1) to afford the title compound (11.3 g, 32.08 mmol, 65% yield) as a colorless oil.

### Intermediate 13b: 4-[(3-bromophenyl)methylene]piperidine hydrochloride

To a solution of hydrochloric acid in methanol (20.0 mL, 80 mmol, 5.64 eq) was added Intermediate 13a (10.0 g, 14.19 mmol, 1.0 eq) at 20° C. and stirred for 4 h. The solution was concentrated and washed by MTBE/MeOH (100 mL, 9:1) to afford the title compound (3.16 g, 10.95 mmol, 77% yield) as a white solid.

### Intermediate 13c: benzyl 4-[(3-bromophenyl)methylene]piperidine-1-carboxylate

A solution of Intermediate 13b (3.16 g, 10.95 mmol, 1.0 eq) and sodium hydrogen carbonate (2.3 g, 27.37 mmol, 2.5 eq) in EtOAc (40 mL)/water (40 mL) was added benzyl chloroformate (1.7 mL, 12.04 mmol, 1.1 eq) at 0° C. and stirred for 2 h. The organic layer was separated out, washed with brine, concentrated and purified by silica column (PE/EtOAc 98/2) to afford the title compound (3.24 g, 8.3 mmol, 77% yield) as a colorless oil.

### Intermediate 55a: benzyl 4-(2-hydroxyethyl)piperazine-1-carboxylate

To a stirred solution of 2-(piperazin-1-yl)ethan-1-ol (4.5 g, 4.24 ml, 34.6 mmol, 1.0 eq) and Et₃N (3.5 g, 4.82 mL, 34.6 mmol, 1.0 eq) in tetrahydrofuran (150 mL) was added dropwise benzyl carbonochloridate (5.9 g, 4.87 mL, 34.6 mmol, 1.0 eq) at 0-5° C. over 15 min. The reaction mixture was stirred for 35 min at 0-5° C. and was then allowed to warm to room temperature. The reaction mixture was then stirred for a further 5 h. A white suspension resulted. The reaction mixture was partitioned between ethyl acetate and water. The layers were separated. The organic layer was washed with brine, dried over anhydrous sodium sulfate and concentrated in vacuo. The crude material was purified by flash chromatography (DCM:MeOH 0-5) to afford the title compound (3.046 g, 11.5 mmol, 33% yield) as a colourless oil. MS (ESI): 265.0 ([M+H]⁺).

### Intermediate 55b: benzyl 4-(2-(2-bromophenoxy)ethyl)piperazine-1-carboxylate

To a stirred solution of 2-bromophenol (1.44 g, 965 μl, 8.32 mmol, 1.1 eq), Intermediate 55a (2 g, 7.57 mmol, 1.0 eq) and triphenylphosphine (2.18 g, 8.32 mmol, 1.1 eq) in tetrahydrofuran (25.2 mL) at room temperature was added di-tert-butyl azodicarboxylate (1.92 g, 8.32 mmol, 1.1 eq). The reaction mixture was stirred for 4 h. The reaction mixture was partitioned between ethyl acetate and water/brine (1:1). The layers were separated. The aqueous layer was extracted with ethyl acetate. The combined organic layers were washed with brine, dried over anhydrous sodium sulfate, and concentrated in vacuo. The crude material was purified by flash chromatography (DCM:MeOH 0-60) to give a mixture of the desired product and TPPO. 20 mL diethyl ether and 20 mL pentane were added. The solids were removed by filtration. The procedure was repeated with half the amount of the solvent. The crude material was purified by flash chromatography (DCM:MeOH 0-5) to afford the title compound (2.28 g, 5.44 mmol, 72% yield) as a colourless oil. MS (ESI): 420.9 ([M+H]⁺).

### Intermediate 68a: benzyl 4-(2-(4-bromophenoxy)ethyl)piperazine-1-carboxylate

The title compound (8.96 g, 21.4 mmol, 72% yield), colourless oil, was prepared in analogy to intermediate 68a using 4-bromophenol (5 g, 28.9 mmol, Eq: 1) and Intermediate 55a after extraction and flash chromatography (DCM/MeOH 0-4). MS (ESI): 421.2 ([M+H]⁺).

### Intermediate 102a: benzyl 4-(3-bromobenzyl)piperazine-1-carboxylate

In a 100 mL round-bottomed flask, 1-bromo-3-(bromomethyl)benzene (6 g, 24 mmol, 1.0 eq) was combined with THF (120 mL). Triethylamine (3.64 g, 5.02 mL, 36 mmol, 1.5 eq) was then added, followed by the addition of benzyl piperazine-1-carboxylate (6.35 g, 5.56 mL, 28.8 mmol, 1.2 eq). The reaction mixture was stirred at room temperature overnight. The solvent was evaporated and the residue was purified by flash chromatography (Heptane/EtOAc 0-100) to afford the title compound (9.26 g, 23.8 mmol, 99% yield) as a colorless oil. MS (ESI): 391.0874 ([M+H]⁺).

### Intermediate 2d: tert-butyl 8-[3-[2-(4-benzyloxycarbonylpiperazin-1-yl)ethoxy]phenyl]-3,8-diazabicyclo[3.2.1]octane-3-carboxylate

A mixture of 3-Boc-3,8-diazabicyclo[3.2.1]octane (11695.16 mg, 55.09 mmol, 1.1 eq), Brettphos Pd G3 (2147.18 mg, 2.5 mmol, 0.05 eq), intermediate 2c (21 g, 50.08 mmol, 1.0 eq) and potassium carbonate (13843.34 mg, 100.16 mmol, 2.0 eq) in tert-butanol (100 mL) was heated at 85° C. for 16 h under N2. The mixture was filtered, then purified by prep-HPLC (base) to afford the title compound (12 g, 21.79 mmol, 33% yield).

### Intermediate 13d: tert-butyl 8-[3-[(1-benzyloxycarbonyl-4-piperidylidene)methyl]phenyl]-3,8-diazabicyclo[3.2.1]octane-3-carboxylate

The title compound (2.11 g, 4.08 mmol, 50% yield), colorless oil, was prepared in analogy to intermediate 2d using 3-Boc-3,8-diazabicyclo[3.2.1]octane and intermediate 13c after extraction and flash chromatography (PE/EA=5:1).

### Intermediate 3a: tert-butyl 8-[3-[[tert-butyl(diphenyl)silyl]oxymethyl]phenyl]-3,8-diazabicyclo[3.2.1]octane-3-carboxylate

The title compound (6.1 g, 10.96 mmol, 23% yield), light yellow oil, was prepared in analogy to intermediate 2d using 3-Boc-3,8-diazabicyclo[3.2.1]octane and (3-bromophenyl)methoxy-tert-butyl-diphenyl-silane after extraction and flash chromatography (PE/EA=10:1).

### Intermediate 20a: tert-butyl 8-[3-[tert-butyl(diphenyl)silyl]oxyphenyl]-3,8-diazabicyclo[3.2.1]octane-3-carboxylate

The title compound (6.9 g, 12.71 mmol, 35% yield), yellow oil, was prepared in analogy to intermediate 2d using 3-Boc-3,8-diazabicyclo[3.2.1]octane and (3-bromophenoxy)-tert-butyl-diphenyl-silane after extraction and flash chromatography (PE/EA=10:1). MS (ESI): 543.2 ([M+H]⁺).

### Intermediate 52a: benzyl 4-[3-[2-(4-tert-butoxycarbonylpiperazin-1-yl)ethoxy]phenyl]-1-oxa-4,9-diazaspiro[5.5]undecane-9-carboxylate

Palladium (II) acetate (33 mg, 147 μmol, 0.2 eq) and Ruphos (34.3 mg, 73.4 μmol, 0.10 eq) were combined in degassed toluene (4 mL) under argon. The reaction mixture was heated to 50° C. and stirred for 20 min. In a separate flask flushed with Argon, intermediate 2a (283 mg, 734 μmol, Eq: 1), benzyl 1-oxa-4,9-diazaspiro[5.5]undecane-9-carboxylate hydrochloride (300 mg, 734 μmol, Eq: 1) and sodium tert-butoxide (212 mg, 2.2 mmol, 3.0 eq) were combined in degassed toluene (4 mL) under argon. The reaction mixture was heated to 50° C. and the catalyst reaction mixture was added via a seringe. The reaction mixture was stirred at 100° C. for 16 h. The reaction mixture was poured into saturated NaHCO₃ and extracted with EtOAc. The organic layers were combined and washed with H₂O and brine. The organic layers were dried over Na₂SO₄ and concentrated in vacuo. The crude material was purified by flash chromatography (Heptane/EtOAc 0 to 100) to afford the title compound (330 mg, 555 μmol, 68% yield) as a light brown oil. MS (ESI): 595.4 ([M+H]⁺).

### Intermediate 55c: tert-butyl (1R,5S)-8-(2-(2-(4-((benzyloxy)carbonyl)piperazin-1-yl)ethoxy)phenyl)-3,8-diazabicyclo[3.2.1]octane-3-carboxylate

The title compound (1.18 g, 2.13 mmol, 51% yield), yellow oil, was prepared in analogy to intermediate 52a using tert-butyl (1R,5S)-3,8-diazabicyclo[3.2.1]octane-3-carboxylate and intermediate 55b after extraction and two flash chromatography (Heptane/EtOAc; DCM/MeOH 98:2). MS (ESI): 551.4 ([M+H]⁺).

### Intermediate 68b: tert-butyl (1R,5S)-8-(4-(2-(4-((benzyloxy)carbonyl)piperazin-1-yl)ethoxy)phenyl)-3,8-diazabicyclo[3.2.1]octane-3-carboxylate

The title compound (1.45 g, 2.63 mmol, 76% yield), orange oil, was prepared in analogy to intermediate 52a using tert-butyl (1R,5S)-3,8-diazabicyclo[3.2.1]octane-3-carboxylate and intermediate 68a after extraction and flash chromatography (DCM/MeOH 0-5). MS (ESI): 551.4 ([M+H]⁺).

### Intermediate 102b: tert-butyl 8-(3-((4-((benzyloxy)carbonyl)piperazin-1-yl)methyl)phenyl)-3,8-diazabicyclo[3.2.1]octane-3-carboxylate

Intermediate 102a (3 g, 7.71 mmol, 1.0 eq), tert-butyl 3,8-diazabicyclo[3.2.1]octane-3-carboxylate (2.45 g, 11.6 mmol, 1.5 eq) and sodium tert-butoxide (1.11 g, 11.6 mmol, 1.5 eq) were combined with toluene (30 mL). The reaction vessel was degassed by purging with argon. RuPhos Pd G3 95% (173 mg, 771 μmol, 0.1 eq) was added. The reaction mixture was heated to 110° C. and stirred for 16 h. The reaction mixture was filtered through celite. The crude material was purified by silica gel flash chromatography (Heptane/EtOAc 0-100) to afford the title compound (3 g, 5.76 mmol, 74% yield) as a colorless oil. MS (ESI): 391.0874 ([M+H]⁺).

### Intermediate 2e: benzyl 4-[2-[3-(3,8-diazabicyclo[3.2.1]octan-8-yl)phenoxy]ethyl]piperazine-1-carboxylate

A mixture of Intermediate 2a (3000 mg, 5.45 mmol, 1.0 eq) and 4 M HCl in dioxane (20.0 mL, 5.45 mmol, 1.0 eq) in methanol (100 mL) was heated at 85° C. for 16 h under N2. The mixture was filtered, then purified by prep-HPLC (base) to afford the title compound (2.5 g, 5.55 mmol, 101% yield).

### Intermediate 102c: benzyl 4-[[3-(3,8-diazabicyclo[3.2.1]octan-8-yl)phenyl]methyl]piperazine-1-carboxylate

Intermediate 102b (3 g, 5.76 mmol, 1.0 eq) was dissolved in dioxane (20 mL) and hydrochlorid acid in methanol (20 mL, 80 mmol, 13.9 eq) was added. The reaction mixture was stirred at room temperature for 2 h. The mixture was filtered, the solid precipitate was washed with the mother liquor and once with diethyl ether. The precipitate was dried to afford the title compound (2.96 g, 6.48 mmol, 112% yield) as a white hydrochloric salt. MS (ESI): 421.2615 ([M+H]⁺).

### Intermediate 3b: [3-(3,8-diazabicyclo[3.2.1]octan-8-yl)phenyl]methanol

The title compound (3.4 g, 13.35 mmol, 86% yield), white hydrochloride salt, was prepared in analogy to intermediate 102b using intermediate 3a and precipitation with MTBE.

### Intermediate 13e: benzyl 4-[[3-(3,8-diazabicyclo[3.2.1]octan-8-yl)phenyl]methylene]piperidine-1-carboxylate

The title compound (1.43 g, 3.15 mmol, 76% yield), white hydrochloride salt, was prepared in analogy to intermediate 102b using intermediate 13d.

### Intermediate 68c: benzyl 4-(2-(4-((1R,5S)-3,8-diazabicyclo[3.2.1]octan-8-yl)phenoxy)ethyl)piperazine-1-carboxylate

To a stirred suspension of intermediate 68b (10.02 g, 18.2 mmol, 1.0 eq) in 1,4-dioxane (30 mL) at room temperature was added 4 M hydrogen chloride in 1,4-dioxane (45.5 ml, 182 mmol, 10.0 eq). The reaction mixture was stirred for 16 h. The product was collected by filtration through a Sartorius filter, washed with tetrahydrofuran and ethyl acetate, and dried in vacuo to afford the title compound (8.667 g, 15.5 mmol, 85% yield) as an off-white trihydrochloride salt. MS (ESI): 451.4 ([M+H]⁺).

### Intermediate 55d: benzyl 4-(2-(2-((1R,5S)-3,8-diazabicyclo[3.2.1]octan-8-yl)phenoxy)ethyl)piperazine-1-carboxylate

The title compound (1.00 g, 1.79 mmol, 89% yield), white trihydrochloride salt, was prepared in analogy to intermediate 68c from intermediate 55c. MS (ESI): 451.2 ([M+H]⁺).

### Intermediate 94a: benzyl 4-[3-(1-tert-butoxycarbonylazetidin-3-yl)oxyphenyl]piperazine-1-carboxylate

A mixture of 1-Boc-3-iodoazetidine (5438.15 mg, 19.21 mmol, 1.2 eq), benzyl 4-(3-hydroxyphenyl)piperazine-1-carboxylate (5000 mg, 16.01 mmol, 1.0 eq), Cs₂CO₃ (7823.18 mg, 24.01 mmol, 1.5 eq) in DMF (100 mL) was stirred at 80° C. for 12 h. The reaction mixture was poured into water, extracted with ethyl acetate, dried over Na₂SO₄ and concentrated to afford the title compound (6000 mg, 12.83 mmol, 80% yield) as a yellow oil.

### Intermediate 94b: benzyl 4-[3-(azetidin-3-yloxy)phenyl]piperazine-1-carboxylate

A mixture of Intermediate 94a (6000 mg, 12.83 mmol, 1.0 eq) in TFA (50 mL, 12.83 mmol, 1.0 eq) and DCM (200 mL) was stirred at 25° C. for 1 h. The reaction mixture was concentrated to afford the title compound (4000 mg, 10.89 mmol, 84% yield) as a yellow oil as the TFA salt.

### Intermediate 103a: benzyl 4-(4-hydroxybenzoyl)piperazine-1-carboxylate

To a solution of 4-hydroxybenzoic acid (10 g, 72.4 mmol, 1.0 eq) in DMF (140 mL) were added 1-Cbz-piperazine (16 g, 72.4 mmol, 1.0 eq), HATU (31 g, 79.64 mmol, 1.1 eq) and triethylamine (20 mL, 144.8 mmol, 2.0 eq), and then the reaction was stirred at 25° C. for 2 h. The mixture was diluted with water (120 mL) and extracted with EtOAc. The combined organic phase was washed with brine, dried over sodium sulfate, filtered and concentrated under reduced pressure. The crude residue was purified by flash chromatography (PE/EtOAc 25-50) to afford the title compound (18 g, 52.88 mmol, 73% yield) as a brown liquid. MS (ESI): 341.1 ([M+H]⁺).

### Intermediate 103b: benzyl 4-[4-(1-tert-butoxycarbonylazetidin-3-yl)oxybenzoyl]piperazine-1-carboxylate

To a solution of intermediate 103a (12 g, 35.26 mmol, 1.0 eq) in DMF (140 mL) was added 1-Boc-3-iodoazetidine (12 g, 42.31 mmol, 1.2 eq) and cesium carbonate (14 g, 42.31 mmol, 1.2 eq) and the reaction was stirred at 80° C. for 12 h. The residue was diluted with water and extracted with EtOAc. The combined organic layer was dried over anhydrous sodium sulfate, filtered and concentrated in vacuo. The crude residue was purified by flash chromatography using (PE/EtOAc 25-50) as eluent to afford the title compound (10 g, 20.18 mmol, 57% yield) as a yellow liquid. MS (ESI): 440.1 ([M−56+H]⁺).

### Intermediate 103c: benzyl 4-[4-(azetidin-3-yloxy)benzoyl]piperazine-1-carboxylate

To a solution of intermediate 103b (10 g, 20.18 mmol, 1.0 eq) in DCM (50 mL) was added trifluoroacetic acid (50.0 mL) and the reaction was stirred at 25° C. for 1 h. The reaction solution was concentrated to afford the title compound (7 g, 17.7 mmol, 87% yield).

### Intermediate 52b: tert-butyl 4-[2-[3-(1-oxa-4,9-diazaspiro[5.5]undecan-4-yl)phenoxy]ethyl]piperazine-1-carboxylate

To a solution of intermediate 52a (500 mg, 841 μmol, 1.0 eq) in methanol (5 mL) was added 10% palladium on charcoal (89.5 mg, 84.1 μmol, 0.1 eq). The reaction mixture was vigorously stirred at room temperature for 3 h under H₂ (baloon). The catalyst was collected by filtration, washing with methanol. The filtrate was concentrated to afford the title compound (390 mg, 840 μmol, 100% yield) as a light brown oil. MS (ESI): 461.4 ([M+H]⁺).

### Intermediate 20b: tert-butyl-[3-(3,8-diazabicyclo[3.2.1]octan-8-yl)phenoxy]-diphenyl-silane

To a mixture of intermediate 20a (6.9 g, 12.71 mmol, 1.0 eq) in methanol (50 mL) was added 4 M hydrochloride in dioxane (50.0 mL, 200 mmol, 15.73 eq) and stirred at 10° C. for 16 h. The mixture was concentrated and purified by flash column (FA) to afford the title compound (2.9 g, 6.05 mmol, 47% yield) as a light yellow hydrochloride salt. MS (ESI): 443.3 ([M+H]⁺).

### Intermediate 83a: tert-butyl 3-(3-formylphenoxy)azetidine-1-carboxylate

A mixture of 1-Boc-3-iodoazetidine (6.03 g, 21.29 mmol, 1.3 eq), 3-hydroxybenzaldehyde (2 g, 16.38 mmol, 1.0 eq) and cesium carbonate (9.6 g, 29.48 mmol, 1.8 eq) in DMF (15 mL) was stirred at 150° C. for 1 h under the microwave condition. The reaction mixture was poured into water, extracted with ethyl acetate, dried over Na₂SO₄ and concentrated in vacuum to afford the title compound (2 g, 7.22 mmol, 44% yield) as a yellow oil.

### Intermediate 83b: benzyl 4-(3-((1-(tert-butoxycarbonyl)azetidin-3-yl)oxy)benzyl)piperazine-1-carboxylate

A mixture of 1-Cbz-piperazine (1.9 g, 8.65 mmol, 1.2 eq), intermediate 83a (2 g, 7.21 mmol, 1.0 eq) and acetic acid (0.5 mL, 7.21 mmol, 1.0 eq) in DME (50 mL) was stirred at 25° C. for 1 h. Then sodium cyanoborohydride (906 mg, 14.42 mmol, 2.0 eq) was added, the reaction mixture was stirred at 25° C. for 12 h. The reaction mixture was concentrated in vacuum and then directly purified by prep-HPLC to afford the title compound (2 g, 4.1 mmol, 57% yield) as a yellow oil. MS (ESI): 482.4 ([M+H]⁺).

### Intermediate 83c: benzyl 4-(3-(azetidin-3-yloxy)benzyl)piperazine-1-carboxylate

A mixture of intermediate 83b (2 g, 4.15 mmol, 1.0 eq) in trifluoroacetic acid (5.0 mL, 44.85 mmol, 11.0 eq) and DCM (20 mL) was stirred at 25° C. for 2 h. The reaction mixture was concentrated in vacuum to afford the title compound (1.5 g, 3.9 mmol, 94% yield) as a yellow oil, TFA salt. MS (ESI): 382.3 ([M+H]⁺).

### Intermediate 99a: benzyl 4-[(1-tert-butoxycarbonyl-4-piperidyl)methylcarbamoyl]-4-phenyl-piperidine-1-carboxylate

To a solution of 1-((benzyloxy)carbonyl)-4-phenylpiperidine-4-carboxylic acid (300 mg, 884 μmol, 1.0 eq), HATU (420 mg, 1.1 mmol, 1.25 eq) and Hunig's base (571 mg, 772 μL, 4.42 mmol, 5.0 eq) in DMF (3 mL) was added tert-butyl 4-(aminomethyl)piperidine-1-carboxylate (227 mg, 224 μL, 1.06 mmol, 1.2 eq). The reaction mixture was stirred at room temperature for 2 h. The reaction mixture was concentrated in vacuo. The crude material was purified by flash chromatography (Heptane/EtOAc 0-100) to afford the title compound (450 mg, 841 μmol, 95% yield) as a yellow oil. MS (ESI): 536.5 ([M+H]⁺).

### Intermediate 99b: tert-butyl 4-[[(4-phenylpiperidine-4-carbonyl)amino]methyl]piperidine-1-carboxylate

To a solution of intermediate 99a (580 mg, 1.08 mmol, 1.0 eq) in methanol (6 mL) was added 10% palladium on charcoal (115 mg, 108 μmol, 0.1 eq). The reaction mixture was vigorously stirred at room temperature for 24 h under H₂ (baloon). The catalyst was collected by filtration, washing with methanol. The filtrate was concentrated to afford the title compound (95 mg, 180 μmol, 91% yield) as a white solid. MS (ESI): 402.4 ([M+H]⁺).

### Intermediate 101a: benzyl 4-(3-hydroxybenzoyl)piperazine-1-carboxylate

To a mixture of 1-Cbz-piperazine (7.97 g, 36.2 mmol, 1.0 eq) and 3-hydroxybenzoic acid (5 g, 36.2 mmol, 1.0 eq) in DCM (50 mL) were added HATU (16.52 g, 43.44 mmol, 1.2 eq) and triethylamine (6.05 mL, 43.44 mmol, 1.2 eq) at 25° C. Then the mixture was stirred at 25° C. for 15 h. The mixture was diluted with water and extracted with DCM. The combined organic layers were dried over anhydrous sodium sulfate, filtered and concentrated in vacuo. The residue was purified by flash chromatography (PE/EtOAc 1:1) to afford the title compound (8 g, 23.5 mmol, 65% yield) as a white solid. MS (ESI): 341.1 ([M+H]⁺).

### Intermediate 101b: benzyl 4-[3-(1-tert-butoxycarbonylazetidin-3-yl)oxybenzoyl]piperazine-1-carboxylate

To a mixture of intermediate 101a (8 g, 23.5 mmol, 1.0 eq) and 1-Boc-3-iodoazetidine (8 g, 28.2 mmol, 1.2 eq) in DMF (30 mL) was added cesium carbonate (9.19 g, 28.2 mmol, 1.2 eq) at 25° C. The mixture was stirred at 80° C. for 4 h. To the mixture was added water and it was extracted with ethyl acetate. The organic layers were combined, dried over anhydrous sodium sulfate, filtered and concentrated in vacuo. The residue was purified by flash chromatography (PE/EtOAc 1:1) to afford the title compound (6 g, 12.11 mmol, 26% yield) as a brown oil. MS (ESI): 496.3 ([M+H]⁺).

### Intermediate 101c: benzyl 4-[3-(azetidin-3-yloxy)benzoyl]piperazine-1-carboxylate

To a mixture of intermediate 101b (6.0 g, 6.05 mmol, 1.0 eq) in DCM (20 mL) was added trifluoroacetic acid (10.0 mL, 129.8 mmol, 21 eq) at 25° C. The mixture was stirred at 25° C. for 4 h and then concentrated in vacuo to afford the title compound (4 g, 7.85 mmol, 66% yield) as a brown viscous oil, TFA salt. MS (ESI): 396.1 ([M+H]⁺).

### Intermediate 110a: tert-butyl 3-(3-benzyloxyphenyl)-3,8-diazabicyclo[3.2.1]octane-8-carboxylate

To a suspension of 1-(benzyloxy)-3-bromobenzene (5 g, 19 mmol, 1.0 eq) and tert-butyl (1R,5S)-3,8-diazabicyclo[3.2.1]octane-8-carboxylate (4.24 g, 20 mmol, 1.05 eq) in t-BuOH (30 mL) was added K₂CO₃ (5.25 g, 38 mmol, 2.0 eq). The reaction was degassed with argon for 5 min. RuPhos Pd G3 (1.34 g, 1.6 mmol, 0.0843 eq) was added. The reaction mixture was stirred at 120° C. overnight. The catalyst was removed by filtration and washed with ethyl acetate. The residue was poured into EtOAc/THF 2:1 and washed with water and brine. The organic layer was dried over Na₂SO₄ and concentrated in vacuo. The crude material was purified by flash chromatography (0% to 18% EtOAc in heptane) to afford the title compound (3.4 g, 8.62 mmol, 45% yield) as a yellow oil. MS (ESI): 395.2358 ([M+H]⁺).

### Intermediate 110b: tert-butyl 3-(3-hydroxyphenyl)-3,8-diazabicyclo[3.2.1]octane-8-carboxylate

To a solution intermediate 110a (3.4 g, 8.62 mmol, 1.0 eq) in methanol (200 mL) was added ammonium formate (10.9 g, 172 mmol, 20.0 eq). The reaction mixture was degassed with argon for 10 min. Pd—C 10% (917 mg, 862 μmol, 0.1 eq) was added. The reaction mixture was stirred for 2 h at 70 C.°. The reaction mixture was filtered through sartorius funnel and concentrated in vacuo. The reaction mixture was poured into AcOEt/THF 1:1 and washed with H₂O and brine. The organic layer was dried over Na₂SO₄ and concentrated in vacuo to afford the title compound (2.57 g, 8.44 mmol, 98% yield) as an off-white solid. MS (ESI): 305.1893 ([M+H]⁺).

### Intermediate 110c: tert-butyl 3-[3-[2-(4-benzyloxycarbonylpiperazin-1-yl)ethoxy]phenyl]-3,8-diazabicyclo[3.2.1]octane-8-carboxylate

To a solution of intermediate 110b (2.56 g, 8.41 mmol, 1.0 eq) and benzyl 4-(2-hydroxyethyl)piperazine-1-carboxylate (2.38 g, 9 mmol, 1.07 eq) in THF (20 mL) was added benzyl 4-(2-hydroxyethyl)piperazine-1-carboxylate (2.38 g, 9 mmol, 1.07 eq). The reaction mixture was stirred for 2 h overnight at 70° C. 2-(trimethylphosphoranylidene)acetonitrile 0.5M in THF (20 mL, 10 mmol, 1.19 eq) was added. The reaction mixture was stirred for 2 h at 70° C. The reaction mixture was poured into THF/AcOEt 3:1 and washed with H₂O/brine. The organic layer was dried over Na₂SO₄ and concentrated in vacuo. The crude material was purified by flash chromatography to afford the title compound (3.66 g, 6.65 mmol, 79% yield) as a brown oil. MS (ESI): 551.3228 ([M+H]⁺).

### Intermediate 110d: benzyl 4-[2-[3-(3,8-diazabicyclo[3.2.1]octan-3-yl)phenoxy]ethyl]piperazine-1-carboxylate

To a solution of intermediate 110c (3.6 g, 6.54 mmol, 1.0 eq) in DCM (25 mL) was added TFA (14.8 g, 10 mL, 130 mmol, 1.0 eq). The reaction mixture was stirred for 2 h at room temperature. The reaction mixture was concentrated in vacuo, poured into AcOEt/THF 1:2 and washed with NaOH 1N/brine 1:1. The organic layer was dried over Na₂SO₄ and concentrated in vacuo to afford the title compound (3.773 g, 8.37 mmol, 128% yield) as a brown oil. MS (ESI): 451.2742 ([M+H]⁺).

### Intermediate 29a: tert-butyl 3-(3-amino-6-chloro-pyridazin-4-yl)-3,8-diazabicyclo[3.2.1]octane-8-carboxylate

To a solution of tert-butyl 3,8-diazabicyclo[3.2.1]octane-8-carboxylate (1.02 g, 4.8 mmol, 1.0 eq) in dioxane (5.5 mL) was added 4-bromo-6-chloropyridazin-3-amine (1.0 g, 4.8 mmol, 1.0 eq) and DIPEA (1.24 g, 1.68 mL, 9.6 μmol, 2.0 eq). The reaction mixture was heated to 100° C. for 48 h. The reaction mixture was concentrated in vacuo and purified by silica gel flash chromatography using ethyl acetate/methanol (0-10%) as eluent to afford the title compound (1.39 g, 4.1 mmol, 85% yield) as an off-white solid. MS (ESI): 340.1 ([M+H]⁺).

### Intermediate 2f: benzyl 4-[2-[3-[3-(3-amino-6-chloro-pyridazin-4-yl)-3,8-diazabicyclo[3.2.1]octan-8-yl]phenoxy]ethyl]piperazine-1-carboxylate

A mixture of 4-bromo-6-chloro-pyridazin-3-amine (1279.67 mg, 6.14 mmol, 1.3 eq), intermediate 2e (2.3 g, 4.72 mmol, 1.0 eq) and triethylamine (1.32 mL, 9.45 mmol, 2.0 eq) in DMF (10 mL) was heated at 85° C. for 16 h. The mixture was poured into water, extracted with ethyl acetate, washed with brine, concentrated in vacuum and the residue was purified by silica column (DCM/EtOAc 2:1) to afford the title compound (2 g, 3.46 mmol, 69% yield) as a light yellow solid.

### Intermediate 3c: [3-[3-(3-amino-6-chloro-pyridazin-4-yl)-3,8-diazabicyclo[3.2.1]octan-8-yl]phenyl]methanol

The title compound (5.5 g, 15.9 mmol, 65% yield), yellow solid, was prepared in analogy to intermediate 2f from intermediate 3b at 90° C. (DCM/EtOAc 1:1).

### Intermediate 4a: Tert-butyl 4-(3-amino-6-chloropyridazin-4-yl)-2-methylpiperazine-1-carboxylate

The title compound (154 mg, 0.47 mmol, 49% yield), brown solid, was prepared in analogy to intermediate 2f from tert-butyl 2-methylpiperazine-1-carboxylate (5.5 eq) (Heptane/EtOAc 0-100). MS (ESI): 328.1 ([M+H]⁺).

### Intermediate 6a: tert-butyl 9-(3-amino-6-chloro-pyridazin-4-yl)-3,9-diazaspiro[5.5]undecane-3-carboxylate

The title compound (5 g, 13.09 mmol, 66% yield) was prepared in analogy to intermediate 2f from tert-butyl 3,9-diazaspiro[5.5]undecane-3-carboxylate (1.1 eq), triethylamine (3 eq) (Heptane/EtOAc 0-100). MS (ESI): 382.3 ([M+H]⁺).

### Intermediate 13f: benzyl 4-[[3-[3-(3-amino-6-chloro-pyridazin-4-yl)-3,8-diazabicyclo[3.2.1]octan-8-yl]phenyl]methylene]piperidine-1-carboxylate

The title compound (1.17 g, 2.15 mmol, 73% yield), yellow solid, was prepared in analogy to intermediate 2f from intermediate 13e, triethylamine (3 eq) (PE/EA 1:1).

### Intermediate 20c: 4-[8-[3-[tert-butyl(diphenyl)silyl]oxyphenyl]-3,8-diazabicyclo[3.2.1]octan-3-yl]-6-chloro-pyridazin-3-amine

The title compound (2 g, 3.51 mmol, 58% yield), off-white solid, was prepared in analogy to intermediate 2f from intermediate 20b, triethylamine (10 eq). MS (ESI): 570.2 ([M+H]⁺).

### Intermediate 83d: benzyl 4-(3-((1-(3-amino-6-chloropyridazin-4-yl)azetidin-3-yl)oxy)benzyl)piperazine-1-carboxylate

The title compound (1.5 g, 2.95 mmol, 54% yield), yellow oil, was prepared in analogy to intermediate 2f from intermediate 83c, triethylamine (3 eq). MS (ESI): 509.3 ([M+H]⁺).

### Intermediate 94c: benzyl 4-[3-[1-(3-amino-6-chloro-pyridazin-4-yl)azetidin-3-yl]oxyphenyl]piperazine-1-carboxylate

The title compound (2 g, 49% yield), yellow oil, was prepared in analogy to intermediate 2f from intermediate 94b, triethylamine (3 eq), prep HPLC.

### Intermediate 101d: benzyl 4-[4-[1-(3-amino-6-chloro-pyridazin-4-yl)azetidin-3-yl]oxybenzoyl]piperazine-1-carboxylate

The title compound (800 mg, 1.53 mmol, 38% yield), brown solid, was prepared in analogy to intermediate 2f from intermediate 101c, triethylamine (2.5 eq), prep HPLC. MS (ESI): 523.1/525.1 ([M+H]⁺).

### Intermediate 103d: benzyl 4-[4-[1-(3-amino-6-chloro-pyridazin-4-yl)azetidin-3-yl]oxybenzoyl]piperazine-1-carboxylate

The title compound (4 g, 7.65 mmol, 43% yield), brown gum, was prepared in analogy to intermediate 2f from intermediate 103c, triethylamine (4 eq), (PE/EtOAc 67-100). MS (ESI): 523.2 ([M+H]⁺).

### Intermediate 12a: tert-butyl 9-(3-amino-6-chloro-pyridazin-4-yl)-1-oxa-4,9-diazaspiro[5.5]undecane-4-carboxylate

To a stirred solution of 4-bromo-6-chloropyridazin-3-amine (600 mg, 2.88 mmol, 1.0 eq) and tert-butyl 1-oxa-4,9-diazaspiro[5.5]undecane-4-carboxylate (812 mg, 3.17 mmol, 1.1 eq) in DMA (8 mL) was added potassium carbonate (1.19 g, 8.64 mmol, 3.0 eq). The reaction mixture was heated to 110° C. and stirred for 20 h. The reaction mixture was poured in H₂O and extracted with EtOAc. The organic layers were combined, washed with sat NaHCO₃, H₂O and brine. The organic layers were dried over Na₂SO₄ and concentrated in vacuo. The crude material was purified by flash chromatography (Heptane/EtOAc 0-100) to afford the title compound (1.02 g, 2.66 mmol, 92% yield) as a light brown solid. MS (ESI): 384.2 ([M+H]⁺).

### Intermediate 16a: tert-butyl 7-(3-amino-6-chloro-pyridazin-4-yl)-4,7-diazaspiro[2.5]octane-4-carboxylate

The title compound (230 mg, 668 μmol, 47% yield), brown solid, was prepared in analogy to intermediate 12a from tert-butyl 4,7-diazaspiro[2.5]octane-4-carboxylate (1.05 eq), (Heptane/EtOAc 0-100). MS (ESI) 340.2 ([M+H]⁺).

### Intermediate 19a: tert-Butyl 3-(3-amino-6-chloro-pyridazin-4-yl)-3,9-diazabicyclo[3.3.1]nonane-9-carboxylate

The title compound (97 mg, 0.27 mmol, 12% yield), light yellow solid, was prepared in analogy to intermediate 12a from tert-butyl 3,9-diazabicyclo[3.3.1]nonane-9-carboxylate (1 eq), K₂CO₃ (1.5 eq) (Heptane/EtOAc 0-100). MS (ESI): 354.2 ([M+H]⁺).

### Intermediate 52c: tert-butyl 4-[2-[3-[9-(3-amino-6-chloro-pyridazin-4-yl)-1-oxa-4,9-diazaspiro[5.5]undecan-4-yl]phenoxy]ethyl]piperazine-1-carboxylate

The title compound (340 mg, 578 μmol, 57% yield), brown oil, was prepared in analogy to intermediate 12a from intermediate 52b (1 eq), K₂CO₃ (2 eq) (DCM/MeOH 0-10). MS (ESI): 588.4 ([M+H]⁺).

### Intermediate 97a: potassium 4-(3-amino-6-chloro-pyridazin-4-yl)-1-phenyl-piperazine-2-carboxylate

The title compound (600 mg, 1.80 mmol, 94% yield), brown oil, was prepared in analogy to intermediate 12a from 1-phenylpiperazine-2-carboxylic acid dihydrochloride (1 eq), K2CO3 (2 eq) (crude). MS (ESI): 334.2 ([M+H]⁺).

### Intermediate 55e: benzyl 4-(2-(2-((1R,5S)-3-(3-amino-6-chloropyridazin-4-yl)-3,8-diazabicyclo[3.2.1]octan-8-yl)phenoxy)ethyl)piperazine-1-carboxylate

To a stirred solution of intermediate 55d (300 mg, 536 μmol, 1.0 eq) and K₂CO₃ (370 mg, 2.68 mmol, 5.0 eq) in DMSO (3 mL) at 110° C. was added 4-bromo-6-chloropyridazin-3-amine (123 mg, 589 μmol, 1.1 eq). The reaction mixture was stirred at 110° C. for 30 h. The reaction mixture was partitioned between ethyl acetate/THF (1:2) and water/brine (1:1). The layers were separated. The aqueous layer was extracted with ethyl acetate/THF (1:1). The combined organic layers were washed with brine, dried over anhydrous sodium sulfate, and concentrated in vacuo. The crude material was purified by flash chromatography (DCM/MeOH 0-10), to afford the title compound (285.8 mg, 494 μmol, 92% yield) as an orange oil. MS (ESI): 578.3 ([M+H]⁺).

### Intermediate 68d: benzyl 4-(2-(4-((1R,5S)-3-(3-amino-6-chloropyridazin-4-yl)-3,8-diazabicyclo[3.2.1]octan-8-yl)phenoxy)ethyl)piperazine-1-carboxylate

The title compound (817 mg, 1.41 mmol, 40% yield), orange solid, was prepared in analogy to intermediate 55e from intermediate 68c (DCM/MeOH 0-10). MS (ESI): 578.2 ([M+H]⁺).

### Intermediate 89a: tert-Butyl N-[3-(3-amino-6-chloro-pyridazin-4-yl)-3-azabicyclo[3.2.1]octan-8-yl]carbamate

The title compound (562 mg, 1.59 mmol, 72% yield), off-white solid, was prepared in analogy to intermediate 55e from tert-butyl N-(3-azabicyclo[3.2.1]octan-8-yl)carbamate (Heptane/EtOAc 0-100). MS (ESI): 354.2 ([M+H]⁺).

### Intermediate 99c: tert-butyl 4-[[[1-(3-amino-6-chloro-pyridazin-4-yl)-4-phenyl-piperidine-4-carbonyl]amino]methyl]piperidine-1-carboxylate

The title compound (365 mg, 691 μmol, 70% yield), brown solid, was prepared in analogy to intermediate 55e from intermediate 99b (DCM/MeOH 0-10). MS (ESI): 529.4 ([M+H]⁺).

### Intermediate 102d: benzyl 4-[[3-[3-(3-amino-6-chloro-pyridazin-4-yl)-3,8-diazabicyclo[3.2.1]octan-8-yl]phenyl]methyl]piperazine-1-carboxylate

The title compound (1.9 g, 3.29 mmol, 46% yield), light brown foam, was prepared in analogy to intermediate 55e from intermediate 102c (EtOAc/MeOH 0-10). MS (ESI): 548.2542 ([M+H]⁺).

### Intermediate 110e: benzyl 4-[2-[3-[8-(3-amino-6-chloro-pyridazin-4-yl)-3,8-diazabicyclo[3.2.1]octan-3-yl]phenoxy]ethyl]piperazine-1-carboxylate

To a solution of intermediate 110d (3.773 g, 6.78 mmol, 1.0 eq) and 4-bromo-6-chloropyridazin-3-amine (1.7 g, 8.14 mmol, 1.2 eq) in DMSO (12 mL) was added K₂CO₃ (4.69 g, 33.9 mmol, 5.0 eq). The reaction mixture was stirred for 16 h at 110° C. The reaction mixture was poured into THF/AcOEt 2:1 and washed with H₂O/brine. The organic layer was dried over Na₂SO₄ and concentrated in vacuo. The crude material was purified by flash chromatography (silica gel, 80 g, 0% to 5% MeOH in DCM) to afford the title compound (2.74 g, 4.55 mmol, 67% yield) as a brown foam. MS (ESI): 578.2665 ([M+H]⁺).

### Intermediate 75a: methyl 2-phenyl-2-(4-(4,4,5,5-tetramethyl-1,3,2-dioxaborolan-2-yl)-1H-pyrazol-1-yl)acetate

To a solution of 4-pyrazoleboronic acid pinacol ester (5 g, 25.77 mmol, 1.0 eq), potassium carbonate (7.12 g, 51.54 mmol, 2.0 eq) in acetonitrile (10 mL) was added α-bromophenylacetic acid methyl ester (5.9 g, 25.77 mmol, 1.0 eq) at 25° C. The reaction was stirred at 80° C. for 12 h. The reaction mixture was concentrated and the crude was purified by silica gel chromatography to afford the title compound (5 g, 14.5 mmol, 56% yield) as a yellow solid. MS (ESI): 343.2 ([M+H]⁺).

### Intermediate 17a: tert-butyl 4-(3-amino-6-chloro-pyridazin-4-yl)-3,6-dihydro-2H-pyridine-1-carboxylate

A solution of 4-bromo-6-chloropyridazin-3-amine (500 mg, 2.4 mmol, 1.0 eq), tert-butyl 4-(4,4,5,5-tetramethyl-1,3,2-dioxaborolan-2-yl)-3,6-dihydropyridine-1(2H)-carboxylate (742 mg, 2.4 mmol, 1.0 eq), tetrakis(triphenylphosphine)palladium (0) (222 mg, 192 μmol, 0.08 eq) and sodium carbonate (508 mg, 4.8 mmol, 2.0 eq) in a mixture of degassed dioxane (30 mL) and H₂O (5 mL) was stirred at 110° C. for 5 h. The reaction mixture was poured in saturated NH₄Cl and extracted with EtOAc. The organic layers were combined, washed with H₂O and brine. The organic layers were dried over Na₂SO₄ and concentrated in vacuo. The crude material was purified by flash chromatography (Heptane/EtOAc 0-70) to afford the title compound (434 mg, 1.4 mmol, 58% yield) as a brown solid. MS (ESI): 311.2 ([M+H]⁺).

### Intermediate 74a: (3-(3-amino-6-chloropyridazin-4-yl)-2-fluorophenyl)methanol

The title compound (246 mg, 0.97 mmol, 39% yield), light brown solid, was prepared in analogy to intermediate 17a from (2-fluoro-3-(hydroxymethyl)phenyl)boronic acid (1.3 eq), K₂CO₃ (2 eq), (Heptane/EtOAc 0-100). MS (ESI): 254.0/255.9 ([M+H]⁺).

### Intermediate 75b: methyl 2-(4-(3-amino-6-chloropyridazin-4-yl)-1H-pyrazol-1-yl)-2-phenylacetate

The title compound (200 mg, 0.58 mmol, 33.1% yield), yellow oil, was prepared in analogy to intermediate 17a from intermediate 75a (1.3 eq), Pd(dppf)Cl₂ (0.1 eq), prep-HPLC. MS (ESI): 344.1/346.1 ([M+H]⁺).

### Intermediate 87a: 4-(3-amino-6-chloropyridazin-4-yl)benzaldehyde

The title compound (791 mg, 3.39 mmol, 70% yield), light yellow solid, was prepared in analogy to intermediate 17a from (4-formylphenyl)boronic acid (1.05 eq), Pd(dppf)Cl₂ (0.1 eq), (Heptane/EtOAc 0-100). MS (ESI): 344.1/346.1 ([M+H]⁺).

### Intermediate 21a: 3-Amino-2-phenylpropan-1-ol

To a suspension of lithium aluminum hydride (10 g, 264.3 mmol) in diethyl ether (200 mL) at 0° C. was added ethyl 2-cyano-2-phenyl-acetate (10 g, 52.9 mmol) in diethyl ether (50 mL) slowly under nitrogen atmosphere and the reaction mixture was stirred at 0° C. for 2 h. The reaction mixture was quenched with saturated sodium sulfate solution, filtered through the celite bed and washed with diethyl ether and methanol-dichloromethane (0-5%), dried over anhydrous sodium sulfate, filtered and concentrated under reduced pressure to afford the title compound (8 g, 42.33 mmol, 80% yield). MS (ESI): 152.1 ([M+H]⁺).

### Intermediate 21b: Ethyl (3-hydroxy-2-phenylpropyl)carbamate

To a solution of intermediate 21a (8 g, 52.9 mmol) in tetrahydrofuran (80 mL) at 0° C. was added ethylchlorofomate (8.6 g, 79.36 mmol) and triethylamine (13.4 g, 132.3 mmol, 18.44 mL) under nitrogen atmosphere and the reaction mixture was stirred at 0° C. for 10 min. The reaction mixture was slowly warmed to room temperature and stirred for 2 h. The reaction mixture was diluted with water and extracted with dichloromethane. The organic layer was dried over anhydrous sodium sulfate, filtered and concentrated under reduced pressure. The crude residue was purified by flash chromatography (Heptane/EtOAc 0-40) to afford the title compound (4.5 g, 19.75 mmol, 37% yield). MS (ESI): 224.1 ([M+H]⁺).

### Intermediate 21c: 3-(Methylamino)-2-phenylpropan-1-ol

To a solution of intermediate 21b (2.5 g, 10.45 mmol) in diethyl ether (50 mL) at 0° C. was added lithium aluminum hydride (2.45 g, 62.7 mmol) slowly in portions for 5 min and the reaction mixture was stirred at room temperature for 30 min, and then heated to 38° C. for 5 h. The reaction mixture was cooled to room temperature, quenched with sodium sulfate solution, passed through celite bed and washed with dichloromethane and methanol. The filtrate was dried over anhydrous sodium sulfate and concentrated under reduced pressure to afford the title compound (1.5 g, 8.90 mmol, 85% yield). MS (ESI): 166.0 ([M+H]⁺).

### Intermediate 21d: tert-Butyl (3-hydroxy-2-phenylpropyl)(methyl)carbamate

To a solution of intermediate 21c (3 g, 18.2 mmol) in tetrahydrofuran (40 mL) was added triethylamine (4.6 g, 45.4 mmol, 6.3 mL) and tertbutoxycarbonyl tert-butyl carbonate (6 g, 27.3 mmol, 6.3 mL) and the reaction mixture was stirred at room temperature for 2 h. The reaction mixture was diluted with water and extracted with ethylacetate. The organic layer was washed with water, brine solution, dried over anhydrous sodium sulfate and concentrated under reduced pressure. The crude residue was purified by flash chromatography (PE/EtOAc 0-20) to afford the title compound (2.7 g, 10.0 mmol, 55% yield). MS (ESI): 166.0 ([(M-Boc)+H]⁺).

### Intermediate 26a: 4-(2-((tert-butyldimethylsilyl)oxy)ethyl)aniline

To a solution of 2-(4-aminophenyl)ethanol (25.0 g, 182.2 mmol) in dichloromethane (10 mL) at 0° C. was added imidazole (13.5 g, 200 mmol) and tert-butyldimethylsilyl chloride (33 g, 219 mmol) and the reaction mixture was stirred at room temperature for 3 h. The reaction mixture was diluted with water and extracted with dichloromethane. The organic layer was dried over anhydrous sodium sulfate, filtered and concentrated under reduced pressure to afford the title compound (28.0 g, 109.1 mmol, 59% yield) as a pale yellow liquid. MS (ESI): 252.1 ([M+H]⁺).

### Intermediate 26b: Ethyl (4-(2-((tert-butyldimethylsilyl)oxy)ethyl)phenyl)carbamate

To a solution of intermediate 26a (1.5 g, 5.97 mmol) in dichloromethane (20 mL) at 0° C. was added triethylamine (905 mg, 8.95 mmol, 1.25 mL) slowly followed by the addition of ethyl chloroformate (1.43 g, 13.20 mmol, 1.26 mL) for 10 min and the reaction mixture was stirred at room temperature for 2 h. The reaction mixture was diluted with water and extracted with dichloromethane. The organic layer was dried over anhydrous sodium sulfate, filtered and concentrated under reduced pressure. The crude residue was purified by flash (PE/EtOAc 0-30) to afford the title compound (0.5 g, 1.51 mmol, 25% yield). MS (ESI): 324.3 ([M+H]⁺).

### Intermediate 26c: 2-(4-(Methylamino)phenyl)ethan-1-ol

To a solution of intermediate 26b (0.5 g, 1.42 mmol) in tetrahydrofuran (10 mL) at 0° C. was added lithium aluminum hydride (54 mg, 1.42 mmol) slowly in portions for 5 min and the reaction mixture was stirred at room temperature for 30 min, and the reaction mixture was heated to 60° C. for 2 h. The reaction mixture was cooled to 0° C., quenched with sodium sulfate solution under nitrogen atmosphere and passed through celite bed, washed with dichloromethane and methanol. The filtrate was dried over anhydrous sodium sulfate and concentrated under reduced pressure to afford the title compound (0.17 g, 865 umol, 60% yield). MS (ESI): 152.2 ([M+H]⁺).

### Intermediate 26d: tert-Butyl (3-((4-(2-hydroxyethyl)phenyl)(methyl)amino)propyl) (methyl)carbamate

To a solution of intermediate 26c (6.9 g, 39.9 mmol) in methanol (50 mL) was added acetic acid (3.2 g, 53.17 mmol, 3.1 mL), and the reaction mixture was stirred at room temperature for 30 min followed by the addition of sodium cyano borohydride (3.35 g, 53.17 mmol). The reaction mixture was stirred at room temperature for 12 h, concentrated under reduced pressure. The crude reaction mixture was dissolved in dichloromethane and washed with water, brine solution, dried over anhydrous sodium sulfate, filtered and concentrated under reduced pressure. The crude residue was purified by flash chromatography (DCM/EtOAc 5-20) to afford the title compound (3.2 g, 5.56 mmol, 20% yield). MS (ESI): 232.3 ([M+H]⁺).

### Intermediate 57a: 3-(2-hydroxyethyl)phenol

To a solution of 2-(3-hydroxyphenyl)acetic acid (0.5 g, 3.29 mmol) in THF (10 mL) was added 1M Borane-tetrahydrofuran complex in THF (3.3 mL, 3.3 mmol) over a period of 30 min at 0° C. and the reaction mixture was stirred at room temperature for 12 h. The reaction was quenched by dropwise addition of concentrated HCl (0.5 mL) at 0° C. and stirred for 15 min. The reaction mixture was neutralized with ammonia. The mixture was diluted with water, extracted with ethyl acetate. The organic layer was washed with water, brine solution, dried over sodium sulphate, filtered and concentrated under reduced pressure. The crude residue was purified by flash chromatography (PE/EtOAc 0-30) to afford the title compound (0.25 g, 1.81 mmol, 55% yield) as a pale yellow liquid. MS (ESI): 137.2 ([M−H]⁺).

### Intermediate 57b: tert-butyl 4-(2-(3-(2-hydroxyethyl)phenoxy)ethyl)piperazine-1-carboxylate

To a solution of intermediate 57a (0.25 g, 1.81 mmol) in acetone (2.5 mL) was added tert-butyl 4-(2-bromoethyl)piperazine-1-carboxylate (636 mg, 2.17 mmol) and potassium carbonate (625 mg, 4.52 mmol), the reaction mixture was heated to 60° C. for 12 h. The reaction was cooled to ambient temperature passed through celite bed, washed with ethyl acetate and concentrated under reduced pressure. The crude residue was purified by flash chromatography (PE/EtOAc 10-90) to afford the title compound (0.36 g, 1.03 mmol, 56% yield) as a pale yellow liquid. MS (ESI): 351.2 ([M+H]⁺).

### Intermediate 105a: 3-(2-hydroxyethyl)benzaldehyde

To a solution of N,N,N′,N′-tetramethylethylenediamine (15.0 mL, 49.74 mmol, 1.0 eq) and dimethylformamide (40 mL, 497.36 mmol, 10.0 eq) in THF (150 mL) was added n-butyllithium (40 mL, 99.47 mmol, 2.0 eq) at −70° C. under N₂ and stirred at −20° C. for 1 h. Then to the reaction mixture was added 3-bromophenethyl alcohol (10 g, 6.7 mL, 49.74 mmol, 1.0 eq) at −70° C. and slowly warmed to 25° C. and stirred for 2 h. The mixture was diluted with water and extracted with ethyl acetate. The combined organic phase was washed with brine, dried over sodium sulfate, filtered and concentrated under reduced pressure. The crude residue was purified by silica gel chromatography (PE/EtOAc 16-25) to afford the title compound (4 g, 26.64 mmol, 53% yield) as a yellow oil.

### Intermediate 105b: tert-butyl 4-[[3-(2-hydroxyethyl)phenyl]methyl]piperazine-1-carboxylate

A solution of intermediate 105a (2.0 g, 13.32 mmol, 1.0 eq), 1-Boc-piperazine (5 g, 26.64 mmol, 2.0 eq) and acetic acid (0.5 mL, 13.32 mmol, 0.1 eq) in DME (100 mL) was stirred at 25° C. for 1 h. Then to the reaction mixture was added sodiumcyanoborohydride (1.67 g, 26.64 mmol, 2.0 eq) and stirred at 25° C. for 11 h. To the residue was added water and it was extracted with ethyl acetate. The combined organic layer were dried over anhydrous sodium sulfate, filtered and concentrated in vacuo. The crude residue was purified by flash chromatography (PE/EtOAc 25-50) to afford the title compound (4 g, 12.48 mmol, 93% yield) as a yellow liquid.

### Intermediate 69a: tert-butyl 4-(2-(3-iodophenoxy)ethyl)piperazine-1-carboxylate

A sealed tube was charged with 3-iodophenol (2.0 g, 9.09 mmol), potassium carbonate (3.14 g, 22.73 mmol, 1.37 mL), tert-butyl 4-(2-bromoethyl)piperazine-1-carboxylate (3.20 g, 10.91 mmol) and acetone (20 mL). The reaction mixture was heated to 60° C. for 12 h. The reaction mixture was cooled to room temperature, filtered on celite bed and washed with ethyl acetate and concentrated under reduced pressure. The crude residue was purified by flash chromatography (PE/EtOAc 0-30) as eluent to afford the title compound (2.5 g, 5.78 mmol, 63% yield). MS (ESI): 433.1 ([M+H]⁺).

### Intermediate 69b: tert-butyl 4-(2-(3-(3-hydroxypiperidin-1-yl)phenoxy)ethyl)piperazine-1-carboxylate

A sealed tube was charged with intermediate 69a (1.0 g, 2.31 mmol), piperidin-3-ol (585 mg, 5.78 mmol), potassium phosphate tribasic anhydrous (1.47 g, 6.94 mmol) and L-proline (133 mg, 1.16 mmol) and DMF (15 mL). The reaction mixture was purged with nitrogen for 15 min and was added copper (I) iodide (220 mg, 1.16 mmol, 39.20 μL), purging was continued for another 5 min and the reaction mixture was heated to 100° C. for 16 h. The reaction was cooled to room temperature passed through celite bed, washed with ethyl acetate and concentrated under reduced pressure. The crude residue was purified by flash chromatography (PE/EtOAc 0-90) to afford the title compound (0.41 g, 1.01 mmol, 43% yield). MS (ESI): 406.3 ([M+H]⁺).

### Intermediate 77a: tert-Butyl 4-(2-(4-iodophenoxy)ethyl)piperazine-1-carboxylate

The title compound (9.5 g, 21.98 mmol, 80% yield), off white solid, was prepared in analogy to intermediate 69a from 4-iodophenol, (PE/EtOAc 0-50). MS (ESI): 433.0 ([M+H]⁺).

### Intermediate 77b: tert-butyl 4-(2-(4-(3-hydroxypiperidin-1-yl)phenoxy)ethyl)piperazine-1-carboxylate

The title compound (4.5 g, 11.1 mmol, 50% yield) was prepared in analogy to intermediate 69b from intermediate 77a, (PE/EtOAc 0-90). MS (ESI): 406.0 ([M+H]⁺).

### Intermediate 90a: 1-(3-iodophenyl)piperazine

To a solution of 3-iodoaniline (1.0 g, 4.57 mmol) in butanol (10 mL) was added sodium carbonate (1.21 g, 11.41 mmol, 478.18 μL) and 2-chloro-N-(2-chloroethyl)ethanamine (650 mg, 4.57 mmol) and the reaction mixture was heated to 120° C. for 36 h. The reaction mixture was cooled to room temperature, diluted with water and extracted with ethyl acetate. The organic layer was washed with water, brine solution, dried over sodium sulphate, filtered and concentrated under reduced pressure to afford the title compound (0.8 g, 2.08 mmol, 45% yield). MS (ESI): 289.0 ([M+H]⁺).

### Intermediate 90b: tert-butyl 4-(3-iodophenyl)piperazine-1-carboxylate

To a solution of intermediate 90a (10.0 g, 34.71 mmol) in tetrahydrofuran (100 mL) was added triethylamine (8.78 g, 86.77 mmol, 12.09 mL), di-tert-butyl dicarbonate (7.57 g, 34.71 mmol, 7.96 mL) at 0° C., the reaction mixture was stirred at room temperature for 16 h. The reaction mixture was diluted with ethyl acetate, washed with water, brine solution, dried over sodium sulphate, filtered and concentrated under reduced pressure. The crude residue was purified by flash chromatography (PE/EtOAc 0-10) to afford the title compound (10 g, 25.76 mmol, 74% yield). MS (ESI): 389.1 ([M+H]⁺).

### Intermediate 90c: tert-butyl 4-(3-(3-hydroxypiperidin-1-yl)phenyl)piperazine-1-carboxylate

The title compound (5.0 g, 13.8 mmol, 53% yield) was prepared in analogy to intermediate 69b from intermediate 90b, (PE/EtOAc 0-80). MS (ESI): 362.3 ([M+H]⁺).

### Intermediate 31a: tert-butyl 4-(4-(3-hydroxypiperidin-1-yl)phenyl)piperazine-1-carboxylate

The title compound (837 mg, 2.32 mmol, 23% yield), brown solid was prepared in analogy to intermediate 69b from tert-butyl 4-(4-iodophenyl)piperazine-1-carboxylate, (PE/EtOAc 0-100). MS (ESI): 362.3 ([M+H]⁺).

### Intermediate 38a: tert-butyl 4-(4-((3-hydroxypiperidin-1-yl)methyl)phenyl)piperazine-1-carboxylate

To a solution of tert-butyl 4-(4-formylphenyl)piperazine-1-carboxylate (1.5 g, 5.17 mmol) and piperidin-3-ol (522 mg, 5.1 mmol) in ethanol (15.0 mL) was added titanium(IV) isopropoxide, 95% (2.95 g, 10.4 mmol, 3.07 mL) at room temperature under nitrogen atmosphere and the reaction mixture was heated at 80° C. for 3 h. The reaction mixture was cooled to room temperature and were added sodium borohydride (293 mg, 7.75 mmol, 274 uL) and triethylamine (1.05 g, 10.33 mmol, 1.44 mL) successively. The reaction mixture was heated at 80° C. for 8 h. The reaction mixture was cooled to room temperature and concentrated under reduced pressure, diluted with water, extracted with ethyl acetate. The organic layer was washed with water, brine solution, dried over sodium sulphate, filtered and concentrated under reduced pressure. The crude residue was purified by flash chromatography (PE/EtOAc 20-80) to afford the title compound (1.45 g, 3.86 mmol, 74% yield) as an off-white semi solid. MS (ESI): 376.3 ([M+H]⁺).

### Intermediate 82a: tert-butyl 4-(3-acetylbenzyl)piperazine-1-carboxylate

A mixture of 3-chloromethylacetophenone (5 g, 29.65 mmol, 1.0 eq), 1-boc-piperazine (7.73 g, 41.51 mmol, 1.4 eq) and potassium carbonate (9.66 g, 29.65 mmol, 1.0 eq) in DMF (10 mL) was stirred at 80° C. for 12 h. The mixture was purified by silica gel to afford the title compound (5 g, 15.7 mmol, 42% yield) as a yellow oil. MS (ESI): 319.4 ([M+H]⁺).

### Intermediate 82b: tert-butyl 4-(3-(1-hydroxyethyl)benzyl)piperazine-1-carboxylate

A mixture of intermediate 82a (1730 mg, 5.43 mmol, 1.0 eq) and sodium borohydride (616 mg, 16.3 mmol, 3.0 eq) in ethanol (10 mL) was stirred at 80° C. for 12 h. The mixture was purified by silica gel to afford the title compound (500 mg, 1.56 mmol, 28% yield) as a yellow oil, MS (ESI): 321.4 ([M+H]⁺).

### Intermediate 21e: tert-butyl (3-((3-amino-6-chloropyridazin-4-yl)oxy)-2-phenylpropyl)(methyl)carbamate

To a solution of intermediate 21d (0.3 g, 1.13 mmol) in dimethylformamide (8 mL) at 0° C. was added sodium hydride (40.7 mg, 1.70 mmol) and it was stirred for 30 min. To this reaction mixture was added 4-bromo-6-chloropyridazin-3-amine (190 mg, 905 umol) in dimethylformamide (1 mL) and it was stirred at room temperature for 4 h. The reaction mixture was quenched with saturated ammonium chloride solution and extracted with ethyl acetate. The organic layer was washed with water, brine solution, dried over sodium sulfate, filtered and concentrated under reduced pressure. The crude residue was purified by flash chromatography (PE/EtOAc 10-60) to afford the title compound (0.1 g, 254.5 umol, 22% yield). MS (ESI): 393.5 ([M+H]⁺).

### Intermediate 26e: tert-Butyl (3-((4-(2-((3-amino-6-chloropyridazin-4-yl)oxy)ethyl)phenyl)(methyl)amino)propyl)(methyl)carbamate

The title compound (0.1 g, 177.79 umol, 17% yield) was prepared in analogy to intermediate 21e from intermediate 26d, (DCM/EtOac 10-50). MS (ESI): 450.2 ([M+H]⁺).

### Intermediate 31b: tert-butyl 4-(4-(3-((3-amino-6-chloropyridazin-4-yl)oxy)piperidin-1-yl)phenyl)piperazine-1-carboxylate

The title compound (0.6 g, 1.23 mmol, 36% yield) was prepared in analogy to intermediate 21e from intermediate 31a, NaH (3 eq), 80° C., bromide (2 eq), (PE/EtOAc 20-80). MS (ESI): 489.2 ([M+H]⁺).

### Intermediate 38b: tert-butyl 4-(4-((3-((3-amino-6-chloropyridazin-4-yl)oxy)piperidin-1-yl)methyl)phenyl)piperazine-1-carboxylate

The title compound (0.85 g, 1.69 mmol, 43% yield), light brown semi solid, was prepared in analogy to intermediate 21e from intermediate 38a, NaH (3 eq), 80° C., bromide (4 eq), (PE/EtOAc 50-90). MS (ESI): 503.3 ([M+H]⁺).

### Intermediate 50a tert-butyl (3-((3-amino-6-chloropyridazin-4-yl)oxy)-2-phenylpropyl)carbamate

To a colourless solution of 3-amino-2-phenylpropan-1-ol (500 mg, 3.14 mmol, 1.36 eq) in DMF (5 mL) was added portionwise under argon with ice bath cooling sodium hydride (126 mg, 3.15 mmol, 1.37 eq). The reaction was stirred for 30 minutes at 0° C. No gas evolution was observed anymore. The off-white suspension was warmed to room temperature and 4-bromo-6-chloropyridazin-3-amine (500 mg, 2.3 mmol, 1.0 eq) was added. The reaction immediately turned brown and was stirred for 5 h at room temperature. A portion of di-tert-butyl dicarbonate (704 mg, 749 μL, 3.22 mmol, 1.4 eq) was added at room temperature and the reaction mixture was left to stir overnight. Water was added to the reaction mixture and was subsequently extracted with EtOAc/THF (1:2) after the addition of brine. The aqueous layer was extracted with EtOAc/THF (1:1). The organic layers were then washed with brine, dried over Na2S04, filtered and concentrated in vacuo. The crude mixture was loaded onto silica and purified by flash chromatography (Heptane/EtOAc 0-100) to afford the title compound (648.9 mg, 1.7 mmol, 74% yield) as an orange oil. MS (ESI): 379.0/381.0 ([M+H]⁺).

### Intermediate 57c: tert-butyl 4-(2-(3-(2-((3-amino-6-chloropyridazin-4yl)oxy)ethyl)phenoxy)ethyl)piperazine-1-carboxylate

The title compound (0.2 g, 418.42 umol, 40% yield), brownish liquid, was prepared in analogy to intermediate 21e from intermediate 57b, (CH2Cl2/MeOH 0-5). MS (ESI): 478.2 ([M+H]⁺).

### Intermediate 62a: tert-butyl (2-((3-amino-6-chloropyridazin-4-yl)oxy)-1-phenylethyl)carbamate

To an orange solution of 2-amino-2-phenylethan-1-ol (494 mg, 3.6 mmol, 1.5 eq) in DMF (5 mL) was added portionwise under argon with ice bath cooling sodium hydride 60% dispersion in mineral oil (144 mg, 3.6 mmol, 1.5 eq). The reaction was stirred for 30 minutes at 0° C. No gas evolution was observed anymore. The orange suspension was warmed to room temperature and at this temperature 4-bromo-6-chloropyridazin-3-amine (500 mg, 2.4 mmol, 1.0 eq) was added. The reaction turned red and it was stirred for an additional 2 h at room temperature. Di-tert-butyl dicarbonate (785 mg, 3.6 mmol, 1.5 eq) was added and the reaction was stirred at room temperature overnight. Water was added and the reaction was extracted with THF/ethyl acetate. The organic layer was dried over sodium sulfate, filtered and the solvent was evaporated. The residue was several times evaporated with heptane to remove DMF and with THF and methanol to remove water to afford the title compound (1.32 g, 3.6 mmol, 90% yield) as a light brown oil. MS (ESI): 365.2/367.1 ([(M+H)⁺], MS (ESI): 409.3/411.3 ([(M+HCO2⁻)⁻].

### Intermediate 64a: rac-tert-butyl (3R,5R)-3-((3-amino-6-chloropyridazin-4-yl)oxy)-5-phenylpiperidine-1-carboxylate

The title compound (250 mg, 0.62 mmol, 44% yield), orange foam, was prepared in analogy to intermediate 21e from rac-tert-butyl (3R,5R)-3-hydroxy-5-phenylpiperidine-1-carboxylate (1.4 eq), (Heptane/EtOAc 0-100). MS (ESI): 405.1/407.1 ([M+H]⁺).

### Intermediate 66a: rac-tert-butyl (3R,5S)-3-((3-amino-6-chloropyridazin-4-yl)oxy)-5-phenylpiperidine-1-carboxylate

The title compound (261 mg, 0.64 mmol, 46% yield), orange gum, was prepared in analogy to intermediate 21e from rac-tert-butyl (3R,5S)-3-hydroxy-5-phenylpiperidine-1-carboxylate (1.4 eq), (Heptane/EtOAc 0-100). MS (ESI): 405.1/407.1 ([M+H]⁺).

### Intermediate 69c: tert-butyl 4-(2-(3-(3-((3-amino-6-chloropyridazin-4-yl)oxy)piperidin-1-yl)phenoxy)ethyl)piperazine-1-carboxylate

The title compound (261 mg, 0.64 mmol, 46% yield), orange gum, was prepared in analogy to intermediate 21e from rac-tert-butyl (3R,5S)-3-hydroxy-5-phenylpiperidine-1-carboxylate (1.4 eq), NaH (3 eq), bromide (2.5 eq), 90° C. 3 h, (PE/EtOAc 50-90). MS (ESI): 533.1 ([M+H]⁺).

### Intermediate 77c: tert-butyl 4-(2-(4-(3-((3-amino-6-chloropyridazin-4-yl)oxy)piperidin-1-yl)phenoxy)ethyl)piperazine-1-carboxylate

The title compound (1.7 g, 2.92 mmol, 26% yield) was prepared in analogy to intermediate 21e from intermediate 77b, NaH (3 eq), bromide (2.5 eq), 90° C. 3 h, (Heptane/EtOAc 50-90). MS (ESI): 533.2 ([M+H]⁺).

### Intermediate 82c: tert-butyl 4-(3-(1-((3-amino-6-chloropyridazin-4-yl)oxy)ethyl)benzyl)piperazine-1-carboxylate

The title compound (600 mg, 1.34 mmol, 42% yield), yellow oil, was prepared in analogy to intermediate 21e from intermediate 82b, 70° C. 12 h, prep-HPLC. MS (ESI): 448.2 ([M+H]⁺).

### Intermediate 90d: tert-butyl 4-(3-(3-((3-amino-6-chloropyridazin-4-yl)oxy)piperidin-1-yl)phenyl)piperazine-1-carboxylate

The title compound (2.4 g, 4.91 mmol, 35% yield), was prepared in analogy to intermediate 21e from intermediate 90c, NaH (1 eq), bromide (3 eq), 90° C. 4 h, (PE/EtOAc 40-90). MS (ESI): 489.3 ([M+H]⁺).

### Intermediate 95a: rac-tert-butyl ((1r,3r)-3-((3-amino-6-chloropyridazin-4-yl)oxy)cyclobutyl)carbamate

The title compound (2.4 g, 4.91 mmol, 35% yield), was prepared in analogy to intermediate 21e from rac-tert-butyl ((1r,3r)-3-hydroxycyclobutyl)carbamate, NaH (1.3 eq), bromide (1 eq), rt over night, (heptane/EtOAc 0-100). MS (ESI): 315.2/317.2 (100/50) ([M+H]⁺).

### Intermediate 105c: tert-butyl 4-[[3-[2-(3-amino-6-chloro-pyridazin-4-yl)oxyethyl]phenyl]methyl]piperazine-1-carboxylate

The title compound (2.4 g, 4.91 mmol, 35% yield), was prepared in analogy to intermediate 21e from intermediate 105b, NaH (1.2 eq), 50° C. 2 h, (PE/EtOAc 25-50). MS (ESI): 448.2 ([M+H]⁺).

### Intermediate 35a: tert-butyl 4-(2-(3-ethynylphenoxy)ethyl)piperazine-1-carboxylate

3-ethynylphenol (1 g, 8.46 mmol, 1.0 eq) was combined with tert-butyl 4-(2-hydroxyethyl)piperazine-1-carboxylate (2.14 g, 9.31 mmol, 1.1 eq) and triphenylphosphine (2.44 g, 9.31 mmol, 1.1 eq) in tetrahydrofuran (20 mL) at room temperature to give a brown solution. Di-tert-butyl azodicarboxylate (2.14 g, 9.31 mmol, 1.1 eq) was added and the brown solution was stirred at room temperature for 2 h. The reaction mixture was poured into ethyl acetate and was extracted with water and brine. The organic layer was dried over sodium sulfate, filtered and evaporated. The crude residue was purified by flash chromatography (heptane/EtOAc 0-100) to afford the title compound (1.987 g, 6.01 mmol, 71% yield) as a light yellow liquid. MS (ESI): 331.1 ([M+H]⁺).

### Intermediate 53a: tert-butyl 4-(3-ethynylbenzyl)piperazine-1-carboxylate

3-ethynylbenzaldehyde (1 g, 7.68 mmol, 1.0 eq) was combined with tert-butyl piperazine-1-carboxylate (1.86 g, 9.99 mmol, 1.3 eq) and acetic acid (600 mg, 572 μL, 9.99 mmol, 1.3 eq) in 1,2-Dichloroethane (20 mL) at room temperature. The yellow solution was stirred for 1 h. Sodium triacetoxyborohydride (6.51 g, 30.7 mmol, 4.0 eq) was added in three portions (slightly exothermic) and the yellow suspension was stirred for 3 h. Aqueous saturated NaHCO₃ was carefully added (strong gas evolution!) until pH=8. This mixture was extracted with dichloromethane. The organic layer was dried over sodium sulfate, filtered and evaporated. The crude residue was purified by flash chromatography (heptane/EtOAc 0-100) to afford the title compound (2.16 g, 7.2 mmol, 94% yield) as a light yellow oil. MS (ESI): 301.1 ([M+H]⁺).

### Intermediate 35b: b) tert-butyl 4-(2-(3-((3-amino-6-chloropyridazin-4-yl)ethynyl)phenoxy)ethyl)piperazine-1-carboxylate

4-bromo-6-chloropyridazin-3-amine (1.25 g, 6 mmol, 1.0 eq) was combined with intermediate 35a (1.98 g, 6 mmol, 1.0 eq), PdCl₂(PPh₃)₂ (168 mg, 240 μmol, 0.04 eq) and triethylamine (6.07 g, 8.36 mL, 60 mmol, 10.0 eq) in tetrahydrofuran (12 mL) at room temperature. The reaction was heated to 60° C. and was stirred for 2 h. The solvent was evaporated and the crude residue was purified by flash chromatography (heptane/EtOAc 0-100) as eluent to afford the title compound (1.98 g, 4.32 mmol, 72% yield) as a light brown solid. MS (ESI): 478.1955 ([M+H]⁺).

### Intermediate 53b: tert-butyl 4-(3-((3-amino-6-chloropyridazin-4-yl)ethynyl)benzyl)piperazine-1-carboxylate

The title compound (2.14 g, 5 mmol, 69.5% yield), light brown foam was prepared in analogy to intermediate 35b from intermediate 53a, (PE/EtOAc 0-100). MS (ESI): 428.4 ([M+H]⁺).

### Intermediate 29b: 6-chloro-4-(3,8-diazabicyclo[3.2.1]octan-3-yl)pyridazin-3-amine

To a cooled (0° C.) solution of Intermediate 29a (580 mg, 1.71 mmol, 1.0 eq) in DCM (6 mL) was added HCl 4M in dioxane (1.28 ml, 5.12 mmol, 3.0 eq). The reaction mixture was allowed to reach room temperature and stirred for 7 h. The reaction mixture was filtered and the solid was dried in vacuo to afford the title compound (460 mg, 1.67 mmol, 98% yield) as a brown hydrochloride salt. MS (ESI): 240.0 ([M+H]⁺).

### Intermediate 29c: tert-butyl 4-[(3-formylphenyl)methyl]piperazine-1-carboxylate

To a solution of 3-(bromomethyl)benzaldehyde (200 mg, 1 mmol, 1.0 eq) and tert-butyl piperazine-1-carboxylate (206 mg, 1.11 mmol, 1.1 eq) in DMF (3 mL) was added potassium carbonate (278 mg, 2.01 mmol, 2.0 eq). The resulting yellow suspension was stirred at room temperature for 16 h. The reaction mixture was poured into saturated NaHCO₃ and extracted with EtOAc. The organic layers were combined, washed with H₂O and brine, dried over Na₂SO₄ and concentrated. The crude material was purified by flash chromatography (heptane/EtOAc 0-60) to afford the title compound (300 mg, 983 μmol, 98% yield) as a brown solid. MS (ESI): 305.3 ([M+H]⁺).

### Intermediate 29d: tert-butyl 4-[[3-[[3-(3-amino-6-chloro-pyridazin-4-yl)-3,8-diazabicyclo[3.2.1]octan-8-yl]methyl]phenyl]methyl]piperazine-1-carboxylate

To a solution of intermediate 29b (180 mg, 576 μmol, 1.0 eq) and Hunig's base (149 mg, 201 μl, 1.15 mmol, 2.0 eq) in DCM (1 mL) was added a solution of intermediate 29c (193 mg, 633 μmol, 1.1 eq) in DCM (4 mL). The resulting suspension was stirred at room temperature for 10 min followed by the addition of sodium triacetoxyborohydride (244 mg, 1.15 mmol, 2.0 eq). The mixture was stirred at room temperature for 16 h. The reaction mixture was poured into saturated NaHCO₃ and extracted with DCM. The organic layers were combined, washed with brine, dried over Na₂SO₄ and concentrated. The crude material was purified by flash chromatography (DCM/MeOH 0-10) to afford the title compound (230 mg, 435 μmol, 75% yield) as a brown solid. MS (ESI): 528.5 ([M+H]⁺).

### Intermediate 20d: 3-[3-(3-amino-6-chloro-pyridazin-4-yl)-3,8-diazabicyclo[3.2.1]octan-8-yl]phenol

A mixture of intermediate 20c (2 g, 3.51 mmol, 1.0 eq) and potassium fluoride (1.02 g, 17.54 mmol, 5.0 eq) in DMF (20 mL) was heated at 60° C. for 16 h. The mixture was poured into water and extracted with ethyl acetate. The organic layer was concentrated in vacuum and the residue was purified by silica column (PE/EA=10:1) to afford the title compound (0.5 g, 1.51 mmol, 43% yield) as a yellow solid. MS (ESI): 332.1 ([M+H]⁺).

### Intermediate 20e: methyl 10-[3-[3-(3-amino-6-chloro-pyridazin-4-yl)-3,8-diazabicyclo[3.2.1]octan-8-yl]phenoxy]decanoate

To a mixture of intermediate 20d (300 mg, 0.900 mmol, 1.0 eq), triphenylphosphine (474 mg, 1.81 mmol, 2.0 eq), methyl 10-hydroxydecanoate (274.35 mg, 1.36 mmol, 1.5 eq) in THF (20 mL) was added diethyl azodicarboxylate (314.92 mg, 1.81 mmol, 2.0 eq) and it was stirred at 15° C. for 16 h. The mixture was concentrated and purified by flash column to afford the title compound (170 mg, 0.330 mmol, 36% yield) as a yellow oil. MS (ESI): 516.3 ([M+H]⁺).

### Intermediate 3d: 3-[3-(3-amino-6-chloro-pyridazin-4-yl)-3,8-diazabicyclo[3.2.1]octan-8-yl]benzaldehyde

To a solution of intermediate 3c (5.5 g, 15.9 mmol, 1.0 eq) in DCM (100 mL) was added manganese dioxide (6.91 g, 79.52 mmol, 5.0 eq) and it was stirred at 35° C. for 120 h. The mixture was filtered, concentrated and purified by silica column (DCM/EtOAC=2:1) to afford the title compound (4.4 g, 12.8 mmol, 80% yield) as a yellow solid.

### Intermediate 97b: tert-butyl 4-[[[4-(3-amino-6-chloro-pyridazin-4-yl)-1-phenyl-piperazine-2-carbonyl]amino]methyl]piperidine-1-carboxylate

To a solution of intermediate 97a (400 mg, 1.2 mmol, 1.0 eq), HATU (684 mg, 1.8 mmol, 1.5 eq) and Hunig's base (774 mg, 1.05 ml, 5.99 mmol, 5.0 eq) in DMF (5 ml) was added tert-butyl 4-(aminomethyl)piperidine-1-carboxylate (385 mg, 1.8 mmol, 1.5 eq). The reaction mixture was stirred at room temperature for 5 h. The reaction mixture was concentrated in vacuo. The crude material was purified by flash chromatography (heptane/EtOAc 0-100) to afford the title compound (500 mg, 945 μmol, 79% yield) as a brown solid. MS (ESI): 530.0 ([M+H]⁺).

### Intermediate 98a: tert-butyl N-[2-[[4-(3-amino-6-chloro-pyridazin-4-yl)-1-phenyl-piperazine-2-carbonyl]amino]ethyl]-N-methyl-carbamate

The title compound (430 mg, 878 μmol, 84% yield), brown oil, was prepared in analogy of intermediate 97b from tert-butyl (2-aminoethyl)(methyl)carbamate, (heptane/EtOAc 0-100). MS (ESI): 490.4 ([M+H]⁺).

### Intermediate 84a: tert-Butyl 3-[3-amino-6-(2-hydroxyphenyl)pyridazin-4-yl]-3,8-diazabicyclo[3.2.1]octane-8-carboxylate

To a solution of intermediate 29a (1.39 g, 4.09 mmol, 1.0 eq) in a mixture of dioxane (15 mL), DMA (0.75 mL) and water (1.5 mL) was added (2-hydroxyphenyl)boronic acid (1.41 g, 10.2 mmol, 2.5 eq) and potassium carbonate (1.7 g, 12.3 mmol, 3.0 eq). The solution was degassed by purging with argon and after addition of RuPhos Pd G3 (0.17 g, 0.21 mmol, 0.05 eq) the reaction mixture was stirred at 90° C. for 2 h. The reaction mixture was poured into a saturated NH₄Cl solution, extracted with ethyl acetate, washed with brine and the organic layer was dried over Na₂SO₄ and concentrated in vacuo. The crude material was purified by flash chromatography (heptane/EtOAc 0-100) to afford the title compound (0.83 g, 2.1 mmol, 51% yield) as a light yellow solid. MS (ESI): 398.3 ([M+H]⁺).

### Intermediate 2g: benzyl 4-[2-[3-[3-[3-amino-6-(2-hydroxyphenyl)pyridazin-4-yl]-3,8-diazabicyclo[3.2.1]octan-8-yl]phenoxy]ethyl]piperazine-1-carboxylate

A mixture of 2-hydroxyphenylboronic acid (35.79 mg, 0.260 mmol, 1.5 eq), Brettphos Pd G3 (14.83 mg, 0.020 mmol, 0.1 eq), sodium carbonate (36.67 mg, 0.350 mmol, 2.0 eq) and intermediate 2f (0.1 g, 0.170 mmol, 1.0 eq) in tert-butanol (3 mL) was stirred under nitrogen at 90° C. for 16 h. The mixture was purified by flash chromatography to afford the title compound (60 mg, 0.090 mmol, 54% yield).

### Intermediate 3e: 3-[3-[3-amino-6-(2-hydroxyphenyl)pyridazin-4-yl]-3,8-diazabicyclo[3.2.1]octan-8-yl]benzaldehyde

A mixture of intermediate 3d (4.3 g, 12.51 mmol, 1.0 eq), 2-hydroxyphenylboronic acid (2.59 g, 18.76 mmol, 1.5 eq), sodium carbonate (2.65 g, 25.01 mmol, 2.0 eq) and [1,1′-bis(diphenylphosphino)ferrocene]dichloropalladium(II) (915.15 mg, 1.25 mmol, 0.1 eq) in 1,4-dioxane (50 mL)/water (5 mL) was stirred under N₂ at 85° C. for 40 h. The mixture was concentrated and purified by Prep-TPC (DCM/EtOAc 1:1) to afford the title compound (2.2 g, 5.48 mmol, 43% yield) as a light yellow solid.

### Intermediate 4b: tert-butyl 4-[3-amino-6-(2-hydroxyphenyl)pyridazin-4-yl]-2-methyl-piperazine-1-carboxylate

Intermediate 4a (154 mg, 470 μmol, 1.0 eq), (2-hydroxyphenyl)boronic acid (64.8 mg, 470 μmol, 1.0 eq) and potassium carbonate (104 mg, 752 μmol, 1.6 eq) were combined with dioxane (5.02 mL) and water under argon. Brettphos Pd G3 (17 mg, 18.8 μmol, 0.04 eq) was added. The reaction mixture was heated to 110° C. and stirred for 20 h. The reaction mixture was diluted with EtOAc and water and filtered through a cartouche. It was washed with EtOAc. The crude reaction mixture was concentrated in vacuo and purified by flash chromatography (heptane/EtOAc 0-60) to afford the title compound (91 mg, 0.24 mmol, 50% yield) as a yellow solid. MS (ESI): 386.2 ([M+H]⁺).

### Intermediate 6b: tert-butyl 9-[3-amino-6-(2-hydroxyphenyl)pyridazin-4-yl]-3,9-diazaspiro[5.5]undecane-3-carboxylate

A mixture of intermediate 6a (5.0 g, 13.09 mmol, 1.0 eq), 2-hydroxyphenylboronic acid (2708.76 mg, 19.64 mmol, 1.5 eq), Brettphos-Pd-G3 (561.31 mg, 0.650 mmol, 0.05 eq) and sodium carbonate (2775.28 mg, 26.18 mmol, 2.0 eq) in 1,4-dioxane (40 mL) and water (4 mL) was stirred under N₂ at 80° C. for 16 h. The mixture was concentrated and purified by silica column (DCM/EtOAc 3:1) to afford the title compound (1.4 g, 3.19 mmol, 14% yield) as a yellow solid. MS (ESI): 440.1 ([M+H]⁺).

### Intermediate 12b: tert-butyl 9-[3-amino-6-(2-hydroxyphenyl)pyridazin-4-yl]-1-oxa-4,9-diazaspiro[5.5]undecane-4-carboxylate

A suspension of intermediate 12a (1.02 g, 2.66 mmol, 1.0 eq), (2-hydroxyphenyl)boronic acid (733 mg, 5.31 mmol, 2.0 eq), K₂CO₃ (1.1 g, 7.97 mmol, 3.0 eq) and Ruphos Pd G3 (111 mg, 133 μmol, 0.05 eq) in a mixture of degassed dioxane (10 mL) and H₂O (1 mL) was stirred at 120° C. for 16 h under argon. The reaction mixture was poured in saturated NaHCO₃ and extracted with EtOAc. The organic layers were combined, washed with H₂O and brine. The organic layers were dried over Na₂SO₄ and concentrated in vacuo. The crude material was purified by flash chromatography (heptane/EtOAc 0-100) to afford the title compound (860 mg, 1.95 mmol, 73% yield) as a light brown solid. MS (ESI): 442.4 ([M+H]⁺).

### Intermediate 13g: benzyl 4-[[3-[3-[3-amino-6-(2-hydroxyphenyl)pyridazin-4-yl]-3,8-diazabicyclo[3.2.1]octan-8-yl]phenyl]methylene]piperidine-1-carboxylate

The title compound (800 mg, 1.33 mmol, 62% yield), yellow solid, was prepared in analogy to intermediate 6b from intermediate 13f, (DCM/EtOAc 3:1).

### Intermediate 16b: tert-butyl 7-[3-amino-6-(2-hydroxyphenyl)pyridazin-4-yl]-4,7-diazaspiro[2.5]octane-4-carboxylate

The title compound (200 mg, 503 μmol, 74% yield), brown solid, was prepared in analogy to intermediate 12b from intermediate 16a, (DCM/MeOH 0-10). MS (ESI): 398.3 ([M+H]⁺).

### Intermediate 17b: tert-butyl 4-[3-amino-6-(2-hydroxyphenyl)pyridazin-4-yl]-3,6-dihydro-2H-pyridine-1-carboxylate

The title compound (451 mg, 1.2 mmol, 88% yield), light brown solid, was prepared in analogy to intermediate 12b from intermediate 17a, (DCM/MeOH 0-10). MS (ESI): 369.3 ([M+H]⁺).

### Intermediate 19b: tert-Butyl 3-[3-amino-6-(2-hydroxyphenyl)pyridazin-4-yl]-3,9-diazabicyclo[3.3.1]nonane-9-carboxylate

The title compound (16.9 mg, 41 μmol, 29% yield), light yellow solid, was prepared in analogy to intermediate 84a from intermediate 19a, 2 eq boronic acid, (Heptane/EtOAc 0-100). MS (ESI): 412.4 ([M+H]⁺).

### Intermediate 20f: methyl 10-[3-[3-[3-amino-6-(2-hydroxyphenyl)pyridazin-4-yl]-3,8-diazabicyclo[3.2.1]octan-8-yl]phenoxy]decanoate

The title compound (41 mg, 0.070 mmol, 21% yield), light yellow solid, was prepared in analogy to intermediate 6b from intermediate 20e, (DCM/EtOAc 3:1) and prep-HPLC. MS (ESI): 574.5 ([M+H]⁺).

### Intermediate 21f: tert-Butyl (3-((3-amino-6-(2-hydroxyphenyl)pyridazin-4-yl)oxy)-2-phenylpropyl)(methyl)carbamate

In a sealed tube, intermediate 21e (0.15 g, 381.8 umol), (2-hydroxyphenyl)boronic acid (58 mg, 420 umol), bis(diphenylphosphino)ferrocene]dichloropalladium(II), complex with dichloromethane (32 mg, 38.2 umol) and potassium carbonate (160 mg, 1.2 mmol) and 1,4-dioxane (4 mL) and water (0.2 mL) were added and the reaction mixture was degassed with nitrogen for 10 min, and heated to 100° C. for 16 h. The reaction was cooled to room temperature, passed through celite bed, washed with ethyl acetate and concentrated under reduced pressure. The crude residue was purified by flash chromatography (Heptane/EtOAc 10-80) as eluent to afford the title compound (0.08 g, 177.57 umol, 46% yield). MS (ESI): 451.2 ([M+H]⁺).

### Intermediate 29e: tert-butyl 4-[[3-[[3-[3-amino-6-(2-hydroxyphenyl)pyridazin-4-yl]-3,8-diazabicyclo[3.2.1]octan-8-yl]methyl]phenyl]methyl]piperazine-1-carboxylate

The title compound (200 mg, 341 μmol, 78% yield), light brown solid, was prepared in analogy to intermediate 84a from intermediate 29d, (DCM/MeOH 0-5) and prep-HPLC. MS (ESI): 586.6 ([M+H]⁺).

### Intermediate 31c: tert-butyl 4-(4-(3-((3-amino-6-(2-hydroxyphenyl)pyridazin-4-yl)oxy)piperidin-1-yl)phenyl)piperazine-1-carboxylate

The title compound (0.52 g, 951 μmol, 47% yield), brown solid, was prepared in analogy to intermediate 21f from intermediate 31b, (PE/MeOH 10-80). MS (ESI): 547.3 ([M+H]⁺).

### Intermediate 26f: tert-Butyl (3-((4-(2-((3-amino-6-(2-hydroxyphenyl)pyridazin-4-yl)oxy)ethyl)phenyl)(methyl)amino)propyl)(methyl)carbamate

The title compound (0.22 g, 407 umol, 23 yield), brown solid, was prepared in analogy to intermediate 21f from intermediate 26e, (PE/MeOH 10-50). MS (ESI): 508.3 ([M+H]⁺).

### Intermediate 35c: tert-butyl 4-(2-(3-((3-amino-6-(2-hydroxyphenyl)pyridazin-4-yl)ethynyl)phenoxy)ethyl)piperazine-1-carboxylate

The title compound (0.94 g, 1.82 mmol, 43.9% yield), yellow solid, was prepared in analogy to intermediate 84a from intermediate 35b, (EtOAc/MeOH 0-10). MS (ESI): 516.2 ([M+H]⁺).

### Intermediate 38c: tert-butyl 4-(4-((3-((3-amino-6-(2-hydroxyphenyl)pyridazin-4-yl)oxy)piperidin-1-yl)methyl)phenyl)piperazine-1-carboxylate

The title compound (600 mg, 1.07 mmol, 63% yield), off-white solid, was prepared in analogy to intermediate 21f from intermediate 38b, (PE/EtOAc 10-90). MS (ESI): 561.3 ([M+H]⁺).

### Intermediate 50b: tert-butyl (3-((3-amino-6-(2-hydroxyphenyl)pyridazin-4-yl)oxy)-2-phenylpropyl)carbamate

The title compound (425 mg, 0.97 mmol, 56% yield), orange gum, was prepared in analogy to intermediate 84a in dioxane/water from intermediate 50a, (DCM/MeOH 0-10). MS (ESI): 437.4 ([M+H]⁺).

### Intermediate 52d: tert-butyl 4-[2-[3-[9-[3-amino-6-(2-hydroxyphenyl)pyridazin-4-yl]-1-oxa-4,9-diazaspiro[5.5]undecan-4-yl]phenoxy]ethyl]piperazine-1-carboxylate

The title compound (120 mg, 186 μmol, 29% yield), yellow solid, was prepared in analogy to intermediate 84a in dioxane/water from intermediate 52c, (DCM/MeOH 0-5). MS (ESI): 646.5 ([M+H]⁺).

### Intermediate 53c: tert-butyl 4-(3-((3-amino-6-(2-hydroxyphenyl)pyridazin-4-yl)ethynyl)benzyl)piperazine-1-carboxylate

The title compound (1.48 g, 3.05 mmol, 60% yield), yellow solid, was prepared in analogy to intermediate 84a from intermediate 53b, (EtOAc/MeOH 0-10). MS (ESI): 486.3 ([M+H]⁺).

### Intermediate 55f: benzyl 4-(2-(2-((1R,5S)-3-(3-amino-6-(2-hydroxyphenyl)pyridazin-4-yl)-3,8-diazabicyclo[3.2.1]octan-8-yl)phenoxy)ethyl)piperazine-1-carboxylate

The title compound (287 mg, 451 μmol, 33% yield), yellow solid, was prepared in analogy to intermediate 84a from intermediate 55e, (amino modified silica gel heptane/EtOAc 0-65). MS (ESI): 636.4 ([M+H]⁺).

### Intermediate 57d: tert-butyl 4-(2-(3-(2-((3-amino-6-(2-hydroxyphenyl)pyridazin-4-yl)oxy)ethyl)phenoxy)ethyl)piperazine-1-carboxylate

The title compound (0.4 g, 746.78 umol, 54% yield), brownish semi-solid, was prepared in analogy to intermediate 21f from intermediate 57c, (PE/EtOAc 0-80). MS (ESI): 536.3 ([M+H]⁺).

### Intermediate 62b: tert-butyl (2-((3-amino-6-(2-hydroxyphenyl)pyridazin-4-yl)oxy)-1-phenylethyl)carbamate

The title compound (100 mg, 0.24 mmol, 10% yield), light yellow solid, was prepared in analogy to intermediate 21f from intermediate 62a, (EtOAc/MeOH 0-10). MS (ESI): 486.3 ([M+H]⁺).

### Intermediate 64b: rac-tert-butyl (3R,5R)-3-((3-amino-6-(2-hydroxyphenyl)pyridazin-4-yl)oxy)-5-phenylpiperidine-1-carboxylate

The title compound (210 mg, 0.45 mmol, 73% yield), brown solid, was prepared in analogy to intermediate 21f from intermediate 64a, (DCM/MeOH 0-4). MS (ESI): 463.4 ([M+H]⁺).

### Intermediate 66b: rac-tert-butyl (3R,5S)-3-((3-amino-6-(2-hydroxyphenyl)pyridazin-4-yl)oxy)-5-phenylpiperidine-1-carboxylate

The title compound (190 mg, 0.41 mmol, 65% yield), light brown solid, was prepared in analogy to intermediate 21f from intermediate 66a, (DCM/MeOH 0-4). MS (ESI): 463.4 ([M+H]⁺).

### Intermediate 68e: benzyl 4-(2-(4-((1R,5S)-3-(3-amino-6-(2-hydroxyphenyl)pyridazin-4-yl)-3,8-diazabicyclo[3.2.1]octan-8-yl)phenoxy)ethyl)piperazine-1-carboxylate

The title compound (309 mg, 486 μmol, 34% yield), yellow solid, was prepared in analogy to intermediate 84a from intermediate 68d, (DCM/MeOH 0-5). MS (ESI): 636.3 ([M+H]⁺).

### Intermediate 69d: tert-butyl 4-(2-(3-(3-((3-amino-6-(2-hydroxyphenyl)pyridazin-4-yl)oxy)piperidin-1-yl)phenoxy)ethyl)piperazine-1-carboxylate

The title compound ((0.2 g, 264.43 μmol, 35% yield), was prepared in analogy to intermediate 21f from intermediate 69c, (PE/EtOAc 0-90). MS (ESI): 591.3 ([M+H]⁺).

### Intermediate 74b: 2-(6-amino-5-(2-fluoro-3-(hydroxymethyl)phenyl)pyridazin-3-yl)phenol

The title compound (192 mg, 0.62 mmol, 57% yield), orange solid, was prepared in analogy to intermediate 84a from intermediate 74a, (heptane/EtOAc 0-100). MS (ESI): 312.1 ([M+H]⁺).

### Intermediate 75: 2-(4-(3-amino-6-(2-hydroxyphenyl)pyridazin-4-yl)-1H-pyrazol-1-yl)-2-phenylacetic acid

A mixture of BrettPhos-Pd-G3 (227 mg, 0.150 mmol, 0.1 eq), sodium carbonate (462 mg, 4.36 mmol, 3.0 eq), intermediate 75b (500 mg, 1.45 mmol, 1.0 eq), 2-hydroxyphenylboronic acid (301 mg, 2.18 mmol, 1.5 eq) in DMSO (10 mL) and water (1 mL) was stirred at 70° C. for 12 h under N₂ atmosphere. The reaction mixture was purified by prep-HPLC to afford the title compound (30 mg, 0.077 mmol, 5% yield) as a white solid. MS (ESI): 388.2 ([M+H]⁺).

### Intermediate 77d: tert-butyl 4-(2-(4-(3-((3-amino-6-(2-hydroxyphenyl)pyridazin-4-yl)oxy)piperidin-1-yl)phenoxy)ethyl)piperazine-1-carboxylate

The title compound (1.1 g, 1.64 mmol, 51% yield), light brown semi solid, was prepared in analogy to intermediate 21f from intermediate 77c, (EtOAc/MeOH 0-3). MS (ESI): 590.0 ([M+H]⁺).

### Intermediate 82d: tert-butyl 4-(3-(1-((3-amino-6-(2-hydroxyphenyl)pyridazin-4-yl)oxy)ethyl)benzyl) piperazine-1-carboxylate

The title compound (300 mg, 0.59 mmol, 85% yield), yellow solid, was prepared in analogy to intermediate 84a from intermediate 82c, prep-HPLC. MS (ESI): 506.4 ([M+H]⁺).

### Intermediate 83e: benzyl 4-(3-((1-(3-amino-6-(2-hydroxyphenyl)pyridazin-4-yl)azetidin-3-yl)oxy)benzyl) piperazine-1-carboxylate

The title compound (300 mg, 0.53 mmol, 89% yield), yellow oil, was prepared in analogy to intermediate 84a from intermediate 83d, prep-HPLC. MS (ESI): 567.4 ([M+H]⁺).

### Intermediate 87b: 4-(3-amino-6-(2-hydroxyphenyl)pyridazin-4-yl)benzaldehyde

The title compound (151 mg, 518 μmol, 60% yield), yellow solid, was prepared in analogy to intermediate 84a from intermediate 87a, (EtOAc/MeOH 0-10). MS (ESI): 292.0 ([M+H]⁺).

### Intermediate 89b: tert-Butyl N-[3-[3-amino-6-(2-hydroxyphenyl)pyridazin-4-yl]-3-azabicyclo[3.2.1]octan-8-yl]carbamate

The title compound (150 mg, 0.36 mmol, 27% yield), off-white solid, was prepared in analogy to intermediate 84a from intermediate 89a, (heptane/EtOAc 0-100). MS (ESI): 412.3 ([M+H]⁺).

### Intermediate 90e: tert-butyl 4-(3-(3-((3-amino-6-(2-hydroxyphenyl)pyridazin-4-yl)oxy)piperidin-1-yl)phenyl)piperazine-1-carboxylate

The title compound (1.2 g, 2.20 mmol, 44% yield), was prepared in analogy to intermediate 21f from intermediate 90d, (DCM/MeOH 0-3). MS (ESI): 547.2 ([M+H]⁺).

### Intermediate 94d: benzyl 4-[3-[1-[3-amino-6-(2-hydroxyphenyl)pyridazin-4-yl]azetidin-3-yl]oxyphenyl]piperazine-1-carboxylate

The title compound (300 mg, 0.540 mmol, 52% yield), white solid, was prepared in analogy to intermediate 84a from intermediate 94c, prep-HPLC.

### Intermediate 95b: rac-tert-butyl ((1r,3r)-3-((3-amino-6-(2-hydroxyphenyl)pyridazin-4-yl)oxy)cyclobutyl)carbamate

The title compound ((27 mg, 22% yield), off-white powder, was prepared in analogy to intermediate 84a from intermediate 95a, (heptane/EtOAc 0-100). MS (ESI): 373.3 6 ([M+H]⁺).

### Intermediate 97c: tert-butyl 4-[[[4-[3-amino-6-(2-hydroxyphenyl)pyridazin-4-yl]-1-phenyl-piperazine-2-carbonyl]amino]methyl]piperidine-1-carboxylate

The title compound (35 mg, 60 μmol, 53% yield), yellow solid, was prepared in analogy to intermediate 84a from intermediate 97b, (heptane/EtOAc 0-100). MS (ESI): 588.5 ([M+H]⁺).

### Intermediate 98b: tert-butyl N-[2-[[4-[3-amino-6-(2-hydroxyphenyl)pyridazin-4-yl]-1-phenyl-piperazine-2-carbonyl]amino]ethyl]-N-methyl-carbamate

The title compound (55 mg, 100 μmol, 15% yield), brown foam, was prepared in analogy to intermediate 84a from intermediate 98a, (DCM/MeOH 0-10). MS (ESI): 548.2 ([M+H]⁺).

### Intermediate 99d: tert-butyl 4-[[[1-[3-amino-6-(2-hydroxyphenyl)pyridazin-4-yl]-4-phenyl-piperidine-4-carbonyl]amino]methyl]piperidine-1-carboxylate

The title compound (300 mg, 512 μmol, 75% yield), brown foam, was prepared in analogy to intermediate 84a from intermediate 99c, (DCM/MeOH 0-5). MS (ESI): 476.4 ([M+H]⁺).

### Intermediate 101: [3-[1-[3-amino-6-(2-hydroxyphenyl)pyridazin-4-yl]azetidin-3-yl]oxyphenyl]-piperazin-1-yl-methanone

The title compound (350 mg, 0.780 mmol, 50% yield), yellow solid, was prepared in analogy to intermediate 84a from intermediate 101d, by prep-HPLC (NH₃). MS (ESI): 447.1 ([M+H]⁺).

### Intermediate 103: [4-[1-[3-amino-6-(2-hydroxyphenyl)pyridazin-4-yl]azetidin-3-yl]oxyphenyl]-piperazin-1-yl-methanone

The title compound (60 mg, 0.130 mmol, 14% yield), yellow solid, was prepared in analogy to intermediate 84a from intermediate 103d, prep-HPLC. MS (ESI): 447.2 ([M+H]⁺).

### Intermediate 105d: tert-butyl 4-[[3-[2-[3-amino-6-(2-hydroxyphenyl)pyridazin-4-yl]oxyethyl]phenyl]methyl]piperazine-1-carboxylate

The title compound (600 mg, 1.19 mmol, 59% yield), yellow solid, was prepared in analogy to intermediate 84a from intermediate 105c, (PE/EtOAc 67-100). MS (ESI): 506.2 ([M+H]⁺).

### Intermediate 107a: benzyl 4-[[3-[3-[3-amino-6-(5-fluoro-2-hydroxy-phenyl)pyridazin-4-yl]-3,8-diazabicyclo[3.2.1]octan-8-yl]phenyl]methyl]piperazine-1-carboxylate

The title compound (827 mg, 1.33 mmol, 72% yield), yellow solid, was prepared in analogy to intermediate 84a from intermediate 102d and (5-fluoro-2-hydroxyphenyl)boronic acid, (EtOAc/MeOH 0-10). MS (ESI): 624.3 ([M+H]⁺).

### Intermediate 108a: tert-butyl 4-(4-(3-((3-amino-6-(5-fluoro-2-hydroxyphenyl)pyridazin-4-yl)oxy)piperidin-1-yl)phenyl)piperazine-1-carboxylate

The title compound (482 mg, 794 μmol, 38% yield), brown foam, was prepared in analogy to intermediate 84a from intermediate 31b and (5-fluoro-2-hydroxyphenyl)boronic acid, (heptane/EtOAc 0-100). MS (ESI): 563.2 ([M+H]⁺).

### Intermediate 100a: tert-butyl 4-(4-(3-((3-amino-6-(2-aminophenyl)pyridazin-4-yl)oxy)piperidin-1-yl)phenyl)piperazine-1-carboxylate

The title compound (345 mg, 632 μmol, 77% yield), light brown foam, was prepared in analogy to intermediate 84a from intermediate 31b and (2-aminophenyl)boronic acid, (heptane/EtOAc 0-100). MS (ESI): 546.3 ([M+H]⁺).

### Intermediate 102e: benzyl 4-[[3-[3-[3-amino-6-(2-aminophenyl)pyridazin-4-yl]-3,8-diazabicyclo[3.2.1]octan-8-yl]phenyl]methyl]piperazine-1-carboxylate

The title compound (427 mg, 706 μmol, 77% yield), off-white solid, was prepared in analogy to intermediate 84a from intermediate 102d and (2-aminophenyl)boronic acid, (EtOAc/MeOH 0-10). MS (ESI): 605.3 ([M+H]⁺).

### Intermediate 110f: benzyl 4-[2-[3-[8-[3-amino-6-(5-fluoro-2-hydroxy-phenyl)pyridazin-4-yl]-3,8-diazabicyclo[3.2.1]octan-3-yl]phenoxy]ethyl]piperazine-1-carboxylate

Intermediate 110e (400 mg, 692 μmol, 1.0 eq) was stirred with (5-fluoro-2-hydroxyphenyl)boronic acid (216 mg, 1.38 mmol, 2.0 eq) and potassium carbonate (239 mg, 1.73 mmol, 2.5 eq) in Dioxane (3 mL), DMA (600 μL) and Water (200 μL). Argon was bubbled trough the reaction for 2 min. Then RuPhos Pd G3 (28.9 mg, 34.6 μmol, 0.05 eq) was added. The reaction mixture was stirred overnight at 130° C. in a closed vessel. The crude material was purified by column chromatography using ethyl acetate/methanol (0-10% methanol) as eluent to afford the title compound (231 mg, 339 μmol, 49% yield) as a dark brown foam, MS (ESI): 654.3228 ([M+H]⁺).

### Intermediate 84b: 2-[6-Amino-5-(3,8-diazabicyclo[3.2.1]octan-3-yl)pyridazin-3-yl]phenol

Intermediate 84a (0.83 g, 2.1 mmol, 1.0 eq) was dissolved in DCM (12 mL) and HCl (5.2 mL, 20.9 mmol, 10.0 eq; 4 M solution in dioxane) was slowly added. The reaction mixture was stirred at ambient temperature for 4 h. The reaction mixture was concentrated in vacuo to afford the title compound (0.70 g, 2.35 mmol, 100% yield) as an off-white hydrochloride salt. MS (ESI): 298.2 ([M+H]⁺).

### Intermediate 74c: 2-(6-amino-5-(3-(chloromethyl)-2-fluorophenyl)pyridazin-3-yl)phenol

To a solution of intermediate 74b (190 mg, 610 μmol, 1.0 eq) and triethylamine (124 mg, 170 μL, 1.22 mmol, 2.0 eq) in anhydrous THF (4 mL) was added dropwise Mesyl-Cl (165 mg, 112 μL, 1.42 mmol, 1.5 eq) at 0° C. under argon gas. After 5 minutes of stirring at 0° C., the water/ice bath was removed to allow the reaction mixture to warm to room temperature. After 1 h, the reaction mixture was cooled to 0° C. for further addition of triethylamine (14.5 mg, 20 μL, 143 μmol, 0.235 eq) and Mesyl-Cl (14.7 mg, 10 μL, 128 μmol, 0.21 eq). The reaction mixture was warmed to room temperature and stirred for a further hour. The reaction mixture was then subsequently cooled to 0° C. for the further addition of Mesyl-Cl (58.8 mg, 40 μL, 513 μmol, 0.841 eq). After a further 30 min, the reaction mixture was concentrated in vacuo to afford the title compound (536 mg, 1.62 mmol, 99% yield) as a crude orange oil. MS (ESI): 330.1/332.1 ([M+H]⁺).

### Intermediate 74d: tert-butyl 4-(3-(3-amino-6-(2-hydroxyphenyl)pyridazin-4-yl)-2-fluorobenzyl)piperazine-1-carboxylate

DIPEA (157 mg, 212 μL, 1.21 mmol, 2.0 eq) was added to a solution of intermediate 74c (200 mg, 607 μmol, 1.0 eq) and tert-butyl piperazine-1-carboxylate (136 mg, 728 μmol, 1.2 eq) in Acetonitrile (2.5 mL) and the reaction mixture was stirred at room temperature. After 4 h stirring, a further portion of tert-butyl piperazine-1-carboxylate (56.5 mg, 303 μmol, 0.5 eq) and DIPEA (78.4 mg, 106 μL, 607 μmol, 1.0 eq) was added. The reaction mixture was then left to stir overnight at room temperature. The reaction mixture was concentrated in vacuo. The crude mixture was loaded onto silica gel and purified by flash chromatography (SiO2, 12 g, 30% to 100% EtOAc in heptane) to afford the title compound (130 mg, 0.27 mmol, 44% yield) as a yellow solid. MS (ESI): 480.4 ([M+H]⁺), 380.3 ([M+H-Boc]⁺)

### Intermediate 20: 10-[3-[3-[3-amino-6-(2-hydroxyphenyl)pyridazin-4-yl]-3,8-diazabicyclo[3.2.1]octan-8-yl]phenoxy]decanoic acid

A mixture of intermediate 20f (41 mg, 0.070 mmol, 1.0 eq) and lithium hydroxide (17 mg, 0.710 mmol, 10.0 eq) in water (1 mL) and THF (1 mL) was stirred at 15° C. for 16 h. The mixture was poured into water (10 mL) and acidified to pH=3 with HCl solution (0.5 N). Then it was extracted with EtOAc, washed with brine and concentrated to afford the title compound (39 mg, 0.070 mmol, 97% yield) as a light yellow oil. MS (ESI): 560.2 ([M+H]⁺).

### Intermediate 2: 2-[6-amino-5-[8-[3-(2-piperazin-1-ylethoxy)phenyl]-3,8-diazabicyclo[3.2.1]octan-3-yl]pyridazin-3-yl]phenol

A mixture of intermediate 2g (400 mg, 0.630 mmol, 1.0 eq) and palladium on carbon (0.07 mL, 0.060 mmol, 0.1 eq) in methanol (10 mL) was stirred under H₂ at 20° C. for 16 h. The mixture was filtered, concentrated and purified by flash column (TFA) to afford the title compound (316 mg, 0.510 mmol, 82% yield) as a yellow solid as a trifluoroacetic acid.

### Intermediate 21: 2-(6-amino-5-(3-(methylamino)-2-phenylpropoxy)pyridazin-3-yl)phenol

To a solution of intermediate 21f (0.08 g, 177.6 umol) in dichloromethane (2 mL) at 0° C. was added trifluoro acetic acid (296.00 mg, 2.60 mmol, 0.2 mL) and stirred at room temperature for 3 h. The reaction mixture was concentrated under reduced pressure, and co-distilled with dichloromethane to afford the title compound (60 mg, 171.23 umol, 96% yield). MS (ESI): 351.5 ([M+H]⁺).

### Intermediate 31: 2-(6-amino-5-((1-(4-(piperazin-1-yl)phenyl)piperidin-3-yl)oxy)pyridazin-3-yl)phenol

To a solution of intermediate 31c (125 mg, 228.66 μmol) in dichloromethane (2 mL) at 0° C. was added trifluoro acetic acid (260 mg, 2.29 mmol, 176.16 μL) and it was stirred at room temperature for 3 h. The reaction mixture was concentrated under reduced pressure, and co-distilled with dichloromethane to afford the title compound (100 mg, 223.94 μmol, 97% yield). MS (ESI): 447.5 ([M+H]⁺).

### Intermediate 90: 2-(6-amino-5-((1-(3-(piperazin-1-yl)phenyl)piperidin-3-yl)oxy)pyridazin-3-yl)phenol

To a solution of intermediate 90e (150 mg, 274.39 μmol) in dichloromethane (2 mL) was added trifluoroacetic acid (313 mg, 2.74 mmol, 211.39 μL) at 0° C. The reaction mixture was warmed to room temperature and stirred for 3 h. The reaction mixture was concentrated under reduced pressure, distilled with chloroform (2×20 mL) to afford the title compound (120 mg, 268.73 μmol, 97% yield). MS (ESI): 447.8 ([M+H]⁺).

### Intermediate 105: 2-[6-amino-5-[2-[3-(piperazin-1-ylmethyl)phenyl]ethoxy]pyridazin-3-yl]phenol

To a solution of intermediate 105d (300 mg, 0.590 mmol, 1.0 eq) in DCM (5 mL) was added TFA (5 mL, 64.89 mmol, 36.0 eq) and the reaction was stirred at 25° C. for 1 h. The reaction solution was concentrated and purified by prep-HPLC to afford the title compound (50 mg, 0.120 mmol, 20% yield) as a white solid. MS (ESI): 406.3 ([M+H]⁺).

### Intermediate 13: 2-[6-amino-5-[8-[3-(4-piperidylidenemethyl)phenyl]-3,8-diazabicyclo[3.2.1]octan-3-yl]pyridazin-3-yl]phenol

To a mixture of intermediate 13g (100 mg, 0.170 mmol, 1.0 eq) in methanol (10 mL)/EtOAc (10 mL) was added palladium on activated charcoal (0.02 mL, 0.020 mmol, 0.1 eq) and stirred under H₂ (1520 mmHg) at 20° C. for 16 h. The mixture was filtered and concentrated to afford the title compound (90 mg, 0.190 mmol, 115% yield) as a yellow oil.

### Intermediate 3f: tert-butyl 4-[[3-[3-[3-amino-6-(2-hydroxyphenyl)pyridazin-4-yl]-3,8-diazabicyclo[3.2.1]octan-8-yl]phenyl]methyl]piperazine-1-carboxylate

A mixture of intermediate 3e (330.0 mg, 0.820 mmol, 1.0 eq) and 1-Boc-piperazine (306.19 mg, 1.64 mmol, 2.0 eq) in 1,2-dichloroethane (10 mL)/methanol (10 mL) was stirred at 25° C. for 0.5 h, then sodium triacetoxyborohydride (348.43 mg, 1.64 mmol, 2.0 eq) was added to the reaction and it was stirred at 10° C. for 16 h. The mixture was concentrated to remove solvent, poured into water, extracted with EtOAc, washed with brine and finally concentrated to afford the title compound (400 mg, 0.70 mmol, 85% yield) as a yellow solid.

### Intermediate 55: 2-(6-amino-5-((1R,5S)-8-(2-(2-(piperazin-1-yl)ethoxy)phenyl)-3,8-diazabicyclo[3.2.1]octan-3-yl)pyridazin-3-yl)phenol

A 100 ml two-necked round-bottomed flask was charged with intermediate 55f (287 mg, 451 μmol, 1.0 eq), methanol (30 mL) and THF (15 mL). The flask was evacuated to approximately 120 mbar until the solvent began to bubble gently and was then back-filled with argon after 60 s. This procedure was repeated twice. After addition of the catalyst 10% palladium on activated charcoal (48 mg, 45.1 μmol, 0.1 eq) the flask was evacuated to 120 mbar, then back-filled with hydrogen and stirred for 15 h under an atmosphere of ˜1 bar of hydrogen gas. The catalyst was removed by filtration through a Sartorius filter and washed with methanol. The filtrate was concentrated in vacuo to afford the title compound (202 mg, 403 μmol, 89% yield) as a yellow solid. MS (ESI): 502.3 ([M+H]⁺).

### Intermediate 68: 2-(6-amino-5-((1R,5S)-8-(4-(2-(piperazin-1-yl)ethoxy)phenyl)-3,8-diazabicyclo[3.2.1]octan-3-yl)pyridazin-3-yl)phenol

The title compound (234 mg, 466 μmol, 96% yield), yellow solid, was prepared in analogy to intermediate 55 from intermediate 68e. MS (ESI): 502.3 ([M+H]⁺).

### Intermediate 83: 2-(6-amino-5-(3-(3-(piperazin-1-ylmethyl)phenoxy)azetidin-1-yl)pyridazin-3-yl)phenol

A mixture of intermediate 83e (300 mg, 0.53 mmol, 1.0 eq) and 10% palladium on active carbon (100 mg) in methanol (10 mL) was stirred at 25° C. for 12 h under H₂ atmosphere (15 psi). The reaction mixture was filtered and the filtrate was purified by prep-HPLC to afford the title compound (150 mg, 0.35 mmol, 65% yield) as a white solid. MS (ESI): 433.3 ([M+H]⁺).

### Intermediate 94: 2-[6-amino-5-[3-(3-piperazin-1-ylphenoxy)azetidin-1-yl]pyridazin-3-yl]phenol

The title compound (120 mg, 0.290 mmol, 62% yield), white solid, was prepared in analogy to intermediate 83 from intermediate 94d.

### Intermediate 95: rac-2-(6-amino-5-((1r,3r)-3-aminocyclobutoxy)pyridazin-3-yl)phenol dihydrochloride

(ELN028111-152, RO7281710-001-001)

To a solution of intermediate 95b (22 mg, 59.1 μmol, Eq: 1) in DCM (500 μL) was added HCl (4M in Dioxane) (500 μL, 2 mmol, Eq: 33.9) at room temperature. The reaction mixture was left to stir for 1 hour before removing the solvent in vacuo. The title compound was obtained as a off-white powder (20 mg, 98%). MS ISP (m/e): 273.2 [(M+H)+].

### Intermediate 102: 6-(2-aminophenyl)-4-[8-[3-(piperazin-1-ylmethyl)phenyl]-3,8-diazabicyclo[3.2.1]octan-3-yl]pyridazin-3-amine

Intermediate 102e (422 mg, 698 μmol, 1.0 eq) was stirred with Pd—C (74.3 mg, 698 μmol, 1.0 eq) under a hydrogen atmosphere in methanol (12 mL) and THF (2.4 mL) at room temperature overnight. The catalyst was filtered off and the solvent was evaporated under reduced pressure then dried under high vacuum to afford the title compound (330 mg, 701 μmol, 100% yield) as an off-white foam. MS (ESI): 471.2 ([M+H]⁺).

### Intermediate 107: 2-[6-amino-5-[8-[3-(piperazin-1-ylmethyl)phenyl]-3,8-diazabicyclo[3.2.1]octan-3-yl]pyridazin-3-yl]-4-fluoro-phenol

The title compound (323 mg, 485 μmol, 36% yield), white solid, was prepared in analogy to intermediate 102f from intermediate 107a. MS (ESI): 490.2712 ([M+H]⁺).

### Intermediate 3: 2-[6-amino-5-[8-[3-(piperazin-1-ylmethyl)phenyl]-3,8-diazabicyclo[3.2.1]octan-3-yl]pyridazin-3-yl]phenol

A mixture of intermediate 3f (0.9 g, 1.57 mmol, 1.0 eq) and hydrochloric acid in MeOH (10 mL, 1.57 mmol, 1.0 eq) in methanol (10 mL) was stirred at 25° C. for 16 h. To the mixture was added MTBE (30 mL) and the solid was filtered to afford the title compound (780 mg, 1.54 mmol, 97% yield) as a white hydrochloride salt.

### Intermediate 6: 2-[6-amino-5-(3,9-diazaspiro[5.5]undecan-3-yl)pyridazin-3-Yl]phenol

To a solution of intermediate 6b (400 mg, 0.910 mmol, 1.0 eq) in methanol (4 mL) was added 4 M HCl in dioxane (3.0 mL) at 25° C. for 16 h. The mixture was concentrated in vacuum to afford the title compound (200 mg, 0.590 mmol, 60% yield) as a hydrochloride salt. MS (ESI): 340.1 ([M+H]⁺).

### Intermediate 12:2-[6-amino-5-(1-oxa-4,9-diazaspiro[5.5]undecan-9-yl)pyridazin-3-yl]phenol

To a cooled (0° C.) solution of intermediate 12b (330 mg, 747 μmol, 1.0 eq) in DCM (4 mL) was added HCl 4M in dioxane (934 μL, 3.74 mmol, 5.0 eq). The reaction mixture was allowed to reach room temperature and stirred for 16 h. The reaction mixture was concentrated in vacuo to afford the title compound (303 mg, 732 μmol, 97% yield) as a white hydrochloride salt. MS (ESI): 342.3 ([M+H]⁺).

### Intermediate 17c: 2-[6-amino-5-(1,2,3,6-tetrahydropyridin-4-yl)pyridazin-3-yl]phenol

The title compound (50 mg, 150 μmol, 99% yield), white solid hydrochloride salt, was prepared in analogy to intermediate 12 from intermediate 17b, 3 eq HCl. MS (ESI): 298.2 ([M+H]⁺).

### Intermediate 16c: 2-[6-amino-5-(4,7-diazaspiro[2.5]octan-7-yl)pyridazin-3-yl]phenol

The title compound (50 mg, 150 μmol, 99% yield), white solid hydrochloride salt, was prepared in analogy to intermediate 12 from intermediate 16b, 3 eq HCl. MS (ESI): 298.2 ([M+H]⁺).

### Intermediate 19: 2-[6-Amino-5-(3,9-diazabicyclo[3.3.1]nonan-3-yl)pyridazin-3-yl]phenol

The title compound (9.5 mg, 30.5 μmol, 70% yield), off-white solid hydrochloride salt, was prepared in analogy to intermediate 12 from intermediate 19b, 10 eq HCl in dioxane. MS (ESI): 312.3 ([M+H]⁺).

### Intermediate 29: 2-[6-amino-5-[8-[[3-(piperazin-1-ylmethyl)phenyl]methyl]-3,8-diazabicyclo[3.2.1]octan-3-yl]pyridazin-3-yl]phenol

To a cooled (0° C.) solution of intermediate 29e (175 mg, 299 μmol, 1.0 eq) in DCM (3 mL) was added 4M HCl in dioxane (224 μL, 896 μmol, 4.0 eq). The reaction mixture was allowed to reach room temperature and stirred for 24 h. The reaction mixture was filtered and the solid was dried in vacuo to afford the title compound (150 mg, 269 μmol, 90% yield) as a white hydrochloride salt. MS (ESI): 486.5 ([M+H]⁺).

### Intermediate 35: 2-(6-amino-5-((3-(2-(piperazin-1-yl)ethoxy)phenyl)ethynyl)pyridazin-3-yl)phenol

The title compound (0.94 g, 1.92 mmol, 106% yield), light yellow dihydrochloride salt, was prepared in analogy to intermediate 12 from intermediate 35c. MS (ESI): 416.21 ([M+H]⁺).

### Intermediate 50: 2-(6-amino-5-(3-amino-2-phenylpropoxy)pyridazin-3-yl)phenol

The title compound (354 mg, 1.05 mmol, 97% yield), off-white hydrochloride salt, was prepared in analogy to intermediate 12 from intermediate 50b, 16 eq HCl. MS (ESI): 337.2 ([M+H]⁺).

### Intermediate 52: 2-[6-amino-5-[4-[3-(2-piperazin-1-ylethoxy)phenyl]-1-oxa-4,9-diazaspiro[5.5]undecan-9-yl]pyridazin-3-yl]phenol

The title compound (100 mg, 183 μmol, 95% yield), white solid hydrochloride salt, was prepared in analogy to intermediate 12 from intermediate 52d, 3 eq HCl. MS (ESI): 546.4 ([M+H]⁺).

### Intermediate 53: 2-(6-amino-5-((3-(piperazin-1-ylmethyl)phenyl)ethynyl)pyridazin-3-yl)phenol

The title compound (1.69 g, 3.28 mmol, 114% yield), yellow dihydrochloride salt, was prepared in analogy to intermediate 12 from intermediate 53c. MS (ESI): 386.3 ([M+H]⁺).

### Intermediate 62: 2-(6-amino-5-(2-amino-2-phenylethoxy)pyridazin-3-yl)phenol

The title compound (120 mg, 0.37 mmol, 25% yield), white solid, was prepared in analogy to intermediate 12 from intermediate 62b, 16.5 eq HCl, prep-HPLC. MS (ESI): 323.2 ([M+H]⁺).

### Intermediate 64: rac-2-(6-amino-5-(((3R,5R)-5-phenylpiperidin-3-yl)oxy)pyridazin-3-yl)phenol

The title compound (172 mg, 433 μmol, 98% yield), off-white hydrochloride salt, was prepared in analogy to intermediate 12 from intermediate 64b, 19 eq HCl, suspended in diethyl ether, filtered. MS (ESI): 363.1 ([M+H]⁺).

### Intermediate 66: rac-2-(6-amino-5-(((3R,5S)-5-phenylpiperidin-3-yl)oxy)pyridazin-3-yl)phenol

To a solution of intermediate 66b (184 mg, 398 μmol, 1.0 eq) in DCM (2 mL) was added 4M HCl in dioxane (2 mL, 8 mmol, 20.1 eq) at room temperature. White precipitate formed after 5 minutes of stirring. The reaction mixture was left to stir over the weekend before the solvent was removed in vacuo. The reaction was incomplete so was added a further amount of DCM (2 mL) and 4M HCl in dioxane (2 mL, 8 mmol, 20.1 eq) and left the reaction to stir at room temperature. The reaction progressed slowly, potentially due to solubility issues. The solvent was removed in vacuo and the residue was subsequently dissolved in DCM (1 mL) and TFA (1.48 g, 1 mL, 13 mmol, 32.6 eq). The reaction mixture was then left to stir at room temperature overnight before the solvent was removed in vacuo to afford the title compound (238 mg, 0.4 mmol, 100% yield) as a brown bis(2,2,2-trifluoroacetate) salt. MS (ESI): 363.1 ([M+H]⁺).

### Intermediate 69e

e) Step 5 (Removal of the Boc) missing

### Intermediate 74: 2-(6-amino-5-(2-fluoro-3-(piperazin-1-ylmethyl)phenyl)pyridazin-3-yl)phenol

The title compound (140 mg, 310 μmol, 98% yield), white dihydrochloride salt, was prepared in analogy to intermediate 12 from intermediate 74d, 16 eq HCl. MS (ESI): 380.3 ([M+H]⁺).

### Intermediate 89: 2-[6-Amino-5-(8-amino-3-azabicyclo[3.2.1]octan-3-yl)pyridazin-3-yl]phenol

The title compound (115 mg, 0.33 mmol, 100% yield), off-white hydrochloride salt, was prepared in analogy to intermediate 12 from intermediate 89b, 10 eq HCl. MS (ESI): 312.2 ([M+H]⁺).

### Intermediate 97:4-[3-amino-6-(2-hydroxyphenyl)pyridazin-4-yl]-1-phenyl-N-(4-piperidylmethyl)piperazine-2-carboxamide

The title compound (70 mg, 133 μmol, 100% yield), white hydrochloride salt, was prepared in analogy to intermediate 12 from intermediate 97c. MS (ESI): 488.4 ([M+H]⁺).

### Intermediate 98: 4-[3-amino-6-(2-hydroxyphenyl)pyridazin-4-yl]-N-[2-(methylamino)ethyl]-1-phenyl-piperazine-2-carboxamide hydrochloride

The title compound (40 mg, 82 μmol, 91% yield), white hydrochloride salt, was prepared in analogy to intermediate 12 from intermediate 98b. MS (ESI): 448.4 ([M+H]⁺).

### Intermediate 99: 1-[3-amino-6-(2-hydroxyphenyl)pyridazin-4-yl]-4-phenyl-N-(4-piperidylmethyl)piperidine-4-carboxamide

The title compound 200 mg, 383 μmol pmol, 75% yield), white hydrochloride salt, was prepared in analogy to intermediate 12 from intermediate 99d, 4 eq HCl. MS (ESI): 487.4 ([M+H]⁺).

### Intermediate 100: 6-(2-aminophenyl)-4-((1-(4-(piperazin-1-yl)phenyl)piperidin-3-yl)oxy)pyridazin-3-amine

Intermediate 100a (340 mg, 623 μmol, 1.0 eq) was stirred in dioxane (3 mL) with 4M HCl in dioxane (1.5 mL, 6 mmol, 9.63 eq) for 3 h at room temperature. The precipitated solid was filtered off and washed with ethyl ether and dried under high vacuum to afford the title compound (342 mg, 660 μmol, 106% yield) as a light brown dihydrochloride salt. MS (ESI): 446.2 ([M+H]⁺).

### Intermediate 108: 2-(6-amino-5-((1-(4-(piperazin-1-yl)phenyl)piperidin-3-yl)oxy)pyridazin-3-yl)-4-fluorophenol

The title compound (540 mg, 985 μmol, 116% yield), an off-white dihydrochloride salt, was prepared in analogy to intermediate 100 from intermediate 108a. MS (ESI): 465.2 ([M+H]⁺).

### Intermediate 26: 2-(6-Amino-5-(4-(methyl(3-(methylamino)propyl)amino)phenethoxy)pyridazin-3-yl)phenol

To a solution of intermediate 26f (70 mg, 137.90 umol) in DCM (3 mL) at 0° C. was added trifluoro acetic acid (47 mg, 413.5 umol, 31.87 uL) and stirred at room temperature for 3 h. The reaction mixture was concentrated under reduced pressure, and co-distilled with dichloromethane to afford the title compound (0.06 g, 101.59 umol, 73% yield). MS (ESI): 408.1 ([M+H]⁺).

### Intermediate 38:2-(6-amino-5-((1-(4-(piperazin-1-yl)benzyl)piperidin-3-yl)oxy)pyridazin-3-yl)phenol

The title compound (200 mg), was prepared in analogy to intermediate 26g from intermediate 38c. MS (ESI): 461.3 ([M+H]⁺).

### Intermediate 57: 2-(6-amino-5-(3-(2-(piperazin-1-yl)ethoxy)phenethoxy)pyridazin-3-yl)phenol

The title compound (0.16 g, 367 umol, 98% yield), was prepared in analogy to intermediate 26g from intermediate 57d. MS (ESI): 436.2 ([M+H]⁺).

### Intermediate 77: 2-(6-amino-5-((1-(4-(2-(piperazin-1-yl)ethoxy)phenyl)piperidin-3-yl)oxy)pyridazin-3-yl)phenol

The title compound (375 mg, 764 μmol, 98% yield), light brown semi solid, was prepared in analogy to intermediate 26g from intermediate 77d. MS (ESI): 491.2 ([M+H]⁺).

### Intermediate 82: 2-(6-amino-5-(1-(3-(piperazin-1-ylmethyl)phenyl)ethoxy)pyridazin-3-yl)phenol

A mixture of intermediate 81d (300 mg, 0.58 mmol, 1.0 eq) in TFA (3 mL) and DCM (3 mL) was stirred at 25° C. for 2 h. The reaction mixture was concentrated in vacuum to afford the title compound (200 mg, 0.49 mmol, 85% yield) as a yellow TFA salt. MS (ESI): 406.2 ([M+H]⁺).

### Intermediate 87c: tert-butyl N-(4-(3-amino-6-(2-hydroxyphenyl)pyridazin-4-yl)benzyl)-N-methylglycinate

Intermediate 87b (145 mg, 498 μmol, 1.0 eq) was stirred with tert-butyl methylglycinate (79.5 mg, 548 μmol, 1.1 eq) and sodium triacetoxyborohydride (148 mg, 697 μmol, 1.4 eq) in 1,2-Dichloroethane (4 mL) at room temperature overnight. Saturated NaHCO₃ solution was added and extracted with dichloromethane, dried over sodium sulfate, filtered and evaporated. The crude material was purified by column chromatography to afford the title compound (91 mg, 216 μmol, 43% yield) as a light yellow solid. MS (ESI): 421.2243 ([M+H]⁺).

### Intermediate 87: N-(4-(3-amino-6-(2-hydroxyphenyl)pyridazin-4-yl)benzyl)-N-methylglycine

Intermediate 87c (81 mg, 193 μmol, 1.0 eq) was stirred with 2,2,2-trifluoroacetic acid (22 mg, 500 μL, 193 μmol, 1.0 eq) in dichloromethane (1 mL) at room temperature overnight. Water was added. The pH was set neutral by addition of saturated NaHCO₃ solution and extracted with dichloromethane. Lot of product stayed in water so all the phases were combined and evaporated and purified by preparative HPLC to afford the title compound (27 mg, 74.1 μmol, 38% yield) as a white solid. MS (ESI): 365.1622 ([M+H]⁺).

### Intermediate 1: 10-[4-[3-amino-6-(2-hydroxyphenyl)pyridazin-4-yl]piperazin-1-yl]-10-oxo-decanoic acid

To a solution of 2-(6-amino-5-piperazin-1-yl-pyridazin-3-yl)phenol (100 mg, 0.370 mmol, 1.0 eq, CAS: 1997319-92-2) in dichloromethane (5 mL) was added sebacic acid (74.54 mg, 0.370 mmol, 1.0 eq), 2-(3H-[1,2,3]triazolo[4,5-b]pyridin-3-yl)-1,1,3,3-tetramethylisouronium hexafluorophosphate(V) (168.17 mg, 0.440 mmol, 1.2 eq), N,N-diisopropylethylamine (0.26 mL, 1.47 mmol, 4.0 eq). The solution was stirred at 0° C. for 2 h. The mixture was concentrated in vacuum, then purified by preparative-HPLC to afford the title compound (51 mg, 112 umol, 26% yield) as a yellow solid. MS (ESI): 456.2 ([M+H]⁺).

### Intermediate 3i: 9-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]-9-oxo-nonanoic acid

The title compound (60 mg, 0.070 mmol, 35% yield), yellow solid, was prepared in analogy to intermediate 1 from intermediate 3h and nonanedioic acid. MS (ESI): 672.3 ([M+H]⁺).

### Intermediate 4: 2-[6-amino-5-(3-methylpiperazin-1-yl)pyridazin-3-yl]phenol

Intermediate 4b (77 mg, 200 μmol, 1.0 eq) and TFA (1.48 g, 1000 μL, 13 mmol, 65.0 eq) were combined with DCM (5 mL). The reaction mixture was stirred at 23° C. for 20 h. The crude reaction mixture was concentrated in vacuo to afford the title compound (76 mg, 190 mmol, 95% yield) as a yellow oil, 2,2,2-trifluoroacetate salt.

### Intermediate 18a: tert-butyl 4-(3-(((1R,5S)-3-(3-amino-6-(2-hydroxyphenyl)pyridazin-4-yl)-3,8-diazabicyclo[3.2.1]octan-8-yl)methyl)benzoyl)piperazine-1-carboxylate

Tert-butyl 4-(3-(bromomethyl)benzoyl)piperazine-1-carboxylate (120 mg, 313 μmol, 1.29 eq), 2-(6-amino-5-((1R,5S)-3,8-diazabicyclo[3.2.1]octan-3-yl)pyridazin-3-yl)phenol 2,2,2-trifluoroacetate (100 mg, 243 μmol, 1.0 eq) and DIPEA (740 mg, 1 mL, 5.73 mmol, 23.6 eq) were combined with DMF (3 mL). The reaction mixture was heated to 23° C. and stirred for 20 h. The crude reaction mixture was concentrated in vacuo and purified by preparative HPLC to afford the title compound (20 mg, 12% yield) as a light yellow powder.

### Intermediate 18: (3-((3-(3-amino-6-(2-hydroxyphenyl)pyridazin-4-yl)-3,8-diazabicyclo[3.2.1]octan-8-yl)methyl)phenyl)(piperazin-1-yl)methanone

Intermediate 18a (20 mg, 33.3 μmol, 1.0 eq) and 4M HCl in dioxane (500p, 2 mmol, 60.0 eq) were combined with MeOH (1 mL) to give a light yellow solution. The reaction mixture was stirred for 2 h. The crude reaction mixture was concentrated in vacuo to afford the title compound, which was used directly in the next step.

### Intermediate 110g: 2-[6-amino-5-[3-[3-(2-piperazin-1-ylethoxy)phenyl]-3,8-diazabicyclo[3.2.1]octan-8-yl]pyridazin-3-yl]-4-fluoro-phenol

intermediate 110f (225 mg, 344 μmol, 1.0 eq) was stirred with palladium (36.6 mg, 34.4 μmol, 0.1 eq) in methanol (10 mL) and tetrahydrofuran (2 mL) under a hydrogen atmosphere at room temperature overnight. The catalyst was filtered off and the solvent was evaporated under reduced pressure, then dried under high vacuum to afford the title compound (166 mg, 300 μmol, 87% yield) as an off-white solid. MS (ESI): 518.2676 ([M−H]⁻).

### Example 1

### (2S,4R)-1-[(2S)-2-[[10-[4-[3-amino-6-(2-hydroxyphenyl)pyridazin-4-yl]piperazin-1-yl]-10-oxo-decanoyl]amino]-3,3-dimethyl-butanoyl]-4-hydroxy-N-[[4-(4-methylthiazol-5-yl)phenyl]methyl]pyrrolidine-2-carboxamide

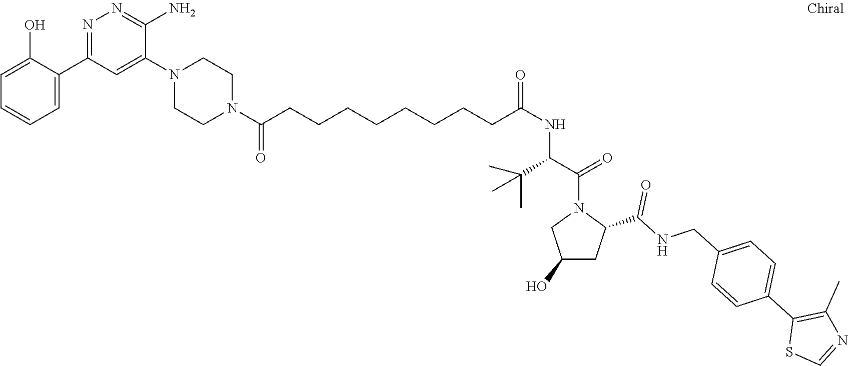 [SMILES: Cc1ncsc1-c1ccc(CNC(=O)[C@@H]2C[C@@H](O)CN2C(=O)[C@@H](NC(=O)CCCCCCCCC(=O)N2CCN(c3cc(-c4ccccc4O)nnc3N)CC2)C(C)(C)C)cc1]

In a screw-cap-tube, intermediate 1 (43 mg, 94.4 μmol, 1.0 eq), Ligase 1 hydrochloride (44.1 mg, 94.4 μmol, 1.0 eq), 2-(3H-[1,2,3]triazolo[4,5-b]pyridin-3-yl)-1,1,3,3-tetramethylisouronium hexafluorophosphate(V) (53.8 mg, 142 μmol, 1.5 eq) and N,N-diisopropylethylamine (48.8 mg, 65.9 μl, 378 μmol, 4 eq) were combined with dimethylformamide (984 μl) to give a yellow solution. The reaction mixture was stirred at room temperature over night. The reaction mixture was purified by preparative HPLC to afford the title compound (50.7 mg, 58.4 umol, 61% yield) as a colorless foam. MS (ESI): 912.8 ([M+H+formiate]⁺).

### Example 2

### (2S,4R)-1-[(2S)-2-[[9-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]-9-oxo-nonanoyl]amino]-3,3-dimethyl-butanoyl]-4-hydroxy-N-[[4-(4-methylthiazol-5-yl)phenyl]methyl]pyrrolidine-2-carboxamide

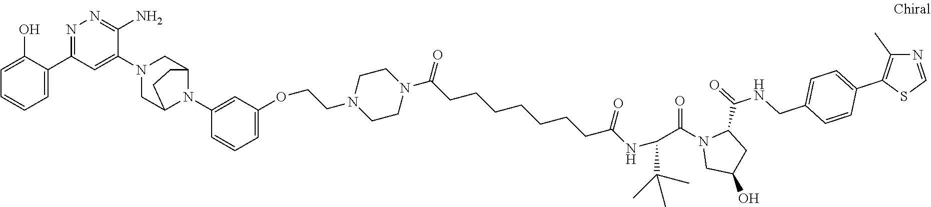 [SMILES: Cc1ncsc1-c1ccc(CNC(=O)[C@@H]2C[C@@H](O)CN2C(=O)[C@@H](NC(=O)CCCCCCCC(=O)N2CCN(CCOc3cccc(N4C5CCC4CN(c4cc(-c6ccccc6O)nnc4N)C5)c3)CC2)C(C)(C)C)cc1]

The title compound (17.2 mg, 0.016 mmol, 34% yield), yellow solid, was prepared in analogy to Example 1 from intermediate 2h. MS (ESI): 1128.9 ([M−H+formiate]⁻), 1083.9 ([M−H]⁻).

### Example 3

### (2S,4R)-1-[(2S)-2-[[9-[4-[[3-[3-[3-amino-6-(2-hydroxyphenyl)pyridazin-4-yl]-3,8-diazabicyclo[3.2.1]octan-8-yl]phenyl]methyl]piperazin-1-yl]-9-oxo-nonanoyl]amino]-3,3-dimethyl-butanoyl]-4-hydroxy-N-[[4-(4-methylthiazol-5-yl)phenyl]methyl]pyrrolidine-2-carboxamide

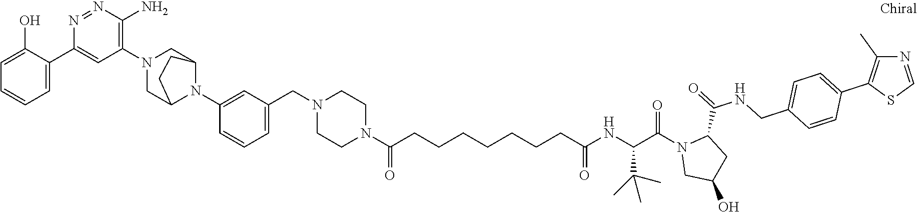 [SMILES: Cc1ncsc1-c1ccc(CNC(=O)[C@@H]2C[C@@H](O)CN2C(=O)[C@@H](NC(=O)CCCCCCCC(=O)N2CCN(Cc3cccc(N4C5CCC4CN(c4cc(-c6ccccc6O)nnc4N)C5)c3)CC2)C(C)(C)C)cc1]

The title compound (22.4 mg, 0.021 mmol, 63% yield), light yellow solid, was prepared in analogy to Example 1 from intermediate 3 and Ligase 10. MS (ESI): 352.6 ([M/3+H]⁺), 1055.0 ([M+H]⁺).

### Example 4

### (2S,4R)-1-[(2S)-2-[[10-[4-[3-amino-6-(2-hydroxyphenyl)pyridazin-4-yl]-2-methyl-piperazin-1-yl]-10-oxo-decanoyl]amino]-3,3-dimethyl-butanoyl]-4-hydroxy-N-[[4-(4-methylthiazol-5-yl)phenyl]methyl]pyrrolidine-2-carboxamide

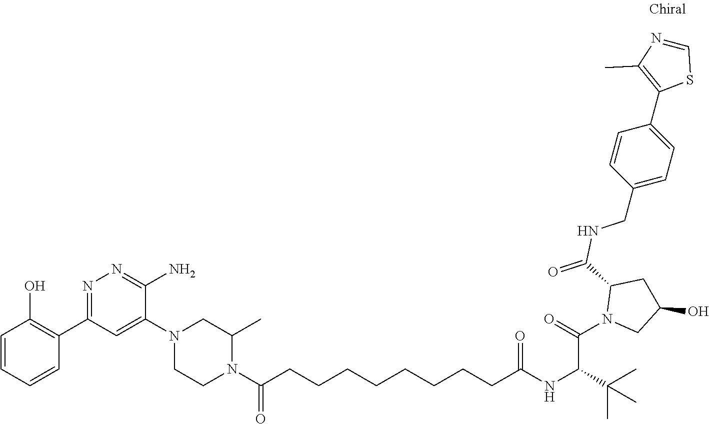 [SMILES: Cc1ncsc1-c1ccc(CNC(=O)[C@@H]2C[C@@H](O)CN2C(=O)[C@@H](NC(=O)CCCCCCCCC(=O)N2CCN(c3cc(-c4ccccc4O)nnc3N)CC2C)C(C)(C)C)cc1]

The title compound (18 mg, 0.02 mmol, 11% yield), white powder, was prepared in analogy to Example 1 from intermediate 4 and Ligase 4. MS (ESI): 882.6 ([M+H]⁺).

### Example 5

### (2S,4R)-1-[(2S)-2-[[10-[3-[3-amino-6-(2-hydroxyphenyl)pyridazin-4-yl]-3,8-diazabicyclo[3.2.1]octan-8-yl]-10-oxo-decanoyl]amino]-3,3-dimethyl-butanoyl]-4-hydroxy-N-[[4-(4-methylthiazol-5-yl)phenyl]methyl]pyrrolidine-2-carboxamide

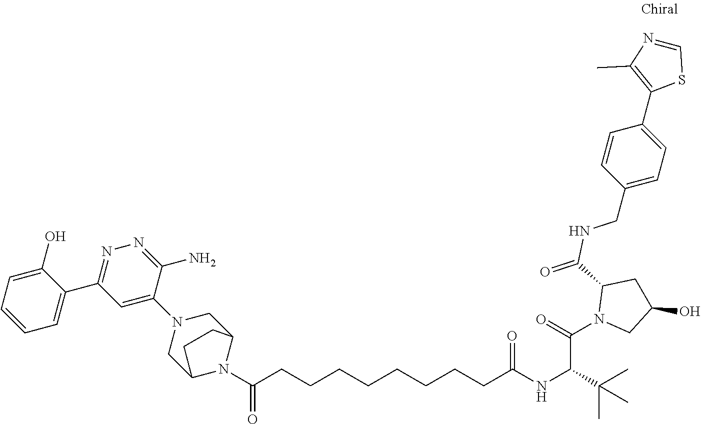 [SMILES: Cc1ncsc1-c1ccc(CNC(=O)[C@@H]2C[C@@H](O)CN2C(=O)[C@@H](NC(=O)CCCCCCCCC(=O)N2C3CCC2CN(c2cc(-c4ccccc4O)nnc2N)C3)C(C)(C)C)cc1]

The title compound (31 mg, 0.035 mmol, 20% yield), light yellow solid, was prepared in analogy to Example 1 from Ligase 4 and 2-(6-amino-5-(3,8-diazabicyclo[3.2.1]octan-3-yl)pyridazin-3-yl)phenol. MS (ESI): 894.7 ([M+H]⁺).

### Example 6

### (2S,4R)-1-[(2R)-2-[[8-[9-[3-amino-6-(2-hydroxyphenyl)pyridazin-4-yl]-3,9-diazaspiro[5.5]undecan-3-yl]-8-oxo-octanoyl]amino]-3,3-dimethyl-butanoyl]-4-hydroxy-N-[[4-(4-methylthiazol-5-yl)phenyl]methyl]pyrrolidine-2-carboxamide

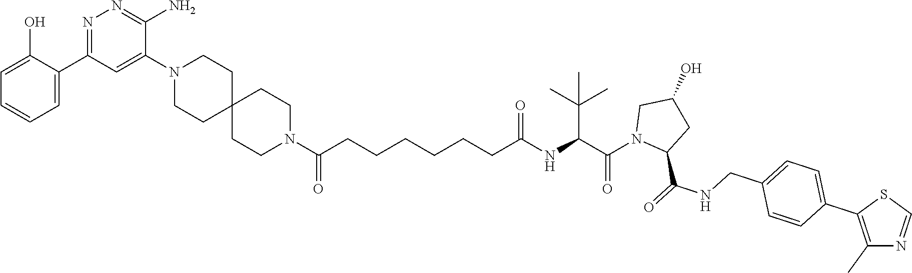 [SMILES: Cc1ncsc1-c1ccc(CNC(=O)[C@@H]2C[C@@H](O)CN2C(=O)[C@@H](NC(=O)CCCCCCC(=O)N2CCC3(CC2)CCN(c2cc(-c4ccccc4O)nnc2N)CC3)C(C)(C)C)cc1]

To a solution of intermediate 6 (28.93 mg, 0.090 mmol, 1.0 eq), triethylamine (0.05 mL, 0.340 mmol, 4.0 eq) and Ligase 2 (50.0 mg, 0.090 mmol, 1.0 eq) in DMF (2 mL) was added 1-propanephosphonic anhydride in EtOAc (108 mg, 0.170 mmol, 2.0 eq) at 0° C., then the mixture was stirred at 25° C. for 2 h. The mixture was poured into water (2 mL), then concentrated in vacuum to give a residue which was purified by prep-HPLC (FA) to afford the title compound (10 mg, 0.010 mmol, 13% yield) as a yellow solid. MS (ESI): 908.5 ([M+H]⁺).

### Example 7

### (2S,4R)-1-[(2S)-2-[[11-[4-[[3-[3-[3-amino-6-(2-hydroxyphenyl)pyridazin-4-yl]-3,8-diazabicyclo[3.2.1]octan-8-yl]phenyl]methyl]piperazin-1-yl]-11-oxo-undecanoyl]amino]-3,3-dimethyl-butanoyl]-4-hydroxy-N-[[4-(4-methylthiazol-5-yl)phenyl]methyl]pyrrolidine-2-carboxamide

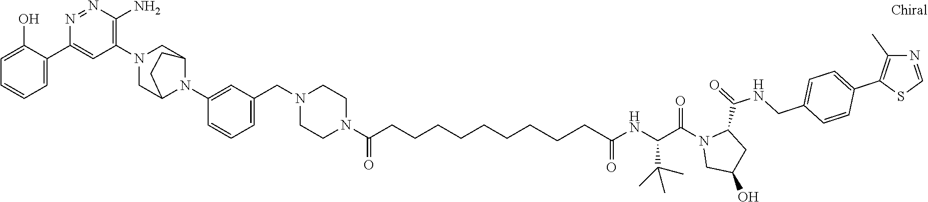 [SMILES: Cc1ncsc1-c1ccc(CNC(=O)[C@@H]2C[C@@H](O)CN2C(=O)[C@@H](NC(=O)CCCCCCCCCC(=O)N2CCN(Cc3cccc(N4C5CCC4CN(c4cc(-c6ccccc6O)nnc4N)C5)c3)CC2)C(C)(C)C)cc1]

The title compound (11 mg, 10.2 μmol, 27% yield), light yellow foam, was prepared in analogy to Example 1 from Ligase 5 and intermediate 3. MS (ESI): 362.1 ([M/3+H]⁺), 542.3 ([M/2+H]⁺), 1083.2 ([M+H]⁺).

### Example 8

### (2S,4R)-1-[(2R)-2-[[8-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]-8-oxo-octanoyl]amino]-3,3-dimethyl-butanoyl]-4-hydroxy-N-[[4-(4-methylthiazol-5-yl)phenyl]methyl]pyrrolidine-2-carboxamide

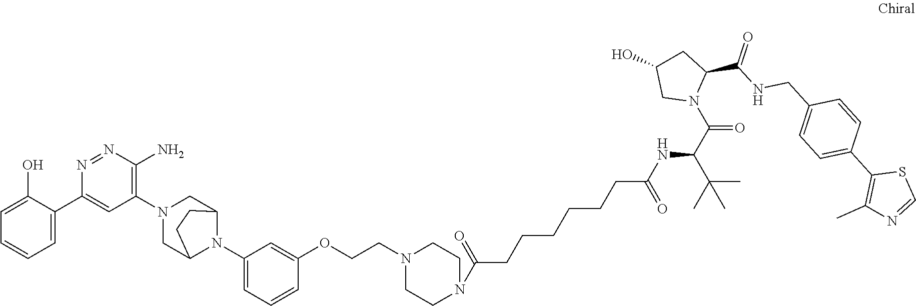 [SMILES: Cc1ncsc1-c1ccc(CNC(=O)[C@@H]2C[C@@H](O)CN2C(=O)[C@H](NC(=O)CCCCCCC(=O)N2CCN(CCOc3cccc(N4C5CCC4CN(c4cc(-c6ccccc6O)nnc4N)C5)c3)CC2)C(C)(C)C)cc1]

The title compound (5 mg, 0.005 mmol, 15% yield), white solid, was prepared in analogy to Example 6 from intermediate 2 and Ligase 2. MS ISP (m/e): 536.1 ([M/2+H]⁺).

### Example 9

### (2S,4R)-1-[(2R)-2-[[7-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]-7-oxo-heptanoyl]amino]-3,3-dimethyl-butanoyl]-4-hydroxy-N-[[4-(4-methylthiazol-5-yl)phenyl]methyl]pyrrolidine-2-carboxamide

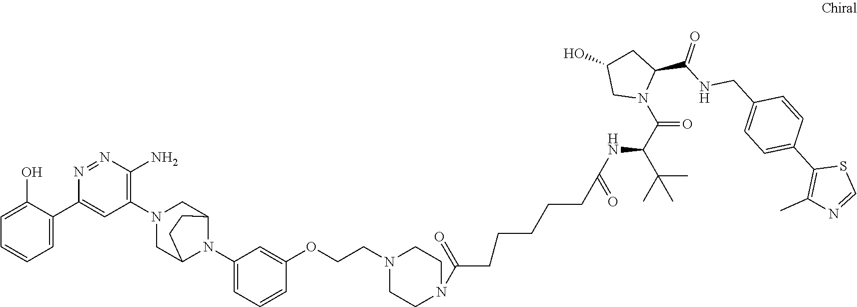 [SMILES: Cc1ncsc1-c1ccc(CNC(=O)[C@@H]2C[C@@H](O)CN2C(=O)[C@H](NC(=O)CCCCCC(=O)N2CCN(CCOc3cccc(N4C5CCC4CN(c4cc(-c6ccccc6O)nnc4N)C5)c3)CC2)C(C)(C)C)cc1]

The title compound (6.6 mg, 0.010 mmol, 20% yield), white solid, was prepared in analogy to Example 1 from Ligase 3 and intermediate 2. MS (ESI): 1056.5 ([M+H]⁺).

### Example 10

### (2S,4R)-1-[(2S)-2-[[10-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]-10-oxo-decanoyl]amino]-3,3-dimethyl-butanoyl]-4-hydroxy-N-[[4-(4-methylthiazol-5-yl)phenyl]methyl]pyrrolidine-2-carboxamide

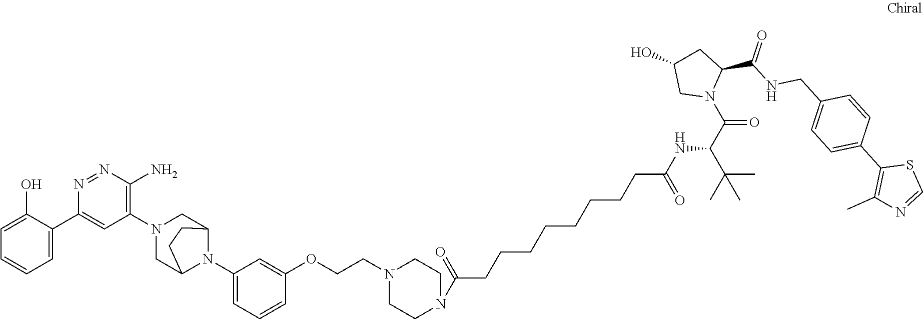 [SMILES: Cc1ncsc1-c1ccc(CNC(=O)[C@@H]2C[C@@H](O)CN2C(=O)[C@@H](NC(=O)CCCCCCCCC(=O)N2CCN(CCOc3cccc(N4C5CCC4CN(c4cc(-c6ccccc6O)nnc4N)C5)c3)CC2)C(C)(C)C)cc1]

The title compound (6.7 mg, 0.010 mmol, 20% yield), white solid, was prepared in analogy to Example 1 from Ligase 4 and intermediate 2. MS (ESI): 1120.7 ([M+Na]+).

### Example 11

### (2S,4R)-1-[(2S)-2-[[11-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]-11-oxo-undecanoyl]amino]-3,3-dimethyl-butanoyl]-4-hydroxy-N-[[4-(4-methylthiazol-5-yl)phenyl]methyl]pyrrolidine-2-carboxamide

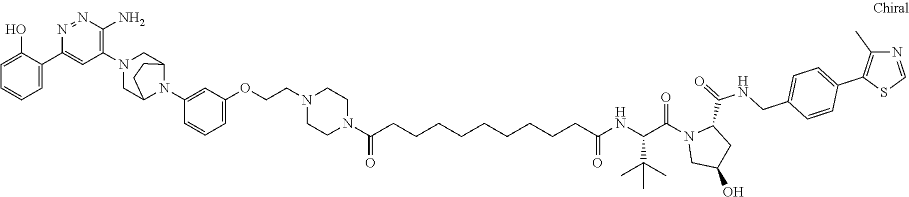 [SMILES: Cc1ncsc1-c1ccc(CNC(=O)[C@@H]2C[C@@H](O)CN2C(=O)[C@@H](NC(=O)CCCCCCCCCC(=O)N2CCN(CCOc3cccc(N4C5CCC4CN(c4cc(-c6ccccc6O)nnc4N)C5)c3)CC2)C(C)(C)C)cc1]

The title compound (6.8 mg, 0.010 mmol, 20% yield), white solid, was prepared in analogy to Example 1 from Ligase 5 and intermediate 2. MS (ESI): 1112.8 ([M+H]⁺).

### Example 12

### (2S,4R)-1-[(2S)-2-[[10-[9-[3-amino-6-(2-hydroxyphenyl)pyridazin-4-yl]-1-oxa-4,9-diazaspiro[5.5]undecan-4-yl]-10-oxo-decanoyl]amino]-3,3-dimethyl-butanoyl]-4-hydroxy-N-[[4-(4-methylthiazol-5-yl)phenyl]methyl]pyrrolidine-2-carboxamide

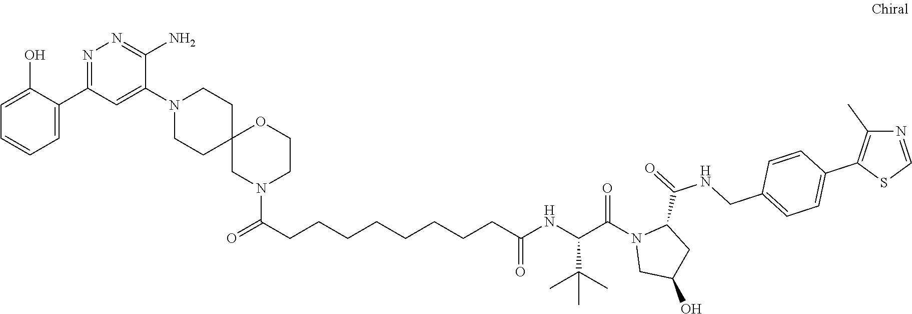 [SMILES: Cc1ncsc1-c1ccc(CNC(=O)[C@@H]2C[C@@H](O)CN2C(=O)[C@@H](NC(=O)CCCCCCCCC(=O)N2CCOC3(CCN(c4cc(-c5ccccc5O)nnc4N)CC3)C2)C(C)(C)C)cc1]

The title compound (35 mg, 0.037 mmol, 29% yield), brown solid, was prepared in analogy to Example 1 from intermediate 12 and Ligase 4. MS (ESI): 938.8 ([M+H]⁺).

### Example 13

### (2S,4S)-1-[(2S)-2-[[9-[4-[[3-[3-[3-amino-6-(2-hydroxyphenyl)pyridazin-4-yl]-3,8-diazabicyclo[3.2.1]octan-8-yl]phenyl]methyl]-1-piperidyl]-9-oxo-nonanoyl]amino]-3,3-dimethyl-butanoyl]-4-hydroxy-N-[[4-(4-methylthiazol-5-yl)phenyl]methyl]pyrrolidine-2-carboxamide

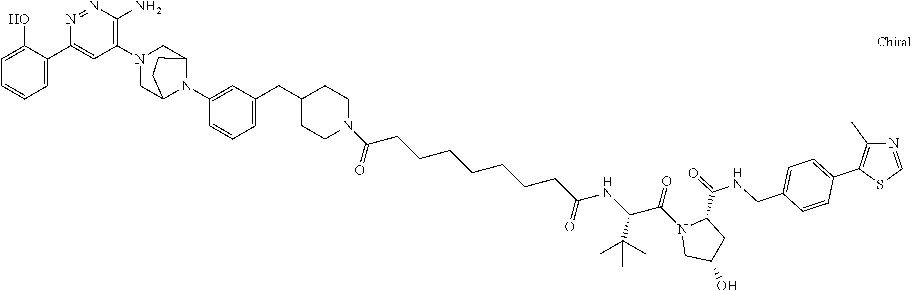 [SMILES: Cc1ncsc1-c1ccc(CNC(=O)[C@@H]2C[C@H](O)CN2C(=O)[C@@H](NC(=O)CCCCCCCC(=O)N2CCC(Cc3cccc(N4C5CCC4CN(c4cc(-c6ccccc6O)nnc4N)C5)c3)CC2)C(C)(C)C)cc1]

The title compound (16.5 mg, 15.7 μmol, 36% yield), yellow solid, was prepared in analogy to Example 1 from intermediate 13 and Ligase 10. MS (ESI): 1053.7 ([M+H]⁺).

### Example 14

### (2S,4S)-1-[(2S)-2-[[7-[4-[[3-[3-[3-amino-6-(2-hydroxyphenyl)pyridazin-4-yl]-3,8-diazabicyclo[3.2.1]octan-8-yl]phenyl]methyl]-1-piperidyl]-7-oxo-heptanoyl]amino]-3,3-dimethyl-butanoyl]-4-hydroxy-N-[[4-(4-methylthiazol-5-yl)phenyl]methyl]pyrrolidine-2-carboxamide

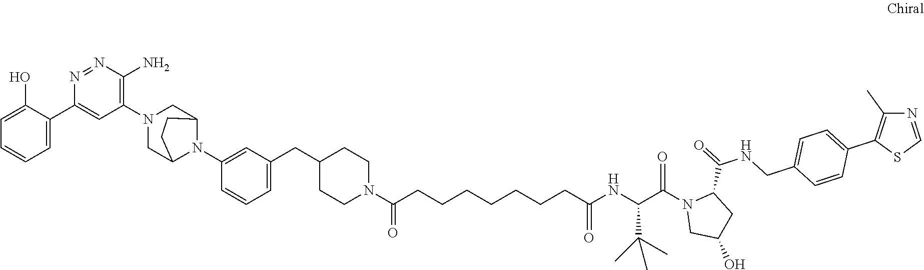 [SMILES: Cc1ncsc1-c1ccc(CNC(=O)[C@@H]2C[C@H](O)CN2C(=O)[C@@H](NC(=O)CCCCCCCC(=O)N2CCC(Cc3cccc(N4C5CCC4CN(c4cc(-c6ccccc6O)nnc4N)C5)c3)CC2)C(C)(C)C)cc1]

The title compound (5.7 mg, 5.6 μmol, 11% yield), yellow solid, was prepared in analogy to Example 1 from intermediate 13 and Ligase 3. MS (ESI): 1025.7 ([M+H]⁺).

### Example 15

### (2S,4S)-1-[(2S)-2-[[11-[4-[[3-[3-[3-amino-6-(2-hydroxyphenyl)pyridazin-4-yl]-3,8-diazabicyclo[3.2.1]octan-8-yl]phenyl]methyl]-1-piperidyl]-11-oxo-undecanoyl]amino]-3,3-dimethyl-butanoyl]-4-hydroxy-N-[[4-(4-methylthiazol-5-yl)phenyl]methyl]pyrrolidine-2-carboxamide

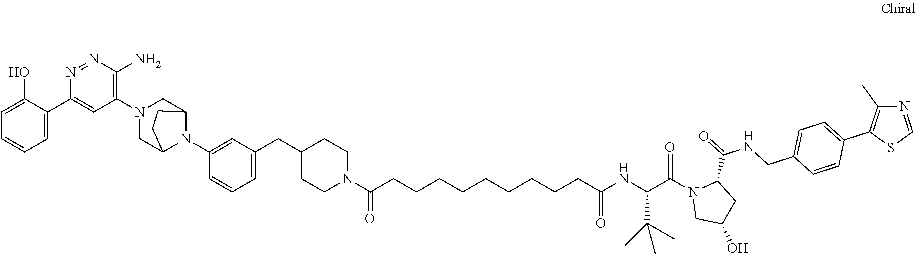 [SMILES: Cc1ncsc1-c1ccc(CNC(=O)[C@@H]2C[C@H](O)CN2C(=O)[C@@H](NC(=O)CCCCCCCCCC(=O)N2CCC(Cc3cccc(N4C5CCC4CN(c4cc(-c6ccccc6O)nnc4N)C5)c3)CC2)C(C)(C)C)cc1]

The title compound (3 mg, 2.8 μmol, 6% yield), yellow oil, was prepared in analogy to Example 1 from intermediate 13 and Ligase 5. MS (ESI): 1081.7 ([M+H]⁺).

### Example 16

### (2S,4R)-1-[(2S)-2-[[10-[7-[3-amino-6-(2-hydroxyphenyl)pyridazin-4-yl]-4,7-diazaspiro[2.5]octan-4-yl]-10-oxo-decanoyl]amino]-3,3-dimethyl-butanoyl]-4-hydroxy-N-[[4-(4-methylthiazol-5-yl)phenyl]methyl]pyrrolidine-2-carboxamide

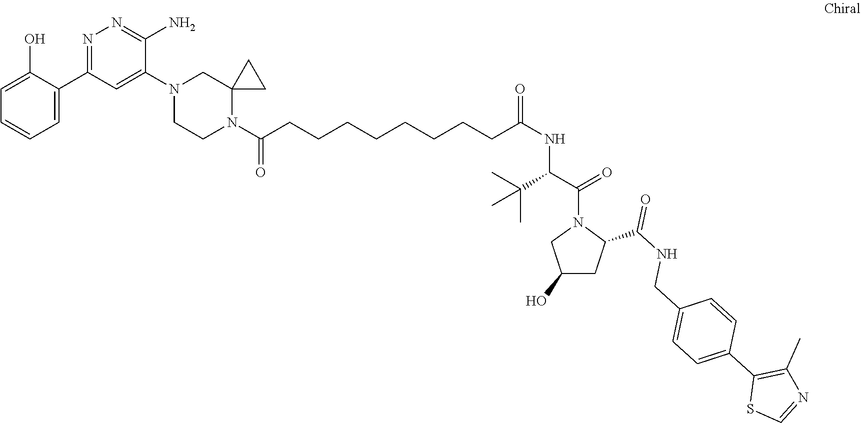 [SMILES: Cc1ncsc1-c1ccc(CNC(=O)[C@@H]2C[C@@H](O)CN2C(=O)[C@@H](NC(=O)CCCCCCCCC(=O)N2CCN(c3cc(-c4ccccc4O)nnc3N)CC23CC3)C(C)(C)C)cc1]

The title compound (20 mg, 0.022 mmol, 90% yield), light brown solid, was prepared in analogy to Example 1 from intermediate 16 and Ligase 4. MS (ESI): 894.8 ([M+H]⁺).

### Example 17

### (2S,4R)-1-[(2S)-2-[[10-[4-[3-amino-6-(2-hydroxyphenyl)pyridazin-4-yl]-3,6-dihydro-2H-pyridin-1-yl]-10-oxo-decanoyl]amino]-3,3-dimethyl-butanoyl]-4-hydroxy-N-[[4-(4-methylthiazol-5-yl)phenyl]methyl]pyrrolidine-2-carboxamide

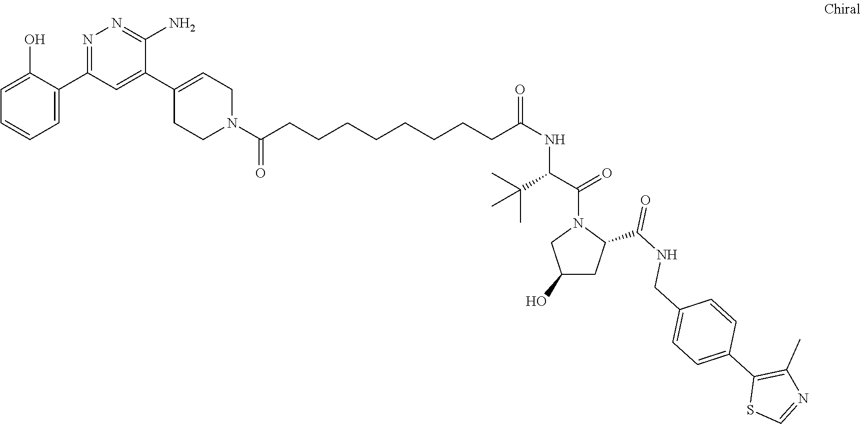 [SMILES: Cc1ncsc1-c1ccc(CNC(=O)[C@@H]2C[C@@H](O)CN2C(=O)[C@@H](NC(=O)CCCCCCCCC(=O)N2CC=C(c3cc(-c4ccccc4O)nnc3N)CC2)C(C)(C)C)cc1]

The title compound (9 mg, 0.010 mmol, 10% yield), light brown solid, was prepared in analogy to Example 1 from intermediate 17 and Ligase 4. MS (ESI): 865.7 ([M+H]⁺).

### Example 18

### (2S,4R)-1-[(2S)-2-[[11-[4-[3-[[3-[3-amino-6-(2-hydroxyphenyl)pyridazin-4-yl]-3,8-diazabicyclo[3.2.1]octan-8-yl]methyl]benzoyl]piperazin-1-yl]-11-oxo-undecanoyl]amino]-3,3-dimethyl-butanoyl]-4-hydroxy-N-[[4-(4-methylthiazol-5-yl)phenyl]methyl]pyrrolidine-2-carboxamide

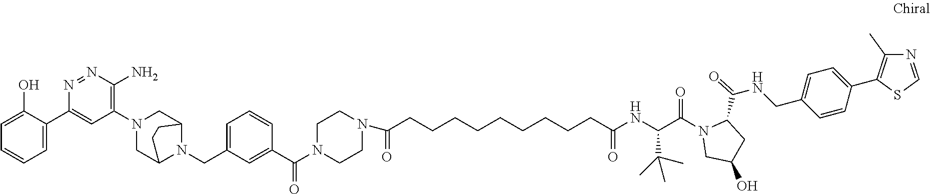 [SMILES: Cc1ncsc1-c1ccc(CNC(=O)[C@@H]2C[C@@H](O)CN2C(=O)[C@@H](NC(=O)CCCCCCCCCC(=O)N2CCN(C(=O)c3cccc(CN4C5CCC4CN(c4cc(-c6ccccc6O)nnc4N)C5)c3)CC2)C(C)(C)C)cc1]

The title compound (13.1 mg, 11.8 μmol, 31% yield), light yellow viscous oil, was prepared in analogy to Example 1 from intermediate 18 and Ligase 5. MS (ESI): 1111.6 ([M+H]⁺).

### Example 19

### (2S,4R)-1-[(2S)-2-[[10-[3-[3-Amino-6-(2-hydroxyphenyl)pyridazin-4-yl]-3,9-diazabicyclo[3.3.1]nonan-9-yl]-10-oxo-decanoyl]amino]-3,3-dimethyl-butanoyl]-4-hydroxy-N-[[4-(4-methylthiazol-5-yl)phenyl]methyl]pyrrolidine-2-carboxamide

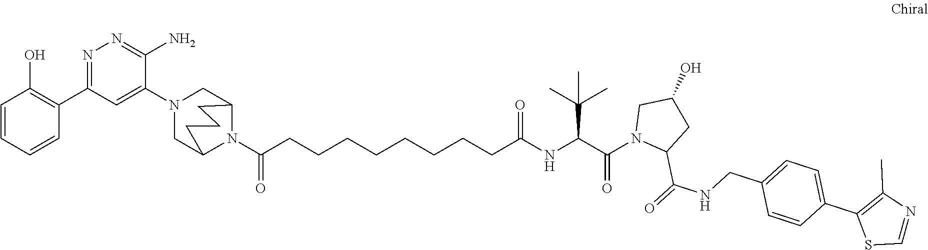 [SMILES: Cc1ncsc1-c1ccc(CNC(=O)C2C[C@@H](O)CN2C(=O)[C@@H](NC(=O)CCCCCCCCC(=O)N2C3CCCC2CN(c2cc(-c4ccccc4O)nnc2N)C3)C(C)(C)C)cc1]

The title compound (39.5 mg, 43.5 μmol, 48% yield), white solid, 2,2,2-trifluoroacetic acid salt, was prepared in analogy to Example 1 from intermediate 19 and Ligase 4. MS (ESI): 908.4 ([M+H]⁺).

### Example 20

### (2S,4R)-1-[(2R)-2-[10-[3-[3-[3-amino-6-(2-hydroxyphenyl)pyridazin-4-yl]-3,8-diazabicyclo[3.2.1]octan-8-yl]phenoxy]decanoylamino]-3,3-dimethyl-butanoyl]-4-hydroxy-N-[[4-(4-methylthiazol-5-yl)phenyl]methyl]pyrrolidine-2-carboxamide

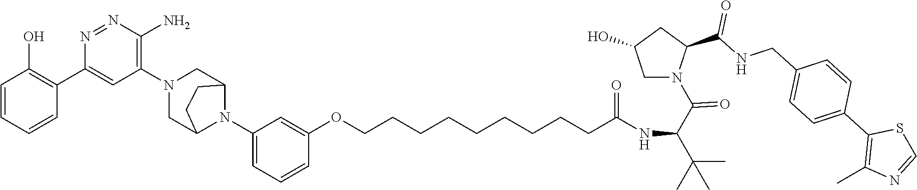 [SMILES: Cc1ncsc1-c1ccc(CNC(=O)[C@@H]2C[C@@H](O)CN2C(=O)[C@H](NC(=O)CCCCCCCCCOc2cccc(N3C4CCC3CN(c3cc(-c5ccccc5O)nnc3N)C4)c2)C(C)(C)C)cc1]

The title compound (1.4 mg, 1.4 μmol, 2% yield), light yellow solid, was prepared in analogy to Example 6 from intermediate 20 and Ligase 1. MS ISP (m/e): 941.5 ([M+H]⁺).

### Example 21

### N′-[3-[3-amino-6-(2-hydroxyphenyl)pyridazin-4-yl]oxy-2-phenyl-propyl]-N′-methyl-N-[rac-(1S)-2,2-dimethyl-1-[rac-(2S,4R)-4-hydroxy-2-[[rac-(1S)-1-[4-(4-methylthiazol-5-yl)phenyl]ethyl]carbamoyl]pyrrolidine-1-carbonyl]propyl]heptanediamide

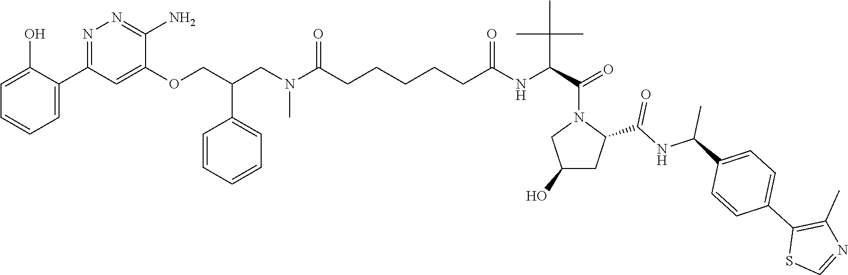 [SMILES: Cc1ncsc1-c1ccc([C@H](C)NC(=O)[C@@H]2C[C@@H](O)CN2C(=O)[C@@H](NC(=O)CCCCCC(=O)N(C)CC(COc2cc(-c3ccccc3O)nnc2N)c2ccccc2)C(C)(C)C)cc1]

The title compound, was prepared in analogy to Example 1 from intermediate 21 and Ligase 11 as the trifluoroacetic acid salt. MS (ESI): 919.95 ([M+H]⁺).

### Example 22

N′-[3-[3-amino-6-(2-hydroxyphenyl)pyridazin-4-yl]oxy-2-phenyl-propyl]-N′-methyl-N-[rac-(1S)-2,2-dimethyl-1-[rac-(2S,4R)-4-hydroxy-2-[[rac-(1S)-1-[4-(4-methylthiazol-5-yl)phenyl]ethyl]carbamoyl]pyrrolidine-1-carbonyl]propyl]dodecanediamide

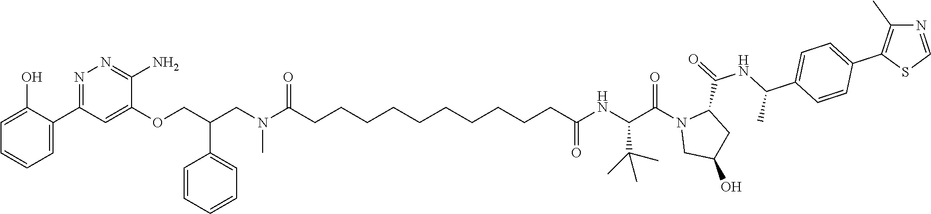 [SMILES: Cc1ncsc1-c1ccc([C@H](C)NC(=O)[C@@H]2C[C@@H](O)CN2C(=O)[C@@H](NC(=O)CCCCCCCCCCC(=O)N(C)CC(COc2cc(-c3ccccc3O)nnc2N)c2ccccc2)C(C)(C)C)cc1]

The title compound, was prepared in analogy to Example 1 from intermediate 21 and Ligase 20 as the trifluoroacetic acid salt. MS (ESI): 999.27 ([M+H]⁺).

### Example 23

N′-[3-[3-amino-6-(2-hydroxyphenyl)pyridazin-4-yl]oxy-2-phenyl-propyl]-N′-methyl-N-[rac-(1S)-2,2-dimethyl-1-[rac-(2S,4R)-4-hydroxy-2-[[rac-(1S)-1-[4-(4-methylthiazol-5-yl)phenyl]ethyl]carbamoyl]pyrrolidine-1-carbonyl]propyl]tetradecanediamide

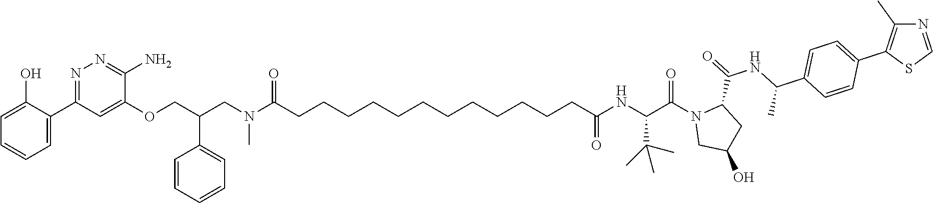 [SMILES: Cc1ncsc1-c1ccc([C@H](C)NC(=O)[C@@H]2C[C@@H](O)CN2C(=O)[C@@H](NC(=O)CCCCCCCCCCCCC(=O)N(C)CC(COc2cc(-c3ccccc3O)nnc2N)c2ccccc2)C(C)(C)C)cc1]

The title compound, was prepared in analogy to Example 1 from intermediate 21 and Ligase 13 as the trifluoroacetic acid salt. MS (ESI): 1017.3 ([M+H]⁺).

### Example 24

### rac-(2S,4R)-4-hydroxy-1-[rac-(2S)-2-[3-[2-[3-[[3-[3-amino-6-(2-hydroxyphenyl)pyridazin-4-yl]oxy-2-phenyl-propyl]-methyl-amino]-3-oxo-propoxy]ethoxy]propanoylamino]-3,3-dimethyl-butanoyl]-N-[rac-(1S)-1-[4-(4-methylthiazol-5-yl)phenyl]ethyl]pyrrolidine-2-carboxamide

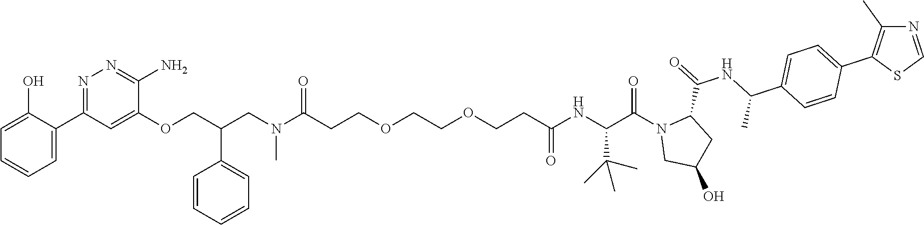 [SMILES: Cc1ncsc1-c1ccc([C@H](C)NC(=O)[C@@H]2C[C@@H](O)CN2C(=O)[C@@H](NC(=O)CCOCCOCCC(=O)N(C)CC(COc2cc(-c3ccccc3O)nnc2N)c2ccccc2)C(C)(C)C)cc1]

The title compound, was prepared in analogy to Example 1 from intermediate 21 and Ligase 14 as the trifluoroacetic acid salt. MS (ESI): 965.1 ([M+H]⁺).

### Example 25

N′-[3-[3-amino-6-(2-hydroxyphenyl)pyridazin-4-yl]oxy-2-phenyl-propyl]-N′-methyl-N-[rac-(1S)-2,2-dimethyl-1-[rac-(2S,4R)-4-hydroxy-2-[[rac-(1S)-1-[4-(4-methylthiazol-5-yl)phenyl]ethyl]carbamoyl]pyrrolidine-1-carbonyl]propyl]decanediamide

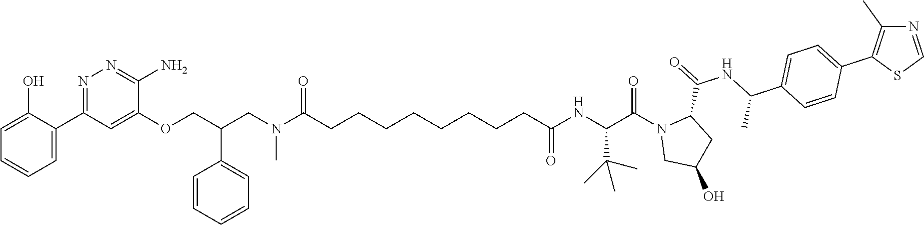 [SMILES: Cc1ncsc1-c1ccc([C@H](C)NC(=O)[C@@H]2C[C@@H](O)CN2C(=O)[C@@H](NC(=O)CCCCCCCCC(=O)N(C)CC(COc2cc(-c3ccccc3O)nnc2N)c2ccccc2)C(C)(C)C)cc1]

The title compound, was prepared in analogy to Example 1 from intermediate 21 and Ligase 15 as the trifluoroacetic acid salt. MS (ESI): 961.2 ([M+H]⁺).

### Example 26

N′-[3-[4-[2-[3-amino-6-(2-hydroxyphenyl)pyridazin-4-yl]oxyethyl]-N-methyl-anilino]propyl]-N′-methyl-N-[rac-(1S)-2,2-dimethyl-1-[rac-(2S,4R)-4-hydroxy-2-[[rac-(1S)-1-[4-(4-methylthiazol-5-yl)phenyl]ethyl]carbamoyl]pyrrolidine-1-carbonyl]propyl]heptanediamide

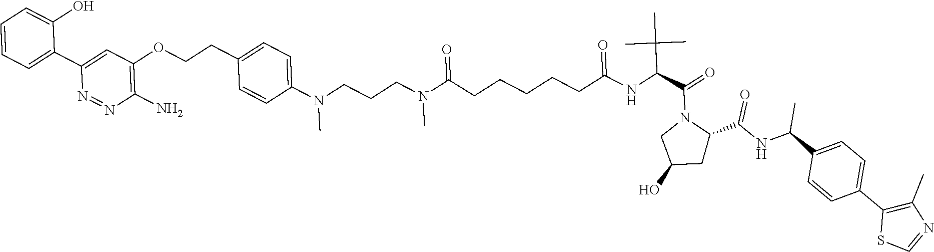 [SMILES: Cc1ncsc1-c1ccc([C@H](C)NC(=O)[C@@H]2C[C@@H](O)CN2C(=O)[C@@H](NC(=O)CCCCCC(=O)N(C)CCCN(C)c2ccc(CCOc3cc(-c4ccccc4O)nnc3N)cc2)C(C)(C)C)cc1]

The title compound, was prepared in analogy to Example 1 from intermediate 26 and Ligase 11 as the trifluoroacetic acid salt. MS (ESI): 977.0 ([M+H]⁺).

### Example 27

### N′-[3-[4-[2-[3-amino-6-(2-hydroxyphenyl)pyridazin-4-yl]oxyethyl]-N-methyl-anilino]propyl]-N′-methyl-N-[rac-(1S)-2,2-dimethyl-1-[rac-(2S,4R)-4-hydroxy-2-[[rac-(1S)-1-[4-(4-methylthiazol-5-yl)phenyl]ethyl]carbamoyl]pyrrolidine-1-carbonyl]propyl]dodecanediamide

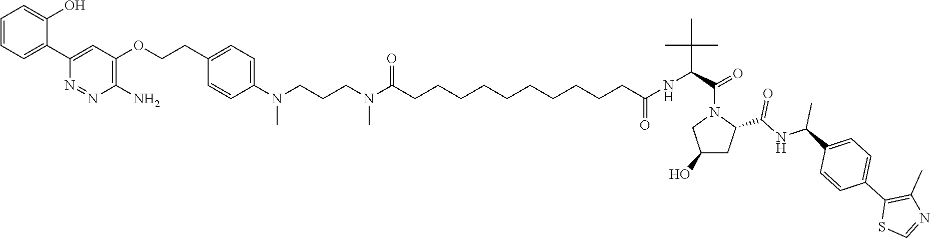 [SMILES: Cc1ncsc1-c1ccc([C@H](C)NC(=O)[C@@H]2C[C@@H](O)CN2C(=O)[C@@H](NC(=O)CCCCCCCCCCC(=O)N(C)CCCN(C)c2ccc(CCOc3cc(-c4ccccc4O)nnc3N)cc2)C(C)(C)C)cc1]

The title compound, was prepared in analogy to Example 1 from intermediate 26 and Ligase 20 as the trifluoroacetic acid salt. MS (ESI): 1047.1 ([M+H]⁺).

### Example 28

### N′-[3-[4-[2-[3-amino-6-(2-hydroxyphenyl)pyridazin-4-yl]oxyethyl]-N-methyl-anilino]propyl]-N′-methyl-N-[rac-(1S)-2,2-dimethyl-1-[rac-(2S,4R)-4-hydroxy-2-[[rac-(1S)-1-[4-(4-methylthiazol-5-yl)phenyl]ethyl]carbamoyl]pyrrolidine-1-carbonyl]propyl]decanediamide

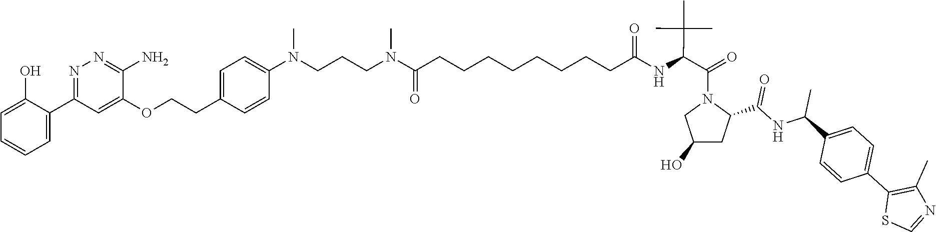 [SMILES: Cc1ncsc1-c1ccc([C@H](C)NC(=O)[C@@H]2C[C@@H](O)CN2C(=O)[C@@H](NC(=O)CCCCCCCCC(=O)N(C)CCCN(C)c2ccc(CCOc3cc(-c4ccccc4O)nnc3N)cc2)C(C)(C)C)cc1]

The title compound, was prepared in analogy to Example 1 from intermediate 26 and Ligase 15 as the trifluoroacetic acid salt. MS (ESI): 1019.1 ([M+H]⁺).

### Example 29

### (2S,4R)-1-[(2S)-2-[[10-[4-[[3-[[3-[3-amino-6-(2-hydroxyphenyl)pyridazin-4-yl]-3,8-diazabicyclo[3.2.1]octan-8-yl]methyl]phenyl]methyl]piperazin-1-yl]-10-oxo-decanoyl]amino]-3,3-dimethyl-butanoyl]-4-hydroxy-N-[[4-(4-methylthiazol-5-yl)phenyl]methyl]pyrrolidine-2-carboxamide

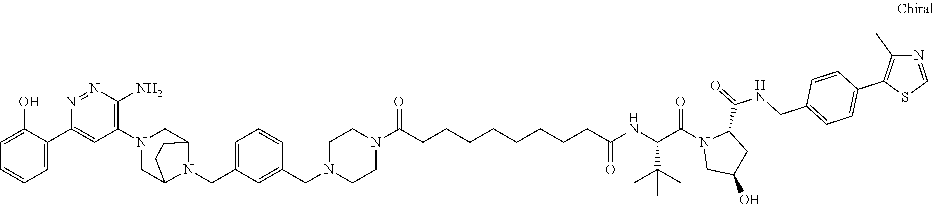 [SMILES: Cc1ncsc1-c1ccc(CNC(=O)[C@@H]2C[C@@H](O)CN2C(=O)[C@@H](NC(=O)CCCCCCCCC(=O)N2CCN(Cc3cccc(CN4C5CCC4CN(c4cc(-c6ccccc6O)nnc4N)C5)c3)CC2)C(C)(C)C)cc1]

The title compound (32 mg, 0.029 mmol, 52% yield), off-white solid, was prepared in analogy to Example 1 from intermediate 29 and Ligase 4. MS (ESI): 1082.8 ([M+H]⁺).

### Example 30

### (2S,4R)-N-[[2-[11-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]-11-oxo-undecoxy]-4-(4-methylthiazol-5-yl)phenyl]methyl]-4-hydroxy-1-[(2S)-3-methyl-2-(1-oxoisoindolin-2-yl)butanoyl]pyrrolidine-2-carboxamide

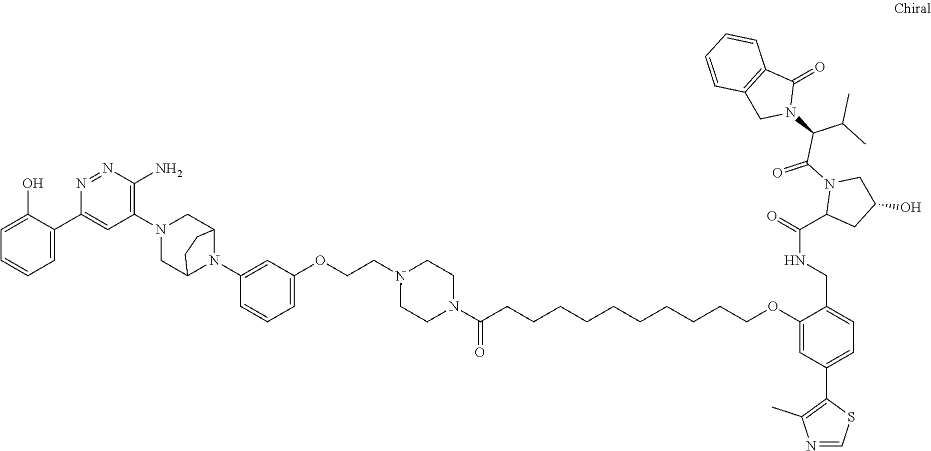 [SMILES: Cc1ncsc1-c1ccc(CNC(=O)C2C[C@@H](O)CN2C(=O)[C@H](C(C)C)N2Cc3ccccc3C2=O)c(OCCCCCCCCCCC(=O)N2CCN(CCOc3cccc(N4C5CCC4CN(c4cc(-c6ccccc6O)nnc4N)C5)c3)CC2)c1]

The title compound (5.9 mg, 0.005 mmol, 15% yield), white solid, was prepared in analogy to Example 1 from Ligase 6 and intermediate 2. MS (ESI): 1217.7 ([M+H]⁺).

### Example 31

### (2R,4S)-1-((2R)-2-(3-(3-(4-(4-(3-((3-amino-6-(2-hydroxyphenyl)pyridazin-4-yl)oxy)piperidin-1-yl)phenyl)piperazin-1-yl)-3-oxopropoxy)propanamido)-3,3-dimethylbutanoyl)-4-hydroxy-N-((R)-1-(4-(4-methylthiazol-5-yl)phenyl)ethyl)pyrrolidine-2-carboxamide

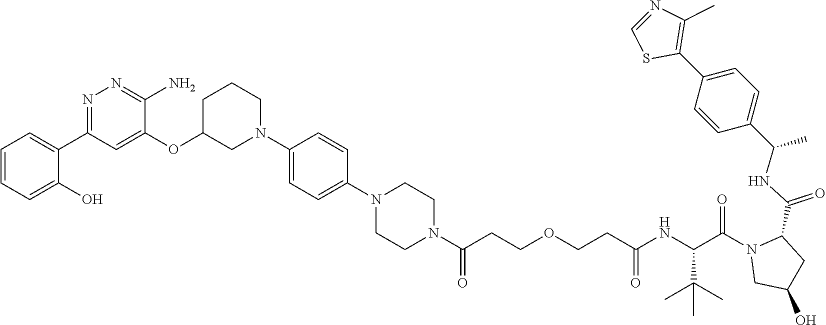 [SMILES: Cc1ncsc1-c1ccc([C@H](C)NC(=O)[C@@H]2C[C@@H](O)CN2C(=O)[C@@H](NC(=O)CCOCCC(=O)N2CCN(c3ccc(N4CCCC(Oc5cc(-c6ccccc6O)nnc5N)C4)cc3)CC2)C(C)(C)C)cc1]

The title compound was prepared in analogy to example 1 from Intermediate 31 and Ligase 16 as the trifluoroacetic acid salt. MS (ESI): 1018.0 ([M+H]⁺).

### Example 32

### rac-(2S,4R)-4-hydroxy-1-[rac-(2S)-2-[[7-[4-[4-[3-[3-amino-6-(2-hydroxyphenyl)pyridazin-4-yl]oxy-1-piperidyl]phenyl]piperazin-1-yl]-7-oxo-heptanoyl]amino]-3,3-dimethyl-butanoyl]-N-[rac-(1S)-1-[4-(4-methylthiazol-5-yl)phenyl]ethyl]pyrrolidine-2-carboxamide

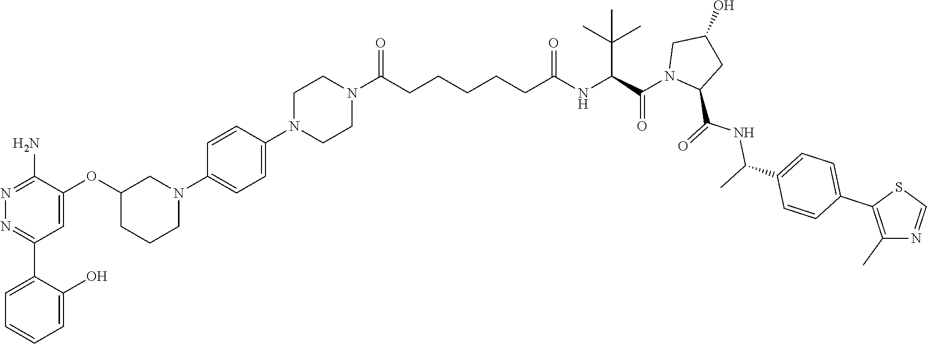 [SMILES: Cc1ncsc1-c1ccc([C@H](C)NC(=O)[C@@H]2C[C@@H](O)CN2C(=O)[C@@H](NC(=O)CCCCCC(=O)N2CCN(c3ccc(N4CCCC(Oc5cc(-c6ccccc6O)nnc5N)C4)cc3)CC2)C(C)(C)C)cc1]

The title compound was prepared in analogy to example 1 from Intermediate 31 and Ligase 11 as the trifluoroacetic acid salt. MS (ESI): 1016.0 ([M+H]⁺).

### Example 33

### rac-(2S,4R)-4-hydroxy-1-[rac-(2S)-2-[[10-[4-[4-[3-[3-amino-6-(2-hydroxyphenyl)pyridazin-4-yl]oxy-1-piperidyl]phenyl]piperazin-1-yl]-10-oxo-decanoyl]amino]-3,3-dimethyl-butanoyl]-N-[rac-(1S)-1-[4-(4-methylthiazol-5-yl)phenyl]ethyl]pyrrolidine-2-carboxamide

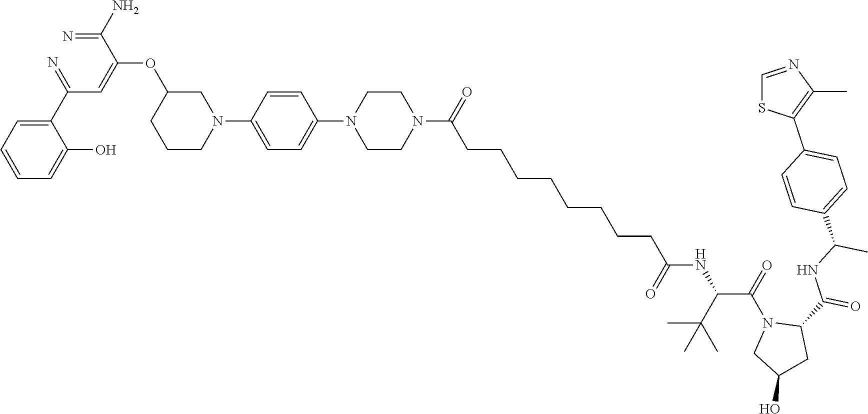 [SMILES: Cc1ncsc1-c1ccc([C@H](C)NC(=O)[C@@H]2C[C@@H](O)CN2C(=O)[C@@H](NC(=O)CCCCCCCCC(=O)N2CCN(c3ccc(N4CCCC(O/C(=C/C(=N)c5ccccc5O)C(=N)N)C4)cc3)CC2)C(C)(C)C)cc1]

The title compound was prepared in analogy to example 1 from Intermediate 31 and Ligase 15 as the trifluoroacetic acid salt. MS (ESI): 1058.4 ([M+H]⁺).

### Example 34

### rac-(2S,4R)-4-hydroxy-1-[rac-(2S)-2-[3-[2-[3-[4-[4-[3-[3-amino-6-(2-hydroxyphenyl)pyridazin-4-yl]oxy-1-piperidyl]phenyl]piperazin-1-yl]-3-oxo-propoxy]ethoxy]propanoylamino]-3,3-dimethyl-butanoyl]-N-[rac-(1S)-1-[4-(4-methylthiazol-5-yl)phenyl]ethyl]pyrrolidine-2-carboxamide

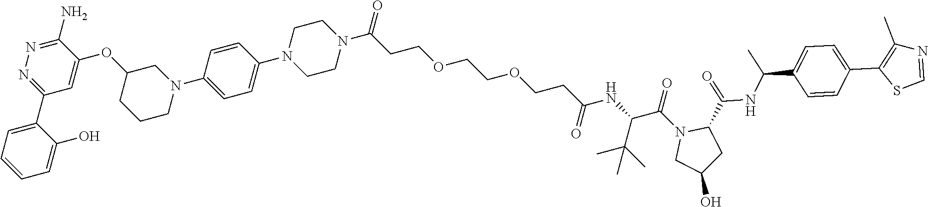 [SMILES: Cc1ncsc1-c1ccc([C@H](C)NC(=O)[C@@H]2C[C@@H](O)CN2C(=O)[C@@H](NC(=O)CCOCCOCCC(=O)N2CCN(c3ccc(N4CCCC(Oc5cc(-c6ccccc6O)nnc5N)C4)cc3)CC2)C(C)(C)C)cc1]

The title compound was prepared in analogy to example 1 from Intermediate 31 and Ligase 14 as the trifluoroacetic acid salt. MS (ESI): 1062.0 ([M+H]⁺).

### Example 35

### (2S,4R)-1-[(2S)-2-[[10-[4-[2-[3-[2-[3-amino-6-(2-hydroxyphenyl)pyridazin-4-yl]ethynyl]phenoxy]ethyl]piperazin-1-yl]-10-oxo-decanoyl]amino]-3,3-dimethyl-butanoyl]-4-hydroxy-N-[[4-(4-methylthiazol-5-yl)phenyl]methyl]pyrrolidine-2-carboxamide

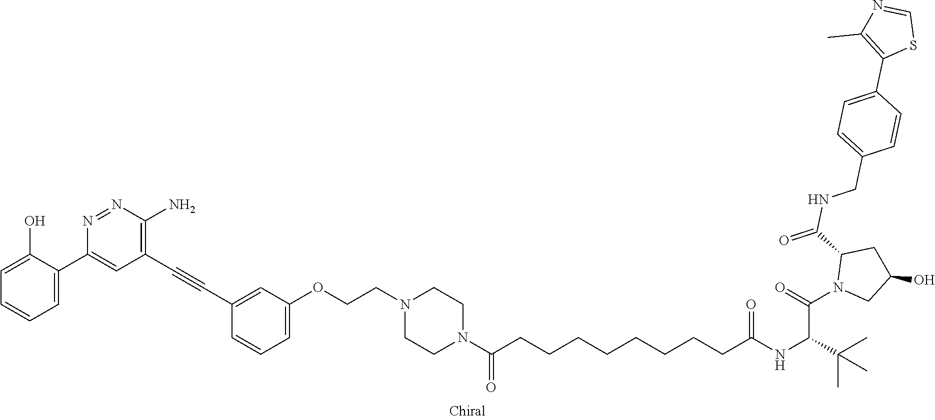 [SMILES: Cc1ncsc1-c1ccc(CNC(=O)[C@@H]2C[C@@H](O)CN2C(=O)[C@@H](NC(=O)CCCCCCCCC(=O)N2CCN(CCOc3cccc(C#Cc4cc(-c5ccccc5O)nnc4N)c3)CC2)C(C)(C)C)cc1]

The title compound (43.7 mg, 43.2 μmol, 52% yield), yellow solid, was prepared in analogy to Example 1 from intermediate 35 and Ligase 4. MS (ESI): 1012.51 ([M+H]⁺).

### Example 36

### (2S,4R)-1-[(2S)-2-[[12-[4-[2-[3-[2-[3-amino-6-(2-hydroxyphenyl)pyridazin-4-yl]ethynyl]phenoxy]ethyl]piperazin-1-yl]-12-oxo-dodecanoyl]amino]-3,3-dimethyl-butanoyl]-4-hydroxy-N-[[4-(4-methylthiazol-5-yl)phenyl]methyl]pyrrolidine-2-carboxamide

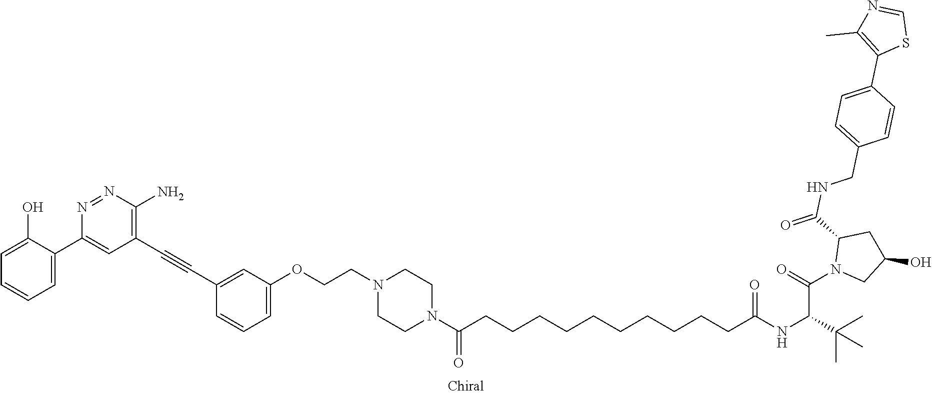 [SMILES: Cc1ncsc1-c1ccc(CNC(=O)[C@@H]2C[C@@H](O)CN2C(=O)[C@@H](NC(=O)CCCCCCCCCCC(=O)N2CCN(CCOc3cccc(C#Cc4cc(-c5ccccc5O)nnc4N)c3)CC2)C(C)(C)C)cc1]

The title compound (65 mg, 62.5 μmol, 76% yield), yellow solid, was prepared in analogy to Example 1 from intermediate 35 and Ligase 12. MS (ESI): 1040.54 ([M+H]⁺).

### Example 37

### (2S,4R)-1-[(2S)-2-[[11-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]-11-oxo-undecanoyl]amino]-3,3-dimethyl-butanoyl]-4-hydroxy-N-[(1S)-1-[4-(4-methylthiazol-5-yl)phenyl]ethyl]pyrrolidine-2-carboxamide

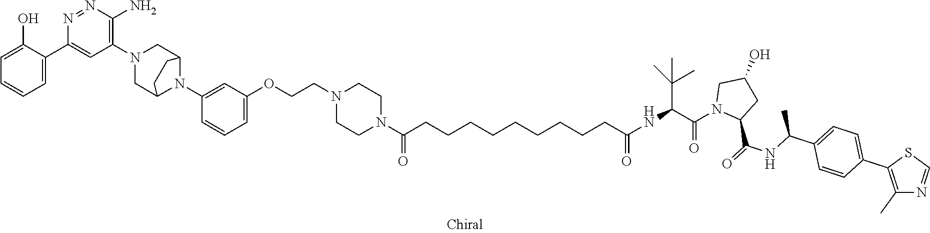 [SMILES: Cc1ncsc1-c1ccc([C@H](C)NC(=O)[C@@H]2C[C@@H](O)CN2C(=O)[C@@H](NC(=O)CCCCCCCCCC(=O)N2CCN(CCOc3cccc(N4C5CCC4CN(c4cc(-c6ccccc6O)nnc4N)C5)c3)CC2)C(C)(C)C)cc1]

The title compound (25.5 mg, 0.023 mmol, 68% yield), white solid, was prepared in analogy to Example 1 from intermediate 4 and Ligase 17. MS (ESI): 1127.3 ([M+H]⁺).

### Example 38

### rac-(2S,4R)-4-hydroxy-1-[rac-(2S)-2-[3-[3-[4-[4-[[3-[3-amino-6-(2-hydroxyphenyl)pyridazin-4-yl]oxy-1-piperidyl]methyl]phenyl]piperazin-1-yl]-3-oxo-propoxy]propanoylamino]-3,3-dimethyl-butanoyl]-N-[rac-(1S)-1-[4-(4-methylthiazol-5-yl)phenyl]ethyl]pyrrolidine-2-carboxamide

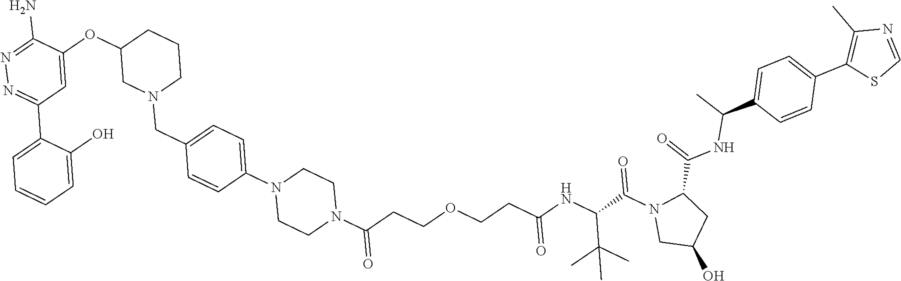 [SMILES: Cc1ncsc1-c1ccc([C@H](C)NC(=O)[C@@H]2C[C@@H](O)CN2C(=O)[C@@H](NC(=O)CCOCCC(=O)N2CCN(c3ccc(CN4CCCC(Oc5cc(-c6ccccc6O)nnc5N)C4)cc3)CC2)C(C)(C)C)cc1]

The title compound, was prepared in analogy to Example 1 from intermediate 38 and Ligase 16 as the trifluoroacetic acid salt. MS (ESI): 1031.9 ([M+H]⁺).

### Example 39

### rac-(2S,4R)-4-hydroxy-1-[rac-(2S)-2-[[4-[4-[4-[[3-[3-amino-6-(2-hydroxyphenyl)pyridazin-4-yl]oxy-1-piperidyl]methyl]phenyl]piperazin-1-yl]-4-oxo-butanoyl]amino]-3,3-dimethyl-butanoyl]-N-[rac-(1S)-1-[4-(4-methylthiazol-5-yl)phenyl]ethyl]pyrrolidine-2-carboxamide

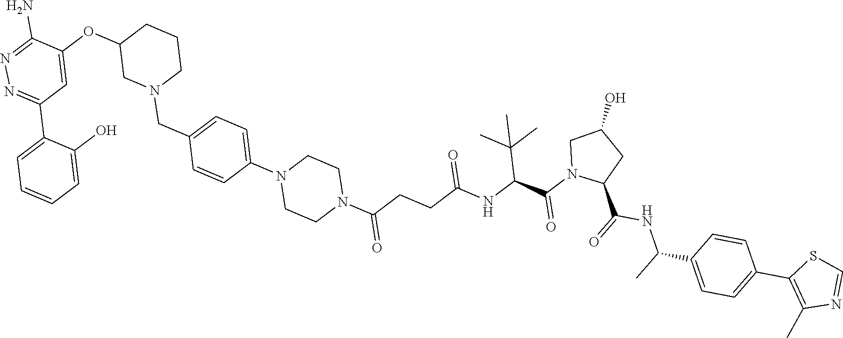 [SMILES: Cc1ncsc1-c1ccc([C@H](C)NC(=O)[C@@H]2C[C@@H](O)CN2C(=O)[C@@H](NC(=O)CCC(=O)N2CCN(c3ccc(CN4CCCC(Oc5cc(-c6ccccc6O)nnc5N)C4)cc3)CC2)C(C)(C)C)cc1]

The title compound, was prepared in analogy to Example 1 from intermediate 38 and Ligase 18 as the trifluoroacetic acid salt. MS (ESI): 987.8 ([M+H]⁺).

### Example 40

### rac-(2S,4R)-4-hydroxy-1-[rac-(2S)-2-[[7-[4-[4-[[3-[3-amino-6-(2-hydroxyphenyl)pyridazin-4-yl]oxy-1-piperidyl]methyl]phenyl]piperazin-1-yl]-7-oxo-heptanoyl]amino]-3,3-dimethyl-butanoyl]-N-[rac-(1S)-1-[4-(4-methylthiazol-5-yl)phenyl]ethyl]pyrrolidine-2-carboxamide

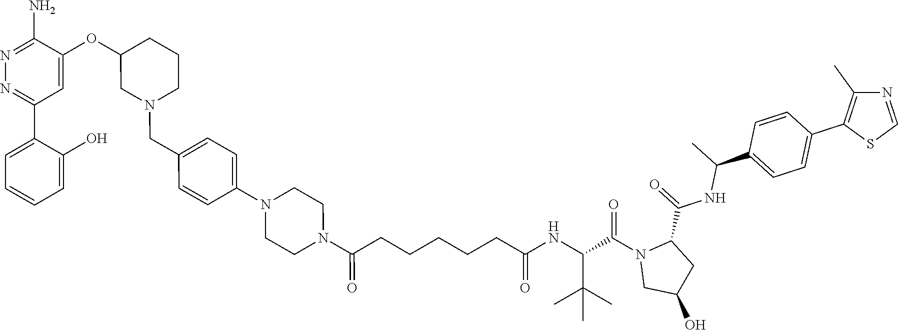 [SMILES: Cc1ncsc1-c1ccc([C@H](C)NC(=O)[C@@H]2C[C@@H](O)CN2C(=O)[C@@H](NC(=O)CCCCCC(=O)N2CCN(c3ccc(CN4CCCC(Oc5cc(-c6ccccc6O)nnc5N)C4)cc3)CC2)C(C)(C)C)cc1]

The title compound, was prepared in analogy to Example 1 from intermediate 38 and Ligase 11 as the trifluoroacetic acid salt. MS (ESI): 1029.9 ([M+H]⁺).

### Example 41

### rac-(2S,4R)-4-hydroxy-1-[rac-(2S)-2-[[10-[4-[4-[[3-[3-amino-6-(2-hydroxyphenyl)pyridazin-4-yl]oxy-1-piperidyl]methyl]phenyl]piperazin-1-yl]-10-oxo-decanoyl]amino]-3,3-dimethyl-butanoyl]-N-[rac-(1S)-1-[4-(4-methylthiazol-5-yl)phenyl]ethyl]pyrrolidine-2-carboxamide

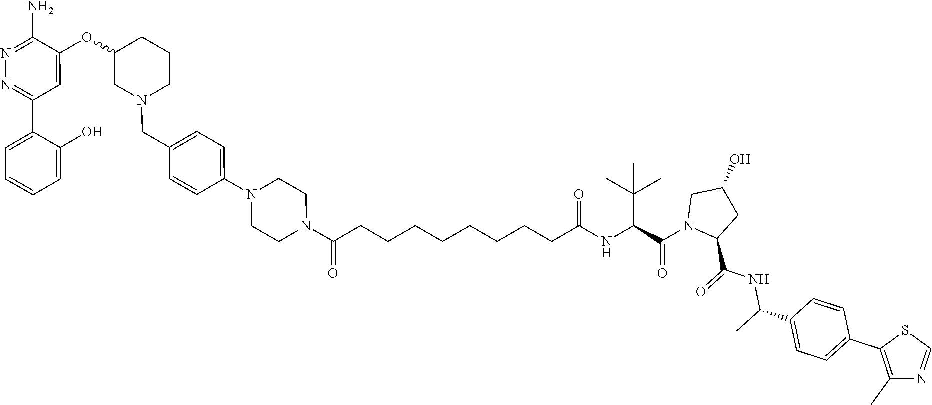 [SMILES: Cc1ncsc1-c1ccc([C@H](C)NC(=O)[C@@H]2C[C@@H](O)CN2C(=O)[C@@H](NC(=O)CCCCCCCCC(=O)N2CCN(c3ccc(CN4CCCC(Oc5cc(-c6ccccc6O)nnc5N)C4)cc3)CC2)C(C)(C)C)cc1]

The title compound, was prepared in analogy to Example 1 from intermediate 38 and Ligase 15 as the trifluoroacetic acid salt. MS (ESI): 1072.0 ([M+H]⁺).

### Example 42

### (2S,4R)-1-[(2S)-2-[[4-[9-[3-amino-6-(2-hydroxyphenyl)pyridazin-4-yl]-1-oxa-4,9-diazaspiro[5.5]undecan-4-yl]-4-oxo-butanoyl]amino]-3,3-dimethyl-butanoyl]-4-hydroxy-N-[(1S)-1-[4-(4-methylthiazol-5-yl)phenyl]ethyl]pyrrolidine-2-carboxamide

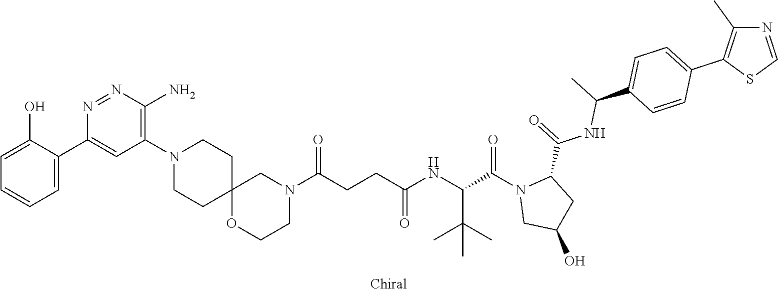 [SMILES: Cc1ncsc1-c1ccc([C@H](C)NC(=O)[C@@H]2C[C@@H](O)CN2C(=O)[C@@H](NC(=O)CCC(=O)N2CCOC3(CCN(c4cc(-c5ccccc5O)nnc4N)CC3)C2)C(C)(C)C)cc1]

The title compound, was prepared in analogy to Example 1 from intermediate 12 and Ligase 18 as the trifluoroacetic acid salt. MS (ESI): 868.7 ([M+H]⁺).

### Example 43

### (2S,4R)-1-[(2S)-2-[[7-[9-[3-amino-6-(2-hydroxyphenyl)pyridazin-4-yl]-1-oxa-4,9-diazaspiro[5.5]undecan-4-yl]-7-oxo-heptanoyl]amino]-3,3-dimethyl-butanoyl]-4-hydroxy-N-[(1S)-1-[4-(4-methylthiazol-5-yl)phenyl]ethyl]pyrrolidine-2-carboxamide

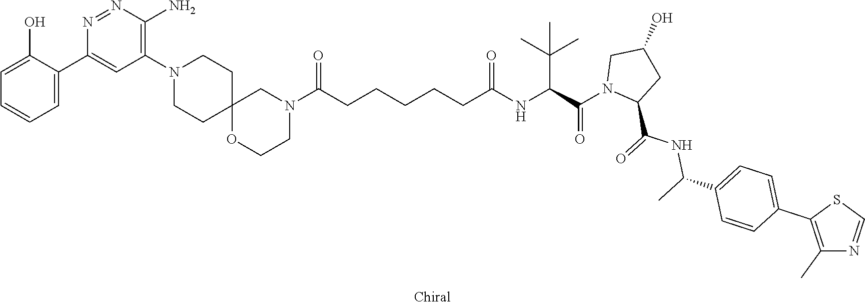 [SMILES: Cc1ncsc1-c1ccc([C@H](C)NC(=O)[C@@H]2C[C@@H](O)CN2C(=O)[C@@H](NC(=O)CCCCCC(=O)N2CCOC3(CCN(c4cc(-c5ccccc5O)nnc4N)CC3)C2)C(C)(C)C)cc1]

The title compound, was prepared in analogy to Example 1 from intermediate 12 and Ligase 11 as the trifluoroacetic acid salt. MS (ESI): 910.8 ([M+H]⁺).

### Example 44

### (2S,4R)-1-[(2S)-2-[[12-[9-[3-amino-6-(2-hydroxyphenyl)pyridazin-4-yl]-1-oxa-4,9-diazaspiro[5.5]undecan-4-yl]-12-oxo-dodecanoyl]amino]-3,3-dimethyl-butanoyl]-4-hydroxy-N-[(1S)-1-[4-(4-methylthiazol-5-yl)phenyl]ethyl]pyrrolidine-2-carboxamide

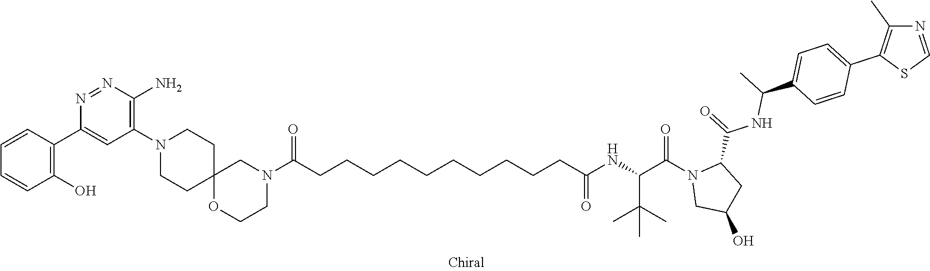 [SMILES: Cc1ncsc1-c1ccc([C@H](C)NC(=O)[C@@H]2C[C@@H](O)CN2C(=O)[C@@H](NC(=O)CCCCCCCCCCC(=O)N2CCOC3(CCN(c4cc(-c5ccccc5O)nnc4N)CC3)C2)C(C)(C)C)cc1]

The title compound, was prepared in analogy to Example 1 from intermediate 12 and Ligase 20 as the trifluoroacetic acid salt. MS (ESI): 980.8 ([M+H]⁺).

### Example 45

### (2S,4R)-1-[(2S)-2-[[14-[9-[3-amino-6-(2-hydroxyphenyl)pyridazin-4-yl]-1-oxa-4,9-diazaspiro[5.5]undecan-4-yl]-14-oxo-tetradecanoyl]amino]-3,3-dimethyl-butanoyl]-4-hydroxy-N-[(1S)-1-[4-(4-methylthiazol-5-yl)phenyl]ethyl]pyrrolidine-2-carboxamide

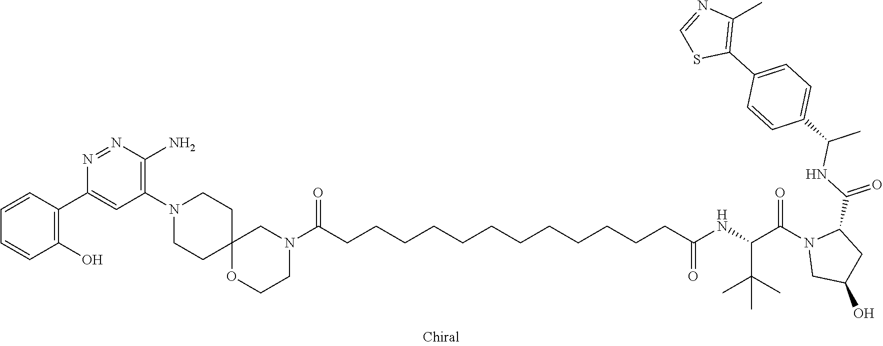 [SMILES: Cc1ncsc1-c1ccc([C@H](C)NC(=O)[C@@H]2C[C@@H](O)CN2C(=O)[C@@H](NC(=O)CCCCCCCCCCCCC(=O)N2CCOC3(CCN(c4cc(-c5ccccc5O)nnc4N)CC3)C2)C(C)(C)C)cc1]

The title compound, was prepared in analogy to Example 1 from intermediate 12 and Ligase 13 as the trifluoroacetic acid salt. MS (ESI): 1008.9 ([M+H]⁺).

### Example 46

### rac-(2S,4R)-4-hydroxy-1-[rac-(2S)-2-[3-[2-[2-[3-[4-[4-[3-[3-amino-6-(2-hydroxyphenyl)pyridazin-4-yl]oxy-1-piperidyl]phenyl]piperazin-1-yl]-3-oxo-propoxy]ethoxy]ethoxy]propanoylamino]-3,3-dimethyl-butanoyl]-N-[rac-(1S)-1-[4-(4-methylthiazol-5-yl)phenyl]ethyl]pyrrolidine-2-carboxamide

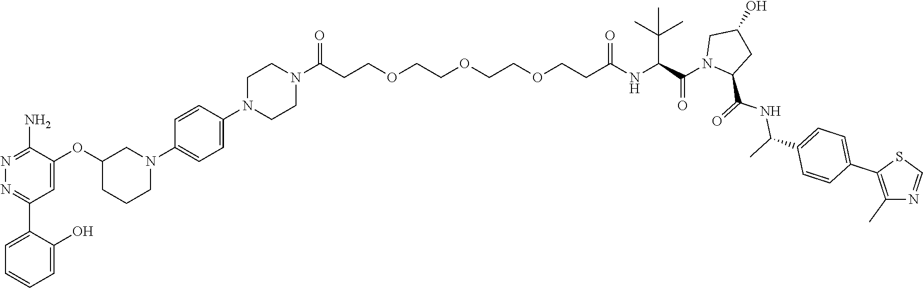 [SMILES: Cc1ncsc1-c1ccc([C@H](C)NC(=O)[C@@H]2C[C@@H](O)CN2C(=O)[C@@H](NC(=O)CCOCCOCCOCCC(=O)N2CCN(c3ccc(N4CCCC(Oc5cc(-c6ccccc6O)nnc5N)C4)cc3)CC2)C(C)(C)C)cc1]

The title compound was prepared in analogy to example 1 from Intermediate 31 and Ligase 19 as the trifluoroacetic acid salt. MS (ESI): 1105.8 ([M+H]⁺).

### Example 47

### rac-(2S,4R)-4-hydroxy-1-[rac-(2S)-2-[[4-[4-[4-[3-[3-amino-6-(2-hydroxyphenyl)pyridazin-4-yl]oxy-1-piperidyl]phenyl]piperazin-1-yl]-4-oxo-butanoyl]amino]-3,3-dimethyl-butanoyl]-N-[rac-(1S)-1-[4-(4-methylthiazol-5-yl)phenyl]ethyl]pyrrolidine-2-carboxamide

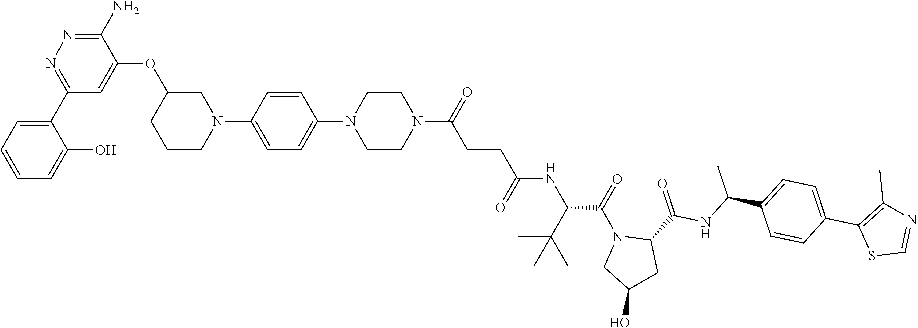 [SMILES: Cc1ncsc1-c1ccc([C@H](C)NC(=O)[C@@H]2C[C@@H](O)CN2C(=O)[C@@H](NC(=O)CCC(=O)N2CCN(c3ccc(N4CCCC(Oc5cc(-c6ccccc6O)nnc5N)C4)cc3)CC2)C(C)(C)C)cc1]

The title compound was prepared in analogy to example 1 from Intermediate 31 and Ligase 18 as the trifluoroacetic acid salt. MS (ESI): 973.7 ([M+H]⁺).

### Example 48

### rac-(2S,4R)-4-hydroxy-1-[rac-(2S)-2-[[12-[4-[4-[3-[3-amino-6-(2-hydroxyphenyl)pyridazin-4-yl]oxy-1-piperidyl]phenyl]piperazin-1-yl]-12-oxo-dodecanoyl]amino]-3,3-dimethyl-butanoyl]-N-[rac-(1S)-1-[4-(4-methylthiazol-5-yl)phenyl]ethyl]pyrrolidine-2-carboxamide

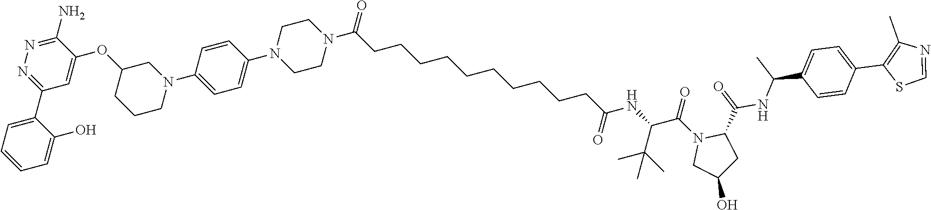 [SMILES: Cc1ncsc1-c1ccc([C@H](C)NC(=O)[C@@H]2C[C@@H](O)CN2C(=O)[C@@H](NC(=O)CCCCCCCCCCC(=O)N2CCN(c3ccc(N4CCCC(Oc5cc(-c6ccccc6O)nnc5N)C4)cc3)CC2)C(C)(C)C)cc1]

The title compound was prepared in analogy to example 1 from Intermediate 31 and Ligase 20 as the trifluoroacetic acid salt. MS (ESI): 1085.8 ([M+H]⁺).

### Example 49

### rac-(2S,4R)-4-hydroxy-1-[rac-(2S)-2-[3-[3-[[3-[3-amino-6-(2-hydroxyphenyl)pyridazin-4-yl]oxy-2-phenyl-propyl]-methyl-amino]-3-oxo-propoxy]propanoylamino]-3,3-dimethyl-butanoyl]-N-[rac-(1S)-1-[4-(4-methylthiazol-5-yl)phenyl]ethyl]pyrrolidine-2-carboxamide

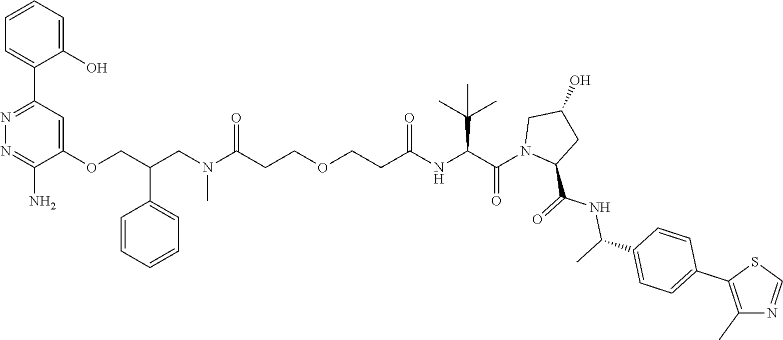 [SMILES: Cc1ncsc1-c1ccc([C@H](C)NC(=O)[C@@H]2C[C@@H](O)CN2C(=O)[C@@H](NC(=O)CCOCCC(=O)N(C)CC(COc2cc(-c3ccccc3O)nnc2N)c2ccccc2)C(C)(C)C)cc1]

The title compound, was prepared in analogy to Example 1 from intermediate 21 and Ligase 16 as the trifluoroacetic acid salt. MS (ESI): 922.7 ([M+H]⁺).

### Example 50

### N1-(3-((3-amino-6-(2-hydroxyphenyl)pyridazin-4-yl)oxy)-2-phenylpropyl)-N10-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)decanediamide

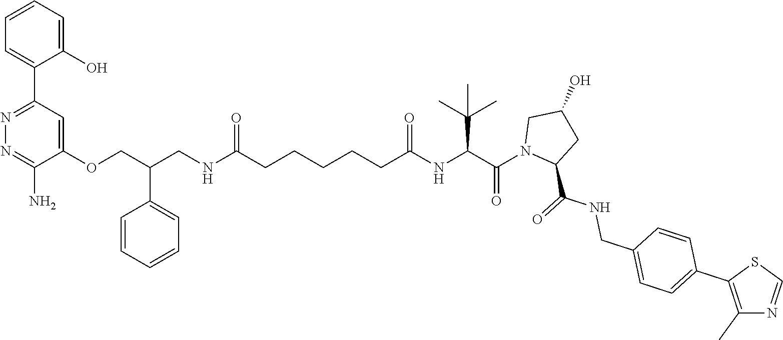 [SMILES: Cc1ncsc1-c1ccc(CNC(=O)[C@@H]2C[C@@H](O)CN2C(=O)[C@@H](NC(=O)CCCCCC(=O)NCC(COc2cc(-c3ccccc3O)nnc2N)c2ccccc2)C(C)(C)C)cc1]

The title compound (24.3 mg, 43% yield), white powder, trifluoroacetic acid salt, was prepared in analogy to Example 1 from intermediate 50 and Ligase 3. MS ISP (m/e): 467.5 [((M/2)+H)+]; 311.9 [((M/3)+H)+].

### Example 51

### N-[3-[3-amino-6-(2-hydroxyphenyl)pyridazin-4-yl]oxy-2-phenyl-propyl]-N′-[rac-(1S)-2,2-dimethyl-1-[rac-(2S,4R)-4-hydroxy-2-[[4-(4-methylthiazol-5-yl)phenyl]methylcarbamoyl]pyrrolidine-1-carbonyl]propyl]decanediamide

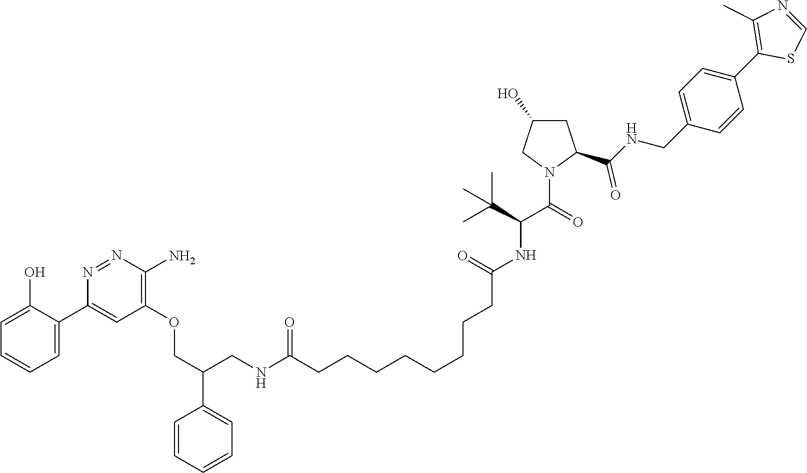 [SMILES: Cc1ncsc1-c1ccc(CNC(=O)[C@@H]2C[C@@H](O)CN2C(=O)[C@@H](NC(=O)CCCCCCCCC(=O)NCC(COc2cc(-c3ccccc3O)nnc2N)c2ccccc2)C(C)(C)C)cc1]

The title compound (24.3 mg, 26 μmol, 43% yield), white solid, trifluoroacetic acid salt, was prepared in analogy to Example 1 from intermediate 50 and Ligase 4. MS (ESI): 467.5 ([(M/2)+H]⁺); 311.9 ([(M/3)+H]⁺).

### Example 52

### (2S,4R)-1-[(2S)-2-[[10-[4-[2-[3-[9-[3-amino-6-(2-hydroxyphenyl)pyridazin-4-yl]-1-oxa-4,9-diazaspiro[5.5]undecan-4-yl]phenoxy]ethyl]piperazin-1-yl]-10-oxo-decanoyl]amino]-3,3-dimethyl-butanoyl]-4-hydroxy-N-[[4-(4-methylthiazol-5-yl)phenyl]methyl]pyrrolidine-2-carboxamide

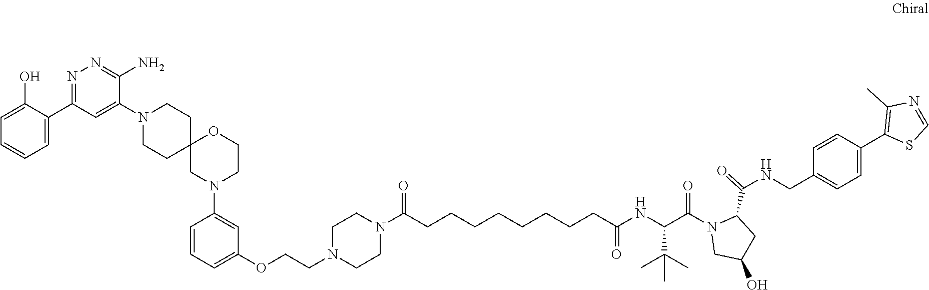 [SMILES: Cc1ncsc1-c1ccc(CNC(=O)[C@@H]2C[C@@H](O)CN2C(=O)[C@@H](NC(=O)CCCCCCCCC(=O)N2CCN(CCOc3cccc(N4CCOC5(CCN(c6cc(-c7ccccc7O)nnc6N)CC5)C4)c3)CC2)C(C)(C)C)cc1]

The title compound (12 mg, 9.55 μmol, 33% yield), off-white solid, trifluoroacetic acid salt, was prepared in analogy to Example 1 from intermediate 52 and Ligase 4. MS (ESI): 1142.8 ([M+H]⁺).

### Example 53

### (2S,4R)-1-[(2S)-2-[[10-[4-[[3-[2-[3-amino-6-(2-hydroxyphenyl)pyridazin-4-yl]ethynyl]phenyl]methyl]piperazin-1-yl]-10-oxo-decanoyl]amino]-3,3-dimethyl-butanoyl]-4-hydroxy-N-[[4-(4-methylthiazol-5-yl)phenyl]methyl]pyrrolidine-2-carboxamide

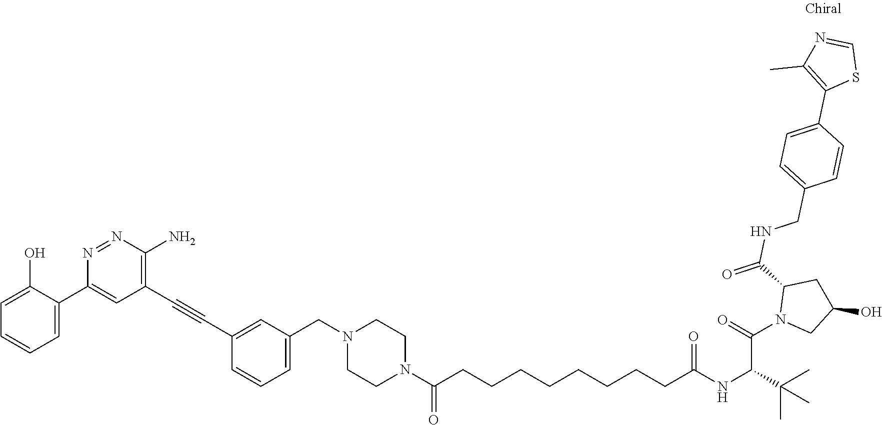 [SMILES: Cc1ncsc1-c1ccc(CNC(=O)[C@@H]2C[C@@H](O)CN2C(=O)[C@@H](NC(=O)CCCCCCCCC(=O)N2CCN(Cc3cccc(C#Cc4cc(-c5ccccc5O)nnc4N)c3)CC2)C(C)(C)C)cc1]

The title compound (11 mg, 9.09 μmol, 16% yield), yellow solid, bis-trifluoroacetic acid salt, was prepared in analogy to Example 1 from intermediate 53 and Ligase 4. MS (ESI): 980.4857 ([M−H]⁻).

### Example 54

### (2S,4R)-1-[(2S)-2-[[12-[4-[[3-[2-[3-amino-6-(2-hydroxyphenyl)pyridazin-4-yl]ethynyl]phenyl]methyl]piperazin-1-yl]-12-oxo-dodecanoyl]amino]-3,3-dimethyl-butanoyl]-4-hydroxy-N-[[4-(4-methylthiazol-5-yl)phenyl]methyl]pyrrolidine-2-carboxamide

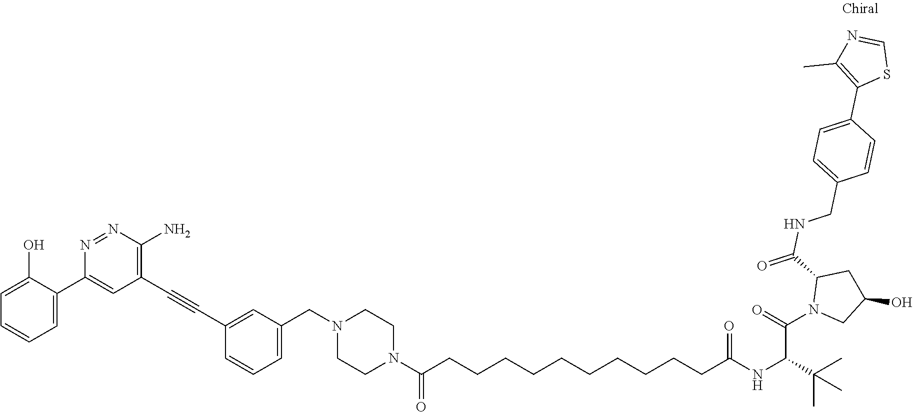 [SMILES: Cc1ncsc1-c1ccc(CNC(=O)[C@@H]2C[C@@H](O)CN2C(=O)[C@@H](NC(=O)CCCCCCCCCCC(=O)N2CCN(Cc3cccc(C#Cc4cc(-c5ccccc5O)nnc4N)c3)CC2)C(C)(C)C)cc1]

The title compound (26 mg, 21 μmol, 38% yield), yellow solid, bis-trifluoroacetic acid salt, was prepared in analogy to Example 1 from intermediate 53 and Ligase 12. MS (ESI): 1010.53 ([M+H]⁺).

### Example 55

### (2S,4R)-1-[(2S)-2-[[10-[4-[2-[2-[3-[3-amino-6-(2-hydroxyphenyl)pyridazin-4-yl]-3,8-diazabicyclo[3.2.1]octan-8-yl]phenoxy]ethyl]piperazin-1-yl]-10-oxo-decanoyl]amino]-3,3-dimethyl-butanoyl]-4-hydroxy-N-[[4-(4-methylthiazol-5-yl)phenyl]methyl]pyrrolidine-2-carboxamide

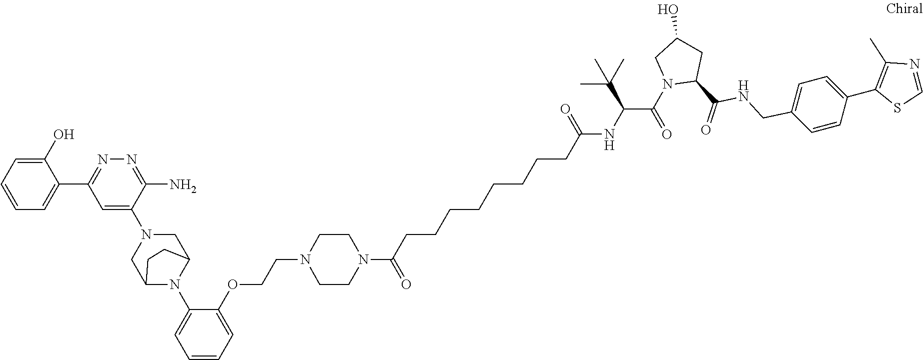 [SMILES: Cc1ncsc1-c1ccc(CNC(=O)[C@@H]2C[C@@H](O)CN2C(=O)[C@@H](NC(=O)CCCCCCCCC(=O)N2CCN(CCOc3ccccc3N3C4CCC3CN(c3cc(-c5ccccc5O)nnc3N)C4)CC2)C(C)(C)C)cc1]

The title compound (16 mg, 14.6 μmol, 17% yield), white solid, bis-trifluoroacetic acid salt, was prepared in analogy to Example 1 from intermediate 55 and Ligase 4. MS (ESI): 1096.5 ([M−H]⁻).

### Example 56

### (2S,4R)-1-[(2S)-2-[[11-[4-[2-[3-[3-[3-amino-6-(2-hydroxyphenyl)pyridazin-4-yl]-3,8-diazabicyclo[3.2.1]octan-8-yl]phenoxy]ethyl]piperazin-1-yl]-11-oxo-undecanoyl]amino]-3-methyl-butanoyl]-4-hydroxy-N-[(1S)-1-[4-(4-methylthiazol-5-yl)phenyl]ethyl]pyrrolidine-2-carboxamide

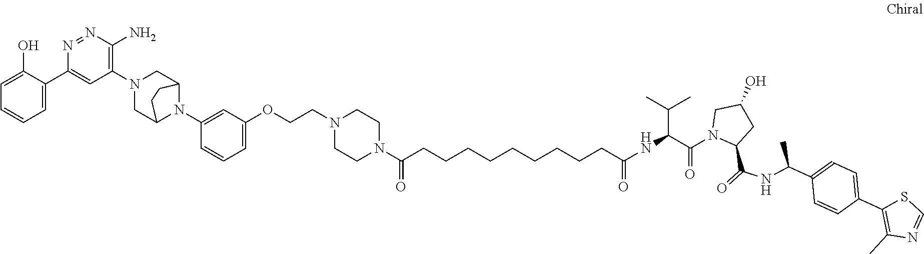 [SMILES: Cc1ncsc1-c1ccc([C@H](C)NC(=O)[C@@H]2C[C@@H](O)CN2C(=O)[C@@H](NC(=O)CCCCCCCCCC(=O)N2CCN(CCOc3cccc(N4C5CCC4CN(c4cc(-c6ccccc6O)nnc4N)C5)c3)CC2)C(C)C)cc1]

The title compound (25.5 mg, 0.020 mmol, 68% yield), white solid, was prepared in analogy to Example 1 from intermediate 2 and Ligase 7. MS (ESI): 1112.8 ([M+H]⁺).

### Example 57

### (2S,4R)-1-[(2S)-2-[[7-[4-[2-[3-[2-[3-amino-6-(2-hydroxyphenyl)pyridazin-4-yl]oxyethyl]phenoxy]ethyl]piperazin-1-yl]-7-oxo-heptanoyl]amino]-3,3-dimethyl-butanoyl]-4-hydroxy-N-[(1S)-1-[4-(4-methylthiazol-5-yl)phenyl]ethyl]pyrrolidine-2-carboxamide

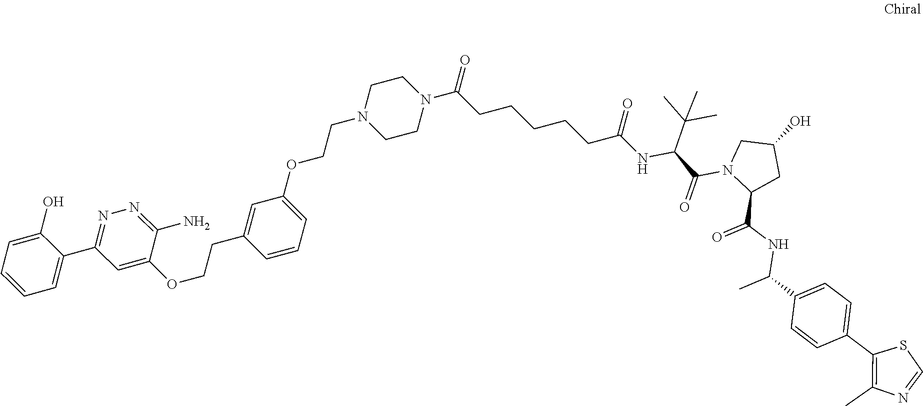 [SMILES: Cc1ncsc1-c1ccc([C@H](C)NC(=O)[C@@H]2C[C@@H](O)CN2C(=O)[C@@H](NC(=O)CCCCCC(=O)N2CCN(CCOc3cccc(CCOc4cc(-c5ccccc5O)nnc4N)c3)CC2)C(C)(C)C)cc1]

The title compound, was prepared in analogy to Example 1 from intermediate 57 and Ligase 11 as the trifluoroacetic acid salt. MS (ESI): 1005.0 ([M+H]⁺).

### Example 58

### (2S,4R)-1-[(2S)-2-[[10-[4-[2-[3-[2-[3-amino-6-(2-hydroxyphenyl)pyridazin-4-yl]oxyethyl]phenoxy]ethyl]piperazin-1-yl]-10-oxo-decanoyl]amino]-3,3-dimethyl-butanoyl]-4-hydroxy-N-[(1S)-1-[4-(4-methylthiazol-5-yl)phenyl]ethyl]pyrrolidine-2-carboxamide

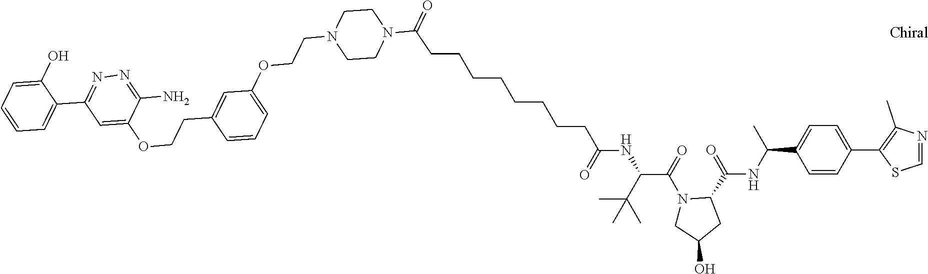 [SMILES: Cc1ncsc1-c1ccc([C@H](C)NC(=O)[C@@H]2C[C@@H](O)CN2C(=O)[C@@H](NC(=O)CCCCCCCCC(=O)N2CCN(CCOc3cccc(CCOc4cc(-c5ccccc5O)nnc4N)c3)CC2)C(C)(C)C)cc1]

The title compound, was prepared in analogy to Example 1 from intermediate 57 and Ligase 15 as the trifluoroacetic acid salt. MS (ESI): 1047.0 ([M+H]⁺).

### Example 59

### (2S,4R)-1-[(2S)-2-[[12-[4-[2-[3-[2-[3-amino-6-(2-hydroxyphenyl)pyridazin-4-yl]oxyethyl]phenoxy]ethyl]piperazin-1-yl]-12-oxo-dodecanoyl]amino]-3,3-dimethyl-butanoyl]-4-hydroxy-N-[(1S)-1-[4-(4-methylthiazol-5-yl)phenyl]ethyl]pyrrolidine-2-carboxamide

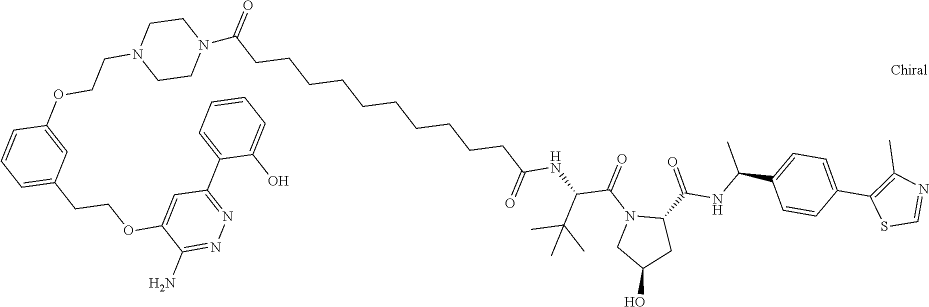 [SMILES: Cc1ncsc1-c1ccc([C@H](C)NC(=O)[C@@H]2C[C@@H](O)CN2C(=O)[C@@H](NC(=O)CCCCCCCCCCC(=O)N2CCN(CCOc3cccc(CCOc4cc(-c5ccccc5O)nnc4N)c3)CC2)C(C)(C)C)cc1]

The title compound, was prepared in analogy to Example 1 from intermediate 57 and Ligase 20 as the trifluoroacetic acid salt. MS (ESI): 1075.0 ([M+H]⁺).

### Example 60

### (2S,4R)-1-[(2S)-2-[[14-[4-[2-[3-[2-[3-amino-6-(2-hydroxyphenyl)pyridazin-4-yl]oxyethyl]phenoxy]ethyl]piperazin-1-yl]-14-oxo-tetradecanoyl]amino]-3,3-dimethyl-butanoyl]-4-hydroxy-N-[(1S)-1-[4-(4-methylthiazol-5-yl)phenyl]ethyl]pyrrolidine-2-carboxamide

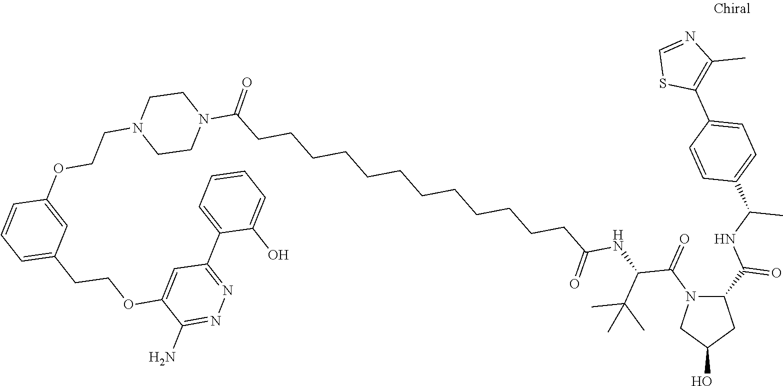 [SMILES: Cc1ncsc1-c1ccc([C@H](C)NC(=O)[C@@H]2C[C@@H](O)CN2C(=O)[C@@H](NC(=O)CCCCCCCCCCCCC(=O)N2CCN(CCOc3cccc(CCOc4cc(-c5ccccc5O)nnc4N)c3)CC2)C(C)(C)C)cc1]

The title compound, was prepared in analogy to Example 1 from intermediate 57 and Ligase 13 as the trifluoroacetic acid salt. MS (ESI): 1103.0 ([M+H]⁺).

### Example 61

### (2S,4R)-1-[(2S)-2-[[7-[4-[[3-[2-[3-amino-6-(2-hydroxyphenyl)pyridazin-4-yl]ethynyl]phenyl]methyl]piperazin-1-yl]-7-oxo-heptanoyl]amino]-3,3-dimethyl-butanoyl]-4-hydroxy-N-[[4-(4-methylthiazol-5-yl)phenyl]methyl]pyrrolidine-2-carboxamide

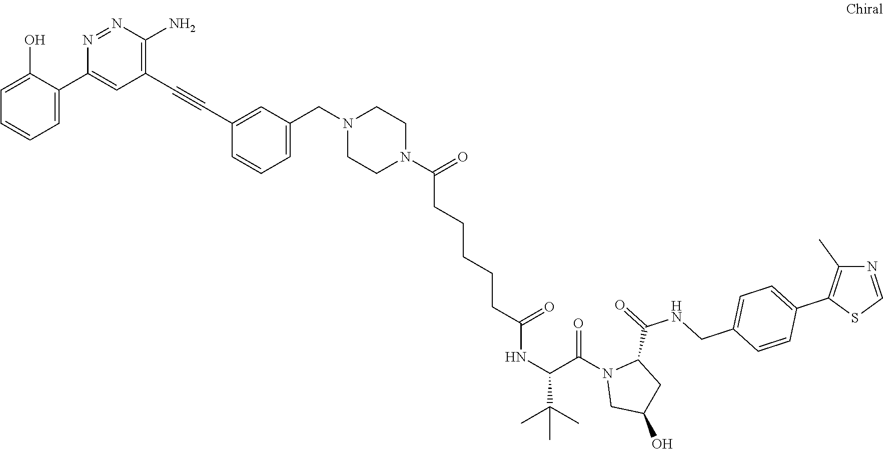 [SMILES: Cc1ncsc1-c1ccc(CNC(=O)[C@@H]2C[C@@H](O)CN2C(=O)[C@@H](NC(=O)CCCCCC(=O)N2CCN(Cc3cccc(C#Cc4cc(-c5ccccc5O)nnc4N)c3)CC2)C(C)(C)C)cc1]

The title compound (28 mg, 24 μmol, 43% yield), yellow solid, bis-trifluoroacetic acid salt, was prepared in analogy to Example 1 from intermediate 53 and Ligase 3. MS (ESI): 940.456 ([M+H]⁺).

### Example 62

### N-[2-[3-amino-6-(2-hydroxyphenyl)pyridazin-4-yl]oxy-1-phenyl-ethyl]-N′-[rac-(1S)-2,2-dimethyl-1-[rac-(2S,4R)-4-hydroxy-2-[[4-(4-methylthiazol-5-yl)phenyl]methylcarbamoyl]pyrrolidine-1-carbonyl]propyl]heptanediamide

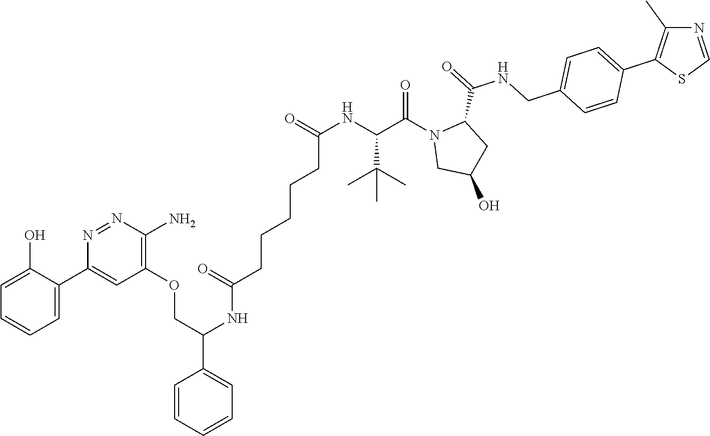 [SMILES: Cc1ncsc1-c1ccc(CNC(=O)[C@@H]2C[C@@H](O)CN2C(=O)[C@@H](NC(=O)CCCCCC(=O)NC(COc2cc(-c3ccccc3O)nnc2N)c2ccccc2)C(C)(C)C)cc1]

The title compound (21.1 mg, 24 μmol, 34% yield), white lyoph powder, trifluoroacetic acid salt, was prepared in analogy to Example 1 from intermediate 62 and Ligase 3. MS (ESI): 877.6 ([M+H]⁺); 439.4 ([(M/2)+H]⁺); 293.2 ([(M/3)+H]⁺).

### Example 63

### N-[2-[3-amino-6-(2-hydroxyphenyl)pyridazin-4-yl]oxy-1-phenyl-ethyl]-N′-[rac-(1S)-2,2-dimethyl-1-[rac-(2S,4R)-4-hydroxy-2-[[4-(4-methylthiazol-5-yl)phenyl]methylcarbamoyl]pyrrolidine-1-carbonyl]propyl]decanediamide

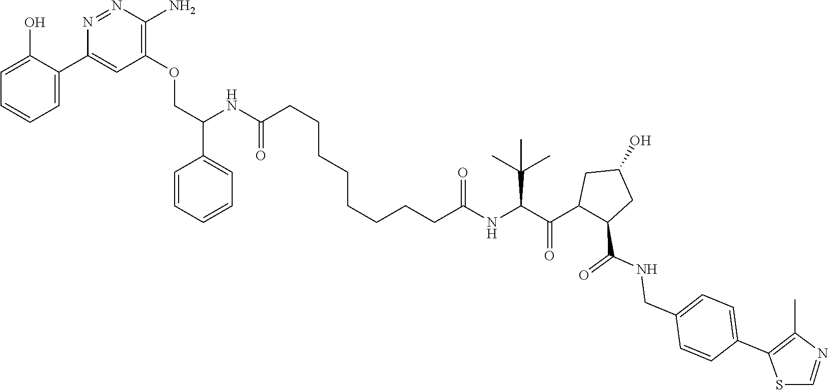 [SMILES: Cc1ncsc1-c1ccc(CNC(=O)[C@@H]2C[C@@H](O)CC2C(=O)[C@@H](NC(=O)CCCCCCCCC(=O)NC(COc2cc(-c3ccccc3O)nnc2N)c2ccccc2)C(C)(C)C)cc1]

The title compound (33.7 mg, 36.6 μmol, 52% yield), white lyoph powder, trifluoroacetic acid salt, was prepared in analogy to Example 1 from intermediate 62 and Ligase 4. MS (ESI): 460.5 ([(M/2)+H]⁺); 307.3 ([(M/3)+H]⁺).

### Example 64

### rac-(2S,4R)-4-hydroxy-N-[[4-(4-methylthiazol-5-yl)phenyl]methyl]-1-[rac-(2S)-2-[[10-[3-[3-amino-6-(2-hydroxyphenyl)pyridazin-4-yl]oxy-5-phenyl-1-piperidyl]-10-oxo-decanoyl]amino]-3,3-dimethyl-butanoyl]pyrrolidine-2-carboxamide

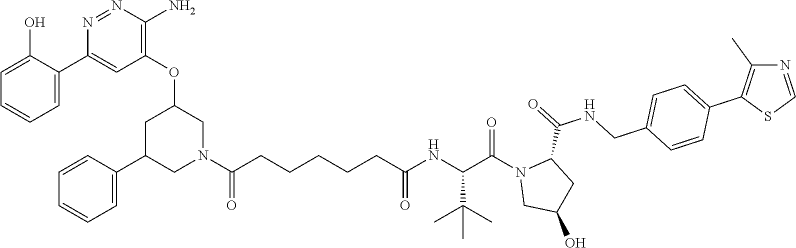 [SMILES: Cc1ncsc1-c1ccc(CNC(=O)[C@@H]2C[C@@H](O)CN2C(=O)[C@@H](NC(=O)CCCCCC(=O)N2CC(Oc3cc(-c4ccccc4O)nnc3N)CC(c3ccccc3)C2)C(C)(C)C)cc1]

The title compound (17.4 mg, 18.9 μmol, 30% yield), white lyoph powder, trifluoroacetic acid salt, was prepared in analogy to Example 1 from intermediate 64 and Ligase 3. MS (ESI): 459.5 ([(M/2)+H]⁺); 306.6 ([(M/3)+H]⁺).

### Example 65

### rac-(2S,4R)-4-hydroxy-N-[[4-(4-methylthiazol-5-yl)phenyl]methyl]-1-[rac-(2S)-2-[[10-[3-[3-amino-6-(2-hydroxyphenyl)pyridazin-4-yl]oxy-5-phenyl-1-piperidyl]-10-oxo-decanoyl]amino]-3,3-dimethyl-butanoyl]pyrrolidine-2-carboxamide

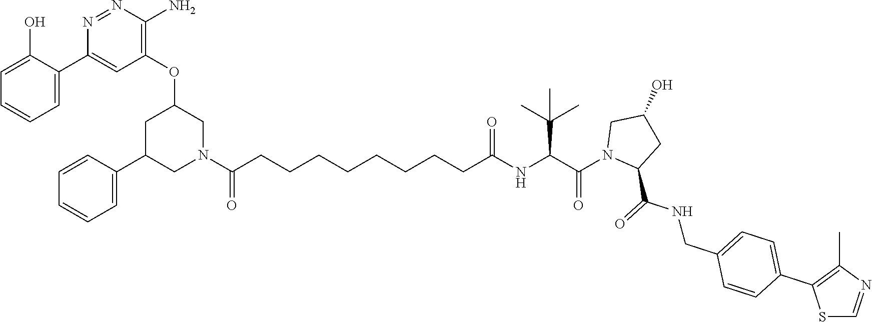 [SMILES: Cc1ncsc1-c1ccc(CNC(=O)[C@@H]2C[C@@H](O)CN2C(=O)[C@@H](NC(=O)CCCCCCCCC(=O)N2CC(Oc3cc(-c4ccccc4O)nnc3N)CC(c3ccccc3)C2)C(C)(C)C)cc1]

The title compound (10.6 mg, 11.0 μmol, 19% yield), white lyoph powder, trifluoroacetic acid salt, was prepared in analogy to Example 1 from intermediate 64 and Ligase 4. MS (ESI): 480.5 ([(M/2)+H]⁺); 320.7 ([(M/3)+H]⁺).

### Example 66

### Trans rac-(2S,4R)-4-hydroxy-N-[[4-(4-methylthiazol-5-yl)phenyl]methyl]-1-[rac-(2S)-2-[[7-[3-[3-amino-6-(2-hydroxyphenyl)pyridazin-4-yl]oxy-5-phenyl-1-piperidyl]-7-oxo-heptanoyl]amino]-3,3-dimethyl-butanoyl]pyrrolidine-2-carboxamide

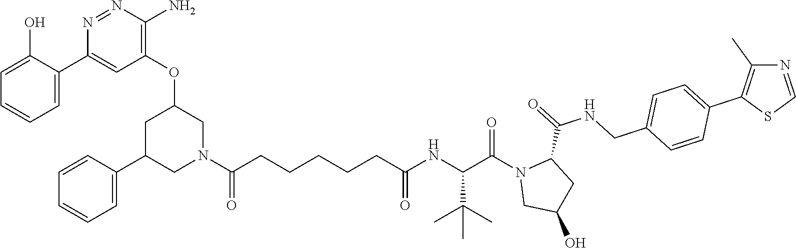 [SMILES: Cc1ncsc1-c1ccc(CNC(=O)[C@@H]2C[C@@H](O)CN2C(=O)[C@@H](NC(=O)CCCCCC(=O)N2CC(Oc3cc(-c4ccccc4O)nnc3N)CC(c3ccccc3)C2)C(C)(C)C)cc1]

The title compound (30.8 mg, 33.5 μmol, 56% yield), white lyoph powder, trifluoroacetic acid salt, was prepared in analogy to Example 1 from intermediate 66 and Ligase 3. MS (ESI): 459.6 ([(M/2)+H]⁺).

### Example 67

### Trans rac-(2S,4R)-4-hydroxy-N-[[4-(4-methylthiazol-5-yl)phenyl]methyl]-1-[rac-(2S)-2-[[10-[3-[3-amino-6-(2-hydroxyphenyl)pyridazin-4-yl]oxy-5-phenyl-1-piperidyl]-10-oxo-decanoyl]amino]-3,3-dimethyl-butanoyl]pyrrolidine-2-carboxamide

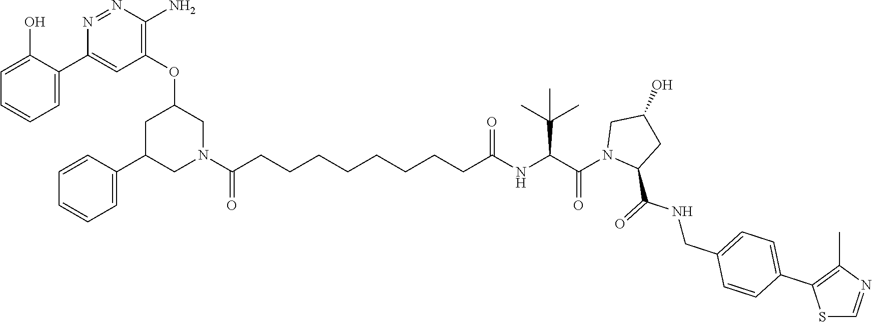 [SMILES: Cc1ncsc1-c1ccc(CNC(=O)[C@@H]2C[C@@H](O)CN2C(=O)[C@@H](NC(=O)CCCCCCCCC(=O)N2CC(Oc3cc(-c4ccccc4O)nnc3N)CC(c3ccccc3)C2)C(C)(C)C)cc1]

The title compound (24.6 mg, 25.6 μmol, 44% yield), white lyoph powder, trifluoroacetic acid salt, was prepared in analogy to Example 1 from intermediate 66 and Ligase 4. MS (ESI): 480.7 ([(M/2)+H]⁺); 320.7 ([(M/3)+H]⁺).

### Example 68

### (2S,4R)-1-[(2S)-2-[[10-[4-[2-[4-[3-[3-amino-6-(2-hydroxyphenyl)pyridazin-4-yl]-3,8-diazabicyclo[3.2.1]octan-8-yl]phenoxy]ethyl]piperazin-1-yl]-10-oxo-decanoyl]amino]-3,3-dimethyl-butanoyl]-4-hydroxy-N-[[4-(4-methylthiazol-5-yl)phenyl]methyl]pyrrolidine-2-carboxamide

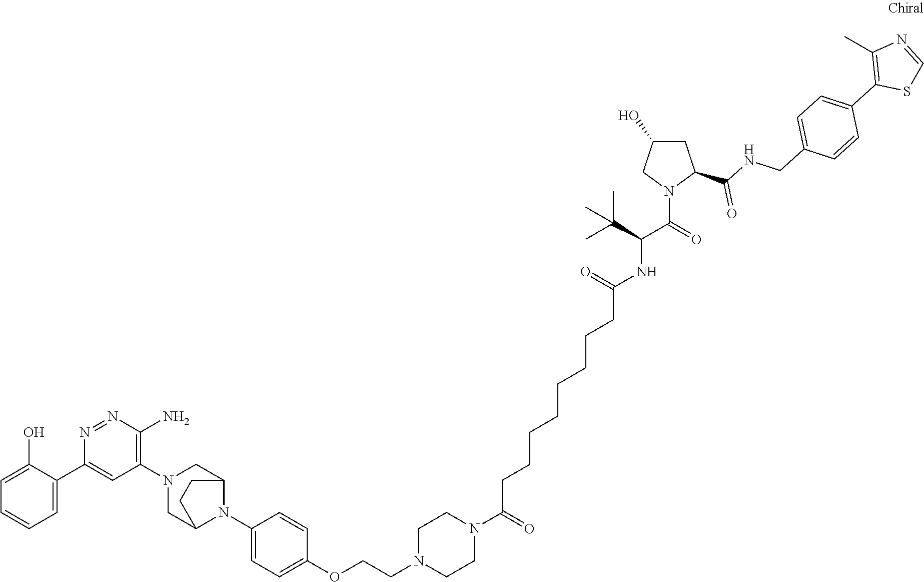 [SMILES: Cc1ncsc1-c1ccc(CNC(=O)[C@@H]2C[C@@H](O)CN2C(=O)[C@@H](NC(=O)CCCCCCCCC(=O)N2CCN(CCOc3ccc(N4C5CCC4CN(c4cc(-c6ccccc6O)nnc4N)C5)cc3)CC2)C(C)(C)C)cc1]

The title compound (17.8 mg, 13.4 μmol, 22% yield), yellow solid, bis-trifluoroacetate salt, was prepared in analogy to Example 1 from intermediate 68 and Ligase 4. MS (ESI): 1098.6 ([M+H]⁺).

### Example 69

### rac-(2S,4R)-4-hydroxy-1-[rac-(2S)-2-[[4-[4-[2-[3-[3-[3-amino-6-(2-hydroxyphenyl)pyridazin-4-yl]oxy-1-piperidyl]phenoxy]ethyl]piperazin-1-yl]-4-oxo-butanoyl]amino]-3,3-dimethyl-butanoyl]-N-[rac-(1S)-1-[4-(4-methylthiazol-5-yl)phenyl]ethyl]pyrrolidine-2-carboxamide

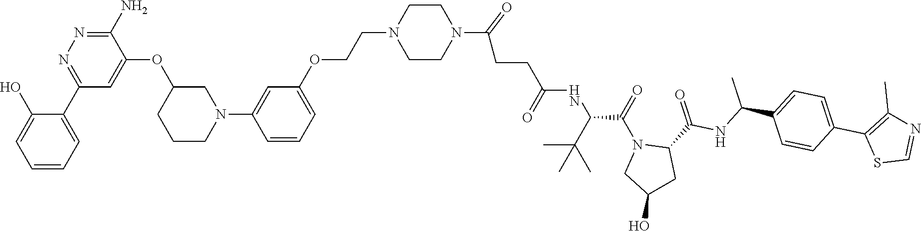 [SMILES: Cc1ncsc1-c1ccc([C@H](C)NC(=O)[C@@H]2C[C@@H](O)CN2C(=O)[C@@H](NC(=O)CCC(=O)N2CCN(CCOc3cccc(N4CCCC(Oc5cc(-c6ccccc6O)nnc5N)C4)c3)CC2)C(C)(C)C)cc1]

The title compound, was prepared in analogy to Example 1 from intermediate 69 and Ligase 18 as the trifluoroacetic acid salt. MS (ESI): 1018.0 ([M+H]⁺).

### Example 70

### rac-(2S,4R)-4-hydroxy-1-[rac-(2S)-2-[[7-[4-[2-[3-[3-[3-amino-6-(2-hydroxyphenyl)pyridazin-4-yl]oxy-1-piperidyl]phenoxy]ethyl]piperazin-1-yl]-7-oxo-heptanoyl]amino]-3,3-dimethyl-butanoyl]-N-[rac-(1S)-1-[4-(4-methylthiazol-5-yl)phenyl]ethyl]pyrrolidine-2-carboxamide

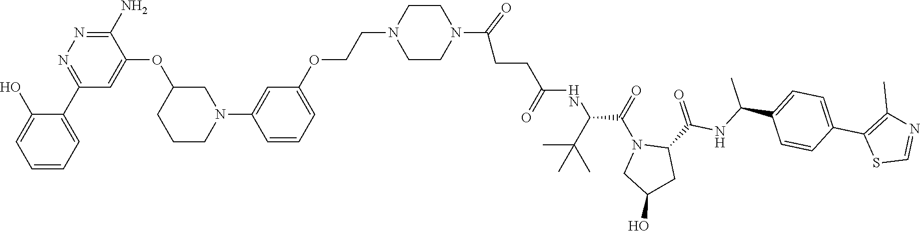 [SMILES: Cc1ncsc1-c1ccc([C@H](C)NC(=O)[C@@H]2C[C@@H](O)CN2C(=O)[C@@H](NC(=O)CCC(=O)N2CCN(CCOc3cccc(N4CCCC(Oc5cc(-c6ccccc6O)nnc5N)C4)c3)CC2)C(C)(C)C)cc1]

The title compound, was prepared in analogy to Example 1 from intermediate 69 and Ligase 11 as the trifluoroacetic acid salt. MS (ESI): 1060.0 ([M+H]⁺).

### Example 71

### rac-(2S,4R)-4-hydroxy-1-[rac-(2S)-2-[[10-[4-[2-[3-[3-[3-amino-6-(2-hydroxyphenyl)pyridazin-4-yl]oxy-1-piperidyl]phenoxy]ethyl]piperazin-1-yl]-10-oxo-decanoyl]amino]-3,3-dimethyl-butanoyl]-N-[rac-(1S)-1-[4-(4-methylthiazol-5-yl)phenyl]ethyl]pyrrolidine-2-carboxamide

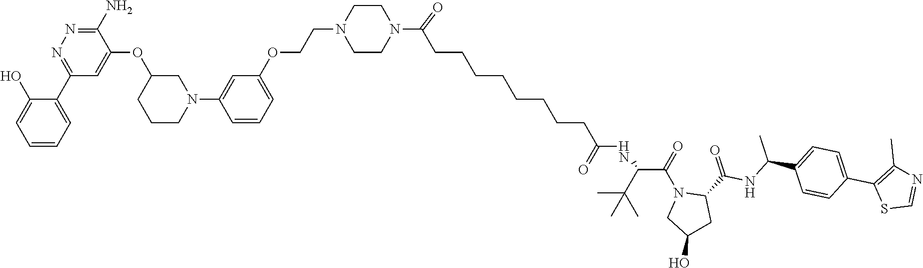 [SMILES: Cc1ncsc1-c1ccc([C@H](C)NC(=O)[C@@H]2C[C@@H](O)CN2C(=O)[C@@H](NC(=O)CCCCCCCCC(=O)N2CCN(CCOc3cccc(N4CCCC(Oc5cc(-c6ccccc6O)nnc5N)C4)c3)CC2)C(C)(C)C)cc1]

The title compound, was prepared in analogy to Example 1 from intermediate 69 and Ligase 15 as the trifluoroacetic acid salt. MS (ESI): 1102.0 ([M+H]⁺).

### Example 72

### rac-(2S,4R)-4-hydroxy-1-[rac-(2S)-2-[[12-[4-[2-[3-[3-[3-amino-6-(2-hydroxyphenyl)pyridazin-4-yl]oxy-1-piperidyl]phenoxy]ethyl]piperazin-1-yl]-12-oxo-dodecanoyl]amino]-3,3-dimethyl-butanoyl]-N-[rac-(1S)-1-[4-(4-methylthiazol-5-yl)phenyl]ethyl]pyrrolidine-2-carboxamide

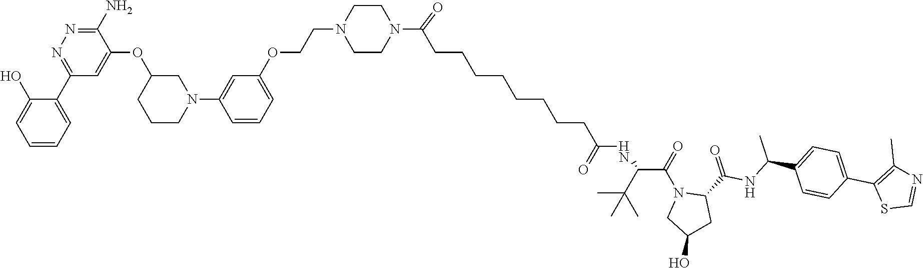 [SMILES: Cc1ncsc1-c1ccc([C@H](C)NC(=O)[C@@H]2C[C@@H](O)CN2C(=O)[C@@H](NC(=O)CCCCCCCCC(=O)N2CCN(CCOc3cccc(N4CCCC(Oc5cc(-c6ccccc6O)nnc5N)C4)c3)CC2)C(C)(C)C)cc1]

The title compound, was prepared in analogy to Example 1 from intermediate 69 and Ligase 20 as the trifluoroacetic acid salt. MS (ESI): 1130.1 ([M+H]⁺).

### Example 73

### (2S,4R)-1-[(2S)-2-[[4-[4-[2-[3-[2-[3-amino-6-(2-hydroxyphenyl)pyridazin-4-yl]oxyethyl]phenoxy]ethyl]piperazin-1-yl]-4-oxo-butanoyl]amino]-3,3-dimethyl-butanoyl]-4-hydroxy-N-[(1S)-1-[4-(4-methylthiazol-5-yl)phenyl]ethyl]pyrrolidine-2-carboxamide

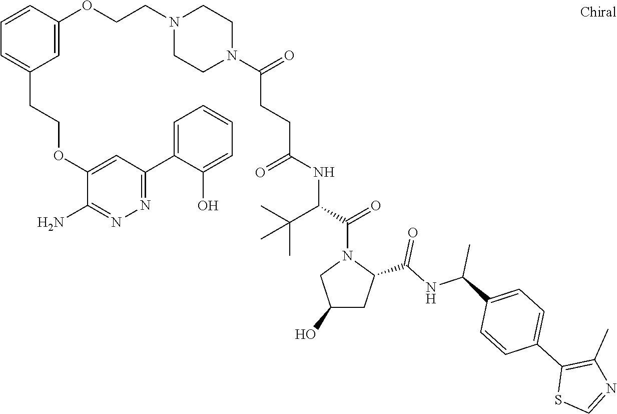 [SMILES: Cc1ncsc1-c1ccc([C@H](C)NC(=O)[C@@H]2C[C@@H](O)CN2C(=O)[C@@H](NC(=O)CCC(=O)N2CCN(CCOc3cccc(CCOc4cc(-c5ccccc5O)nnc4N)c3)CC2)C(C)(C)C)cc1]

The title compound, was prepared in analogy to Example 1 from intermediate 69 and Ligase 18 as the trifluoroacetic acid salt. MS (ESI): 963.0 ([M+H]⁺).

### Example 74

### (2S,4R)-1-[(2S)-2-[[10-[4-[[3-[3-amino-6-(2-hydroxyphenyl)pyridazin-4-yl]-2-fluoro-phenyl]methyl]piperazin-1-yl]-10-oxo-decanoyl]amino]-3,3-dimethyl-butanoyl]-4-hydroxy-N-[[4-(4-methylthiazol-5-yl)phenyl]methyl]pyrrolidine-2-carboxamide

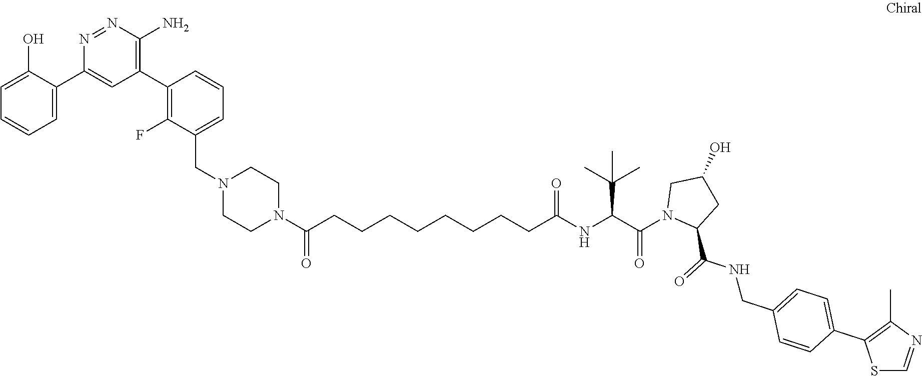 [SMILES: Cc1ncsc1-c1ccc(CNC(=O)[C@@H]2C[C@@H](O)CN2C(=O)[C@@H](NC(=O)CCCCCCCCC(=O)N2CCN(Cc3cccc(-c4cc(-c5ccccc5O)nnc4N)c3F)CC2)C(C)(C)C)cc1]

The title compound (28.3 mg, 28.9 μmol, 42.5% yield), white lyoph powder, bis-trifluoroacetate salt, was prepared in analogy to Example 1 from intermediate 74 and Ligase 4. MS (ESI): 489.1 ([(M/2)+H]⁺), 326.5 ([(M/3)+H]⁺).

### Example 75

### rac-(2S,4R)-4-hydroxy-1-[rac-(2S)-2-[7-[[2-[4-[3-amino-6-(2-hydroxyphenyl)pyridazin-4-yl]pyrazol-1-yl]-2-phenyl-acetyl]amino]heptanoylamino]-3,3-dimethyl-butanoyl]-N-[rac-(1S)-1-[4-(4-methylthiazol-5-yl)phenyl]ethyl]pyrrolidine-2-carboxamide

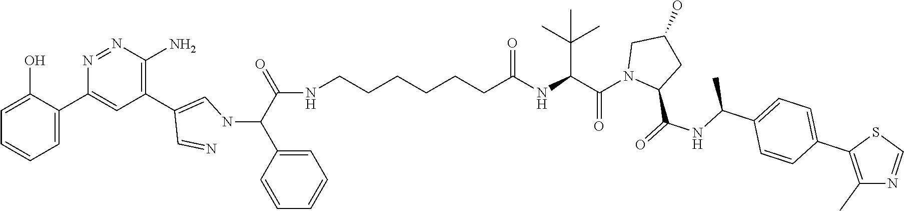 [SMILES: Cc1ncsc1-c1ccc([C@H](C)NC(=O)[C@@H]2C[C@@H](O)CN2C(=O)[C@@H](NC(=O)CCCCCCNC(=O)C(N/C=C(\C=N)c2cc(-c3ccccc3O)nnc2N)c2ccccc2)C(C)(C)C)cc1]

The title compound (5.8 mg, 0.006 mmol, 15% yield), white solid, was prepared in analogy to Example 6 from intermediate 75 and Ligase 8. MS ISP (m/e): 941.5 ([M+H]⁺).

### Example 76

### rac-(2S,4R)-4-hydroxy-1-[rac-(2S)-2-[[14-[4-[4-[3-[3-amino-6-(2-hydroxyphenyl)pyridazin-4-yl]oxy-1-piperidyl]phenyl]piperazin-1-yl]-14-oxo-tetradecanoyl]amino]-3,3-dimethyl-butanoyl]-N-[rac-(1S)-1-[4-(4-methylthiazol-5-yl)phenyl]ethyl]pyrrolidine-2-carboxamide

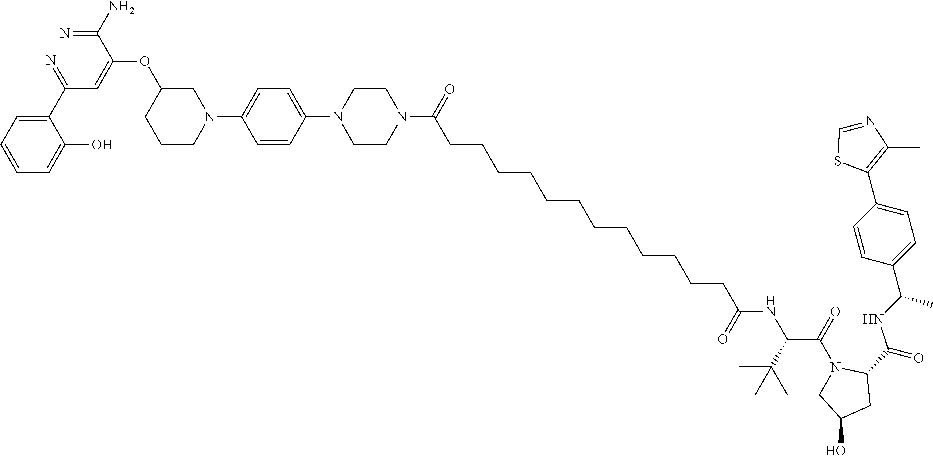 [SMILES: Cc1ncsc1-c1ccc([C@H](C)NC(=O)[C@@H]2C[C@@H](O)CN2C(=O)[C@@H](NC(=O)CCCCCCCCCCCCC(=O)N2CCN(c3ccc(N4CCCC(O/C(=C/C(=N)c5ccccc5O)C(=N)N)C4)cc3)CC2)C(C)(C)C)cc1]

The title compound was prepared in analogy to example 1 from Intermediate 31 and Ligase 13 as the trifluoroacetic acid salt. MS (ESI): 1114.0 ([M+H]⁺).

### Example 77

### rac-(2S,4R)-4-hydroxy-1-[rac-(2S)-2-[[7-[4-[2-[4-[3-[3-amino-6-(2-hydroxyphenyl)pyridazin-4-yl]oxy-1-piperidyl]phenoxy]ethyl]piperazin-1-yl]-7-oxo-heptanoyl]amino]-3,3-dimethyl-butanoyl]-N-[rac-(1S)-1-[4-(4-methylthiazol-5-yl)phenyl]ethyl]pyrrolidine-2-carboxamide

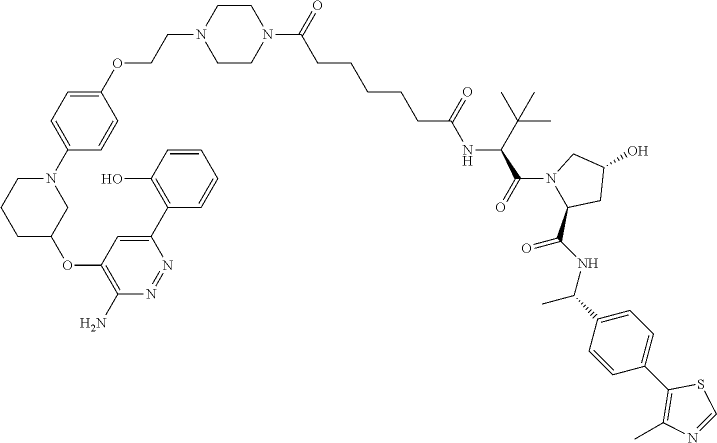 [SMILES: Cc1ncsc1-c1ccc([C@H](C)NC(=O)[C@@H]2C[C@@H](O)CN2C(=O)[C@@H](NC(=O)CCCCCC(=O)N2CCN(CCOc3ccc(N4CCCC(Oc5cc(-c6ccccc6O)nnc5N)C4)cc3)CC2)C(C)(C)C)cc1]

The title compound was prepared in analogy to example 1 from intermediate 77 and Ligase 11 as the trifluoroacetic acid salt. MS (ESI): 1060.1 ([M+H]⁺).

### Example 78

### rac-(2S,4R)-4-hydroxy-1-[rac-(2S)-2-[[10-[4-[2-[4-[3-[3-amino-6-(2-hydroxyphenyl)pyridazin-4-yl]oxy-1-piperidyl]phenoxy]ethyl]piperazin-1-yl]-10-oxo-decanoyl]amino]-3,3-dimethyl-butanoyl]-N-[rac-(1S)-1-[4-(4-methylthiazol-5-yl)phenyl]ethyl]pyrrolidine-2-carboxamide

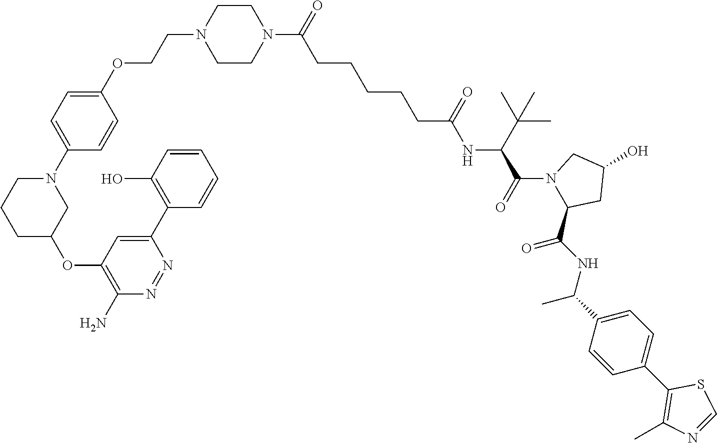 [SMILES: Cc1ncsc1-c1ccc([C@H](C)NC(=O)[C@@H]2C[C@@H](O)CN2C(=O)[C@@H](NC(=O)CCCCCC(=O)N2CCN(CCOc3ccc(N4CCCC(Oc5cc(-c6ccccc6O)nnc5N)C4)cc3)CC2)C(C)(C)C)cc1]

The title compound was prepared in analogy to example 1 from intermediate 77 and Ligase 15 as the trifluoroacetic acid salt. MS (ESI): 1102.1 ([M+H]⁺).

### Example 79

### rac-(2S,4R)-4-hydroxy-1-[rac-(2S)-2-[[12-[4-[2-[4-[3-[3-amino-6-(2-hydroxyphenyl)pyridazin-4-yl]oxy-1-piperidyl]phenoxy]ethyl]piperazin-1-yl]-12-oxo-dodecanoyl]amino]-3,3-dimethyl-butanoyl]-N-[rac-(1S)-1-[4-(4-methylthiazol-5-yl)phenyl]ethyl]pyrrolidine-2-carboxamide

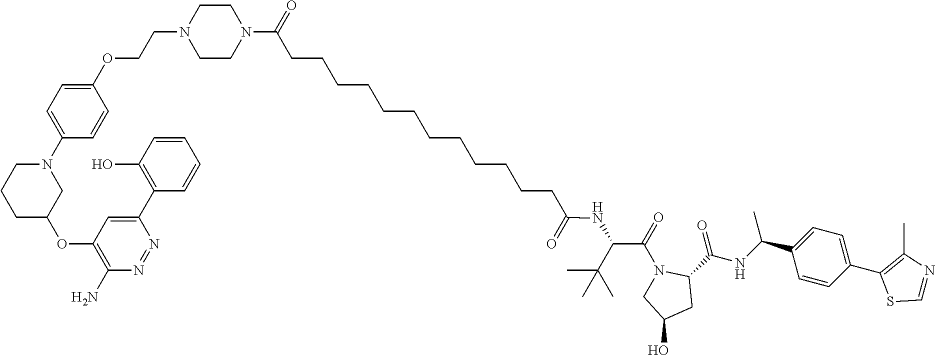 [SMILES: Cc1ncsc1-c1ccc([C@H](C)NC(=O)[C@@H]2C[C@@H](O)CN2C(=O)[C@@H](NC(=O)CCCCCCCCCCCCC(=O)N2CCN(CCOc3ccc(N4CCCC(Oc5cc(-c6ccccc6O)nnc5N)C4)cc3)CC2)C(C)(C)C)cc1]

The title compound was prepared in analogy to example 1 from intermediate 77 and Ligase 20 as the trifluoroacetic acid salt. MS (ESI): 1130.1 ([M+H]⁺).

### Example 80

### rac-(2S,4R)-4-hydroxy-1-[rac-(2S)-2-[[14-[4-[2-[4-[3-[3-amino-6-(2-hydroxyphenyl)pyridazin-4-yl]oxy-1-piperidyl]phenoxy]ethyl]piperazin-1-yl]-14-oxo-tetradecanoyl]amino]-3,3-dimethyl-butanoyl]-N-[rac-(1S)-1-[4-(4-methylthiazol-5-yl)phenyl]ethyl]pyrrolidine-2-carboxamide

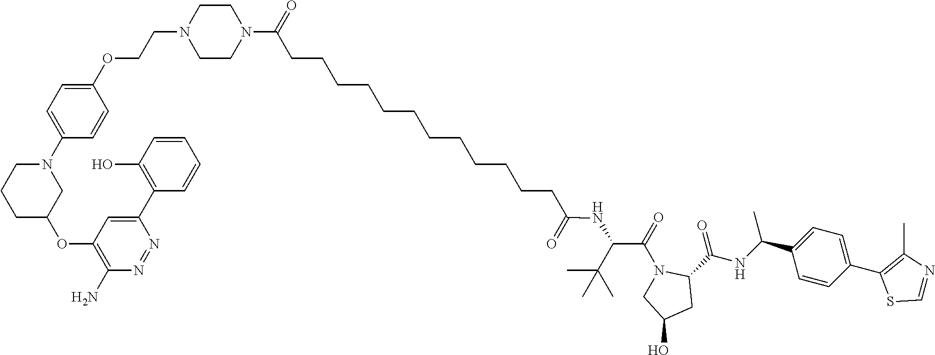 [SMILES: Cc1ncsc1-c1ccc([C@H](C)NC(=O)[C@@H]2C[C@@H](O)CN2C(=O)[C@@H](NC(=O)CCCCCCCCCCCCC(=O)N2CCN(CCOc3ccc(N4CCCC(Oc5cc(-c6ccccc6O)nnc5N)C4)cc3)CC2)C(C)(C)C)cc1]

The title compound was prepared in analogy to example 1 from intermediate 77 and Ligase 13 as the trifluoroacetic acid salt. MS (ESI): 1158.2 ([M+H]⁺).

### Example 81

### rac-(2S,4R)-4-hydroxy-1-[rac-(2S)-2-[[14-[4-[2-[3-[3-[3-amino-6-(2-hydroxyphenyl)pyridazin-4-yl]oxy-1-piperidyl]phenoxy]ethyl]piperazin-1-yl]-14-oxo-tetradecanoyl]amino]-3,3-dimethyl-butanoyl]-N-[rac-(1S)-1-[4-(4-methylthiazol-5-yl)phenyl]ethyl]pyrrolidine-2-carboxamide

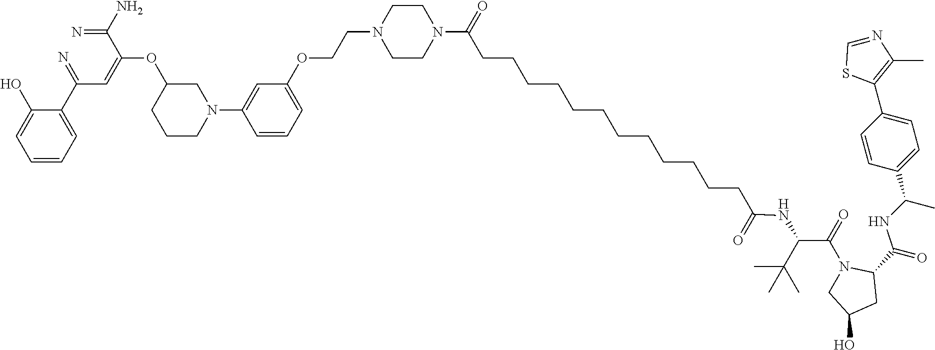 [SMILES: Cc1ncsc1-c1ccc([C@H](C)NC(=O)[C@@H]2C[C@@H](O)CN2C(=O)[C@@H](NC(=O)CCCCCCCCCCCCC(=O)N2CCN(CCOc3cccc(N4CCCC(O/C(=C/C(=N)c5ccccc5O)C(=N)N)C4)c3)CC2)C(C)(C)C)cc1]

The title compound, was prepared in analogy to Example 1 from intermediate 69 and Ligase 13 as the trifluoroacetic acid salt. MS (ESI): 1158.1 ([M+H]⁺)

### Example 82

### rac-(2S,4R)-4-hydroxy-1-[rac-(2S)-2-[[10-[4-[[3-[l-[3-amino-6-(2-hydroxyphenyl)pyridazin-4-yl]oxyethyl]phenyl]methyl]piperazin-1-yl]-10-oxo-decanoyl]amino]-3,3-dimethyl-butanoyl]-N-[rac-(1S)-1-[4-(4-methylthiazol-5-yl)phenyl]ethyl]pyrrolidine-2-carboxamide

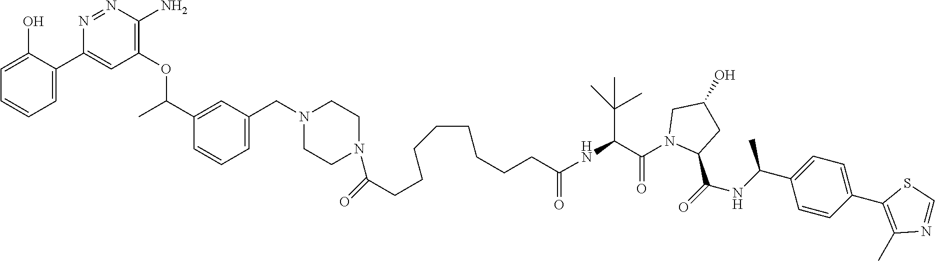 [SMILES: Cc1ncsc1-c1ccc([C@H](C)NC(=O)[C@@H]2C[C@@H](O)CN2C(=O)[C@@H](NC(=O)CCCCCCCCC(=O)N2CCN(Cc3cccc(C(C)Oc4cc(-c5ccccc5O)nnc4N)c3)CC2)C(C)(C)C)cc1]

The title compound (6.1 mg, 0.010 mmol), white solid, was prepared in analogy to Example 6 from intermediate 82 and Ligase 15. MS ISP (m/e): 509.3 ([M/2+H]⁺).

### Example 83

### (2S,4R)-1-[(2S)-2-[[7-[4-[[3-[1-[3-amino-6-(2-hydroxyphenyl)pyridazin-4-yl]azetidin-3-yl]oxyphenyl]methyl]piperazin-1-yl]-7-oxo-heptanoyl]amino]-3,3-dimethyl-butanoyl]-4-hydroxy-N-[(1S)-1-[4-(4-methylthiazol-5-yl)phenyl]ethyl]pyrrolidine-2-carboxamide

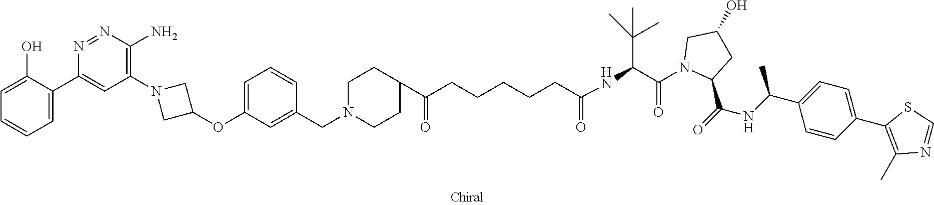 [SMILES: Cc1ncsc1-c1ccc([C@H](C)NC(=O)[C@@H]2C[C@@H](O)CN2C(=O)[C@@H](NC(=O)CCCCCC(=O)C2CCN(Cc3cccc(OC4CN(c5cc(-c6ccccc6O)nnc5N)C4)c3)CC2)C(C)(C)C)cc1]

The title compound (6.5 mg, 0.006 mmol, 12% yield), white solid, was prepared in analogy to Example 6 from intermediate 83 and Ligase 11. MS ISP (m/e): 1001.6 ([M+H]⁺).

### Example 84

### (2S,4R)-1-[(2S)-2-[[12-[3-[3-Amino-6-(2-hydroxyphenyl)pyridazin-4-yl]-3,8-diazabicyclo[3.2.1]octan-8-yl]-12-oxo-dodecanoyl]amino]-3,3-dimethyl-butanoyl]-4-hydroxy-N-[[4-(4-methylthiazol-5-yl)phenyl]methyl]pyrrolidine-2-carboxamide

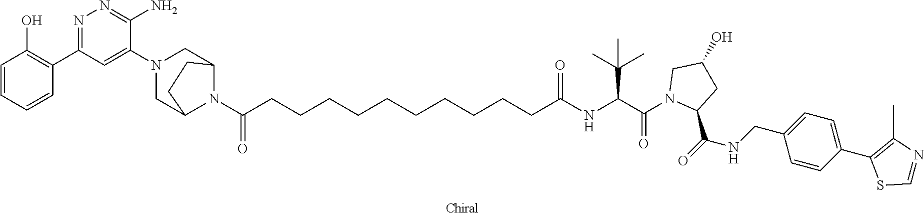 [SMILES: Cc1ncsc1-c1ccc(CNC(=O)[C@@H]2C[C@@H](O)CN2C(=O)[C@@H](NC(=O)CCCCCCCCCCC(=O)N2C3CCC2CN(c2cc(-c4ccccc4O)nnc2N)C3)C(C)(C)C)cc1]

The title compound (44 mg, 42.5 μmol, 47% yield), white solid, trifluoroacetic acid salt, was prepared in analogy to Example 1 from intermediate 84 and Ligase 12. MS (ESI): 922.5 ([M+H]⁺).

### Example 85

### (2S,4R)-1-[(2S)-2-[[7-[3-[3-Amino-6-(2-hydroxyphenyl)pyridazin-4-yl]-3,8-diazabicyclo[3.2.1]octan-8-yl]-7-oxo-heptanoyl]amino]-3,3-dimethyl-butanoyl]-4-hydroxy-N-[[4-(4-methylthiazol-5-yl)phenyl]methyl]pyrrolidine-2-carboxamide

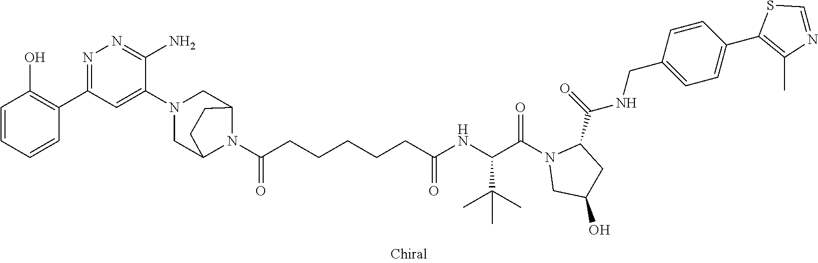 [SMILES: Cc1ncsc1-c1ccc(CNC(=O)[C@@H]2C[C@@H](O)CN2C(=O)[C@@H](NC(=O)CCCCCC(=O)N2C3CCC2CN(c2cc(-c4ccccc4O)nnc2N)C3)C(C)(C)C)cc1]

The title compound (46.3 mg, 47.9 μmol, 53% yield), white solid, trifluoroacetic acid salt, was prepared in analogy to Example 1 from intermediate 84 and Ligase 3. MS (ESI): 852.4 ([M+H]⁺).

### Example 86

### (2S,4R)-1-[(2S)-2-[[10-[4-[[3-[1-[3-amino-6-(2-hydroxyphenyl)pyridazin-4-yl]azetidin-3-yl]oxyphenyl]methyl]piperazin-1-yl]-10-oxo-decanoyl]amino]-3,3-dimethyl-butanoyl]-4-hydroxy-N-[(1S)-1-[4-(4-methylthiazol-5-yl)phenyl]ethyl]pyrrolidine-2-carboxamide

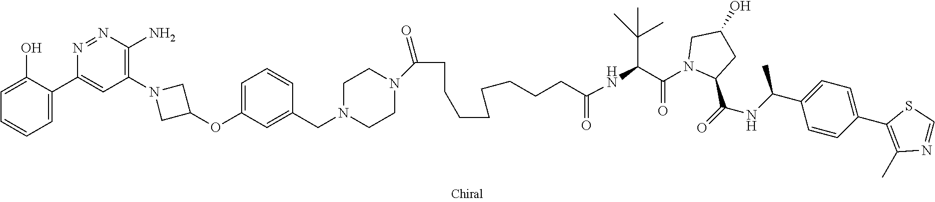 [SMILES: Cc1ncsc1-c1ccc([C@H](C)NC(=O)[C@@H]2C[C@@H](O)CN2C(=O)[C@@H](NC(=O)CCCCCCCCC(=O)N2CCN(Cc3cccc(OC4CN(c5cc(-c6ccccc6O)nnc5N)C4)c3)CC2)C(C)(C)C)cc1]

The title compound (5.2 mg, 4.9 μmol, 9% yield), white solid, was prepared in analogy to Example 6 from intermediate 83 and Ligase 15. MS ISP (m/e): 522.7 ([M/2+H]⁺).

### Example 87

### (2S,4R)-1-[(2S)-2-[8-[4-[2-[[4-[3-amino-6-(2-hydroxyphenyl)pyridazin-4-yl]phenyl]methyl-methyl-amino]acetyl]piperazin-1-yl]octanoylamino]-3,3-dimethyl-butanoyl]-4-hydroxy-N-[[4-(4-methylthiazol-5-yl)phenyl]methyl]pyrrolidine-2-carboxamide

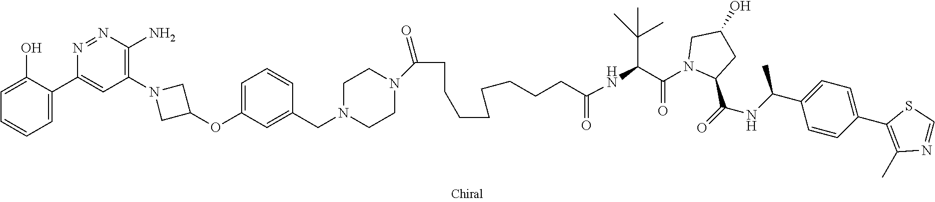 [SMILES: Cc1ncsc1-c1ccc([C@H](C)NC(=O)[C@@H]2C[C@@H](O)CN2C(=O)[C@@H](NC(=O)CCCCCCCCC(=O)N2CCN(Cc3cccc(OC4CN(c5cc(-c6ccccc6O)nnc5N)C4)c3)CC2)C(C)(C)C)cc1]

The title compound (7 mg, 6.36 μmol, 17% yield), white solid, trifluoroacetic acid salt, was prepared in analogy to Example 1 from intermediate 87 and Ligase 21. MS (ESI): 987.5301 ([M+H]⁺).

### Example 88

### rac-(2S,4R)-4-hydroxy-1-[rac-(2S)-2-[10-[[2-[4-[3-amino-6-(2-hydroxyphenyl)pyridazin-4-yl]pyrazol-1-yl]-2-phenyl-acetyl]amino]decanoylamino]-3,3-dimethyl-butanoyl]-N-[rac-(1S)-1-[4-(4-methylthiazol-5-yl)phenyl]ethyl]pyrrolidine-2-carboxamide

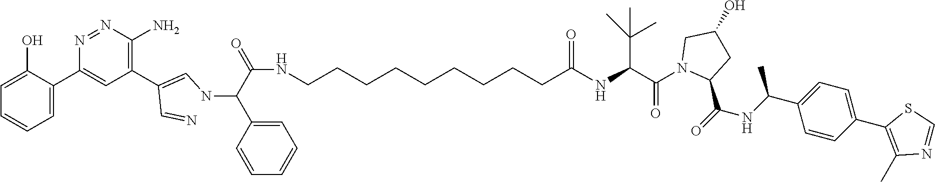 [SMILES: Cc1ncsc1-c1ccc([C@H](C)NC(=O)[C@@H]2C[C@@H](O)CN2C(=O)[C@@H](NC(=O)CCCCCCCCCNC(=O)C(N/C=C(\C=N)c2cc(-c3ccccc3O)nnc2N)c2ccccc2)C(C)(C)C)cc1]

The title compound (4 mg, 0.004 mmol, 10% yield), white solid, was prepared in analogy to Example 6 from intermediate 75 and Ligase 9. MS ISP (m/e): 983.7 ([M+H]⁺).

### Example 89

### N-[3-[3-amino-6-(2-hydroxyphenyl)pyridazin-4-yl]-3-azabicyclo[3.2.1]octan-8-yl]-N′-[(1S)-1-[(2S,4R)-4-hydroxy-2-[[4-(4-methylthiazol-5-yl)phenyl]methylcarbamoyl]pyrrolidine-1-carbonyl]-2,2-dimethyl-propyl]decanediamide

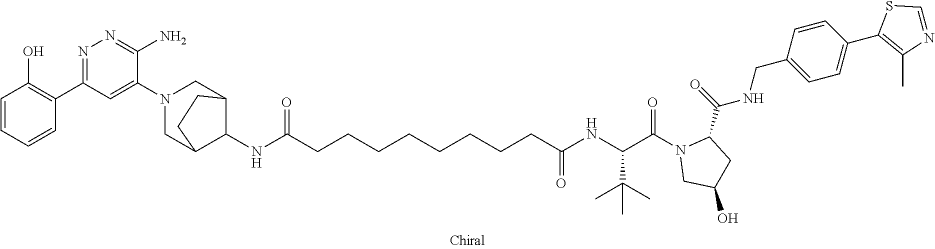 [SMILES: Cc1ncsc1-c1ccc(CNC(=O)[C@@H]2C[C@@H](O)CN2C(=O)[C@@H](NC(=O)CCCCCCCCC(=O)NC2C3CCC2CN(c2cc(-c4ccccc4O)nnc2N)C3)C(C)(C)C)cc1]

The title compound (23 mg, 40.5 μmol, 31% yield), off-white solid, trifluoroacetic acid salt, was prepared in analogy to Example 1 from intermediate 89 and Ligase 4. MS (ESI): 455.2 ([M+2H]²⁺).

### Example 90

### rac-(2S,4R)-4-hydroxy-1-[rac-(2S)-2-[[7-[4-[3-[3-[3-amino-6-(2-hydroxyphenyl)pyridazin-4-yl]oxy-1-piperidyl]phenyl]piperazin-1-yl]-7-oxo-heptanoyl]amino]-3,3-dimethyl-butanoyl]-N-[rac-(1S)-1-[4-(4-methylthiazol-5-yl)phenyl]ethyl]pyrrolidine-2-carboxamide

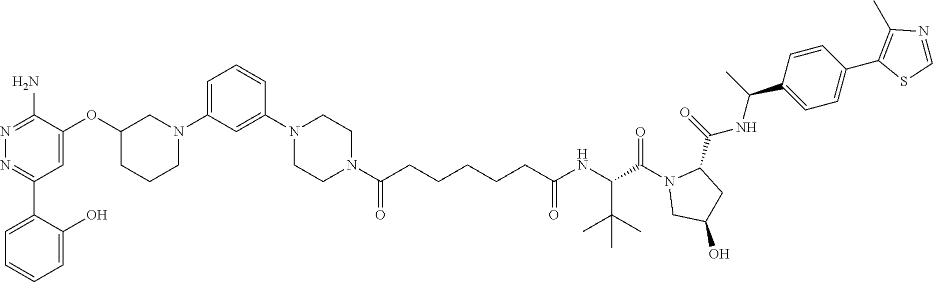 [SMILES: Cc1ncsc1-c1ccc([C@H](C)NC(=O)[C@@H]2C[C@@H](O)CN2C(=O)[C@@H](NC(=O)CCCCCC(=O)N2CCN(c3cccc(N4CCCC(Oc5cc(-c6ccccc6O)nnc5N)C4)c3)CC2)C(C)(C)C)cc1]

The title compound, was prepared in analogy to Example 1 from intermediate 90 and Ligase 11 as the trifluoroacetic acid salt. MS (ESI): 1016.1 ([M+H]⁺).

### Example 91

### rac-(2S,4R)-4-hydroxy-1-[rac-(2S)-2-[[10-[4-[3-[3-[3-amino-6-(2-hydroxyphenyl)pyridazin-4-yl]oxy-1-piperidyl]phenyl]piperazin-1-yl]-10-oxo-decanoyl]amino]-3,3-dimethyl-butanoyl]-N-[rac-(1S)-1-[4-(4-methylthiazol-5-yl)phenyl]ethyl]pyrrolidine-2-carboxamide

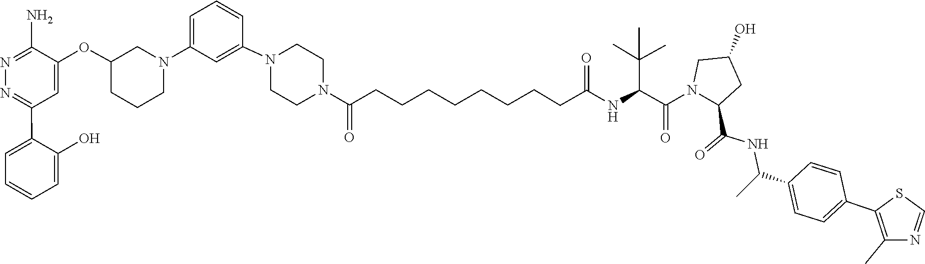

The title compound, was prepared in analogy to Example 1 from intermediate 90 and Ligase 15 as the trifluoroacetic acid salt. MS (ESI): 1058.1 ([M+H]⁺).

### Example 92

### rac-(2S,4R)-4-hydroxy-1-[rac-(2S)-2-[[12-[4-[3-[3-[3-amino-6-(2-hydroxyphenyl)pyridazin-4-yl]oxy-1-piperidyl]phenyl]piperazin-1-yl]-12-oxo-dodecanoyl]amino]-3,3-dimethyl-butanoyl]-N-[rac-(1S)-1-[4-(4-methylthiazol-5-yl)phenyl]ethyl]pyrrolidine-2-carboxamide

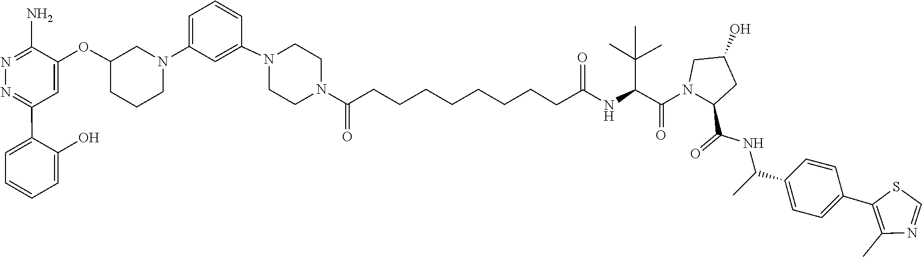

The title compound, was prepared in analogy to Example 1 from intermediate 90 and Ligase 20 as the trifluoroacetic acid salt. MS (ESI): 1086.1 ([M+H]⁺).

### Example 93

### rac-(2S,4R)-4-hydroxy-1-[rac-(2S)-2-[[14-[4-[3-[3-[3-amino-6-(2-hydroxyphenyl)pyridazin-4-yl]oxy-1-piperidyl]phenyl]piperazin-1-yl]-14-oxo-tetradecanoyl]amino]-3,3-dimethyl-butanoyl]-N-[rac-(1S)-1-[4-(4-methylthiazol-5-yl)phenyl]ethyl]pyrrolidine-2-carboxamide

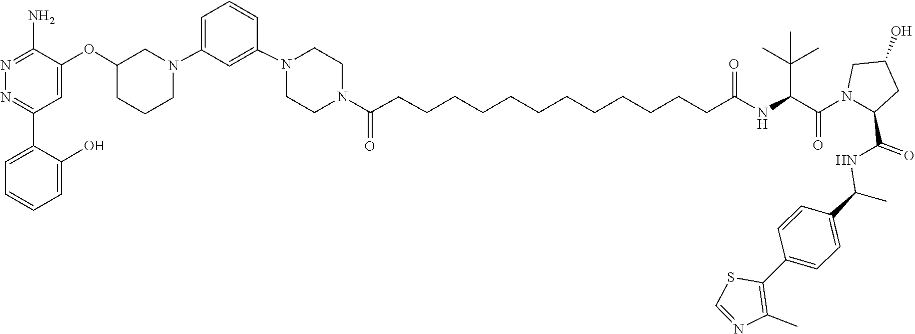 [SMILES: Cc1ncsc1-c1ccc([C@H](C)NC(=O)[C@@H]2C[C@@H](O)CN2C(=O)[C@@H](NC(=O)CCCCCCCCCCCCC(=O)N2CCN(c3cccc(N4CCCC(Oc5cc(-c6ccccc6O)nnc5N)C4)c3)CC2)C(C)(C)C)cc1]

The title compound, was prepared in analogy to Example 1 from intermediate 90 and Ligase 13 as the trifluoroacetic acid salt. MS (ESI): 1114.2 ([M+H]⁺).

### Example 94

### (2S,4R)-1-[(2S)-2-[[7-[4-[3-[1-[3-amino-6-(2-hydroxyphenyl)pyridazin-4-yl]azetidin-3-yl]oxyphenyl]piperazin-1-yl]-7-oxo-heptanoyl]amino]-3,3-dimethyl-butanoyl]-4-hydroxy-N-[(1S)-1-[4-(4-methylthiazol-5-yl)phenyl]ethyl]pyrrolidine-2-carboxamide

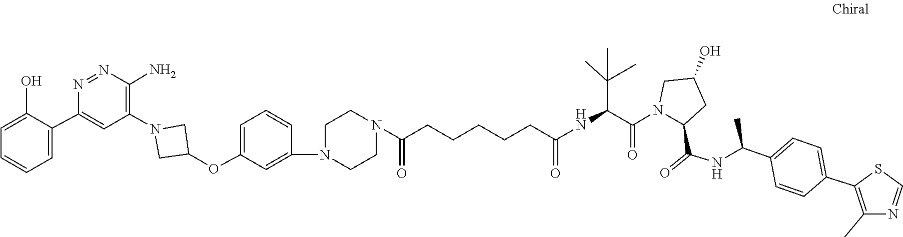 [SMILES: Cc1ncsc1-c1ccc([C@H](C)NC(=O)[C@@H]2C[C@@H](O)CN2C(=O)[C@@H](NC(=O)CCCCCC(=O)N2CCN(c3cccc(OC4CN(c5cc(-c6ccccc6O)nnc5N)C4)c3)CC2)C(C)(C)C)cc1]

The title compound (6% yield), white solid, was prepared in analogy to Example 6 from intermediate 94 and Ligase 11. MS ISP (m/e): 987.6 ([M+H]⁺).

### Example 95

### N-[3-[3-amino-6-(2-hydroxyphenyl)pyridazin-4-yl]oxycyclobutyl]-N′-[(1S)-1-[(2S,4R)-4-hydroxy-2-[[4-(4-methylthiazol-5-yl)phenyl]methylcarbamoyl]pyrrolidine-1-carbonyl]-2,2-dimethyl-propyl]decanediamide

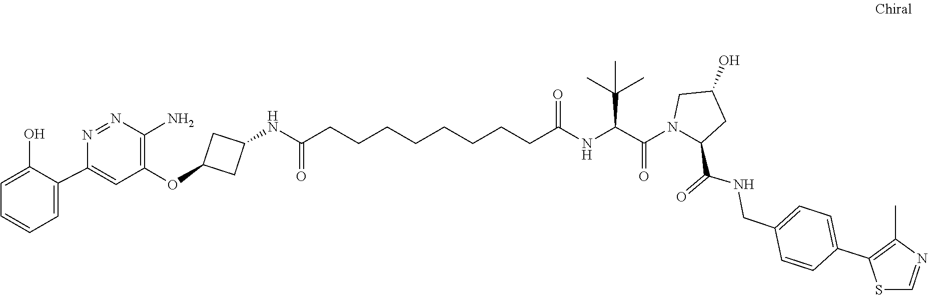 [SMILES: Cc1ncsc1-c1ccc(CNC(=O)[C@@H]2C[C@@H](O)CN2C(=O)[C@@H](NC(=O)CCCCCCCCC(=O)N[C@H]2C[C@H](Oc3cc(-c4ccccc4O)nnc3N)C2)C(C)(C)C)cc1]

The title compound (27.8 mg, 51% yield), white lyoph powder, was prepared in analogy to Example 1 from intermediate 95 and Ligase 4. MS ISP (m/e): 869.8 (5%) [(M+H)+]; 435.5 (100%) [((M+2H)2+].

### Example 96

### (2S,4R)-1-[(2S)-2-[[10-[4-[3-[1-[3-amino-6-(2-hydroxyphenyl)pyridazin-4-yl]azetidin-3-yl]oxyphenyl]piperazin-1-yl]-10-oxo-decanoyl]amino]-3,3-dimethyl-butanoyl]-4-hydroxy-N-[(1S)-1-[4-(4-methylthiazol-5-yl)phenyl]ethyl]pyrrolidine-2-carboxamide

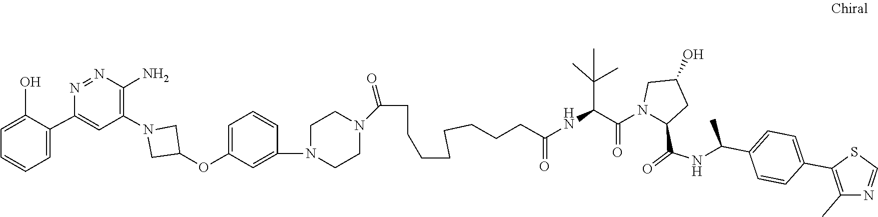 [SMILES: Cc1ncsc1-c1ccc([C@H](C)NC(=O)[C@@H]2C[C@@H](O)CN2C(=O)[C@@H](NC(=O)CCCCCCCCC(=O)N2CCN(c3cccc(OC4CN(c5cc(-c6ccccc6O)nnc5N)C4)c3)CC2)C(C)(C)C)cc1]

The title compound (4.8 mg, 0.004 mmol, 12% yield), white solid, was prepared in analogy to Example 6 from intermediate 94 and Ligase 15. MS ISP (m/e): 515.6 ([(M/2)+H]⁺).

### Example 97

### 4-[3-amino-6-(2-hydroxyphenyl)pyridazin-4-yl]-N-[[1-[10-oxo-10-[[rac-(1S)-2,2-dimethyl-1-[rac-(2S,4R)-4-hydroxy-2-[[4-(4-methylthiazol-5-yl)phenyl]methylcarbamoyl]pyrrolidine-1-carbonyl]propyl]amino]decanoyl]-4-piperidyl]methyl]-1-phenyl-piperazine-2-carboxamide

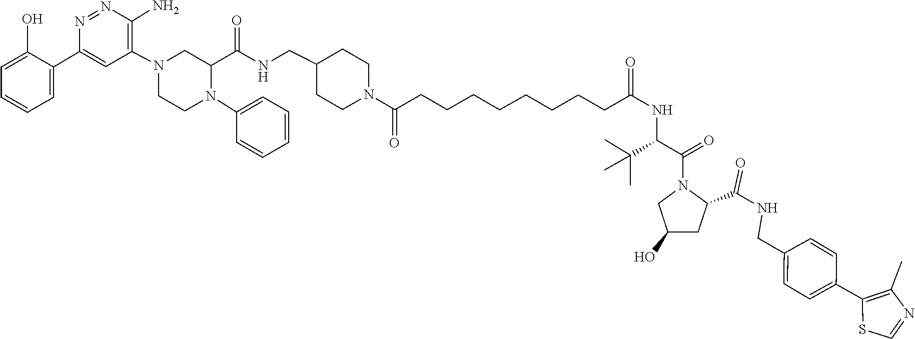 [SMILES: Cc1ncsc1-c1ccc(CNC(=O)[C@@H]2C[C@@H](O)CN2C(=O)[C@@H](NC(=O)CCCCCCCCC(=O)N2CCC(CNC(=O)C3CN(c4cc(-c5ccccc5O)nnc4N)CCN3c3ccccc3)CC2)C(C)(C)C)cc1]

The title compound (6 mg, 5 μmol, 17% yield), light brown solid, trifluoroacetic acid salt, was prepared in analogy to Example 1 from intermediate 97 and Ligase 4. MS ISP (m/e): 1084.9 ([M+H]⁺).

### Example 98

### N′-[2-[[4-[3-amino-6-(2-hydroxyphenyl)pyridazin-4-yl]-1-phenyl-piperazine-2-carbonyl]amino]ethyl]-N′-methyl-N-[rac-(1S)-2,2-dimethyl-1-[rac-(2S,4R)-4-hydroxy-2-[[4-(4-methylthiazol-5-yl)phenyl]methylcarbamoyl]pyrrolidine-1-carbonyl]propyl]decanediamide

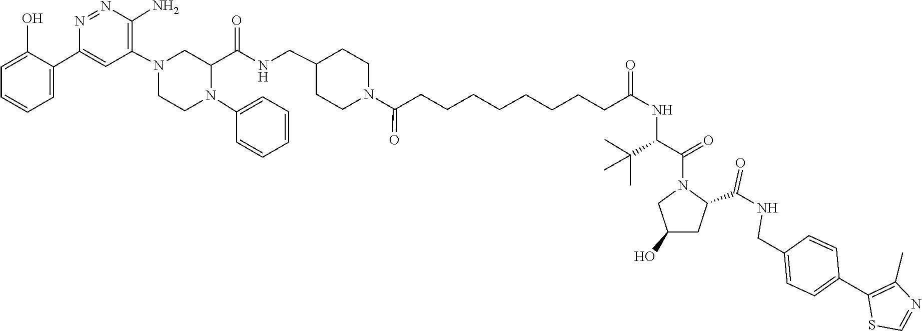 [SMILES: Cc1ncsc1-c1ccc(CNC(=O)[C@@H]2C[C@@H](O)CN2C(=O)[C@@H](NC(=O)CCCCCCCCC(=O)N2CCC(CNC(=O)C3CN(c4cc(-c5ccccc5O)nnc4N)CCN3c3ccccc3)CC2)C(C)(C)C)cc1]

The title compound (6 mg, 5 μmol, 16% yield), light brown solid, trifluoroacetic acid salt, was prepared in analogy to Example 1 from intermediate 98 and Ligase 4. MS ISP (m/e): 1044.9 ([M+H]⁺).

### Example 99

### 1-[3-amino-6-(2-hydroxyphenyl)pyridazin-4-yl]-N-[[1-[10-[[(1S)-1-[(2S,4R)-4-hydroxy-2-[[4-(4-methylthiazol-5-yl)phenyl]methylcarbamoyl]pyrrolidine-1-carbonyl]-2,2-dimethyl-propyl]amino]-10-oxo-decanoyl]-4-piperidyl]methyl]-4-phenyl-piperidine-4-carboxamide

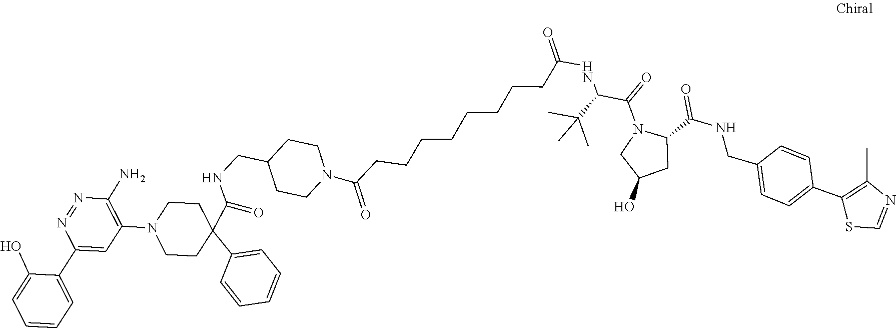 [SMILES: Cc1ncsc1-c1ccc(CNC(=O)[C@@H]2C[C@@H](O)CN2C(=O)[C@@H](NC(=O)CCCCCCCCC(=O)N2CCC(CNC(=O)C3(c4ccccc4)CCN(c4cc(-c5ccccc5O)nnc4N)CC3)CC2)C(C)(C)C)cc1]

The title compound (15 mg, 0.012 mmol, 43% yield), off-white solid, trifluoroacetic acid salt, was prepared in analogy to Example 1 from intermediate 99 and Ligase 4. MS ISP (m/e): 1084.0 ([M+H]⁺).

### Example 100

### (2S,4R)-1-[(2S)-2-[[10-[4-[4-[3-[3-amino-6-(2-aminophenyl)pyridazin-4-yl]oxy-1-piperidyl]phenyl]piperazin-1-yl]-10-oxo-decanoyl]amino]-3,3-dimethyl-butanoyl]-4-hydroxy-N-[[4-(4-methylthiazol-5-yl)phenyl]methyl]pyrrolidine-2-carboxamide

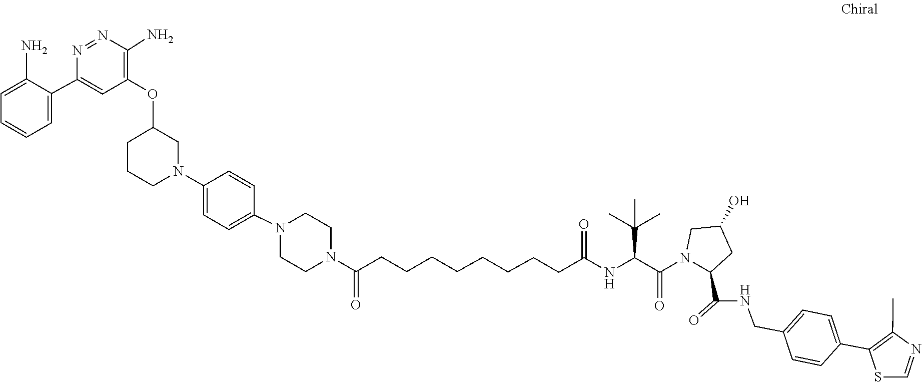 [SMILES: Cc1ncsc1-c1ccc(CNC(=O)[C@@H]2C[C@@H](O)CN2C(=O)[C@@H](NC(=O)CCCCCCCCC(=O)N2CCN(c3ccc(N4CCCC(Oc5cc(-c6ccccc6N)nnc5N)C4)cc3)CC2)C(C)(C)C)cc1]

The title compound (16 mg, 12.6 μmol, 13% yield), white solid, trifluoroacetic acid salt, was prepared in analogy to Example 1 from intermediate 100 and Ligase 4. MS ISP (m/e): 1042.5692 ([M+H]⁺).

### Example 101

### (2S,4R)-1-[(2S)-2-[[10-[4-[3-[1-[3-amino-6-(2-hydroxyphenyl)pyridazin-4-yl]azetidin-3-yl]oxybenzoyl]piperazin-1-yl]-10-oxo-decanoyl]amino]-3,3-dimethyl-butanoyl]-4-hydroxy-N-[(1S)-1-[4-(4-methylthiazol-5-yl)phenyl]ethyl]pyrrolidine-2-carboxamide

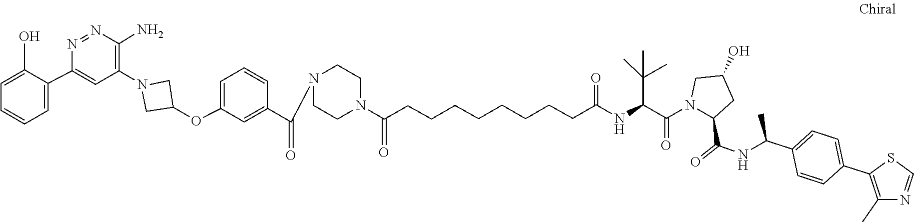 [SMILES: Cc1ncsc1-c1ccc([C@H](C)NC(=O)[C@@H]2C[C@@H](O)CN2C(=O)[C@@H](NC(=O)CCCCCCCCC(=O)N2CCN(C(=O)c3cccc(OC4CN(c5cc(-c6ccccc6O)nnc5N)C4)c3)CC2)C(C)(C)C)cc1]

The title compound (10.5 mg, 0.010 mmol, 13% yield), white solid, was prepared in analogy to Example 6 from intermediate 101 and Ligase 15. MS ISP (m/e): 1057.4 ([M+H]⁺).

### Example 102

### (2S,4R)-1-[(2S)-2-[[11-[4-[[3-[3-[3-amino-6-(2-aminophenyl)pyridazin-4-yl]-3,8-diazabicyclo[3.2.1]octan-8-yl]phenyl]methyl]piperazin-1-yl]-11-oxo-undecanoyl]amino]-3,3-dimethyl-butanoyl]-4-hydroxy-N-[[4-(4-methylthiazol-5-yl)phenyl]methyl]pyrrolidine-2-carboxamide

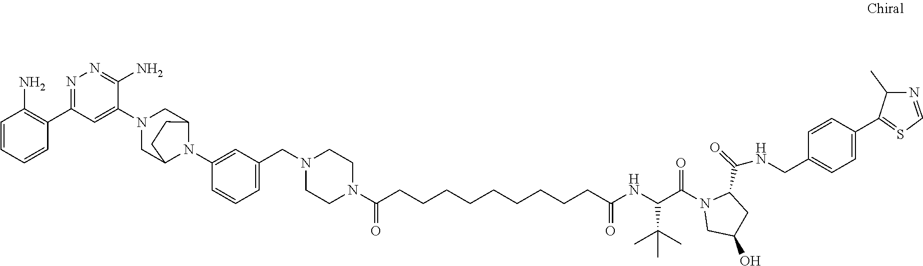

The title compound (13.5 mg, 10.3 μmol, 12% yield), light yellow solid, bis-trifluoroacetate salt, was prepared in analogy to Example 1 from intermediate 102 and Ligase 5. MS ISP (m/e): 1079.6031 ([M−H]⁻).

### Example 103

### (2S,4R)-1-[(2S)-2-[[7-[4-[4-[1-[3-amino-6-(2-hydroxyphenyl)pyridazin-4-yl]azetidin-3-yl]oxybenzoyl]piperazin-1-yl]-7-oxo-heptanoyl]amino]-3,3-dimethyl-butanoyl]-4-hydroxy-N-[(1S)-1-[4-(4-methylthiazol-5-yl)phenyl]ethyl]pyrrolidine-2-carboxamide

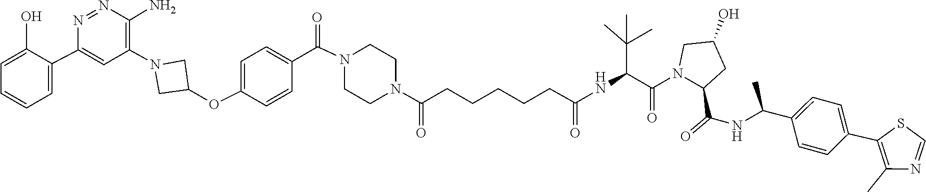 [SMILES: Cc1ncsc1-c1ccc([C@H](C)NC(=O)[C@@H]2C[C@@H](O)CN2C(=O)[C@@H](NC(=O)CCCCCC(=O)N2CCN(C(=O)c3ccc(OC4CN(c5cc(-c6ccccc6O)nnc5N)C4)cc3)CC2)C(C)(C)C)cc1]

The title compound (6.2 mg, 0.010 mmol, 10% yield), white solid, was prepared in analogy to Example 6 from intermediate 103 and Ligase 11. MS ISP (m/e): 1015.5 ([M+H]⁺).

### Example 104

### (2S,4R)-1-[(2S)-2-[[10-[4-[4-[1-[3-amino-6-(2-hydroxyphenyl)pyridazin-4-yl]azetidin-3-yl]oxybenzoyl]piperazin-1-yl]-10-oxo-decanoyl]amino]-3,3-dimethyl-butanoyl]-4-hydroxy-N-[(1S)-1-[4-(4-methylthiazol-5-yl)phenyl]ethyl]pyrrolidine-2-carboxamide

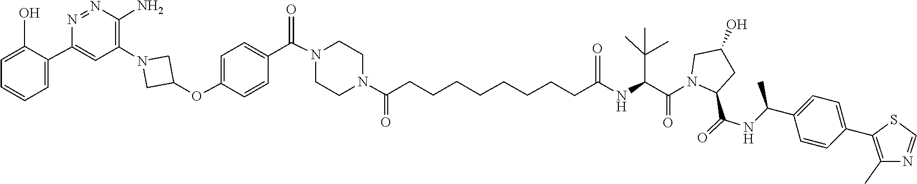 [SMILES: Cc1ncsc1-c1ccc([C@H](C)NC(=O)[C@@H]2C[C@@H](O)CN2C(=O)[C@@H](NC(=O)CCCCCCCCC(=O)N2CCN(C(=O)c3ccc(OC4CN(c5cc(-c6ccccc6O)nnc5N)C4)cc3)CC2)C(C)(C)C)cc1]

The title compound (14.5 mg, 0.010 mmol, 24% yield), white solid, was prepared in analogy to Example 6 from intermediate 103 and Ligase 15. MS ISP (m/e): 1057.5 ([M+H]⁺).

### Example 105

### (2S,4R)-1-[(2S)-2-[[7-[4-[[3-[2-[3-amino-6-(2-hydroxyphenyl)pyridazin-4-yl]oxyethyl]phenyl]methyl]piperazin-1-yl]-7-oxo-heptanoyl]amino]-3,3-dimethyl-butanoyl]-4-hydroxy-N-[(1S)-1-[4-(4-methylthiazol-5-yl)phenyl]ethyl]pyrrolidine-2-carboxamide

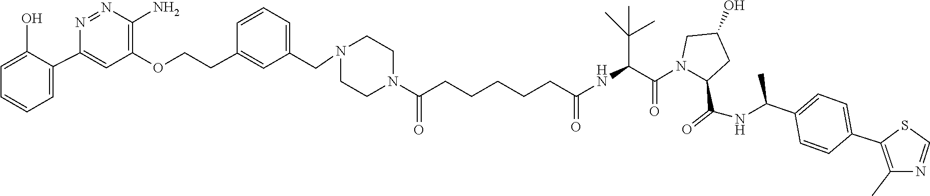 [SMILES: Cc1ncsc1-c1ccc([C@H](C)NC(=O)[C@@H]2C[C@@H](O)CN2C(=O)[C@@H](NC(=O)CCCCCC(=O)N2CCN(Cc3cccc(CCOc4cc(-c5ccccc5O)nnc4N)c3)CC2)C(C)(C)C)cc1]

The title compound (33.7 mg, 0.030 mmol, 52% yield), white solid, was prepared in analogy to Example 6 from intermediate 105 and Ligase 11. MS ISP (m/e): 974.5 ([M+H]⁺).

### Example 106

### (2S,4R)-1-[(2S)-2-[[10-[4-[[3-[2-[3-amino-6-(2-hydroxyphenyl)pyridazin-4-yl]oxyethyl]phenyl]methyl]piperazin-1-yl]-10-oxo-decanoyl]amino]-3,3-dimethyl-butanoyl]-4-hydroxy-N-[(1S)-1-[4-(4-methylthiazol-5-yl)phenyl]ethyl]pyrrolidine-2-carboxamide

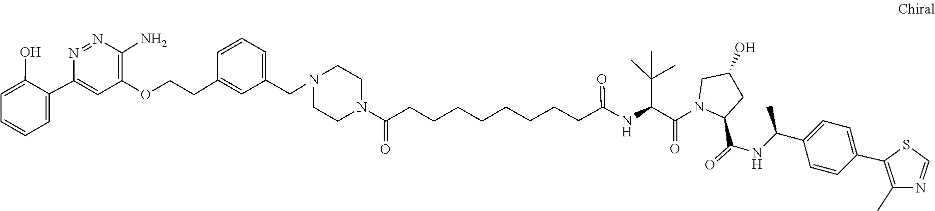 [SMILES: Cc1ncsc1-c1ccc([C@H](C)NC(=O)[C@@H]2C[C@@H](O)CN2C(=O)[C@@H](NC(=O)CCCCCCCCC(=O)N2CCN(Cc3cccc(CCOc4cc(-c5ccccc5O)nnc4N)c3)CC2)C(C)(C)C)cc1]

The title compound (14.8 mg, 0.010 mmol, 23% yield), white solid, was prepared in analogy to Example 6 from intermediate 105 and Ligase 15. MS ISP (m/e): 1016.6 ([M+H]⁺).

### Example 107

### (2S,4R)-1-[(2S)-2-[[10-[4-[[3-[3-[3-amino-6-(5-fluoro-2-hydroxy-phenyl)pyridazin-4-yl]-3,8-diazabicyclo[3.2.1]octan-8-yl]phenyl]methyl]piperazin-1-yl]-10-oxo-decanoyl]amino]-3,3-dimethyl-butanoyl]-4-hydroxy-N-[[4-(4-methylthiazol-5-yl)phenyl]methyl]pyrrolidine-2-carboxamide

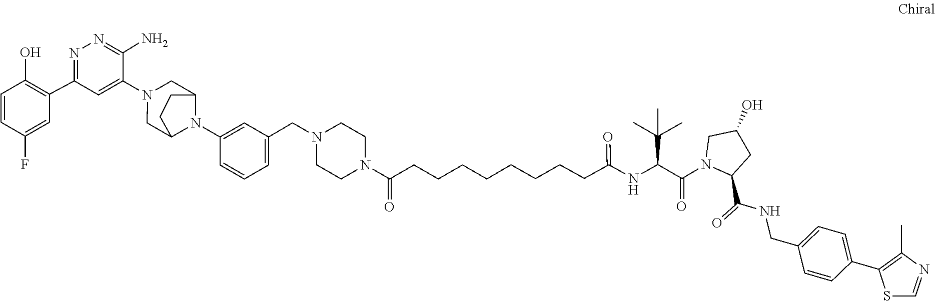 [SMILES: Cc1ncsc1-c1ccc(CNC(=O)[C@@H]2C[C@@H](O)CN2C(=O)[C@@H](NC(=O)CCCCCCCCC(=O)N2CCN(Cc3cccc(N4C5CCC4CN(c4cc(-c6cc(F)ccc6O)nnc4N)C5)c3)CC2)C(C)(C)C)cc1]

The title compound (15.1 mg, 10.7 μmol, 10% yield), light yellow solid, bis-trifluoroacetate salt, was prepared in analogy to Example 1 from intermediate 107 and Ligase 4. MS ISP (m/e): 1086.5768 ([M+H]⁺).

### Example 108

### rac-(2S,4R)-4-hydroxy-N-[[4-(4-methylthiazol-5-yl)phenyl]methyl]-1-[rac-(2S)-2-[[10-[4-[4-[3-[3-amino-6-(5-fluoro-2-hydroxy-phenyl)pyridazin-4-yl]oxy-1-piperidyl]phenyl]piperazin-1-yl]-10-oxo-decanoyl]amino]-3,3-dimethyl-butanoyl]pyrrolidine-2-carboxamide

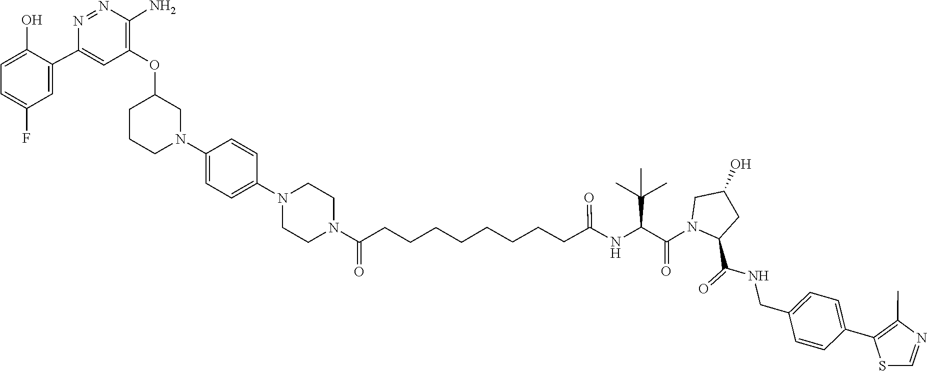 [SMILES: Cc1ncsc1-c1ccc(CNC(=O)[C@@H]2C[C@@H](O)CN2C(=O)[C@@H](NC(=O)CCCCCCCCC(=O)N2CCN(c3ccc(N4CCCC(Oc5cc(-c6cc(F)ccc6O)nnc5N)C4)cc3)CC2)C(C)(C)C)cc1]

The title compound (47.2 mg, 36.6 μmol, 39% yield), light blue solid, trifluoroacetic acid salt, was prepared in analogy to Example 1 from intermediate 108 and Ligase 4. MS ISP (m/e): 1059.5299 ([M+H]⁺).

### Example 109

### rac-(2S,4R)-4-hydroxy-N-[[4-(4-methylthiazol-5-yl)phenyl]methyl]-1-[rac-(2S)-2-[[7-[4-[4-[3-[3-amino-6-(5-fluoro-2-hydroxy-phenyl)pyridazin-4-yl]oxy-1-piperidyl]phenyl]piperazin-1-yl]-7-oxo-heptanoyl]amino]-3,3-dimethyl-butanoyl]pyrrolidine-2-carboxamide

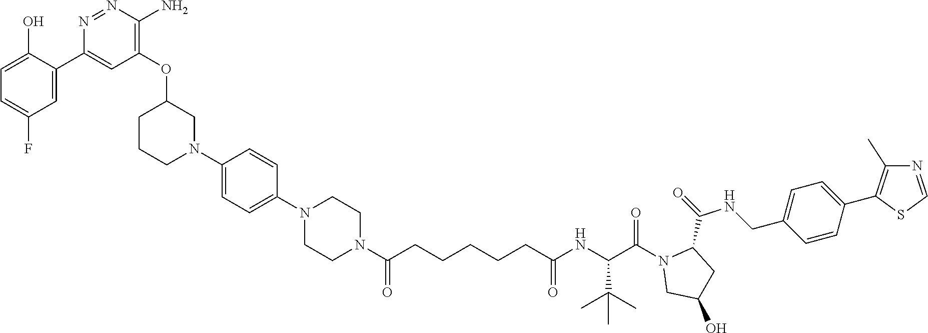 [SMILES: Cc1ncsc1-c1ccc(CNC(=O)[C@@H]2C[C@@H](O)CN2C(=O)[C@@H](NC(=O)CCCCCC(=O)N2CCN(c3ccc(N4CCCC(Oc5cc(-c6cc(F)ccc6O)nnc5N)C4)cc3)CC2)C(C)(C)C)cc1]

The title compound (54.8 mg, 37.8 μmol, 40% yield), light blue solid, trifluoroacetic acid salt, was prepared in analogy to Example 1 from intermediate 108 and Ligase 3. MS ISP (m/e): 1019.4961 ([M+H]⁺).

### Example 110

### (2S,4R)-1-[(2S)-2-[[11-[4-[2-[3-[8-[3-amino-6-(5-fluoro-2-hydroxy-phenyl)pyridazin-4-yl]-3,8-diazabicyclo[3.2.1]octan-3-yl]phenoxy]ethyl]piperazin-1-yl]-11-oxo-undecanoyl]amino]-3,3-dimethyl-butanoyl]-4-hydroxy-N-[[4-(4-methylthiazol-5-yl)phenyl]methyl]pyrrolidine-2-carboxamide

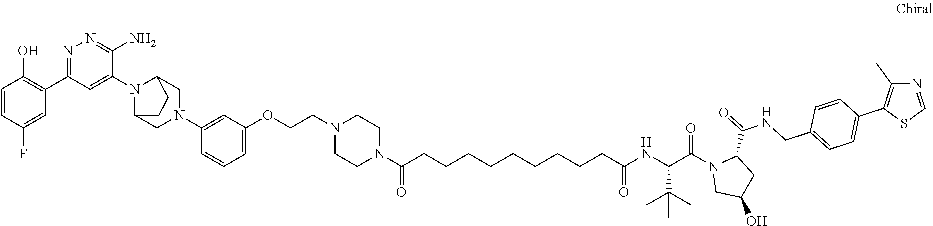 [SMILES: Cc1ncsc1-c1ccc(CNC(=O)[C@@H]2C[C@@H](O)CN2C(=O)[C@@H](NC(=O)CCCCCCCCCC(=O)N2CCN(CCOc3cccc(N4CC5CCC(C4)N5c4cc(-c5cc(F)ccc5O)nnc4N)c3)CC2)C(C)(C)C)cc1]

The title compound (26 mg, 19.8 μmol, 25% yield), white solid, trifluoroacetic acid salt, was prepared in analogy to Example 1 from intermediate 110g and Ligase 5. MS (ESI): 1130.6040 ([M+H]⁺).

1. A compound of formula (I) 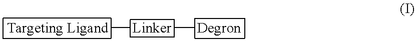
or a pharmaceutically acceptable salt thereof, wherein:
said targeting ligand is of formula (TL): 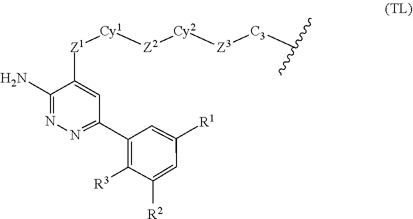
wherein:
R¹ and R² are each independently selected from hydrogen and halogen;
R³ is selected from hydroxy and amino;
Z¹ is:
(i) absent;
(ii) —O—;
(iii) —O—C₁-C₆-alkyldiyl-;
(iv) —O(CH₂)ₙCH(R⁴)(CH₂)ₚ—; or
(v) C₂-C₆-alkynyldiyl;
Cy¹ is:
(i) absent;
(ii) C₆-C₁₀-aryl optionally substituted with 1-3 substitutents R⁵;
(iii) C₃-C₁₀-cycloalkyl optionally substituted with 1-3 substitutents R⁶;
(iv) 5-14 membered heteroaryl optionally substituted with 1-3 substitutents R⁷; or
(v) 3-14 membered heterocyclyl optionally substituted with 1-3 substitutents R⁸;
Z² is:
(i) absent;
(ii) —C(O)NH—C₁-C₆-alkyldiyl-;
(iii) —CH(R⁹)—;
(iv) C₁-C₆-alkyldiyl;
(v) —C₁-C₆-alkyldiyl-N(C₁-C₆-alkyl)-C₁-C₆-alkyldiyl-C(O)—;
(vi) —O—; or
(vii) —O—C₁-C₆-alkyldiyl-;
Cy² is:
(i) absent;
(ii) C₆-C₁₀-aryl substituted with optionally substituted with 1-3 substitutents R¹⁰; or
(iii) 3-14 membered heterocyclyl optionally substituted with 1-3 substitutents R¹¹;
Z³ is:
(i) absent;
(ii) carbonyl;
(iii) C₁-C₆-alkyldiyl;
(iv) —O—; or
(v) —O—C₁-C₆-alkyldiyl-;
Cy³ is:
(i) absent; or
(ii) 3-14 membered heterocyclyl optionally substituted with 1-3 substitutents R¹²;
n and p are each independently an integer selected from 0, 1, 2, 3, 4, 5 and 6;
R⁴ and R⁹ are independently selected from C₁-C₆-alkyl and C₆-C₁₀-aryl;
R⁵, R⁶, R⁷, R¹, R¹⁰, R¹¹ and R¹² are independently selected from halogen, cyano, C₁-C₆-alkyl, C₁-C₆-alkoxy, halo-C₁-C₆-alkyl, halo-C₁-C₆-alkoxy and C₆-C₁₀-aryl; and
the wavy line indicates the point of attachment to the linker;
said linker is selected from formulae (L-1), (L-2), (L-3), (L-4), (L-5), (L-6), (L-7) and (L-8): 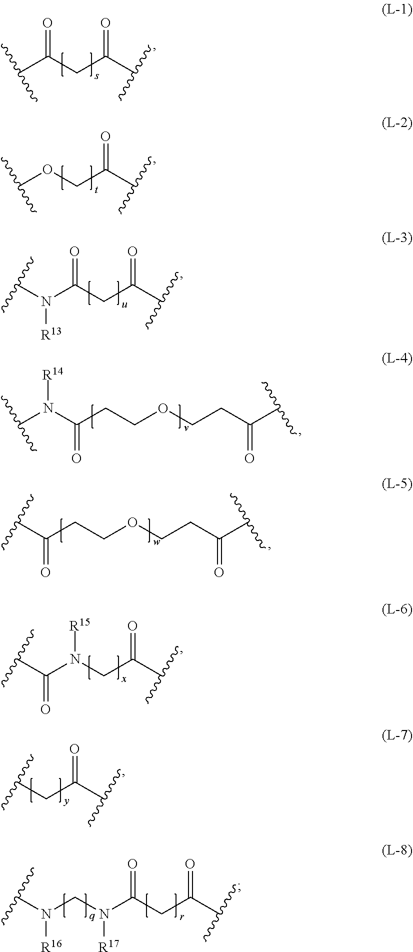
wherein:
R¹³, R¹⁴, R¹⁵ and R¹⁶ are independently selected from hydrogen and C₁-C₆-alkyl;
q, r, s, t, u, v, w, x and y are independently an integer selected from 1, 2, 3, 4, 5, 6, 7, 8, 9, 10, 11 and 12; and
a wavy line indicates the point of attachment to the targeting ligand or the degron; and
said degron is of formula (DG-1) or (DG-2): 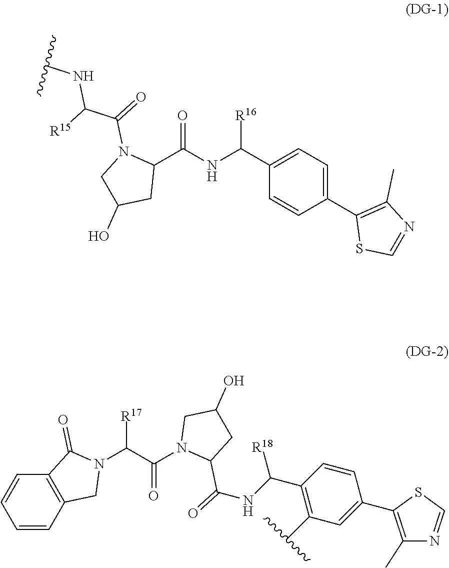
wherein:
R¹⁵, R¹⁶, R¹⁷ and R¹⁸ are independently selected from hydrogen and C₁-C₆-alkyl; and
the wavy line indicates the point of attachment to the linker.

2. The compound of formula (I) according to claim 1, or a pharmaceutically acceptable salt thereof, wherein said targeting ligand is of formula (TL), wherein:
R¹ is selected from hydrogen and halogen;
R² is hydrogen;
R³ is selected from hydroxy and amino;
Z¹ is:
(i) absent;
(ii) —O—;
(iii) —O—C₁-C₆-alkyldiyl-;
(iv) —O(CH₂)ₙCH(R⁴)(CH₂)ₚ—; or
(v) C₂-C₆-alkynyldiyl;
Cy¹ is:
(i) absent;
(ii) C₆-C₁₀-aryl optionally substituted with R⁵;
(iii) C₃-C₁₀-cycloalkyl;
(iv) 5-14 membered heteroaryl; or
(v) 3-14 membered heterocyclyl optionally substituted R⁸;
Z² is:
(i) absent;
(ii) —C(O)NH—C₁-C₆-alkyldiyl-;
(iii) —CH(R⁹)—;
(iv) C₁-C₆-alkyldiyl;
(v) —C₁-C₆-alkyldiyl-N(C₁-C₆-alkyl)-C₁-C₆-alkyldiyl-C(O)—;
(vi) —O—; or
(vii) —O—C₁-C₆-alkyldiyl-;
Cy² is:
(i) absent;
(ii) C₆-C₁₀-aryl; or
(iii) 3-14 membered heterocyclyl;
Z³ is:
(i) absent;
(ii) carbonyl;
(iii) C₁-C₆-alkyldiyl; or
(iv) —O—C₁-C₆-alkyldiyl-;
Cy³ is:
(i) absent; or
(ii) 3-14 membered heterocyclyl;
n and p are each independently an integer selected from 0 and 1;
R⁴ is selected from C₁-C₆-alkyl and C₆-C₁₀-aryl;
R⁵ is halogen;
R⁶ is selected from C₁-C₆-alkyl and C₆-C₁₀-aryl;
R⁹ is C₆-C₁₀-aryl; and
the wavy line indicates the point of attachment to the linker.

3. The compound of formula (I) according to claim 1, or a pharmaceutically acceptable salt thereof, wherein said targeting ligand is of formula (TL), wherein:
R¹ and R² are both hydrogen;
R³ is hydroxy;
Z¹ is:
(i) absent;
(ii) —O—; or
(iii) —O—C₁-C₆-alkyldiyl-;
Cy¹ is:
(i) C₆-C₁₀-aryl; or
(ii) 3-14 membered heterocyclyl;
Z² is:
(i) absent;
(ii) —O—; or
(iii) —O—C₁-C₆-alkyldiyl-;
Cy² is:
(i) absent;
(ii) C₆-C₁₀-aryl; or
(iii) 3-14 membered heterocyclyl;
Z³ is:
(i) absent;
(ii) carbonyl; or
(iii) —O—C₁-C₆-alkyldiyl-;
Cy³ is:
(i) absent; or
(ii) 3-14 membered heterocyclyl; and
the wavy line indicates the point of attachment to the linker.

4. The compound of formula (I) according to claim 1, or a pharmaceutically acceptable salt thereof, wherein said targeting ligand is of formula (TL), wherein:
R¹ and R² are both hydrogen;
R³ is hydroxy;
Z¹ is:
(i) absent;
(ii) —O—; or
(iii) —OCH₂CH₂—;
Cy¹ is selected from the group consisting of: 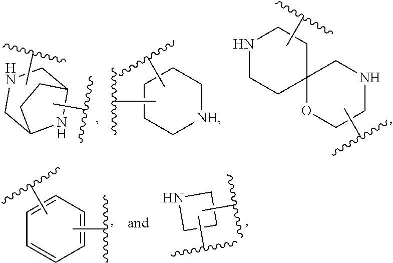
wherein a wavy line indicates the point of attachment to Z¹ or Z²;
Z² is:
(i) absent;
(ii) —O—; or
(iii) —OCH₂CH₂—;
Cy² is absent or selected from the group consisting of: 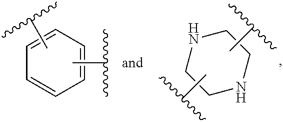
wherein a wavy line indicates the point of attachment to Z² or Z³;
Z³ is:
(i) absent;
(ii) carbonyl; or
(iii) —OCH₂CH₂—; and
Cy³ is absent or 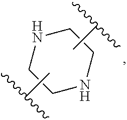
wherein a wavy line indicates the point of attachment to Z³ or the linker.

5. The compound of formula (I) according to claim 1, or a pharmaceutically acceptable salt thereof, wherein said linker is selected from formulae (L-1), (L-2), (L-3), (L-4), (L-5), (L-6), (L-7) and (L-8), wherein:
R¹³ is selected from hydrogen and C₁-C₆-alkyl;
R¹⁴, R¹⁶ and R¹⁷ are independently C₁-C₆-alkyl;
R¹⁵ is hydrogen;
q is 3;
r is an integer selected from 5, 8 and 10;
s is an integer selected from 2, 5, 6, 7, 8, 9, 10 and 12;
t is an integer selected from 9 and 10;
u is an integer selected from 5, 8, 10 and 12;
v is an integer selected from 1 and 2;
w is an integer selected from 1, 2 and 3;
x is an integer selected from 6 and 9;
y is 7; and
a wavy line indicates the point of attachment to the targeting ligand or the degron.

6. The compound of formula (I) according to claim 1, or a pharmaceutically acceptable salt thereof, wherein said linker is of formula (L-1), wherein s is an integer selected from 5, 8, 9, 10 and 12; and
a wavy line indicates the point of attachment to the targeting ligand or the degron.

7. The compound of formula (I) according to claim 1, or a pharmaceutically acceptable salt thereof, wherein said degron is of formula (DG-1) or (DG-2), wherein:
R¹⁵ is C₁-C₆-alkyl;
R¹⁶ is selected from hydrogen and C₁-C₆-alkyl;
R¹⁷ is C₁-C₆-alkyl;
R¹⁸ is hydrogen; and
the wavy line indicates the point of attachment to the linker.

8. The compound of formula (I) according to claim 1, or a pharmaceutically acceptable salt thereof, wherein said degron is of formula (DG-1) wherein:
R¹⁵ is C₁-C₆-alkyl;
R¹⁶ is selected from hydrogen and C₁-C₆-alkyl; and
the wavy line indicates the point of attachment to the linker.

9. The compound of formula (I) according to claim 1, or a pharmaceutically acceptable salt thereof, wherein said degron is of formula (DG-1) wherein:
R¹⁵ is tert-butyl or isopropyl;
R¹⁶ is selected from hydrogen and methyl; and
the wavy line indicates the point of attachment to the linker.

10. The compound of formula (I) according to claim 1, or a pharmaceutically acceptable salt thereof, wherein said targeting ligand is of formula (TL), wherein:
R¹ is selected from hydrogen and halogen;
R² is hydrogen;
R³ is selected from hydroxy and amino;
Z¹ is:
(i) absent;
(ii) —O—;
(iii) —O—C₁-C₆-alkyldiyl-;
(iv) —O(CH₂)ₙCH(R⁴)(CH₂)ₚ—; or
(v) C₂-C₆-alkynyldiyl;
Cy¹ is:
(i) absent;
(ii) C₆-C₁₀-aryl optionally substituted with R⁵;
(iii) C₃-C₁₀-cycloalkyl;
(iv) 5-14 membered heteroaryl; or
(v) 3-14 membered heterocyclyl optionally substituted R⁸;
Z² is:
(i) absent;
(ii) —C(O)NH—C₁-C₆-alkyldiyl-;
(iii) —CH(R⁹)—;
(iv) C₁-C₆-alkyldiyl;
(v) —C₁-C₆-alkyldiyl-N(C₁-C₆-alkyl)-C₁-C₆-alkyldiyl-C(O)—;
(vi) —O—; or
(vii) —O—C₁-C₆-alkyldiyl-;
Cy² is:
(i) absent;
(ii) C₆-C₁₀-aryl; or
(iii) 3-14 membered heterocyclyl;
Z³ is:
(i) absent;
(ii) carbonyl;
(iii) C₁-C₆-alkyldiyl; or
(iv) —O—C₁-C₆-alkyldiyl-;
Cy³ is:
(i) absent; or
(ii) 3-14 membered heterocyclyl;
n and p are each independently an integer selected from 0 and 1;
R⁴ is selected from C₁-C₆-alkyl and C₆-C₁₀-aryl;
R⁵ is halogen;
R⁸ is selected from C₁-C₆-alkyl and C₆-C₁₀-aryl;
R⁹ is C₆-C₁₀-aryl; and
the wavy line indicates the point of attachment to the linker;
said linker is selected from formulae (L-1), (L-2), (L-3), (L-4), (L-5), (L-6), (L-7) and (L-8), wherein:
R¹³ is selected from hydrogen and C₁-C₆-alkyl;
R¹⁴, R¹⁶ and R¹⁷ are independently C₁-C₆-alkyl;
R^{L}S is hydrogen;
q is 3;
r is an integer selected from 5, 8 and 10;
s is an integer selected from 2, 5, 6, 7, 8, 9, 10 and 12;
t is an integer selected from 9 and 10;
u is an integer selected from 5, 8, 10 and 12;
v is an integer selected from 1 and 2;
w is an integer selected from 1, 2 and 3;
x is an integer selected from 6 and 9;
y is 7; and
a wavy line indicates the point of attachment to the targeting ligand or the degron; and
said degron is of formula (DG-1) or (DG-2), wherein:
R¹⁵ is C₁-C₆-alkyl;
R¹⁶ is selected from hydrogen and C₁-C₆-alkyl;
R¹⁷ is C₁-C₆-alkyl;
R¹⁸ is hydrogen; and
the wavy line indicates the point of attachment to the linker.

11. The compound of formula (I) according to claim 1, or a pharmaceutically acceptable salt thereof, wherein said targeting ligand is of formula (TL), wherein:
R¹ and R² are both hydrogen;
R³ is hydroxy;
Z¹ is:
(i) absent;
(ii) —O—; or
(iii) —O—C₁-C₆-alkyldiyl-;
Cy¹ is:
(i) C₆-C₁₀-aryl; or
(ii) 3-14 membered heterocyclyl;
Z² is:
(i) absent;
(ii) —O—; or
(iii) —O—C₁-C₆-alkyldiyl-;
Cy² is:
(i) absent;
(ii) C₆-C₁₀-aryl; or
(iii) 3-14 membered heterocyclyl;
Z³ is:
(i) absent;
(ii) carbonyl; or
(iii) —O—C₁-C₆-alkyldiyl-;
Cy³ is:
(i) absent; or
(ii) 3-14 membered heterocyclyl; and
the wavy line indicates the point of attachment to the linker;
said linker is of formula (L-1), wherein s is an integer selected from 5, 8, 9, 10 and 12; and
a wavy line indicates the point of attachment to the targeting ligand or the degron; and
said degron is of formula (DG-1) wherein:
R¹⁵ is C₁-C₆-alkyl;
R¹⁶ is selected from hydrogen and C₁-C₆-alkyl; and
the wavy line indicates the point of attachment to the linker.

12. The compound of formula (I) according to claim 1, or a pharmaceutically acceptable salt thereof, wherein said targeting ligand is of formula (TL), wherein:
R¹ and R² are both hydrogen;
R³ is hydroxy;
Z¹ is:
(i) absent;
(ii) —O—; or
(iii) —OCH₂CH₂—;
Cy¹ is selected from the group consisting of: 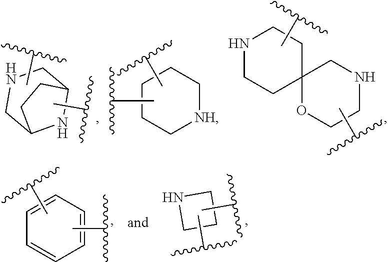
wherein a wavy line indicates the point of attachment to Z¹ or Z²;
Z² is:
(i) absent;
(ii) —O—; or
(iii) —OCH₂CH₂—;
Cy² is absent or selected from the group consisting of: 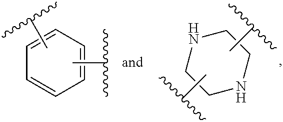
wherein a wavy line indicates the point of attachment to Z² or Z³;
Z³ is:
(i) absent;
(ii) carbonyl; or
(iii) —OCH₂CH₂—; and
Cy³ is absent or 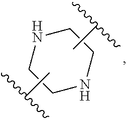
wherein a wavy line indicates the point of attachment to Z³ or the linker;
said linker is of formula (L-1), wherein s is an integer selected from 5, 8, 9, 10 and 12; and
a wavy line indicates the point of attachment to the targeting ligand or the degron; and
said degron is of formula (DG-1) wherein:
R¹⁵ is tert-butyl or isopropyl;
R¹⁶ is selected from hydrogen and methyl; and
the wavy line indicates the point of attachment to the linker.

13. The compound of formula (I) according to claim 1, or a pharmaceutically acceptable salt thereof, wherein said compound of formula (I) is selected from Examples 1 to 111.

14. The compound of formula (I) according to claim 1, or a pharmaceutically acceptable salt thereof, wherein said compound of formula (I) is selected from Examples 11, 32, 33, 37, 45, 48, 56, 58, 78, 79, 96 and 101.

15. A therapeutically active substance, comprising the compound of formula (I) according to claim 1, or a pharmaceutically acceptable salt thereof.

16. A pharmaceutical composition comprising a compound of formula (I) according to claim 1, or a pharmaceutically acceptable salt thereof, and a therapeutically inert carrier.

17. The composition according to claim 16, further comprising an additional therapeutic agent.

18. The composition according to claim 16, wherein the additional therapeutic agent is a chemotherapeutic agent.

19. (canceled)

20. The method according to claim 23, wherein said SMARCA2-mediated disorder is cancer.

21. The method according to claim 20, wherein said cancer is selected from the group consisting of acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, diffuse large B-cell lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, glioblastoma, gliosarcoma, heavy chain disease, hemangioblastoma, hepatoma, hepatocellular cancer, hormone insensitive prostate cancer, leiomyosarcoma, leukemia, liposarcoma, liver cancer, lung cancer, lymphagioendotheliosarcoma, lymphangiosarcoma, lymphoblastic leukemia, lymphoma (Hodgkin's and non-Hodgkin's; Burkitt's), malignancies and hyperproliferative disorders of the bladder, breast, colon, lung, ovaries, pancreas, prostate, skin and uterus, lymphoid malignancies of T-cell or B-cell origin, medullary carcinoma, medulloblastoma, melanoma, meningioma, mesothelioma, multiple myeloma, myelogenous leukemia, myeloma, myxosarcoma, neuroblastoma, NUT midline carcinoma (NMC), non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, malignant rhabdoid tumor (MRT), rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer and Wilms' tumor.

22. The method according to claim 20, wherein said cancer is selected from the group consisting of hepatocellular cancer, malignancies and hyperproliferative disorders of the colon (e.g. colon cancer), lung cancer, breast cancer, prostate cancer, melanoma, and ovarian cancer.

23. A method of treating SMARCA2-mediated disorders in a subject, comprising administering a compound of formula (I) according to claim 1, or a pharmaceutically acceptable salt thereof, to the subject.

24. (canceled)

25. (canceled)

26. (canceled)
